(19) United States
(12) Patent Application Publication
(10) Pub. No.: US 2026/0234657 A1
(43) Pub. Date: Aug. 13, 2026

(54) COMPONENTS FOR GENOMIC EDITING

(71) Applicant: Kano Therapeutics Inc., Cambridge, MA (US)

(72) Inventors: Gregory Benjamin Foley, Somerville, MA (US); Floris Alaska Silvia Engelhardt, Boston, MA (US); Stephanie Barr, Watertown, MA (US)

(21) Appl. No.: 19/471,362

(22) PCT Filed: Mar. 29, 2024
(86) PCT No.: PCT/US2024/022353

§ 371 Date: Sep. 30, 2025

Related U.S. Application Data
(60) Provisional application No. 63/493,586, filed on Mar. 31, 2023.

(51) Int. Cl. C12N 15/85; C12N 1/12; C12N 1/16; C12N 1/20; C12N 5/071; C12N 9/22; C12N 15/10; C12N 15/113; C12N 15/90

(52) U.S. Cl. CPC C12N 15/85; C12N 1/12; C12N 1/16; C12N 1/20; C12N 5/0602; C12N 9/226; C12N 15/102; C12N 15/1138; C12N 15/90; C12N 2310/20; C12N 2310/532

(57) ABSTRACT

The present disclosure provides, among other things, homology directed repair (HDR) using single stranded DNA (ssDNA), e.g., circular single stranded DNA (cssDNA) and compositions for use in the same.

### CROSS REFERENCE TO RELATED APPLICATIONS

This application claims the benefit of U.S. Provisional Application No. 63/493,586, filed Mar. 31, 2023, the contents of which is hereby incorporated by reference in its entirety.

### BACKGROUND

The precise editing of double stranded DNA is an unmet need in both the therapeutic setting and the synthetic biology setting. Some of the many challenges that are presented in the dsDNA editing field include difficulties arising from the toxicity of dsDNA, the ability to control the location of the insertion of the dsDNA, the stability of DNA intended to be inserted, and the fidelity of the DNA manufactured to be inserted. The following disclosure is directed to overcoming at least some of these challenges.

### SUMMARY

Described herein are circular single stranded DNA (cssDNA) molecules that include a co-location sequence that functions to locate the cssDNA molecules near the site of a desired nuclease cut site (FIG. 2). Co-location sequences are nucleic acid sequences that bind to amino acid sequences. In some examples, a co-location sequence is a region of reverse complementarity within the cssDNA molecule that an amino acid sequence binds to and the amino acid sequence can be described as a synthetic binding domain or a synthetic DNA binding domain. An amino acid sequence that binds to a co-location sequence can either directly, or indirectly, bind to a synthetic binding domain that is included in a nuclease. A co-location sequence is considered directly bound to a synthetic nuclease in examples where a synthetic binding domain is encoded with the synthetic nuclease such that the synthetic binding domain is within the same polypeptide as the synthetic nuclease. This allows that synthetic binding domain to bind directly to the co-location sequence and the synthetic nuclease is brought into proximity of the cssDNA. A co-location sequence is considered to be indirectly bound to a synthetic nuclease in examples where the synthetic binding domain within the synthetic nuclease binds to the co-location sequence through a protein:protein interaction.

In examples where a co-location sequence is indirectly bound to a synthetic binding domain within a nuclease, the co-location sequence can be bound to an amino acid sequence that forms a multimer with one or more additional amino acid sequences and the one or more additional amino acid sequences can be a synthetic binding domain in a nuclease. See FIG. 5 which illustrates a nuclease engineered to express a protein which binds to a second protein and the second protein is bound to the colocation sequence. Those skilled in the art, reading the present disclosure, will appreciate that its teachings are applicable to any of a variety of nucleases capable of being engineered to associate with the co-location sequence directly or indirectly.

Nucleases that are designed to include one or more additional amino acid sequences for the purpose of either directly, or indirectly, binding to the co-location sequence are referred to as nucleases that include a synthetic binding domain, or synthetic nucleases. Those skilled in the art, reading the present disclosure, will be familiar with a variety of technologies that may be used to include or introduce a synthetic binding domain in a nuclease as described herein. For example, in some embodiments, a known dsDNA binding domain (e.g., from a known amino acid sequence) can be inserted into the coding sequence of a nuclease at one or the other end of the amino acid sequence, in which case a linker sequence (e.g., of 1-50 amino acids, e.g., SEQ ID NO: 20) can optionally be included between the synthetic binding domain and the nuclease. Alternatively, in some embodiments, the synthetic binding domain can be included within the nuclease such that it is dispersed within the amino acid sequence of the nuclease, but yet still adds the functionality of binding directly or indirectly to the co-location sequence while not substantially interfering with the nuclease activity.

Synthetic DNA binding domains are amino acid sequences that nucleases such as restriction endonucleases, Cas proteins, zinc finger nucleases (ZFN), meganucleases, homing endonucleases, transcription factor like effector nucleases (TALEN), and the like can be designed to incorporate. The synthetic binding domains are termed synthetic because they are designed and engineered to be included in such nucleases and upon inclusion function to locate the nuclease in proximity to the cssDNA through association with the co-location sequence.

Those skilled in the art, reading the present disclosure, will be familiar with a variety of technologies that can be used to produce cssDNA molecules as described herein. A particularly useful method of manufacturing cssDNA is through the use of bacteriophage sequences and production strains. Generally, a cssDNA phage genome is divided so that at least a portion of the genes necessary for producing and packaging the phage particles are expressed either from a genome of a production strain or from an extrachromosomal vector such as a helper virus or plasmid. A template sequence lacking at least a portion of the phage genes necessary for phage production is transformed into the production strain and the production strain provides the missing phage proteins necessary for the production and packaging of the phage particles. cssDNA molecules made using phage particle production techniques can include in addition to the co-location sequence, a packaging signal, a designed sequence and a selectable sequence.

Designed sequences can be useful for editing dsDNA within a target cell. Target cells can be prokaryotic or eukaryotic cells. Designed sequences can vary in length, for example from 1 bp to about 40,000 bp, depending upon the intended function of the designed sequence. In instances where an entire gene is to be inserted into a target site in a dsDNA molecule, an engineered version of a gene can be created synthetically such that intervening sequences, such as introns, are removed and a promoter and terminator can be added. The addition of such synthetic gene sequences can be used to add function to the cell into which the synthetic gene is introduced. Designed sequences can also be used to introduce entire operons into a target dsDNA molecule, which is useful in many synthetic biology applications. Similarly, cell function can be also altered through the addition of as little as 1 bp when such 1 bp change alters a codon in a coding sequence, or alters the functionality of a control sequence.

Designed sequences can also include one or more regions of homology that are homologous to a desired region of the target dsDNA. Regions of homology can be, e.g., from about 4 bp to about 100 bp, from about 5 bp to about 100 bp, from about 5 bp to about 80 bp, from about 5 bp to about 50 bp, from about 20 bp to about 5000 bp, and from about 15 bp to about 3000 bp. One of ordinary skill in the art will appreciate that regions of homology do not need share 100% sequence identity with the target dsDNA, rather the region of homology merely needs to share enough sequence identity with the target dsDNA such that the region of homology binds to the complementary region within the dsDNA. Regions of homology that share greater than 70%, 80%, 90%, 95% or 99% with the target dsDNA can provide the benefit of site specific integration of the designed sequence and also allow a single cssDNA to be made that will be useful for editing target dsDNAs that are diverse in their nucleic acid sequences.

Methods of using and producing the cssDNA that includes a co-location sequence are also described. Such methods include manufacturing the cssDNA using recombinant phage techniques as well as non-phage production techniques. As mentioned, once made the cssDNA in combination with a synthetic nuclease can be used to alter the genome of any cell. In some methods, the synthetic nuclease and the cssDNA can be delivered to the cell on plasmids which then express the cssDNA and the synthetic nuclease. The cssDNA and the synthetic nuclease can also be manufactured and combined through transformation into the cell. One of ordinary skill in the art will appreciate that there are a variety of variations that can be used to deliver the cssDNA and the synthetic nuclease to the target dsDNA.

Kits that include an expression cassette for the synthetic nuclease and an expression cassette for the cssDNA are provided. Alternative kits can include combinations of expression cassettes and the protein form of the synthetic nucleases. In yet additional embodiments, kits are described that include cssDNA and the associated synthetic nucleases that are ready for altering dsDNA.

The present disclosure provides, among other things, composition comprising a cssDNA molecule, wherein the cssDNA molecule comprises a co-location sequence, a selectable sequence, a packaging signal, and a designed sequence, wherein the co-location sequence is capable of associating with a synthetic nuclease. In some embodiments, the synthetic nuclease is associated with a synthetic binding domain and the cssDNA associates with the synthetic nuclease through the synthetic binding domain binding to the co-location sequence.

In another aspect, the present disclosure provides a composition comprising: a cell, wherein the cell comprises a synthetic nuclease, wherein the synthetic nuclease is associated with a synthetic binding domain, wherein the synthetic binding domain is capable of associating with a co-location sequence, and a cssDNA molecule, wherein the cssDNA molecule comprises the co-location sequence.

In some embodiments, the cssDNA molecule further comprises a designed sequence. In some embodiments, the cell is a eukaryotic cell. In some embodiments, the cell is a mammalian cell. In some embodiments, the cell is selected from a yeast cell or a fungal cell. In some embodiments, the cell is an algal cell. In some embodiments, the cell is a bacterial cell.

In some embodiments, the cssDNA molecule does not contain a packaging signal. In some embodiments, the cssDNA molecule further comprises a packaging signal. In some embodiments, the cssDNA molecule comprises a selectable sequence.

In some embodiments, the synthetic nuclease is linked to a first protein and the synthetic binding domain is linked to a second protein, wherein the first protein and second protein form a dimer, thereby associating the synthetic nuclease with the synthetic binding domain. In some embodiments, the first protein comprises a first portion of a GFP protein and the second protein comprises a second portion of a GFP protein. In some embodiments, the first portion of the GFP protein comprises beta strand peptides 1-10 of GFP and the second portion of the GFP protein comprises beta strand peptide 11 of the GFP protein, and wherein GFP beta strand peptide 11 and GFP beta strand peptides 1-10 associate with each other to form a complete GFP protein.

In some embodiments, the synthetic nuclease is linked to the synthetic binding domain through a linker sequence. In some embodiments, the synthetic nuclease is linked to the synthetic binding domain through a flexible linker. In some embodiments, the linker comprises an amino acid sequence according to SEQ ID NO: 20 or 21.

In some embodiments, the synthetic binding domain comprises a DNA binding domain.

In some embodiments, the synthetic binding domain comprises an amino acid sequence capable of forming a protein:protein interaction with a second amino acid sequence. In some embodiments, the second amino acid sequence additionally comprises a DNA binding domain.

In some embodiments, the DNA binding domain binds to the co-location sequence.

In some embodiments, the co-location sequence comprises a Zinc Finger DNA binding motif. In some embodiments, the co-location sequence comprises a Zinc Finger DNA binding motif sequence that is predicted to form a stem and loop secondary structure. In some embodiments, the co-location sequence comprises a Zinc Finger 5 DNA binding motif (ZFDBM5). In some embodiments, the ZFDBM5 comprises a sequence that includes a nucleic acid sequence according to SEQ ID NO: 46. In some embodiments, the ZFDBM5 sequence is predicted to form a stem and loop secondary structure that comprises a nucleic acid sequence according to SEQ ID NO: 24 or 26. In some embodiments, the ZFDBM5 comprises a nucleic acid sequence according to SEQ ID NO: 23 or 25.

In some embodiments, the synthetic binding domain is a dsDNA binding domain. In some embodiments, the synthetic binding domain is a ssDNA binding domain. In some embodiments, the synthetic binding domain is derived from an amino acid sequence selected from the group consisting of: a Cas protein, a zinc finger nuclease (ZFN), a meganuclease, a homing endonuclease, a transcription factor like effector nuclease (TALE); and a restriction endonuclease. In some embodiments, the synthetic binding domain comprises a ZF. In some embodiments, the ZF comprises ZF5 (e.g., as shown in SEQ ID NO: 22). In some embodiments, the synthetic nuclease is selected from the group consisting of: a Cas protein, a zinc finger nuclease (ZFN), a meganuclease, a homing endonuclease, a transcription factor like effector nuclease (TALEN), or another nuclease capable of creating double- or single-strand DNA breaks. In some embodiments, the synthetic nuclease comprises a Cas9 protein. In some embodiments, the Cas9 protein comprises an amino acid sequence according to SEQ ID NO: 16 or 18.

In some embodiments, the synthetic binding domain comprises ZF5 and the synthetic nuclease comprises Cas9, wherein the ZF5 is linked to the Cas9 through a flexible linker. In some embodiments, the ZF5 linked to a Cas9 nuclease comprises an amino acid sequence comprises SEQ ID NO: 51 or 53.

In another aspect, the present disclosure provides a vector comprising a nucleic acid sequence that encodes a ZF5-Cas9 fusion protein comprising an amino acid sequence according to SEQ ID NO: 51 or 53. In some embodiments, the vector comprises a nucleic acid sequence according to SEQ ID NO: 6 or 7.

In some embodiments, the cssDNA molecule comprises at least one region of homology, wherein the region of homology targets a region in the genome of a target cell. In some embodiments, the region in the genome of a target cell comprises a region within or including the TRAC locus (NC_000014.9). In some embodiments, the region of homology comprises two homology arms comprising an upstream homology arm (UHA) and a downstream homology arm (DHA), wherein the UHA comprises a nucleic acid sequence according to SEQ ID NO: 27 and the DHA comprises a nucleic acid sequence according to SEQ ID NO: 28. In some embodiments, the cssDNA molecule comprises a nucleic acid sequence according to any one of SEQ ID NOs: 3-5, and 10.

In some embodiments, a composition described herein further comprises a gRNA. In some embodiments, the gRNA sequence comprises a sequence that is targeted to a region within or including the TRAC locus. In some embodiments, the region within or including the TRAC locus comprises the g526 protospacer sequence according to SEQ ID NO: 42. In some embodiments, the gRNA sequence comprises SEQ ID NO: 29. In some embodiments, the designed sequence functions to alter the genome of a mammalian cell.

In some embodiments, the designed sequence comprises a control sequence. In some embodiments, the control sequence is selected from the group consisting of: introns, promoters, DNA binding sites, RNA binding sites, repressor binding sites, enhancer binding sites, transcription modifiers, and translation modifiers. In some embodiments, the designed sequence alters a coding sequence in a gene. In some embodiments, the designed sequence is greater than 3 kb. In some embodiments, the designed sequence comprises a coding region within any one of genes listing in Example 7.

The present disclosure provides, in another aspect, a method of engineering a target dsDNA in a cell comprising: contacting a target dsDNA with a synthetic nuclease and a cssDNA comprising a co-location sequence; and culturing the cell.

The present disclosure provides, in another aspect, a method of making a kit for editing dsDNA, comprising: selecting a cssDNA comprising a co-location sequence; selecting an synthetic nuclease wherein the synthetic nuclease comprises a synthetic binding domain and wherein the synthetic binding domain associates with the co-location sequence; and assembling the cssDNA and synthetic nuclease into a kit.

The present disclosure provides, in another aspect, a kit made by the method according to methods described herein.

The present disclosure provides, in another aspect, fusion protein comprising a Cas9 nuclease linked to a zinc finger (ZF) via a flexible linker. In some embodiments, the fusion protein according to claim 58, wherein the ZF comprises ZF5.

The present disclosure provides, in another aspect, complex comprising: a Cas nuclease linked to a ZF via a flexible linker or through a protein:protein interaction; and a cssDNA molecule comprising a designed sequence and a co-location sequence; wherein the co-location sequence comprises a ZF DNA binding motif (ZFDBM); and wherein the the ZF binds to the ZFDBM, thereby forming the complex.

### BRIEF DESCRIPTION OF THE FIGURES

FIG. 1 shows linear, single-stranded DNA homology directed repair templates (HDRTs) that have Cas9 target sequences (CTSs) appended to either end (see Shy et al., High-yield genome engineering in primary cells using a hybrid ssDNA repair template and small-molecule cocktails. Nat Biotechnol (2022))

FIG. 2 shows a phagemid comprising an F1 origin of replication (F1); “A” denoting a selectable sequence; “B” denoting a designed sequence; a region of reverse complementarity and an optional plasmid origin of replication.

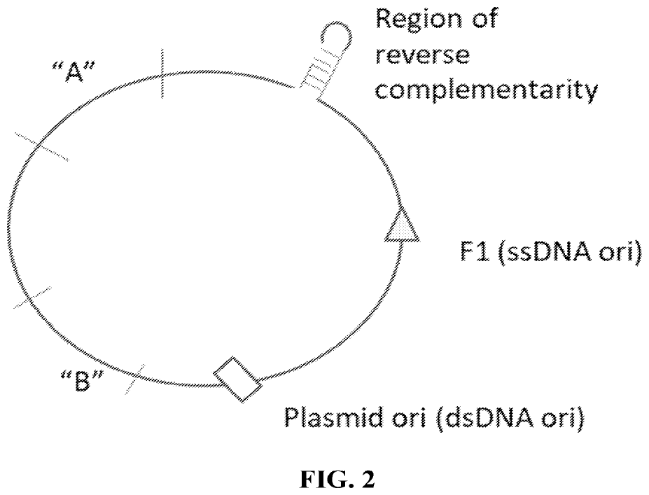

FIGS. 3A-B shows exemplary designs of ZF arrays that bind to co-location sequences. Panel (A) shows Cys2-His2 zinc fingers (ZFs) are small (~30 amino acid) domains that recognize ~3-bp DNA sequences. Panel (B) shows design and assembly of ZF arrays. By linking two-finger units (each recognizing 6-bp subsites) using flexible, ‘disrupted’ linkers, it is possible to construct functional six-finger arrays capable of specifically recognizing 20-bp DNA binding motifs (DBM) (18-bp core+2-bp flanking). Israni D V, Li H, Gagnon K A, et al. Clinically-driven design of synthetic gene regulatory programs in human cells. bioRxiv; 2021, which is herein incorporated by reference in its entirety. Amino acid sequences encoding zinc finger arrays can additionally encode amino acid sequences that form dimers with other amino acid sequences associated with synthetic nucleases.

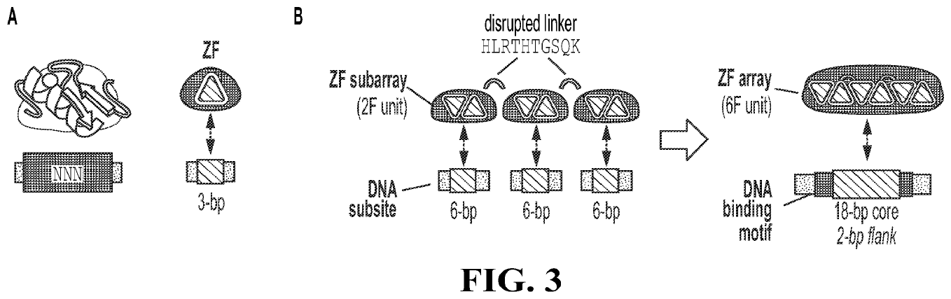

FIG. 4 shows a schematic of a synthetic nuclease including a linker and a synthetic DNA binding domain (6×ZF array). Also illustrated is a cssDNA that includes a co-location sequence (DBM5) and its association with synthetic DNA binding domain in the synthetic nuclease.

FIG. 5 shows a schematic of a synthetic nuclease including a linker and a synthetic binding domain that indirectly associates the synthetic nuclease with a protein that is associated with the co-location sequence. In this example, Cas9 is expressed as a transcriptional fusion with GFP peptide 11. Zinc finger five is separately expressed as a different transcriptional fusion with GFP peptides 1-10. When incubated together in solution, the two protein fusions are bound together by the interaction between GFP peptide 11 and GFP peptides 1-10.

FIG. 6 shows a schematic of a nucleic acid sequence encoding an exemplary nuclease-DNA-binding domain fusion protein described herein. The nucleic acid sequence is included in an in vitro expression cassette that is codon optimized, synthesized as DNA and cloned into a plasmid backbone by Biomatik (Kitchener, Ontario, Canada). The in vitro expression cassette consists of the SV40 nuclear localization signal and the R691A high fidelity mutant of Cas9. R691A high fidelity (HiFi) Cas9 is chosen as the nuclease due its low off-target effects.

FIG. 7 shows a schematic of a DBM5 co-location sequence.

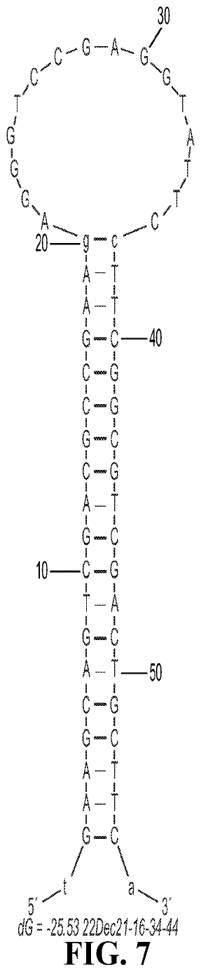

FIG. 8 shows a schematic of a negative control co-location sequence.

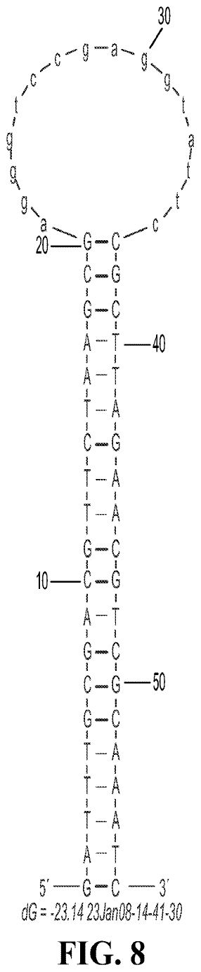

FIG. 9 shows a schematic of an exemplary phagemid backbone that includes a plasmid origin of replication pUC and packaging signal from M13K07, an the auxotrophic selection marker pyrF, a p19658 promoter, a L3S2P21 synthetic terminator, a designed sequence that includes a pair of homology arms targeting the homology directed repair event to the human T-cell receptor α constant (TRAC) locus, and a polyA signal is from the sequence downstream of the herpes simplex virus thymidine kinase (HSVtk) gene.

FIG. 10 shows a schematic of an exemplary phagemid backbone that contains a dystrophin designed sequence.

FIGS. 11A-B shows an example of microscopic images of myoblast cell cultures expressing the wild type DMD gene (panel A) and a diseased mutant DMD gene (panel B) (see Nesmith et al., J Cell Biol. 2016 Oct. 10; 215(1):47-56).

FIG. 12 shows a schematic of an exemplary phagemid backbone that contains a utrophin designed sequence.

FIG. 13 shows shows a schematic of the TRAC amplicon utilized in an vitro cleavage assay in Example 9.

FIG. 14 shows a gel image from an in vitro cleavage assay utilized in Example 9.

FIG. 15 shows a schematic of cdsDNA221 without zinc finger DNA binding motif number 5.

FIG. 16 shows a schematic of cdsDNA222 with zinc finger DNA binding motif number 5.

FIG. 17 shows a schematic of the zinc finger DNA binding motif number 5 of cdsDNA222.

FIG. 18 shows a schematic of the secondary structure of zinc finger DNA binding motif number 5 of cdsDNA222.

FIG. 19 shows a schematic of a gel image from an in vitro zinc finger binding assay utilized in Example 10.

FIG. 20 shows a schematic of cdsDNA166 Standard Cas9 mammalian expression vector from Vector Builder.

FIG. 21 shows a schematic of a Zinc finger 5 gBlock.

FIG. 22 shows a schematic of cdsDNA177 Cas9 ZF5 mammalian expression vector.

FIG. 23 shows a schematic of cdsDNA164 g526 sgRNA mammalian expression vector.

FIG. 24 shows the viability of HEK293T cells after transfection with only buffer, only cssDNA, only RNPs, only expression vector, RNPs and cssDNA, and expression vector and cssDNA.

FIG. 25 shows that a Cas9:ZF fusion protein described herein integrates cssDNA into the HEK293T genome.

### DETAILED DESCRIPTION

### Introduction

The present disclosure addresses certain challenges associated with DNA homology directed repair templates (HDRTs).

For example, HDRTs are often toxic to host cells into which they are introduced, resulting in low viability when large quantities of HDRTs are transfected into such cells. In principle, higher amounts of HDRT may be desirable because they may be more likely to achieve double strand break repair through homologous recombination, but the present disclosure appreciates that such higher levels risk generating unacceptably high cell mortality. Furthermore, longer HDRTs, which may be desirable due to their increased ability to integrate into genomes than shorter HDRTs, are also typically more toxic than the shorter HDRTs and, therefore, result in lower viability. Researchers therefore have therefore faced the conundrum of a tradeoff between viability and integration rates.

Specific additional challenges can be associated with different types of HDRTs. For instance, double-stranded DNA HDRTs generally have most of their bases bound to bases on the complementary strand through base-pairing hydrogen bonds, making them inaccessible to form new hydrogen bonds with homologous bases in the genome.

Also, linear, double-stranded and linear-single-stranded HDRTs are subject to degradation by host exonucleases.

Furthermore, DNA HDRTs may not enter the nucleus of mammalian cells. Eukaryotic nuclei are surrounded by two porous lipid bilayer membranes which selectively block molecules from entering or exiting the nucleus. Proteins require nuclear localization signals and ATP to cross the nuclear envelope. mRNA is actively exported across the nuclear envelope after it interacts with the transport receptor Tap-p15. Other RNAs such as tRNA, microRNA, small non-coding RNA are transported across the nuclear envelope by the importin/karyopherin-β family of proteins. See, for example, Katahira J. Nuclear export of messenger RNA. Genes (Basel). 2015 Mar. 31; 6(2):163-84. DNA does not appear to have an active transport mechanism similar to either proteins or RNA. Indeed, part of the function of the nuclear envelope is to separate the DNA of the genome from the rest of the cell. The kinetics of DNA diffusion across the nuclear envelope are much slower than would be expected from hydrodynamic considerations alone, suggesting that the nuclear pores pose somewhat of a barrier to DNA. See, for example, Salman et al., Kinetics and mechanism of DNA uptake into the cell nucleus, PNAS, Jun. 5, 2001, 98 (13) 7247-7252.

The present disclosure specifically appreciates and addresses a challenge that, with many conventional HDRT technologies, the DNA HDRT may not be near the site of the double-strand break. Even if some DNA does enter the nucleus, most CRISPR/Cas genome engineering techniques rely on simple diffusion of the HDRT through the nucleus. While the Cas9 nuclease is actively directed to the desired site of genome editing by the gRNA and PAM sequence, there is no such active means of directing the HDRT to this site. The present disclosure appreciates that this failure to co-localize may be part of the reason that double-strand DNA breaks are observed at the desired location more often than homology directed repair.

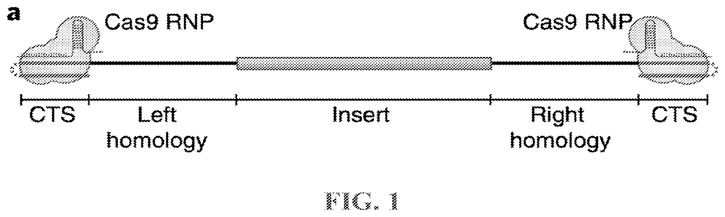

Recently, strategies have been developed to address some of these limitations. For example, in one approach, linear, single-stranded HDRTs that have Cas9 target sequences (CTSs) appended to either end. The CTS regions are dsDNA formed at the ends of the homology regions, while the remainder of the HDRT remains ssDNA. The linear, single-stranded DNA is less toxic to the host than traditional double-stranded DNA and the fact that it is single-stranded also makes its bases more available to bind through hydrogen bond base pairing with homologous DNA in the genome. Furthermore, the CTS sequences allow the Cas9/gRNA ribonucleoprotein (RNP) complex to interact directly with the HDRT by binding to the CTS sites. This binding ensures that the HDRT is transported along with the RNP through the nuclear envelope due to the inclusion of at least one nuclear localization signal fused to the Cas9 protein. Furthermore, it ensures that the HDRT is transported along with the RNP to the site of the double-strand break thus making it readily available to repair the double-strand break through homologous recombination because of its physical proximity. By employing this method, homology directed repair rates of up to 90% were achieved in primary T cells. See, FIG. 1, Shy et al., High-yield genome engineering in primary cells using a hybrid ssDNA repair template and small-molecule cocktails. Nat Biotechnol (2022).

The present disclosure appreciates that even this approach has some limitations. The linear single-stranded DNA sequences were limited to about 3000 nucleotides. Although there are many ways to produce linear, single-stranded DNA sequences, all of them suffer from high costs, low fidelity and low yield especially when the sequences are greater than 3,000 to 5,000 nucleotides. This limits the amount of DNA that can be produced, which is a concern especially in CAR-T cell therapy where large quantities of HDRT are needed to produce large quantities of engineered T cells for patient therapy. It also limits fidelity, which is a concern for cell and gene therapies. Low fidelity HDRTs will introduce mutations into patients which reduces the safety of these therapies and may affect FDA approval. Lastly, the CTSs need to be designed de novo for each new integration site. While this is a minor consideration, it does require some design work and some DNA synthesis that must be repeated for each new application reducing the scalability of the method.

Joung et al., 2015 WO2016054326A1 described a method of fusing a nuclease to a DNA binding domain which is programmed to bind to both the homology arm of the HDRT and the homologous sequence in the genome. This solved some of the problems associated with CRISPR/Cas HDR stated above. For example, tethering the HDRT to the nuclease ensured that the HDRT would be present in the nucleus near the site of the double-strand break. The present disclosure appreciates, however, that this approach may also have some drawbacks. Thus, among other things, the present disclosure identifies the source of the problem with certain conventional technologies. For example, the present disclosure appreciates that linear, single or double-stranded HDRTs can both be degraded by host exonucleases and in the case of linear, double-stranded HDRTs, are highly toxic to the host cell and do not recombine with the host genome at high rates. Furthermore, the present disclosure appreciates that circular, double-stranded HDRTs, like their linear counterpart, are also highly toxic to the host cell and do not recombine with the host genome at high rates. Furthermore, this approach requires reprogramming the DNA binding domain for each new genomic locus, which is not trivial in the case of zinc fingers (ZFs) and transcription activator-like effectors (TALEs). Also, the fact that the DNA binding domain can bind to either the homology arm on the HDRT or its homologous sequence in the genome means that the DNA binding domain may be bound to the HDRT as desired only about 50% of the time, while it will be bound to the homologous sequence in the genome the other 50% of the time. This may reduce the desired effect by 50%.

Cas9 has also been fused to many other functional proteins through genetically encoding flexible linker sequences in between the open reading frame for Cas9 and the protein one desires to link to Cas9. (Cas9 has been fused to VP16 activation domains to activate gene expression near the gRNA binding site, (La Russa M F, Qi L S. The New State of the Art: Cas9 for Gene Activation and Repression. Mol Cell Biol. 2015 November; 35(22):3800-9), transcriptional repressors to repress gene expression (Yeo et al., An enhanced CRISPR repressor for targeted mammalian gene regulation. Nat Methods. 2018 August; 15(8):611-616), deaminases to edit bases (Komor et al., Programmable editing of a target base in genomic DNA without double-stranded DNA cleavage. Nature. 2016 May 19; 533(7603):420-4), and reverse transcriptases to transcribe the HDRT (Anzalone, A. V., Randolph, P. B., Davis, J. R. et al. Search-and-replace genome editing without double-strand breaks or donor DNA. Nature 576, 149-157 (2019)).

Iyer et. al, discloses homology directed repair rates of 5-30% using circular single-stranded DNA (cssDNA) HDRTs in HEK293 and K562 cells. Iyer et al., Efficient Homology-Directed Repair with Circular Single-Stranded DNA Donors. CRISPR J. 2022 October; 5(5):685-701. Notably, this approach did not include any means of binding the HDRT to the nuclease unlike the approaches of Shy et al., and Joung et al., described above, yet was still able to demonstrate the efficacy of cssDNA for use as HDRTs in mammalian cells achieving high rates of homology directed repair in multiple cell lines with multiple reporter constructs.

Here, the inventors have proposed a new solution where the nuclease is fused to a DNA binding domain that is programmed to recognize a constant co-location sequence on a circular-single-stranded DNA HDRT. This accomplishes the some of the goals described above in that it tethers the HDRT to the nuclease ensuring that the HDRT enters the nuclease and is present near the site of the double-strand break, and in addition, it also lacks some of the drawbacks. The fact that the co-location sequence is constant obviates any need to synthesize new CTS oligos as described in Shy et al, described above. It also obviates the need to reprogram ZF, TALE or Cas proteins to recognize new homology arm sequences at each new genomic locus targeted. This means that the nuclease-DNA binding domain fusion protein can be cloned and in vitro transcribed once and then be used in many genome engineering experiments or productions at many loci and in many organisms. The fact that the HDRT is circular-single-stranded DNA reduces toxicity to the cells, reduces the probability that the HDRT will be degraded by host exo- or endonucleases and increases the chance it will recombine with homologous bases in the genome.

Zinc fingers are DNA binding domains of proteins of approximately 30 amino acids that each recognize a 3-bp DNA sequence. Zinc fingers can be linked through genetic protein fusions to form complexes of zinc fingers that recognize longer DNA sequences such as 3 or 6 or 9 or more bases. Israni et al., constructed a zinc finger fusion protein consisting of 6 zinc fingers that recognizes an 18 bp core sequence.

It is possible that fusing Cas9 to another nuclease such as a zinc finger or TAL has not drawn much attention because Cas9, which has its own programmable DNA binding domain was such a revolutionary discovery that immediately most people considered zinc fingers and TALs to be obsolete technology. Researchers may have focused on new uses for Cas9's programmable DNA binding domain while failing to think of uses for zinc finger and TAL programmable DNA binding domains because of the difficulty in programming these older technologies. Researchers may not have been motivated to invent a fusion between Cas9 and older DNA binding proteins because Cas9 alone could accomplish so many of their goals with its single DNA binding domain. After about a decade of research on Cas9 however, modern researchers are realizing that there is a limit a to the rate of HDR achievable in cell and gene therapies using Cas9 alone and that limit is posing an obstacle to the ability of these gene and cell therapies to move from the research and development phase to becoming approved clinical therapies.

This solution of transcriptionally fusing Cas9 (e.g., according to SEQ ID NO: 16 or 18, or a sequence having at least 85%, at least 90%, at least 95%, at least 99% identity the SEQ ID NO: 16 or 18) to zinc finger 5 (e.g., according to SEQ ID NO: 22 or a sequence having at least 85%, at least 90%, at least 95%, at least 99% identity the SEQ ID NO: 22) and binding zinc finger 5 to the cssDNA HDRT by including a DNA binding domain 5 dsDNA hairpin (e.g., as shown in SEQ ID NOs: 23-26 and 46-48, or a sequence having at least 85%, at least 90%, at least 95%, at least 99% identity the SEQ ID NOs: 23-26 and 46-48) within the cssDNA HDRT is one possible solution to the problems with CRISPR gene and cell therapy described above.

Those skilled in the art will recognize that Cas9 is not the only programmable nuclease that can be used to achieve these goals. In previous years, researchers would have turned to zinc fingers or TALs as the programmable nucleases of choice to create double stranded DNA breaks at precise locations in genomes and those remain viable options. More recently researchers have become of aware of the broad of Cas proteins that exist in nature, some of which have been harnessed for genome editing including Cas12a, Cas13a and Cas13b as well as others. Yan et al., CRISPR-Cas12 and Cas13: the lesser known siblings of CRISPR-Cas9. Cell Biol Toxicol 35, 489-492 (2019), which is herein incorporated by reference in its entirety.

Likewise, those skilled in the art will be aware that zinc finger 5 is not the only DNA binding protein that can be used to associate a nuclease with a cssDNA HDRT. Other zinc fingers, TALs or Cas genes could be programmed to bind to a specific DNA sequence within the HDRT and the DNA co-location sequence itself could be modified to facilitate binding to any of these other DNA binding proteins. In fact, any sequence-specific DNA binding protein could be used. This includes restriction enzyme meganucleases if their nuclease activity were ablated.

Furthermore, a transcriptional fusion between a nuclease and a DNA binding domain is not the only way of associating two protein domains with each other. Those skilled in the art will be aware that proteins can also be associated to each other through protein:protein interaction or isopeptide bonds. Example 8 below describes two such examples, namely. split GFP and spy catcher/spy tag to associate two proteins together, but those skilled in the art will be aware of many instances in nature and biotechnology of protein domains that interact with each other through dimerization and other forms of binding. Any of these protein domains that are known to interact with each other could be used to establish the physical connection between a programmable nuclease and DNA binding protein.

This disclosure describes, in some embodiments, cssDNA HDRTs that are produced in E. coli which expresses M13 phage proteins that work with E. coli proteins to replicate and package the cssDNA into a phage capsid enabling easy purification of the cssDNA HDRT from the E. coli DNA, proteins and other contaminants, but those skilled in the art will recognize that this is not the only way to create cssDNA HDRTs. They can also be created in vitro using rolling circle amplification or through chemical or enzymatic synthesis. Mohsen et al., Acc. Chem. Res. 2016, 49, 11, 2540-2550 Publication Date: Oct. 24, 2016, Hao et al., Current and Emerging Methods for the Synthesis of Single-Stranded DNA. Genes (Basel). 2020 Jan. 21; 11(2):116, wherein are herein incorporated by reference in their entirety.

The technology described in this disclosure could also be applied to other DNA HDRTs including linear dsDNA, circular dsDNA, and linear ssDNA.

### Gene Therapy

One of the ultimate goals of developing a system with improved genomic integration rates is its use in gene therapy applications. Some of the problems currently limiting the application of gene therapies are low integration rates, small cargo sizes and cytotoxicity. The system described in the present disclosure addresses all three of these issues allowing new genetic diseases to become accessible to gene therapy treatments. Creating a physical bond between a Cas9 nuclease and an HDRT through the zinc finger fusion to Cas9 and zinc finger binding to the HDRT increases integration rates. The use of cssDNA rather than dsDNA reduces cytotoxicity. The production of the cssDNA HDRT using m13 phage allows packaging of user defined HDRT sequences up to around 30,000 nt.

One such disease which has remained recalcitrant to gene therapy is Duchenne muscular dystrophy or DMD, which is the most common form of genetic muscular dystrophy affecting 1 in 3,500 to 1 in 5,000 live male births globally. Emery et al., Population frequencies of inherited neuromuscular diseases—a world survey. Neuromuscul Disord. 1991; 1:19-29. It is a debilitating disease. Affected children usually present with gross motor delay and/or decline by age 5, becoming wheelchair dependent by age 13 (see You E M, Kornberg A J, Duchenne muscular dystrophy. J Paediatr Child Health. 2015; 51:759-64 and Koeks Z, et al., Clinical outcomes in Duchenne Muscular Dystrophy: a Study of 5345 patients from the TREAT-NMD DMD Global Database. J Neuromuscul Dis. 2017:4:293-306). Mean survival is limited mainly by cardiorespiratory complications, to 29 years of age (see Landfeldt E, et al., Life expectancy at birth in Duchenne muscular dystrophy: a systematic review and meta-analysis. Eur J Epidemiol. 2020; 35:643-53).

Despite the prevalence and severity of the disease, gene therapies have yet to be successfully developed. One of the primary obstacles to curing DMD through gene therapy is the size of the DMD gene, which codes for the dystrophin protein. In fact, DMD is the largest known human gene at 2.5 megabases. The coding sequence is made up of 86 exons. The full-length mRNA is 14,000 nt and the amino acid sequence of the mature protein is 3685 residues. Moreover, the mutations that lead to DMD and the related Becker muscular dystrophy (BMD) are various and scattered throughout the gene. Any mutation that leads to lost or diminished function of the dystrophin protein will result in either DMD or BMD in males that have only one copy of the DMD gene on their lone X chromosome (see Muntoni et al., Dystrophin and mutations: one gene, several proteins, multiple phenotypes. Lancet Neurol. 2003).

Due to the length of the DMD open reading frame and the 4,700 nt packaging limitation of the adeno associated viruses or AAV's that are widely used in gene therapy. pharmaceutical companies have sought to treat DMD by introducing a micro-dystrophin gene. In this approach, researchers attempt to engineer a small transgene that can be packaged into the AAV. They attempt to code for the important domains of the full length DMD gene while omitting unnecessary sequences to allow the transgene to fit within the strict packaging limit of the AAV while still compensating for the function of the disabled DMD gene. This approach, however, has produced poor results in clinical trials.

The trial of Sarepta's therapy, SRP-9001, was the first placebo-controlled study of a muscular dystrophy gene therapy. The study enrolled 4 to 7-year-old boys, testing whether treatment led to increased dystrophin production and improvement in muscle function. Tests showed the treatment did result in micro-dystrophin protein production, but not in improved motor function implying that expression of the micro-dystrophin gene does not fully compensate for the loss of the native DMD gene. Pfizer and Solid Biosciences are working on similar, micro-dystrophin therapies, but have yet to release phase III efficacy data (see Jonathan Gardner, Jan. 7, 2021, Sarepta gene therapy misses goal in key muscular dystrophy study BioPharma Dive, www.biopharmadive.com/news/sarepta-gene-therapy-duchenne-study-data/593024/).

One major advantage of the approach described in this patent is that production of the cssDNA HDRT using the E. coli/m13 phagemid system allows packaging of DNA sequences up to around 30,000 nt rather than the 4700 nt allowed by AAV's.

This same approach can be used to treat many important genetic diseases affecting humans. Similar to Duchenne muscular dystrophy, cystic fibrosis also affects a large number of people (1 out of 3000-4000 live births) and is caused by various mutations in the cystic fibrosis transmembrane conductance regulator (CFTR) gene. The mutations occur across a 6 kb open reading frame that is also too long to fit in an AAV. The defects in this gene could be corrected by encoding the entire open reading frame along with homology arms targeting it to the endogenous CFTR locus on a cssDNA that also contains a DNA binding domain that allows the CRISPR complex to associate with the cssDNA HDRT.

Likewise, mutations in the BRCA1 and BRCA2 genes could be corrected with this technology. About 1 in 1000 people have mutations in these genes. In women, mutations in either gene raise the chance of developing breast cancer from 7% to 45% or 65% depending on which gene is affected. BRCA1 has a 6 kb open reading frame and BRCA2 has a 10 kb open reading frame which are each too long to fit in and AAV. Mutations in both genes can occur at any base across the length of the gene making them excellent candidates for full length gene replacement at the endogenous locus using a cssDNA HDRT encoding a full correct version of the either the BRCA1 or BRCA2 open reading frame, homology arms targeting each gene to its native locus, a DNA binding domain that allows the cssDNA HDRT to interact with a CRISPR complex.

Indeed, there are many examples of genetic disorders that could be cured with this technology and some but not all of them are listed in Example 7.

### Definitions

Unless otherwise noted, technical terms are used according to conventional usage. Definitions of common terms in molecular biology can be found in Benjamin Lewin, Genes XII, Jones & Bartlett Learning; 12th edition (Mar. 16, 2017) and other similar references. As used herein, the singular forms “a,” “an,” and “the,” refer to both the singular as well as plural, unless the context clearly indicates otherwise. For example, the term “a peptide” includes single or plural antigens and can be considered equivalent to the phrase “at least one peptide.” As used herein, the term “comprises” means “includes.” Thus, “comprising a protein” means “including a protein” without excluding other elements. It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting. To facilitate review of the various embodiments, the following explanations of terms are provided:

In general, the term “agent”, as used herein, is used to refer to an entity (e.g., for example, a lipid, metal, nucleic acid, polypeptide, polysaccharide, small molecule, etc., or complex, combination, mixture or system [e.g., cell, tissue, organism] thereof), or phenomenon (e.g., heat, electric current or field, magnetic force or field, etc.).

The terms “amino acid sequence”, “polypeptide”, “peptide” and “protein” are used interchangeably to refer to a polymer of amino acid residues. The term also applies to amino acid polymers in which one or more amino acids are chemical analogues or modified derivatives of corresponding naturally-occurring amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids. L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications may be selected from the group consisting of acetylation, amidation, lipidation, methylation. pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a relevant polypeptide may comprise or consist of a fragment of a parent polypeptide. In some embodiments, a useful polypeptide as may comprise or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.

Two events or entities are referred to as “associated” with one another, as that term is used herein, if the presence, level, degree, type and/or form of one is correlated with that of the other. For example, a particular entity (e.g., polypeptide, genetic signature, metabolite, microbe, etc.) is considered to be associated with a particular disease, disorder, or condition, if its presence, level and/or form correlates with incidence of, susceptibility to, severity of, stage of, etc. the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another am covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

The use of the term “binding” as described herein refers to binding properties such as, e.g., binding affinity, binding specificity, and binding avidity. See David J. King, Applications and Engineering of Monoclonal Antibodies, pp. 240 (1998). Binding refers to the length of time a synthetic binding domain is bound to another protein through protein-protein interactions or the length of time a synthetic binding domain is bound to a nucleic acid sequence such as a co-location sequence. As used herein a synthetic binding domain refers to a domain that selectively binds to either its intended protein partner or the co-location sequence to a greater extent than it binds to another protein or nucleic acid sequence.

As used herein, the phrase “characteristic sequence element” refers to a sequence element found in a polymer (e.g., in a polypeptide or nucleic acid) that represents a characteristic portion of that polymer. In some embodiments, presence of a characteristic sequence element correlates with presence or level of a particular activity or property of the polymer. In some embodiments, presence (or absence) of a characteristic sequence element defines a particular polymer as a member (or not a member) of a particular family or group of such polymers. A characteristic sequence element typically comprises at least two monomers (e.g., amino acids or nucleotides). In some embodiments, a characteristic sequence element includes at least 2, 3, 4, 5, 6, 7, 8, 9, 10, 11, 12, 13, 14, 15, 20, 25, 30, 35, 40, 45, 50. or more monomers (e.g., contiguously linked monomers). In some embodiments, a characteristic sequence element includes at least first and second stretches of contiguous monomers spaced apart by one or more spacer regions whose length may or may not vary across polymers that share the sequence element.

“Circular single stranded deoxyribonucleic acid or cssDNA” as used herein refers to a circular single stranded DNA that includes a co-location sequence that can specifically be bound to an amino acid sequence such as a synthetic binding domain or a synthetic DNA binding domain. One of ordinary skill in the art will appreciate that cssDNA can be produced using any method known in the art, for example combinations of polymerase chain reaction can be used to create a dsDNA fragment and one of the two strands can selectively be degraded. In some examples, the cssDNA can be produced using phage technology. In such instances, the cssDNA will have additional features that can be useful in phage particle production.

As used herein, the term “comparable” refers to two or more agents, entities, situations, sets of conditions. etc., that may not be identical to one another but that are sufficiently similar to permit comparison therebetween so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions. etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied. For instance, in some embodiments control sequences are used in engineered nucleic acid sequences to express a coding sequence, the control sequences used are not the same as the control sequences that are found in the natural gene that expresses the protein in a cell. The use of control sequences that are distinct from those found in nature can be referred to as non-endogenous as compared to the coding sequence. In such instances the activity of the natural gene expression cassette can be compared to the engineered version of the gene made with the non-endogenous control sequences. In such instances the natural gene expression cassette can be referred to as the reference. In such comparisons the non-endogenous control sequences can be found to be active or inactive as compared to the natural gene expression cassette when both expression cassettes are expressed under substantially the same experimental conditions.

The term “conservative” refers to instances describing a conservative amino acid substitution, including a substitution of an amino acid residue by another amino acid residue having a side chain R group with similar structural, chemical (e.g., charge or hydrophobicity). and/or functional properties. In general, a conservative amino acid substitution will not substantially change functional properties of interest of a protein, for example, ability of a receptor to bind to a ligand. Examples of groups of amino acids that have side chains with similar chemical properties include: aliphatic side chains such as glycine (Gly, G), alanine (Ala, A). valine (Val, V), leucine (Leu, L), and isoleucine (Ile, I); aliphatic-hydroxyl side chains such as serine (Ser, S) and threonine (Thr, T); amide-containing side chains such as asparagine (Asn, N) and glutamine (Gln, Q); aromatic side chains such as phenylalanine (Phe, F), tyrosine (Tyr, Y), and tryptophan (Trp, W); basic side chains such as lysine (Lys, K), arginine (Arg, R), and histidine (His, H); acidic side chains such as aspartic acid (Asp, D) and glutamic acid (Glu, E); and sulfur-containing side chains such as cysteine (Cys, C) and methionine (Met, M). Conservative amino acids substitution groups include, for example, valine/leucine/isoleucine (Val/Leu/Ille, V/L/I), phenylalanine/tyrosine (Phe/Tyr, F/Y), lysine/arginine (Lys/Arg, K/R), alanine/valine (Ala/Val. A/V), glutamate/aspartate (Glu/Asp, E/D), and asparagine/glutamine (Asn/Gln, N/Q). In some embodiments, a conservative amino acid substitution can be a substitution of any native residue in a protein with alanine, as used in. for example, alanine scanning mutagenesis. In some embodiments, a conservative substitution is made that has a positive value in the PAM250 log-likelihood matrix disclosed in Gonnet et al., Science 256:1443, 1992, which is incorporated herein by reference in its entirety. In some embodiments, a substitution is a moderately conservative substitution wherein the substitution has a nonnegative value in the PAM250 log-likelihood matrix. One skilled in the art would appreciate that a change (e.g., substitution, addition, deletion, etc.) of amino acids that are not conserved between the same protein from different species is less likely to have an effect on the function of a protein and therefore, these amino acids should be selected for mutation. Amino acids that are conserved between the same protein from different species should not be changed (e.g., deleted, added, substituted, etc.), as these mutations are more likely to result in a change in function of a protein. In some embodiments, a “conservative” substitution is considered a “homologous” residue for purposes of calculating percent homology between amino acid sequences.

“Control sequences” refer to nucleic acid sequences that regulate the expression of a nucleic acid sequence to which the control sequence is operatively linked. Expression control sequences are operatively linked to a nucleic acid sequence when the expression control sequences control and regulate the transcription and, as appropriate, translation of the nucleic acid sequence. Thus, the term “expression control sequences” can refer to elements such as include appropriate promoters, enhancers, transcription terminators, ribosome binding sites and start codons (ATG) in front of a protein-encoding gene, splicing signal for introns, maintenance of the correct reading frame of that gene to permit proper translation of mRNA, and stop codons, etc. The term “control sequences” is intended to include, at a minimum, components whose presence can influence expression, and can also include additional components whose presence is advantageous, for example, leader sequences and fusion partner sequences. Expression control sequences can include a promoter.

“Designed sequences” or therapeutic sequences as used herein are engineered nucleic acid sequences included in cssDNA or ssDNA that are intended to be inserted into a target dsDNA molecule, in some instances a target dsDNA is in a cell genome. In some instances a designed sequence can include one or more regions that are homologous to the target dsDNA. In some instances a designed sequence can contain an entire gene expression cassette, for example, a promoter, open reading frame and a terminator. In other embodiments a designed sequence can include a gene or a fraction of a gene such as intron, exon, or the like. A designed sequence can be made using any method known in the art for example, designed sequences can be made using chemical synthesis, recombinant biology techniques such as PCR, cloning using phages and plasmids or a combination of such techniques that allow for the construction of the ssDNA or cssDNA that includes a designed sequence with a co-location sequence. A designed sequence in a phagemid can be designed to alter the genome of the target cell such that the function of an endogenous nucleic acid sequence in the target cell is replaced or lengthened by the designed sequence. In some instances, a designed sequence can be designed to delete, down-regulate or diminish the activity of an endogenous genomic product of the target cell. In others instances a designed sequence can be designed to up-regulate or increase expression of an amino acid sequence in the target cell. In yet other embodiments, the designed sequence can be designed to repair a defective allele in a target cell. Designed sequences can include for example, sequences that upon incorporation into a target dsDNA produce siRNA, ribozymes, antisense sequences, RNAi, genes, corrective sequences for various genetic diseases, or beneficial genes or other sequences. cssDNA sequences can be produced that have high fidelity and are relatively large, for example, greater than 3 kb, 4 kb, 5 kb, 6 kb, 7 kb, 8 kb, 9 kb, 10 kb, 11 kb, 12 kb, 13 kb, 14 kb, 15 kb, 16 kb. 17 kb, 18 kb, 19 kb, 20 kb 21 kb, 22 kb, 23 kb, 24 kb, 25 kb. 26 kb, 27 kb, 28 kb, 29 kb, or 30 kb. This size allows for a single designed sequence to be made that replaces a whole gene in a target cell. The ability for such whole gene replacement to be made allows a single design sequence to be manufactured and used to treat a patient population that has the same disease symptoms, but different genetic mutations in the gene causing the disease symptoms. In some instances when a designed sequence is intended to provide a therapeutic benefit to an organism, the designed sequence is referred to as a therapeutic sequence.

“DNA binding domain” refers to any amino acid sequence capable of binding to a DNA sequence motif, for example a specific dsDNA sequence or a specific ssDNA sequence. DNA binding domains can be selected such that they bind to a particular co-location sequence in a designed sequence. In particular examples, dsDNA binding domains include amino acid sequences that are derived from natural proteins that have been discovered to specifically target distinct sequences of dsDNA. Such dsDNA binding domains are derived from such sequences through engineering the natural proteins to not have undesired activity while maintaining the dsDNA binding activity. One of ordinary skill in the art will appreciate that such engineering can be accomplished through the deletion, substitution and/or addition of amino acids. Additionally, dsDNA binding domains can be associated with amino acid sequences through linker. As used herein a DNA binding domain when incorporated into another amino acid sequence is termed a synthetic DNA binding domain and is considered a type of synthetic binding domain that can be included in a synthetic nuclease.

In general, the term “engineered” or “synthetic” refers to the aspect of having been manipulated by the hand of man. For example, a polynucleotide is considered to be “engineered” when two or more sequences that are not linked together in that order in nature are manipulated by the hand of man to be directly linked to one another in the engineered polynucleotide and/or when a particular residue in a polynucleotide is non-naturally occurring and/or is caused through action of the hand of man to be linked with an entity or moiety with which it is not linked in nature. For example, in some embodiments described and/or utilized herein, an engineered polynucleotide comprises a regulatory sequence that is found in nature in operative association with a first coding sequence but not in operative association with a second coding sequence, is linked by the hand of man so that it is operatively associated with the second coding sequence. Comparably, a polypeptide may be considered to be “engineered” if encoded by or expressed from an engineered polynucleotide, and/or if produced other than natural expression in a cell. Analogously, a cell or organism is considered to be “engineered” if it has been subjected to a manipulation, so that it's genetic, epigenetic, and/or phenotypic identity is altered relative to an appropriate reference cell such as otherwise identical cell that has not been so manipulated. In some embodiments, the manipulation is or comprises a genetic manipulation, so that its genetic information is altered (e.g., new genetic material not previously present has been introduced, for example by transformation, mating, somatic hybridization, transfection, transduction, or other mechanism, or previously present genetic material is altered or removed, for example by substitution or deletion mutation, or by mating protocols). In some embodiments, an engineered cell is one that has been manipulated so that it contains and/or expresses a particular agent of interest (e.g., a protein, a nucleic acid. and/or a particular form thereof) in an altered amount and/or according to altered timing relative to such an appropriate reference cell. As is common practice and is understood by those in the art, progeny of an engineered polynucleotide or cell are typically still referred to as “engineered” even though the actual manipulation was performed on a prior entity. Other examples of the use of the term “synthetic” include synthetic nucleases. Synthetic nucleases are nucleases that additionally include a domain that directly, or indirectly, associates with a co-location sequence.

As used herein, the term “expression” of a nucleic acid sequence refers to the generation of any gene product from the nucleic acid sequence. In some embodiments, a gene product can be a transcript. In some embodiments, a gene product can be a polypeptide. In some embodiments, expression of a nucleic acid sequence involves one or more of the following: (1) production of an RNA template from a DNA sequence (e.g., by transcription); (2) processing of an RNA transcript (e.g., by splicing, editing, etc.); (3) translation of an RNA into a polypeptide or protein; and/or (4) post-translational modification of a polypeptide or protein.

As used herein, the term “identity” refers to overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of percent identity of two nucleic acid or polypeptide sequences, for example, can be performed by aligning two sequences for optimal comparison purposes (e.g., gaps can be introduced in one or both of a first and a second sequences for optimal alignment and non-identical sequences can be disregarded for comparison purposes). In some embodiments, a length of a sequence aligned for comparison purposes is at least 30%, at least 40%, at least 50%, at least 60%, at least 70%, at least 80%, at least 90%, at least 95%, or substantially 100% of length of a reference sequence; residues at corresponding positions are then compared. When a position in the first sequence is occupied by the same residue (e.g., nucleotide or amino acid) as a corresponding position in the second sequence, then the two molecules (i.e., first and second) are identical at that position. Percent identity between two sequences is a function of the number of identical positions shared by the two sequences being compared, taking into account the number of gaps, and the length of each gap, which needs to be introduced for optimal alignment of the two sequences. Comparison of sequences and determination of percent identity between two sequences can be accomplished using a mathematical algorithm. For example, percent identity between two nucleotide sequences can be determined using the algorithm of Meyers and Miller (CABIOS, 1989, 4: 11-17, which is herein incorporated by reference in its entirety), which has been incorporated into the ALIGN program (version 2.0). In some embodiments, nucleic acid sequence comparisons made with the ALIGN program use a PAM120 weight residue table, a gap length penalty of 12 and a gap penalty of 4.

“Introduced” refers to the movement of a nucleic acid sequence or protein into a cell through infection of a phage (transduction), conjugation, transformation with a molecular biology technique, such as electroporation or heat shock of chemically competent cells.

“Linkers” are short amino acid sequences that function to link the synthetic binding domain to nucleases directly, or to link a DNA binding domain to another amino acid sequence that can associate with the synthetic binding domain in a nuclease.

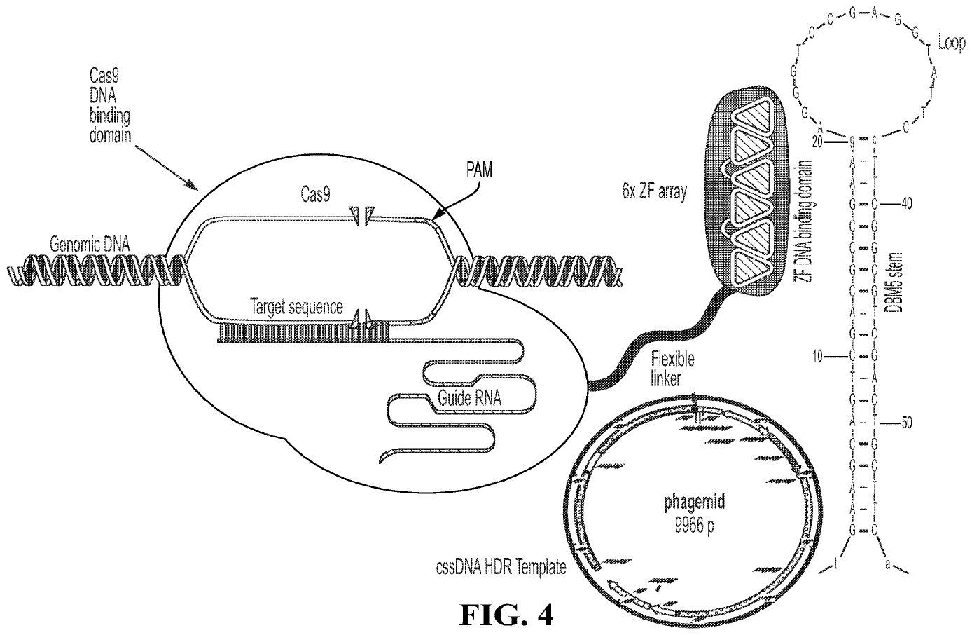

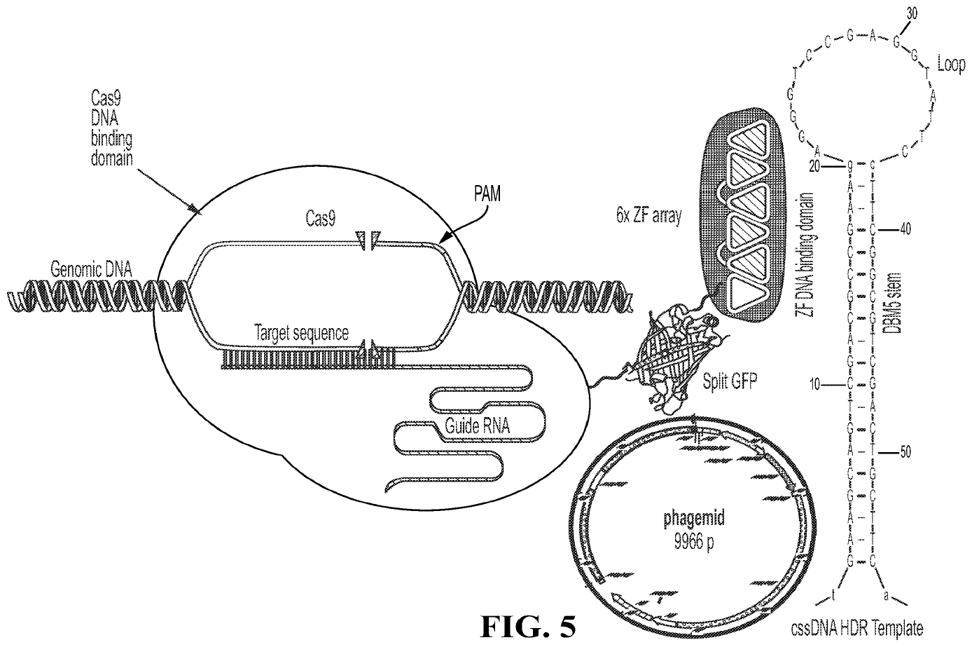

“Non-covalent linkages”, “dimers”, or “multimers” refers to associations of two or more molecules other than through a covalent bond. As previously described, a synthetic nuclease can bind to a co-location sequence through the use of a synthetic DNA binding domain that is engineered to be included with the nuclease coding sequence. The co-location sequence can also associate with a synthetic nuclease through non-covalent associations. In such instances that co-location sequence can be recognized by a DNA binding domain that is additionally associated with an amino acid sequence capable of forming a multimer with at least one additional amino acid sequence. At least one additional sequence can be part of a synthetic nuclease. See FIG. 4 and FIG. 5. These dimerization domains (the one recognizing the co-location sequence and the one that is associated with the synthetic nuclease) might constitutively interact with one another (e.g., leucine zipper motifs (Feuerstein et al, Proc. Natl. Acad. Sci. USA 91: 10655-10659, 1994), Fc domains) or might interact only in the presence of an effector such as a small molecule or stimulation by light. A number of dimerization domains are known in the art, e.g., cysteines that are capable of forming an intermolecular disulfide bond with a cysteine on the partner fusion protein, a coiled-coil domain, an acid patch, a zinc finger domain, a calcium hand domain, a CHI region, a CL region, a leucine zipper domain, an SH2 (src homology 2) domain, an SH3 (src Homology 3) domain, a PTB (phosphotyrosine binding) domain, a WW domain, a PDZ domain, a 14-3-3 domain, a WD40 domain, an EH domain, a Lim domain, an isoleucine zipper domain, and a dimerization domain of a receptor dimer pair (see, e.g., US20140170141; US20130259806; US20130253040; and US20120178647). Suitable dimerization domains can be selected from any protein that is known to exist as a multimer or dimer, or any protein known to possess such multimerization or dimerization activity. Examples of suitable domains include the dimerization element of Gal4, leucine zipper domains, STAT protein N-terminal domains, FK506 binding proteins, and randomized peptides selected for Zf dimerization activity (see, e.g., Bryan et al., 1999, Proc. Natl. Acad. Sci. USA, 96:9568; Pomerantz et al, 1998, Biochemistry, 37:965-970; Wolfe et al, 2000, Structure, 8: 739-750; O'Shea, 1991, Science, 254:539; Barahmand-Pour et al, 1996, Curr. Top. Microbiol. Immunol, 211: 121-128; Klemm et al, 1998, Annu Rev. Immunol, 16:569-592; Ho et al, 1996, Nature, 382:822-826). Furthermore, some zinc finger proteins themselves have dimerization activity. For example, the zinc fingers from the transcription factor Ikaros have dimerization activity (McCarty et al, 2003, Mol. Cell, 11:459-470). Thus, if the engineered Zf proteins themselves have dimerization function there will be no need to fuse an additional dimerization domain to these proteins. In some embodiments the endonuclease domain itself possesses dimerization activity. For example, the nuclease domain of Fok I, which has intrinsic dimerization activity, can be used (Kim et al, 1996, Proc. Natl. Acad. Sci., 93: 1156-60). In some embodiments, “conditional” dimerization technology can be used. For example, this can be accomplished using FK506 and FKBP interactions. FK506 binding domains are attached to the proteins to be dimerized. These proteins will remain separate in the absence of a dimerizer. Upon addition of a dimerizer, such as the synthetic ligand FK1012, the two proteins will fuse.

The term “nuclease” refers to any peptide that functions to separate two nucleotides that are adjacent to each other in a nucleic acid sequence from each other. The result of such separation is a break in either a ssDNA polymer or a dsDNA polymer. Nucleases can be described as exonucleases or endonucleases depending upon if they separate the nucleotide molecule from a terminal end of the polymer (exonuclease) or separate the nucleotide molecule from within the polymer (endonuclease). One of ordinary skill in the art will appreciate that some nucleases are site specific, meaning that they facilitate a break in a nucleic acid sequence at a specific cite associated with a specific sequence in the nucleic acid polymer. Restriction enzymes are an example of such a nucleases. Other nucleases include CAS related proteins that function to separate two nucleotides in a nucleic acid polymer at a specific location in the polymer through the use of a ribonucleic acid protein complex. The RNA associated with such CAS proteins is referred to as the guide RNA (gRNA). Other exemplary nucleases include zinc finger nuclease (ZFN), a meganuclease, a homing endonuclease, and a transcription factor like effector nuclease (TALEN).

The terms “nucleic acid”, “polynucleotide”, and “oligonucleotide” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones, locked nucleic acid). In general and unless otherwise specified, an analogue of a particular nucleotide has the same base-pairing specificity; i.e., an analogue of adenine will base-pair with thymine. When double-stranded DNA is described, the DNA can be described according to the conformation adopted by the helical DNA, as either A-DNA, B-DNA, or Z-DNA. The B-DNA described by James Watson and Francis Crick is believed to predominate in cells, and extends about 34 Å per 10 bp of sequence; A-DNA extends about 23 Å per 10 bp of sequence, and Z-DNA extends about 38 Å per 10 bp of sequence.

In some cases nucleotide sequences are provided using character representations recommended by the International Union of Pure and Applied Chemistry (IUPAC) or a subset thereof. IUPAC nucleotide codes used herein include, A=Adenine, C=Cytosine, G=Guanine, T=Thymine, U=Uracil, R=A or G, Y=C or T, S=G or C, W=A or T, K=G or T, M=A or C, B=C or G or T, D=A or G or T, H=A or C or T, V=A or C or G, N=any base, “.” or “-”=gap. In some embodiments the set of characters is (A, C, G, T, U) for adenosine, cytidine, guanosine, thymidine, and uridine respectively.

Nucleotide refers to a molecule that contains a base moiety, a sugar moiety and a phosphate moiety. Nucleotides can be linked together through their phosphate moieties and sugar moieties creating an inter-nucleotide linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate). There are many varieties of these types of molecules available in the art and available herein.

“Oligonucleotide or a polynucleotide” refer to synthetic or isolated nucleic acid polymers including a plurality of nucleotide subunits.

“Operably linked” as used herein, refers to the position of two or more components wherein the components described are in a relationship permitting them to function in their intended manner. A control element “operably linked” to a functional element is associated in such a way that expression and/or activity of the functional element is achieved under conditions compatible with the control element. In some embodiments, “operably linked” control elements are contiguous (e.g., covalently linked) with the coding elements of interest; in some embodiments, control elements act in trans to or otherwise at a from the functional element of interest.

A “packaging signal” as used herein is any nucleic acid sequence that can trigger the formation of the phage coat. The phage coat includes one or more phage proteins that surround the cssDNA during cssDNA production.

A “phagemid” as used herein comprises a bacteriophage origin of replication, a selection sequence, a designed sequence, and a packaging signal. In some embodiments a phagemid can also include a plasmid origin of replication and/or an antibiotic resistance marker. The sequence of the phagemid will not include all of the phage protein coding sequences so that a cell, upon infection by the phagemid, will not produce additional phage particles, unless the cell additionally includes coding sequences for the necessary phage proteins. The cell can include a helper virus or a plasmid that encodes some or all of the phage proteins necessary for phage replication and packaging.

A “phage particle” as used herein refers to a phagemid sequence that is coated with phage proteins, but yet does not include all of the coding sequences necessary for producing phage proteins.

The term “phage proteins” as used herein refer to the proteins that are encoded by a native bacteriophage that are needed for phage replication in a host. Useful phage proteins include those from the realm of Monodnaviria that includes phages Ff, Fd, F1 and M13 among others. Phage proteins include phage proteins that have nucleic acid sequence modifications that can lead to changes in the amino acid sequence as compared to the native sequence. Such modified phage proteins shall maintain substantially similar functionality as compared to the native phage protein sequence, but the sequence identity shared can be greater than 50%, 60%, 70%, 75%, 80%, 90% or 95%. For example, a phage protein that additionally includes a tag sequence, the phage protein will still be capable of performing its native function to at least some degree. For example, a phage protein comprising a tag may have looser binding to other phage proteins when a tag is included, but yet it is still considered a phage protein. Phage proteins that are specifically described herein include the M13 phage proteins and nucleic acid sequences provided in the sequence listing. The proteins are referred to as p1, p2, p3, p4, p5, p6, p7, p8, p9, p10 and p11 and the genes are referred to using the respective roman numerals (I, II, III, IV, V, VI, VII, VIII, IX, X and XI).

“Pharmaceutical formulations” as used herein refer to any acceptable pharmaceutical delivery system that can be used to deliver a synthetic nuclease in peptide form, a designed sequence and/or a nucleic acid sequence encoding a synthetic nuclease to a target cell. Pharmaceutical formulations can include excipients such as relatively inert substances that facilitate administration of a synthetic nuclease and/or a designed sequence and can be supplied as liquid solutions or suspensions, as emulsions, or as solid forms suitable for dissolution or suspension in liquid prior to use. For example, an excipient can give form or consistency, or act as a diluent. Suitable excipients include but are not limited to stabilizing agents, wetting and emulsifying agents, salts for varying osmolarity, encapsulating agents, pH buffering substances, and buffers. Pharmaceutically acceptable excipients include, but are not limited to, sorbitol, any of the various TWEEN compounds, and liquids such as water, saline, glycerol and ethanol. Pharmaceutically acceptable salts can be included therein. for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients is available in REMINGTON'S PHARMACEUTICAL SCIENCES (Mack Pub. Co., N.J. 1991). Particularly useful pharmaceutically acceptable excipients include lipid nanoparticles, nanostructured lipid carriers, solid lipid nanoparticles, liposomes, including cationic or anionic liposomes and liposomes that include various forms of polyethylene glycol and other liposome modification known to increase bioavailability of proteins and nucleic acids. Tenchov, R., Bird, R., Curtze, A. E., & Zhou, Q. (2021). Lipid nanoparticles—from liposomes to mRNA vaccine delivery, a landscape of research diversity and advancement. ACS nano, 15(11), 16982-17015.

The term “production strain” as used herein refers to a bacterial cell that can be used to produce the cssDNA. Production strains can include helper viruses or helper plasmids that can encode for phage proteins necessary for phage replication and packaging. In some embodiments a production strain can include at least two phage proteins integrated into the genome of the production strain. The at least two phage proteins can be differentially expressed as compared to when the phage proteins are expressed from their native control sequences, for example the native control sequences found in the wild type M13 phage. For example, one or more of the at least two sequences can be under the control of an inducible promoter so that expression is not triggered by the same factors as compared to when the expression is controlled by the native control sequences. In some embodiments the production strain is a strain of E. coli, for example the non-toxic E. coli described in U.S. Pat. No. 8,303,964 (which is herein incorporated by reference), which is additionally engineered to reduce the presence of the 3-deoxy-d-manno-oct-2-ulosonic acid (Kdo) component of the cell capsule.

A “promoter” is a control sequence that includes a minimal sequence sufficient to direct transcription. Also included are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters are included (see for example, Bitter et al., Methods in Enzymology 153:516-544, 1987). For example, when cloning in bacterial systems, inducible promoters such as pL of bacteriophage lambda, plac, ptrp, ptac (ptrp-lac hybrid promoter) and the like may be used.

As used herein, the term “reference” describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

“Selectable sequences” refers to sequences of nucleic acids or amino acids that upon inclusion in the phagemid sequence allow the phagemid to be retained within the production cell population. Selectable sequences include for example antibiotic resistance sequences, engineered tRNA sequences, auxotrophic markers, antitoxin genes, or sequences that alter transcription or translation. Examples of selectable sequences include, without limitation, genes encoding proteins that increase or decrease either resistance or sensitivity to antibiotics (e.g., ampicillin resistance genes, kanamycin resistance genes, neomycin resistance genes, tetracycline resistance genes, chloramphenicol resistance genes, and spectinomycin resistance genes) or other compounds. Additional examples of selectable markers include, without limitation, genes encoding proteins that enable the cell to grow in media deficient in an otherwise essential nutrient such as uracil, leucine, adenine, histidine, arginine, lysine, tryptophan, methionine or other essential metabolites.

The term “source” as used herein, typically refers to a context in which an agent of interest (e.g., that may be or comprise a carbohydrate, a lipid, a nucleic acid, a metal. polypeptide, a small molecule, or a combination thereof) may be found in nature, or from which such agent can be or has been obtained (e.g., isolated). In some embodiments, a source may be or comprise a biological source (e.g., an organism, tissue, or cell, or sample thereof); in some embodiments a source may be an environmental source. In some embodiments, a source may be or comprise a primary sample from an organism (e.g., which may be or comprise a tissue or fluid of such organism, and/or may be or comprise cell(s) of such organism). In some embodiments. an organism may be or comprise a prokaryotic organism (e.g., a bacterium) or a eukaryotic organism (e.g., a fungus or yeast. an insect, a mammal, a plant, a reptile. etc.). In some embodiments, an infectious agent such as a virus or phage may be considered an organism for purposes of this disclosure, and in particular with respect to being a source. In some embodiments, a source may be or comprise an engineered source, such as a cell line or culture, an in vitro system, etc.

“Synthetic binding domain” as used herein refers to an amino acid sequence that can associate with a single stranded nucleic acid sequence, a double stranded nucleic acid sequence, or another amino acid sequence through protein-protein interactions. Synthetic binding domains include any amino acid sequence that can function to associate the co-location sequence with a synthetic nuclease. The use of the word synthetic in the phrase synthetic binding domain refers to the engineering of the synthetic binding domain into the amino acid sequence of the synthetic nuclease. One of ordinary skill in the art will appreciate that synthetic binding domains can be created through molecular evolution designed to preserve amino acid sequences that bind to a co-location sequence and selectively discarding amino acid sequences that do not bind. Selective binding sequences can also be chosen based on already known binding domains within already characterized amino acid sequences. Such already known binding domains can be cloned into an expression cassette that encodes a synthetic nuclease. Synthetic binding domains that bind to DNA can be described as synthetic DNA binding domains.

“Synthetic nuclease” as used herein refers to nucleases that have been engineered to include a synthetic binding domain that functions to associate the nuclease with a co-location sequence. For additional information refer to the definition of nuclease provided herein.

“Target cells” or “host cells” as used herein refer to cells into which the designed sequence will be introduced by the synthetic nuclease. Target cells can be prokaryotic or eukaryotic cells. Eukaryotic cells can be selected from mammalian cells, yeast, fungi algal, and plant cells. One of ordinary skill in the art will appreciate that many synthetic biology applications require the use of yeast (eukaryotic) or bacterial (prokaryotic) cells that are engineered. However, many of the strains that are useful in synthetic biology are not easily engineered, therefore, the synthetic nuclease described herein and the cssDNA described herein are desirable for such applications. In some examples, target cells can be mammalian cells including mammalian cells that are extracted from an organism and cultured. After the cells are engineered they can be reintroduced into a body or into a tissue. Target cells can also be within a body and the synthetic nuclease and designed sequence can be introduced through intravenous treatment, injection, or the like. In instances where the target cells are located in a body a formulation including for example, liposomes, buffers and other excipients can be used to facilitate delivery. Transformation of the synthetic nuclease and designed sequence into a target cell can be through any means known to one or ordinary skill in the art. One of ordinary skill in the art will appreciate that there are a variety of pharmaceutically acceptable formulations that can be used.

“Target dsDNA” as used herein refers to a dsDNA sequence that will be altered by a designed sequence. One of ordinary skill in the art will appreciate that depending upon the designed sequence the target dsDNA can be altered to contain a deletion or insertion of dsDNA. To form a deletion as compared to the original target dsDNA, the designed sequence can be engineered to include homology arms and an intervening sequence between the homology arms that is shorter than the original target dsDNA. In instances where the designed sequence is intended to replace one or more nucleic acids in the target dsDNA sequence the designed sequence can be engineered to change the nucleic acid sequence, but yet not alter the overall length of the target dsDNA. In some embodiments, the designed sequence will include an engineered sequence that increases the overall length of the target dsDNA.

“Template cssDNA” refers to circular single stranded DNA. In some embodiments, a template cssDNA may include one or more elements such as a packaging sequence, designed sequence, and a phage ori. In some embodiments, upon introduction into a bacterial cell, such as an E. coli cell, a template cssDNA is replicated into a replicative form using the bacterial cell proteins and nucleic acid sequences, and then is packaged into phage particles.

“Variant” refers to an entity that shows significant structural identity with a reference entity but differs structurally from the reference entity in the presence or level of one or more chemical moieties as compared with the reference entity. In many embodiments, a variant also differs functionally from its reference entity. In general, whether a particular entity is properly considered to be a “variant” of a reference entity is based on its degree of structural identity with the reference entity. As will be appreciated by those skilled in the art, any biological or chemical reference entity has certain characteristic structural elements. A variant. by definition, is a distinct chemical entity that shares one or more such characteristic structural elements. To give but a few examples, a small molecule may have a characteristic core structural element (e.g., a macrocycle core) and/or one or more characteristic pendent moieties so that a variant of the small molecule is one that shares the core structural element and the characteristic pendent moieties but differs in other pendent moieties and/or in types of bonds present (single vs double, E vs Z, etc.) within the core, a polypeptide may have a characteristic sequence element comprised of a plurality of amino acids having designated positions relative to one another in linear or three-dimensional space and/or contributing to a particular biological function, a nucleic acid may have a characteristic sequence element comprised of a plurality of nucleotide residues having designated positions relative to on another in linear or three-dimensional space. For example, a variant polypeptide may differ from a reference polypeptide as a result of one or more differences in amino acid sequence and/or one or more differences in chemical moieties (e.g., carbohydrates, lipids. etc.) covalently attached to the polypeptide backbone. In some embodiments, a variant polypeptide shows an overall sequence identity with a reference polypeptide that is at least 85%, 86%, 87%, 88%, 89%, 90%, 91%, 92%, 93%, 94%, 95%, 96%, 97%, or 99%, optionally other than conservative amino acid substitutions. Alternatively or additionally, in some embodiments, a variant polypeptide does not share at least one characteristic sequence element with a reference polypeptide. In some embodiments, the reference polypeptide has one or more biological activities. In some embodiments, a variant polypeptide shares one or more of the biological activities of the reference polypeptide. In some embodiments, a variant polypeptide lacks one or more of the biological activities of the reference polypeptide. In some embodiments, a variant polypeptide shows a reduced level of one or more biological activities as compared with the reference polypeptide. In many embodiments, a polypeptide of interest is considered to be a “variant” of a parent or reference polypeptide if the polypeptide of interest has an amino acid sequence that is identical to that of the parent but for a small number of sequence alterations at particular positions. Typically, fewer than 20%, 15%, 10%, 9%, 8%, 7%, 6%, 5%, 4%, 3%, 2% of the residues in a variant are substituted as compared with the parent. In some embodiments, a variant has 10, 9, 8, 7, 6, 5, 4, 3, 2, or 1 substituted residue(s) as compared with a parent. Often, a variant has a very small number (e.g., fewer than 5, 4, 3, 2, or 1) number of substituted functional residues (i.e., residues that participate in a particular biological activity). Furthermore, a variant typically has not more than 5, 4, 3, 2, or 1 additions or deletions, and often has no additions or deletions, as compared with the parent. Moreover, any additions or deletions are typically fewer than about 25, about 20, about 19, about 18, about 17, about 16, about 15, about 14, about 13, about 10, about 9, about 8, about 7, about 6, and commonly are fewer than about 5, about 4, about 3, or about 2 residues. In some embodiments, a parent or reference polypeptide is one found in nature.

Making Synthetic Nucleases and cssDNA

The discovery of nucleases has transformed biology enabling the precise manipulation of DNA in eukaryotes and prokaryotes. More recently, the discovery of the CRISPR/Cas bacterial immune system and its application to gene and cell therapy as well as microbial and mammalian genome engineering for synthetic biology applications has made new areas of research, therapy and biotechnology easily accessible. The CRISPR/Cas system has proven highly efficient at introducing double-strand DNA breaks at genomic loci specified by the gRNA, routinely achieving 50-100% knockout efficiency in host organisms across multiple kingdoms.

The system struggles, however, to achieve the same rates of homology directed repair knock-ins especially in mammalian cells. Typical knock-in rates in mammalian cells are 0.1-10% with rates above 10% considered exceptional. The discrepancy between the CRISPR/Cas cutting efficiency and the homology directed repair frequency is caused by multiple factors.

The compositions and the methods described herein overcome many of the problems associated with targeting genomic edits in both eukaryotic and prokaryotic cells.

### EXEMPLARY NUMBERED EMBODIMENTS

Embodiment 1. A composition comprising cssDNA molecule, wherein the cssDNA comprises a co-location sequence wherein the co-location sequence is capable of associating with a synthetic nuclease, a selectable sequence, a packaging signal, and a designed sequence.

Embodiment 2. A composition comprising
a cell comprising
a synthetic nuclease wherein the synthetic nuclease comprises a synthetic binding domain, wherein the synthetic binding domain is capable of associating with a co-location sequence, and
a cssDNA molecule, wherein the cssDNA comprises the co-location sequence.

Embodiment 3. The composition according to embodiment 2, wherein the cell is a eukaryotic cell.

Embodiment 4. The composition according to embodiment 3, wherein the cell is a mammalian cell.

Embodiment 5. The composition according to embodiment 3, wherein the cell is selected from a yeast cell or a fungal cell.

Embodiment 6. The composition according to embodiment 2, wherein the cell is a bacterial cell.

Embodiment 7. The composition according to embodiment 2, wherein the synthetic binding domain comprises a DNA binding domain.

Embodiment 8. The composition according to embodiment 2, wherein the synthetic binding domain comprises an amino acid sequence capable of forming a protein protein interaction with a second amino acid sequence.

Embodiment 9. The composition according to embodiment 8, wherein the second amino acid sequence additionally comprises a DNA binding domain.

Embodiment 10. The composition according to embodiment 9, wherein the DNA binding domain binds to a co-location sequence.

Embodiment 11. The composition according to embodiment 2, wherein the cssDNA molecule does not contain a packaging signal.

Embodiment 12. The composition according to embodiment 2, wherein the cssDNA molecule additionally comprises a selectable sequence.

Embodiment 13. The composition according to embodiment 2, wherein the cssDNA molecule additionally comprises a designed sequence.

Embodiment 14. The composition according to embodiment 2, wherein the synthetic binding domain comprises a dsDNA binding domain.

Embodiment 15. The composition according to embodiment 2, wherein the synthetic binding domain comprises an amino acid sequence capable of forming a multimeric complex with at least one additional amino acid sequence.

Embodiment 16. The composition according to embodiment 1, further comprising a synthetic nuclease comprising a DNA binding domain.

Embodiment 17. The composition of embodiment 1 or 2, wherein the synthetic nuclease is selected from the group consisting of: a Cas protein, a zinc finger nuclease (ZFN), a meganuclease, a homing endonuclease, a transcription factor like effector nuclease (TALEN) or another nuclease capable of creating double- or single-strand DNA breaks.

Embodiment 18. The composition of embodiment 1 or 2, wherein the cssDNA additionally comprises at least one region of homology, wherein the region of homology targets a region in the genome of a cell.

Embodiment 19. The composition according to embodiment 2, further comprising a gRNA.

Embodiment 20. The composition of embodiment 1 or 13, wherein the designed sequence comprises a therapeutic sequence.

Embodiment 21. The composition according to embodiment 20, wherein the therapeutic sequence functions to alter the genome of a mammalian cell.

Embodiment 22. The composition according to embodiment 21, wherein the therapeutic sequence comprises a control sequence.

Embodiment 23. The composition according to embodiment 22, wherein the control sequence is selected from the group consisting of: introns, promoters, DNA binding sites, RNA binding sites, repressor binding sites, enhancer binding sites, transcription modifiers, and translation modifiers.

Embodiment 24. The composition according to embodiment 20, wherein the therapeutic sequence alters a coding sequence in a gene.

Embodiment 25. The composition according to embodiment 20, wherein the therapeutic sequence is greater than 3 kb.

Embodiment 26. The composition of embodiment 2, wherein the synthetic binding domain is connected to the synthetic nuclease with a linker.

Embodiment 27. The composition of any one of embodiments 2-26, wherein the synthetic binding domain is a dsDNA binding domain.

Embodiment 28. The composition according to embodiment 27, wherein the synthetic binding domain is derived from an amino acid sequence selected from the group consisting of: a Cas protein, a zinc finger nuclease (ZFN), a meganuclease, a homing endonuclease, a transcription factor like effector nuclease (TALEN); and a restriction endonuclease.

Embodiment 29. The composition according to embodiment 2, wherein the synthetic binding domain associated with the nuclease comprises a first amino acid sequence, wherein the first amino acid sequence forms a dimer with a second amino acid sequence, wherein the second amino acid sequence binds to the co-location sequence.

Embodiment 30. A method of engineering a target dsDNA in a cell comprising: contacting a target dsDNA with a synthetic nuclease and a cssDNA comprising a co-location sequence; and culturing the cell.

Embodiment 31. A method of making a kit for editing dsDNA, comprising: selecting a cssDNA comprising a co-location sequence; selecting an synthetic nuclease wherein the synthetic nuclease comprises a synthetic binding domain and wherein the synthetic binding domain associates with the co-location sequence; and assembling the cssDNA and synthetic nuclease into a kit.

Embodiment 32. A kit made by the method according to embodiment 31.

### EXAMPLES

### Example 1: Making Nuclease-DNA-Binding Domain Fusion Proteins

A nucleic acid sequence encoding nuclease-DNA-binding domain fusion protein was (cdsDNA188; as shown in SEQ ID NO: 6) synthesized, cloned and expressed as follows.

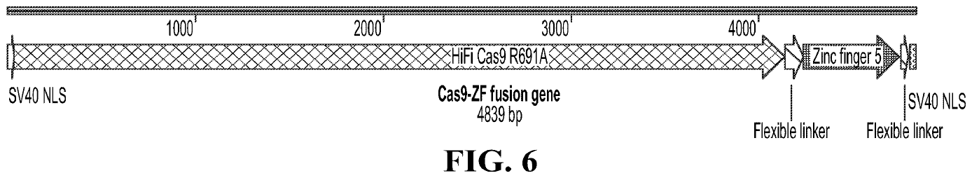

The in vitro expression cassette was codon optimized, synthesized as DNA and cloned into a plasmid backbone by Genscript (Jiangsu Province, P.R. China). The in vitro expression cassette consists of the SV40 nuclear localization signal and the R691A high fidelity mutant of Cas9. R691A high fidelity (HiFi) Cas9 was chosen as the nuclease due its low off-target effects. This mutant Cas9 demonstrates superior on-target editing with RNP delivery. Vakulskas et al., a high-fidelity Cas9 mutant delivered as a ribonucleoprotein complex enables efficient gene editing in human hematopoietic stem and progenitor cells. Nat Med. 2018 August; 24(8):1216-1224. The cassette also contains the flexible linker sequence that was used to link Cas9 to cytosine and adenine deaminases to form base editors in Levy et al., Cytosine and adenine base editing of the brain, liver, retina, heart and skeletal muscle of mice via adeno-associated viruses. Nat Biomed Eng. 2020 January; 4(1):97-110. The sequence of the linker is SGGSSGGSSGSETPGTSESATPESSGGSSGGS (SEQ ID NO: 20), but those skilled in the art will be aware that there are many possible flexible linker sequences that could have been chosen and likely would have worked just as well. For example, Chen et al. wrote a review article in 2013 describing many possible variants of linker sequences. Chen et al., Fusion protein linkers: property, design and functionality. Adv Drug Deliv Rev. 2013 October; 65(10):1357-69. iGem lists dozens of linker sequences in their BioBricks database. http://parts.igem.org/Protein_domains/Linker. The Centre for Integrative Bioinformatics at the University of Amsterdam lists over 1200 linker sequences in their database https://www.ibi.vu.nl/programs/linkerdbwww/. Flexible linkers are found commonly in natural proteins and synthetic proteins alike. They generally consist of short glycine and serine rich sequences in between two active domains. The flexibility allows each domain to function independently to the function of the other domain. The particular flexible linker chosen in this instance, connects Cas9 to zinc finger 5 (SEQ ID NO: 22) which is a fusion of 6 zinc fingers that recognizes an 18 bp core sequence. Israni et al., Clinically-driven design of synthetic gene regulatory programs in human cells bioRxiv 2021.02.22.432371. There is an additional flexible linker sequence (SEQ ID NO: 21) Chen et al., Fusion Protein Linkers: Property, Design and Functionality, Adv Drug Deliv Rev. 2013 October 15; 65(10): 1357-1369, that links the zinc finger to another SV40 nuclear localization sequence (SEQ ID NO: 31) and finally a stop codon. A his tag can be included upstream of the stop codon to aid in purification (e.g., SEQ ID NO: 40). This open reading frame (as shown in FIG. 6 and shown in SEQ ID NO: 1) was cloned into a standard E. coli expression plasmid by Genscript and expressed in an E. coli culture. The E. coli cells were then lysed and the protein was purified using standard affinity tag methods. An additional protein construct consisting of HiFi SpCas9 with two nuclear localization sequences (IDT catalog number 1081060) was also purchased as a control from Integrated DNA Technologies (Coralville, Iowa). That construct was made as described above except that the zinc finger 5 sequence is not included (zinc finger negative control).

In addition to these two proteins described above, HiFi SpCas9 with and without a zinc finger fusion were also cloned into mammalian expression vectors and accessioned as cdsDNA166 (SEQ ID NO: 8) for HiFi SpCas9 without a zinc finger fusion and as cdsDNA177 (SEQ ID NO: 7) for HiFi SpCas9 fused to the zinc finger.

### Example 2: Making Circular Single Stranded DNA that Binds Nuclease-DNA-Binding Domain Fusion Proteins

A phagemid (cdsDNA193; SEQ ID NO: 5) was cloned to produce circular single stranded DNA (cssDNA) that interacts with the Cas9-zinc finger fusion protein described in Example 1 above, and integrates into the human genome, in this example, at the TRAC locus. The phagemid contains a co-location sequence that is designed to be bound by zinc finger 5 (ZF5) described above. The co-location sequence consists of a DNA binding domain referred to as DNA binding motif 5 (DBM5) and contains a core, 18-bp binding motif (SEQ ID NO: 46 GAAGCAGTCGACGCCGAA) flanked by 1 bp on either end that are also necessary to maximize binding of ZF5 to DBM5 (SEQ ID NO: 47). Israni et al., Clinically-driven design of synthetic gene regulatory programs in human cells bioRxiv 2021.02.22.432371. Because the phagemid is produced as circular, single-stranded DNA (cssDNA) by the E. coli strain expressing the phage genes, the cssDNA must also contain the reverse complement of DBM5 and a few non-complementary bases so that DBM5 and its reverse complement can fold over on each other to form a stem and loop secondary structure creating a short, 20 bp sequence of double-stranded DNA within the long, 200-30,000 nt sequence of the cssDNA phagemid. Here the stem is formed by DBM5 (SEQ ID NO: 47) and its reverse complement (SEQ ID NO: 48) while the loop sequence (SEQ ID NO: 24) is taken from Varkonyi-Gasic et al., Quantitative stem-loop RT-PCR for detection of microRNAs, April 2011, Methods in molecular biology (Clifton, N.J.) 744:145-57.

Another phagemid (cdsDNA136; SEQ ID NO: 10) was also cloned as a control. It is identical to cdsDNA193 described above except that it lacks the ZFDBM5 stem and loop sequence.

Two more phagemids cdsDNA222 and cdsDNA221 were also cloned. They are identical to cdsDNA193 and cdsDNA136 described above except that they lack the 16 nt Cas9 Target Sequence that is included on cdsDNA193 and cdsDNA136. The Cas9 Target sequence (CTS; SEQ ID NO: 49) was designed to create an interaction between a Cas9 ribonucleoprotein and the cssDNA based on base pairing with the gRNA. Removing the Cas9 Target Sequences from the phagemids allowed for differentiation between interactions between the Cas9 ribonucleoprotein and the cssDNA that are caused by the Cas9 Target Sequence and those that are caused by the ZFDBM5.

The phagemid backbone consists of a plasmid origin of replication so that it could be easily cloned, transformed into bacteria and replicated as dsDNA, a phage origin of replication and packaging signal so that it can be replicated as cssDNA and packaged into a phage capsid and a selectable marker so that it can be maintained in a population of bacteria. In this example, the plasmid origin of replication was the high copy number pUC origin Saha; et al. (2004). “A naturally occurring point mutation in the 13-mer R repeat affects the oriC function of the large chromosome of Vibrio cholerae O1 classical biotype”. Archives of Microbiology. 182 (5): 421-427, which is herein incorporated by reference. The phage origin of replication and packaging signal were from M13KO7 Vieira et al., Production of single-stranded plasmid DNA. Methods Enzymol. 1987; 153:3-11, which is herein incorporated by reference. In some cases, the pUC origin of replication may include the inc1 and inc2 high copy mutations described in Tomizawa J, Som T. 1984. Control of ColE1 plasmid replication—enhancement of binding of RNA-4 to the primer transcript by the Rom protein. Cell 38:871-878.

In this case the selection marker was the auxotrophic marker is the ampicillin resistance gene.

The phagemid also contained a designed sequence. In this case, the sequence is a pair of homology arms targeting the homology directed repair event to the human T-cell receptor α constant (TRAC) locus, which is relevant for CAR-T cell therapy. The pair of homology arms are shown in SEQ ID NOs: 27 and 28; Eyquem et al., Targeting a CAR to the TRAC locus with CRISPR/Cas9 enhances tumor rejection. Nature. 2017 March 2:543(7643):113-117, which is herein incorporated by reference. The homology arms flank a green fluorescent protein reporter cassette. Expression of the the reporter is driven by the human GRP78 promoter (SEQ ID NO: 38). The reporter is emerald GFP (SEQ ID NO: 34) which is a constitutively fluorescent dimer derived from Aequorea victoria Cubitt A B, Woollenweber L A, Heim R. Understanding structure-function relationships in the Aequorea victoria green fluorescent protein. Methods Cell Biol. 1999; 58:19-30. The polyA signal is from the sequence downstream of the mouse beta globin gene (SEQ ID NO: 39).

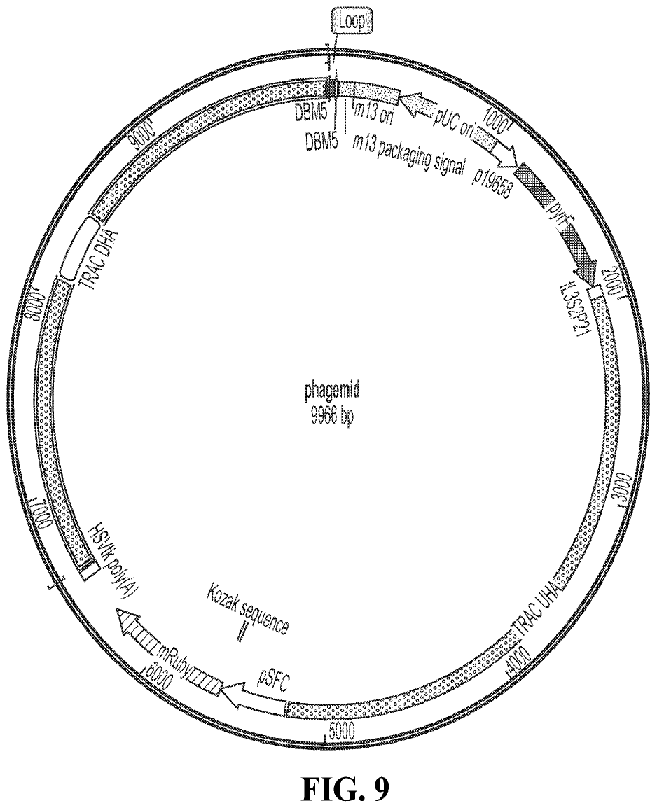

The phagemid (shown in FIG. 9 and SEQ ID NO: 2) was co-transformed into a DH5alpha E. coli host (New England Biolabs C2987U along with a helper plasmid encoding the entire M13 phage genome along with a p15A origin of replication and a kanamycin resistance marker (cdsDNA114; SEQ ID NO: 11). The presence of the helper plasmid makes the E. coli host capable of replicating and packaging cssDNA into a phage capsid. The transformation was accomplished using a standard chemical transformation method https://www.neb.com/en-us/protocols/0001/01/01/high-efficiency-transformation-protocol-c2987. In short, the cells were incubated on ice in the presence of the two plasmids for 5 minutes then heat shocked for 30 seconds at 42 C. The cells were allowed to recover in SOC medium for 1 hour at 37 C with shaking before they are plated on LB agar supplemented with 100 μg/ml carbenicillin and 50 μg/ml kanamycin. Plates were incubated overnight at 37 C. Single colonies were picked and inoculated into 4 ml 2×YT supplemented with 100 μg/ml carbenicillin and 50 μg/ml kanamycin and grown overnight at 30 C. In the morning, the optical density (OD) is measured and the culture was diluted to OD 0.05 in 750 ml 2×YT supplemented with 100 μg/ml carbenicillin and 50 μg/ml kanamycin. This culture was then incubated for 16 hours at 30 C.

The culture was centrifuged to remove whole cells and the phage particles were collected in the supernatant. Phage particles were purified from the fermentation medium by polyethylene glycol and sodium chloride precipitation (3% weight by volume each) followed by centrifugation at 5000 g. The phage particle pellet was then lysed using a solution of 1% sodium dodecyl sulfate and 200 mM sodium hydroxide. The lysed phage particle debris was pelleted by centrifugation at 12,000 g and the supernatant containing the cssDNA was retained. Endotoxin lipopolysaccharides were removed by adding triton X-100 detergent, mixing thoroughly and then discarding the resulting detergent layer. The cssDNA was then precipitated by adding 100% ethanol at −20 C to the solution, incubating on ice for a half hour, and then pelleting by centrifugation at 12,000 g. The cssDNA pellet was then washed with 75% ethanol at 4 C, incubated on ice for 20 minutes, and spun down again at 12,000 g to remove residual salts. The resulting cssDNA pellet was dried to remove residual ethanol and re-suspended in a buffered solution of tris-EDTA (TE) or nuclease-free water.

cssDNA was quantitated using the Qubit commercial assay kit and instrument following the manufacturers recommended protocol. Nucleic acid purity was quantitated using an Agilent Bioanalyzer. Endotoxin levels were quantitated via the Endosafe® nexgen-PTS™ handheld spectrophotometer and cartridge assay (Catalog number MCS150K, Charles River Laboratories, Wilmington, MA, USA) following the manufacturer's suggested protocol which utilizes a kinetic limulus amoebocyte lysate assay according to www.criver.com/sites/default/files/resources/Endosafe-PTS-Regulatory-Requirements-USPBET85-EPBET2.6.14.pdf.

### Example 3: Making Guide RNA

A single guide RNA with the spacer sequence of CAGGGTTCTGGATATCTGT (SEQ ID NO: 42), targeting the human TRAC locus was in vitro transcribed (Integrated DNA Technologies, Coralville, IA, USA). Mueller et al. Production and characterization of virus-free, CRISPR-CAR T cells capable of inducing solid tumor regression. Journal for ImmunoTherapy of Cancer 2022; 10:c004446.

### Example 4: Genomic Editing of Humans CD4+ T Cells

CD4+ T cells (Catalog #200-0165, StemCell Technologies, Vancouver, BC, Canada) are prepared for transfection. Cells are seeded at 1e6 cells per ml in Immunocult-XF serum-free medium (Catalog #10981 StemCell Technologies, Vancouver, BC, Canada) supplemented with 10 ng/ml recombinant human interleukin-2 (rhIL-2) (Catalog number 78036.3 StemCell Technologies, Vancouver, BC, Canada) and CD3/CD28 T Cell Activators (Catalog number 10971 StemCell Technologies, Vancouver, BC, Canada). Cells are incubated for 3 days in a humidified incubator at 37 C and 5% carbon dioxide, then diluted 1:4 into ImmunoCult XF supplemented with 10 ng/ml rhIL-2 and incubated for 2 more days. Cell titer is determined by hemocytometer. Cells are harvested via centrifugation at 200 RCF for 10 minutes at room temperature then resuspended at 1.3e7 cells per ml in P3 Primary Cell 4D-Nucleofector™ Solution plus supplement (Lonza Cologne, North Rhine-Westphalia, Germany).

cssDNA at 1.5 μM, sgRNA at 124 μM, and Cas9-ZF5 fusion at 62 μM are mixed 20:1:1 by volume and then incubated together at 37 C for 30 minutes to form a 1.4 μM complex between the three molecules of DNA, RNA and protein with RNA being in excess and DNA and protein being the limiting reagents. This complex is transfected into human peripheral blood CD4+ T cells. Other cells can also be used in this example such as Jurkat cells (Catalog number TIB-152, American Type Culture Collection, Manassas, VA, USA), or peripheral blood cells isolated from patients.

Transfections are performed with a Lonza 4D Nucleofector Core Unit and X Unit (Lonza Cologne, North Rhine-Westphalia, Germany) following the Amaxa™ 4D-Nucleofector™Protocol for stimulated Human T Cells (Lonza Cologne, North Rhine-Westphalia, Germany). 78 μl of the cell and buffer suspension containing 1e6 cells is mixed with 22 μl of the 1.4 μM DNA:RNA:protein complex and added to a 100 μl cuvette. The cuvette is inserted into the Lonza X Unit and electroporated according to program EO-115. Immediately following the electroporation event, 500 μl of pre-warmed ImmunoCult-XF medium supplemented with 10 ng/ml rhIL-2 is added to the cuvette to re-suspend the cells. The resulting 600 μl mixture of cells and media is pipetted into a 12-well plate containing 1.4 ml pre-warmed ImmunoCult-XF medium supplemented with 10 ng/ml rhIL-2. Cells are incubated for 2 days at 37 C and then passaged every 2-3 days.

The positive control for the electroporation protocol is a transient GFP plasmid with constitutive expression in mammalian cells that contains a DBD5 site and has been incubated with the sgRNA and the Cas9-ZF5 fusion protein.

Negative controls are no cssDNA, no sgRNA, no Cas9, and no zinc finger.

The GFP and mRuby signals are analyzed via flow cytometry (Attune NxT, Thermo Fisher, Waltham, MA) on day 2 after electroporation. The percent GFP positive cells in the population is a proxy for the percent of cells that were transfected with the transient GFP expression plasmid. The percent of mRuby positive cells in the population is a proxy for the percent of cells that were transfected with the DNA:RNA:protein complex.

The GFP and mRuby signals are measured again by flow cytometry after 7-12 days. The percent of GFP positive cells is a proxy for the percent of cells that integrated the GFP expression cassette into their genomes. Since the GFP plasmid lacks any homology to the genome and there is no double-strand break induced in the GFP control samples, the percent of cells expressing GFP protein is expected to be extremely low. This sample serves as a control for protein expression from the transient plasmid. Protein expression from the transient plasmid is expected to be low one week after transfection. If significant GFP signal is seen, it raises questions about the validity of the mRuby integration data. If low or no GFP signal is observed, it can be assumed that any mRuby signal observed is an indication of genomic integration of the mRuby expression cassette.

The percent of mRuby positive cells is a proxy for the percent of cells that correctly integrated the mRuby expression cassette at the TRAC locus. This correlation between mRuby signal and integration of the mRuby cassette is verified by isolating single, mRuby positive cells into separate wells of a 96-well plate and performing colony PCR using primers that bind in the genome at the TRAC locus and in the mRuby cassette. PCR products will only be formed if mRuby is in fact integrated at the TRAC locus.

### Example 5: Synthetic Biology

This example describes the use of designed sequences and synthetic nucleases in synthetic biology applications. It is known in the art that many bacterial cells that are otherwise desirable as targets for genomic editing are recalcitrant to such editing. For example, strains from the genus Paenibacillus have desirable agricultural characteristics, but have proven difficult to engineer. Bach et al, How to transform a recalcitrant Paenibacillus strain: From culture medium to restriction barrier, Journal of Microbiological Methods, 131, 135-143, 2016, which is herein incorporated by reference in its entirety.

A plasmid that includes an antibiotic resistance gene, a gene encoding a synthetic nuclease such as the high fidelity R691A variant of Cas9 from Streptococcus pyogenes Vakulskas et al., A high-fidelity Cas9 mutant delivered as a ribonucleoprotein complex enables efficient gene editing in human hematopoietic stem and progenitor cells. Nat Med. 2018 August; 24(8):1216-1224., and a gene producing one or more copies of a guide RNA is electroporated into the Paenibacillus strain simultaneously with a cssDNA that includes a co-location sequence, homology arms to a target dsDNA in the Paenibacillus strain and a gene encoding GFP. The resulting strain produces GFP and has the GFP gene incorporated into the genome of the Paenibacillus strain.

### Example 6: Duchenne Muscular Dystrophy Therapeutic Application

In this example, GenScript Biotech Corporation (Piscataway, New Jersey, U.S) is used to synthesize a 11,055 nt full length dystrophin transgene encoding all 3685 amino acids of the mature dystrophin protein. The 729 nt, SP-301, muscle-tissue-specific promoter control sequence (Skopenkova et al., Muscle-Specific Promoters for Gene Therapy. Acta Naturae. 2021 January-March; 13(1):47-58), a 60 nt flexible linker, an 18 nt 6-his tag, a 292 nt rabbit β-globin polyadenylation signal (Gil and Proudfoot, 1987) and two homology arms that target the integration of the full-length dystrophin cassette to the safe harbor AAVS1 locus Hayashi et at. Efficient viral delivery of Cas9 into human safe harbor. Sci Rep 10, 21474 (2020). The entire synthesized construct totalling 13,795 nt is cloned at GenScript into the m13 phagemid backbone described above which includes two, 20 nt DBM5 reverse complement stem sequences separated by a 16 nt loop sequence, and m13 origin of replication and packaging signal, and a pyrF expression cassette to allow selection in E. coli in the absence of uracil.

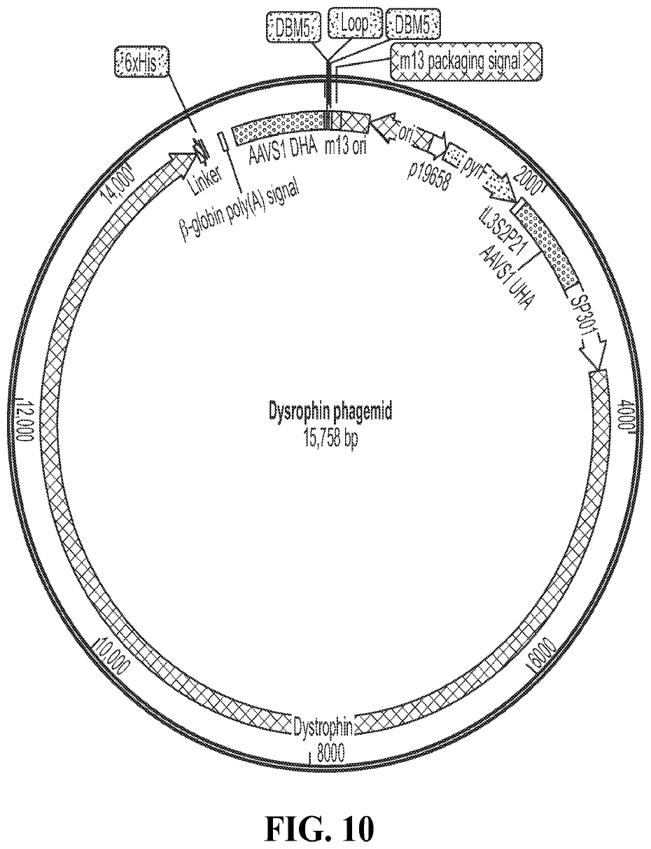

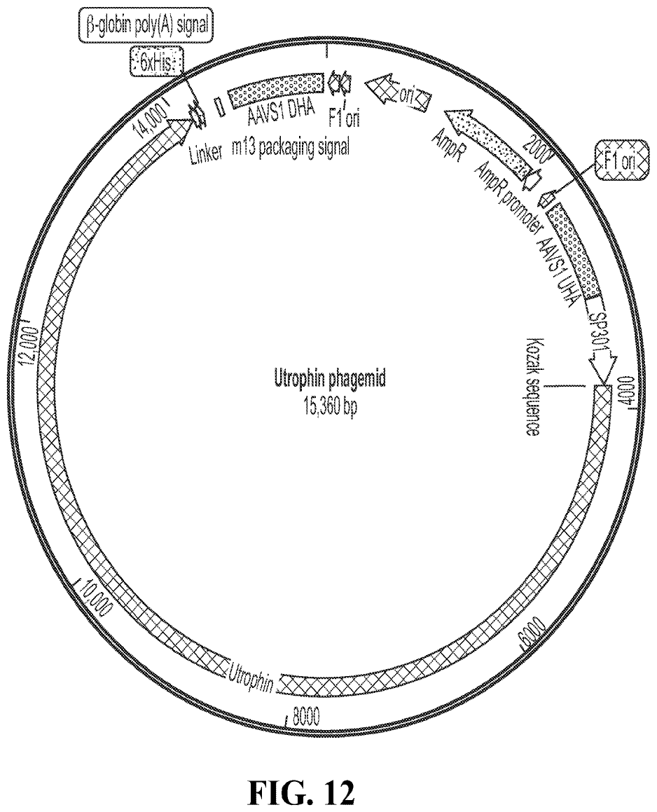

The dystrophin phagemid (shown in FIG. 10) in one experiment, and the utrophin phagemid (shown in FIG. 12) in another experiment, is transformed into Kano's E. coli cssDNA production using standard DNA transformation protocols and cssDNA is produced from the dsDNA template described above according to Kano's proprietary cssDNA production protocols. In short, the E. coli production strain harboring the m13 genes and the dsDNA phagemid is cultured in defined medium lacking uracil. The culture is centrifuged and the E. coli pellet is discarded and the phage particles are precipitated from the supernatant. The phage particles are lysed and their packaged cssDNA is purified through ethanol precipitation. Endotoxins are removed using a triton X-100 bilayer.

The concentration of the purified cssDNA is quantitated using the Qubit ssDNA fluorometric assay. The purity of the desired cssDNA product relative to potential contaminants such as bacterial mRNA and bacterial gDNA is assessed by running the cssDNA on a BioAnalyzer microfluidic agarose gel. The fidelity of the produced cssDNA relative to the dsDNA template sequence is assessed through Illumina next gen sequencing. Endotoxin levels are quantitated via the Endosafe® nexgen-PTS™ handheld spectrophotometer and cartridge assay (Catalog number MCS150K, Charles River Laboratories, Wilmington, MA, USA) following the manufacturers suggested protocol which utilizes a kinetic limulus amoebocyte lysate assay according to USP BET<85> and EP BET<2.6.14> https://www.criver.com/sites/default/files/resources/Endosafe-PTS-Regulatory-Requirements-USPBET85-EPBET2.6.14.pdf.

The cssDNA is co-incubated with the Cas9:ZF5 fusion protein and sgRNA produced at Integrated DNA Technologies (Coralville, Iowa, US) to form a cssDNA:ribonucleoprotein complex. The sgRNA sequence is GGGGCCACTAGGGACAGGAT (SEQ ID NO: 50) Mali et al., RNA-Guided Human Genome Engineering via Cas9. Science. 2013 Jan. 3. 10.1126. The complex is transfected into a myoblast cell culture model for DMD with a disrupted DMD gene using a Lonza Nucleofector 4D system (Lonza bioscience, Morrisville, NC, USA) and standard Lonza electroporation protocols. Soblechero-Martín et al. Duchenne muscular dystrophy cell culture models created by CRISPR/Cas9 gene editing and their application in drug screening. Sci Rep 11, 18188 (2021). Cells are passaged every 2-3 days for 7 to 10 days and then single cells are sorted into individual wells of a 96-well plate. Cultures are incubated until confluent at which time a sample is taken from each well for analysis by colony PCR. Cells are lysed by incubation for 10 minutes at 99 degrees C. 1 μl of the lysed cells is added to a PCR reaction including primers that bind to the dystrophin transgene and the AAVS1 locus outside of the homology arms used on the cssDNA HDRT such that an amplicon will only be created if there is an integration of the dystrophin transgene at the desired AAVS1 locus. The genomic DNA of the lysed cells is also amplified with primers that each bind in the AAVS1 locus outside the homology arms used in the cssDNA HDRT such that an amplicon will only be formed if there is no integration of the large dystrophin at the target AAVS1 locus. Wells that are positive for the first amplicon and negative for the second amplicon are expanded for further analysis.

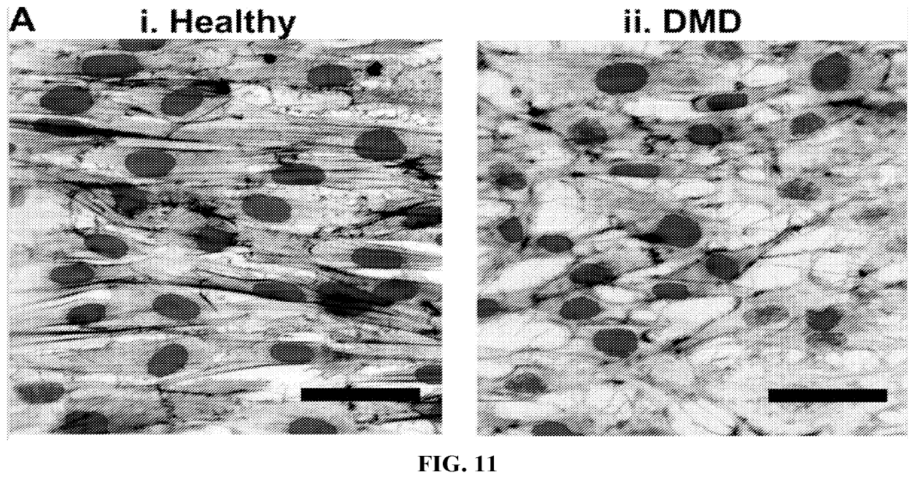

Presence of the dystrophin transgene protein is detected through Western blotting using antibodies that bind to the 6×his tag. The effect of the dystrophin protein is tested through quantitatively scoring microscopic images of the myoblast cell cultures. Images of cultures of cells expressing the full length DMD transgene are compared to control cultures with a disrupted native DMD gene and control cultures with a functional DMD native DMD gene. (FIG. 11, see Nesmith et al., Human in vitro model of Duchenne muscular dystrophy muscle formation and contractility. J Cell Biol. 2016 Oct. 10; 215(1):47-56.

An example of microscopic images of myoblast cell cultures expressing the wild type DMD gene is shown in FIG. 11A and a diseased mutant DMD gene FIG. 11B. The alignment of the actin, the roundness of the nuclei and the integrity of cell membranes can be analyzed and quantitatively scored by image processing software. Nesmith et al., Human in vitro model of Duchenne muscular dystrophy muscle formation and contractility. J Cell Biol. 2016 Oct. 10; 215(1):47-56.

### Example 7: Other Therapeutic Genomic Edits

Additional therapeutic designed sequences can be developed and delivered using a cssDNA that includes the DBM5 sequence or an alternative DNA binding motif that can bind to a DNA binding region of an amino acid sequence. The therapeutic sequences can be designed with associated homology arms to repair or replace various genes or non-coding sequences, such as control sequences, in the genome of a mammal, such as a human or other animal. Genes that are of particular interest for targeted repair using the system described herein include gene products directly or indirectly associated with muscle disease and injury. Skeletal, cardiac, or smooth muscle gene products are directly or indirectly associated with genetic diseases include, for example, genes encoding any of the following gene products: dystrophin, including mini- and microdystrophins (DMD; e.g., GenBank Accession Number NP_003997.1); titin (TTN); titin cap (titin cap, (TCAP), α-sarcoglycan (SGCA), β-sarcoglycan (SGCB), γ-sarcoglycan (SGCG) or δ-sarcoglycan (SGCD); alpha-1-antitrypsin (A1-AT); myosin heavy chain 6 (MYH6); myosin heavy chain 7 (MYH7); myosin heavy chain 11 (MYH11); myosin light chain 2 (ML2); myosin light chain 3 (ML3); myosin light chain kinase 2 (MYLK2); myosin-binding protein C (MYBPC3); desmin (DES); dynamin 2 (DNM2); laminin α2 (LAMA2); lamin A/C (LMNA); lamin B (LMNB); lamin B receptor (LBR); dysferlin (DYSF); emerin (EMD); insulin; blood clotting factors, including, but not limited to: factor VIII and factor IX; erythropoietin (EPO, EPO); lipoprotein lipases (LPL); sarcoplasmic reticulum Ca2++-ATPase (SERCA2A), calcium-binding protein S100 A1 (S100A1); myotubular (MTM); protein kinase DM1 (DMPK; eg GenBank Accession Number NG_009784.1); glycogen phosphorylase L (PYGL); muscle-associated glycogen phosphorylase (PYGM; eg GenBank Accession Number NP_005600.1); glycogen synthase 1 (GYS1); glycogen synthase 2 (GYS2); α-galactosidase A (GLA; eg GenBank Accession Number NP_000160.1); α-N-acetylgalactosaminidase (NAGA); acid α-glucosidase (GAA; eg GenBank Accession Number NP_000143.2), sphingomyelinase phosphodiesterase 1 (SMPD1); lysosomal acid lipase (LIPA); collagen type I α1 chain (COL1A1); collagen type I α2 chain (COL1A2); collagen type III α1 chain (COL3A1); collagen type V α1 chain (COL5A1); collagen type V α2 chain (COL5A2); collagen type VI α1 chain (COL6A1); collagen type VI α2 chain (COL6A2); collagen type VI α3 chain (COL6A3); procollagen-lysine-2-oxoglutarate-5-dioxygenase (PLOD1); lysosomal acid lipase (LIPA); frataxin (FXN; eg GenBank Accession Number NP_000135.2); myostatin (MSTN); β-N-acetylhexosaminidase A (HEXA); β-N-acetylhexosaminidase B (HEXB); β-glucocerebrosidase (GBA); adenosine monophosphate deaminase 1 (AMPD1); β-globin (HBB); iduronidase (IDUA); iduronate-2-sulfate (IDS); troponin 1 (TNNI3); troponin T2 (TNNT2); troponin C (TNNC1); tropomyosin 1 (TPM1); tropomyosin 3 (TPM3); N-acetyl-α-glucosaminidase (NAGLU); N-sulphoglucosamine sulfohydrolase (SGSH); heparan-α-glucosaminide N-acetyltransferase (HGSNAT); α7 integrin (IGTA7); integrin α9 (IGTA9); glucosamine (N-acetyl)-6-sulfatase (GNS); galactosamine (N-acetyl)-6-sulfatase (GALNS); β-galactosidase (GLB1); β-glucuronidase (GUSB); hyaluronoglucosaminidase 1 (HYAL1); acid ceramidase (ASAH1); galactosylcermidase (GALC); cathepsin A (CTSA); cathepsin D (CTSA); cathepsin K (CTSK); GM2 ganglioside activator (GM2A); arylsulfatase A (ARSA); arylsulfatase B (ARSB); formylglycine generating enzyme (SUMF1); neuraminidase 1 (NEU1); N-acetylglucosamine-1-phosphate transferase α (GNPTA); N-acetylglucosamine-1-phosphate transferase β (GNPTB); N-acetylglucosamine-1-phosphate transferase γ (GNPTG); mucolipin-1 (MCOLN1); NPC intracellular transporter 1 (NPC1); NPC intracellular transporter 2 (NPC2); ceroid lipofuscinosis 5 (CLN5); ceroid lipofuscinosis 6 (CLN6); ceroid lipofuscinosis 8 (CLN8); palmitoyl protein thioesterase 1 (PPT1); tripeptidyl peptidase 1 (TPP1); battenin (CLN3); DNAJ protein of the heat shock protein family 40 member C5 (DNAJC5); protein 8 containing the domain of the superfamily of the main mediators (gene MFSD8); mannosidase α class 2B member 1 (MAN2B1); mannosidase β (MANBA); aspartyl glucosaminidase (AGA); α-L-fucosidase (FUCA1); cystinosine, lysosomal cysteine transporter (CTNS); sialin; soluble carrier family 2, member 10 (SLC2A10); soluble carrier family 17, member 5 (SLC17A5); soluble carrier family 6, member 19 (SLC6A19); soluble carrier family 22, member 5 (SLC22A5); soluble carrier family 37, member 4 (SLC37A4); lysosome-associated membrane protein 2 (LAMP2); voltage-gated sodium channel, α subunit 4 (SCN4A); voltage-gated sodium channel, β subunit 4 (SCN4B); voltage-gated sodium channel, α subunit 5 (SCN5A); voltage-gated sodium channel, α subunit 4 (SCN4A); voltage-gated calcium channel, α1c subunit (CACNAIC); voltage-gated calcium channel, α1s subunit (CACNlS); phosphoglycerate kinase 1 (PGK1); phosphoglycerate mutase 2 (PGAM2); amyl-α-glucosidase, 4-α-glucanotransferase (AGL); voltage-gated potassium channel, ISK-associated subfamily member 1 (KCNE1); voltage-gated potassium channel, ISK-associated subfamily member 2 (KCNE2); voltage-gated potassium channel, subfamily J, member 2 (KCNJ2); voltage-gated potassium channel, subfamily J, member 5 (KCNJ5); voltage-gated potassium channel, subfamily H, member 2 (KCNH2); voltage-gated potassium channel, KQT-like subfamily member 1 (KCNQ1); cyclic nucleotide-gated hyperpolarization-activated channel 4 (HCN4); voltage-gated chloride channel 1 (CLCN1); carnitine palmitoyltransferase 1A (CPT1A); ryanodine receptor 1 (RYR1); ryanodine receptor 2 (RYR2); bridge integrator 1 (BIN1); LARGE xylosyl and glucuronyl transferase 1 (LARGE1); docking protein 7 (DOK7); fukutin (FKTN); fucutin-related protein (FKRP); selenoprotein N (SELENON); protein O-mannosyltransferase 1 (POMT1); protein O-mannosyltransferase 2 (POMT2); protein O-linked mannose N-acetylglucosaminyltransferase 1 (POMGNT1); protein O-linked mannose N-acetylglucosaminyltransferase 2 (POMGNT2); protein O-mannose kinase (POMK); containing isoprenoid synthase domain (ISPD); plectin (PLEC); cholinergic receptor, nicotinic epsilon subunit (CHRNE); choline O-acetyltransferase (CHAT); choline kinase β (CHKB); collagen-like “tail” subunit of asymmetric acetylcholinesterase (COLQ); receptor-associated protein synapse (RAPSN); protein 1 “four and a half LIM domains” (FHL1); β-1,4-glucuronyltransferase 1 (B4GAT1); β-1,3-N-acetylgalactosaminyltransferase 2 (B3GALNT2); dystroglycan 1 (DAG1); transmembrane protein 5 (TMEM5); transmembrane protein 43 (TMEM43); SECIS binding protein 2 (SECISBP2); glucosamine (UDP-N-acetyl)-2-epimerase/N-acetylmannosamine kinase (GNE); anoctamine 5 (ANO5); protein 1 containing a flexible hinge domain of structural support of chromosomes (SMCHD1); lactate dehydrogenase A (LDHA); lactate dehydrogenase B (LHDB); calpain 3 (CAPN3); caveolin 3 (CAV3); protein 32 containing tripartite motif 32 (TRIM32); CCHC type zinc finger protein binding nucleic acid (CNBP); nebulin (NEB); actin, α1, skeletal muscle (ACTA1); actin, α1, cardiac muscle (ACTC1); actinin α2 (ACTN2); poly(A)-binding nuclear protein 1 (PABPN1); protein 3 containing the LEM domain (LEMD3); zinc metalloproteinase STE24 (ZMPSTE24); microsomal triglyceride transfer protein (MTTP); cholinergic nicotinic receptor, α1 subunit (CHRNA1); cholinergic nicotinic receptor, α2 subunit (CHRNA2); cholinergic nicotinic receptor, α3 subunit (CHRNA3); cholinergic nicotinic receptor, α4 subunit (CHRNA4); cholinergic nicotinic receptor, 5 subunit (CHRNA5); cholinergic nicotinic receptor, α6 (CHRNA6) subunit; cholinergic nicotinic receptor, α7 subunit (CHRNA7); cholinergic nicotinic receptor, α8 subunit (CHRNA8); cholinergic nicotinic receptor, α9 subunit (CHRNA9); cholinergic nicotinic receptor, α10 subunit (CHRNA10); cholinergic nicotinic receptor, β1 subunit (CHRNB1); cholinergic nicotinic receptor, β2 subunit (CHRNB2); cholinergic nicotinic receptor, β3 subunit (CHRNB3); cholinergic nicotinic receptor, β4 subunit (CHRNB4); cholinergic nicotinic receptor, subunit γ (CHRNG1); cholinergic nicotinic receptor subunit ∂ (CHRND); cholinergic nicotinic receptor, subunit ε (CHRNE1); subfamily A of the ATP binding cassette, member 1 (ABCA1); subfamily C of the ATP binding cassette, member 6 (ABCC6); subfamily A of the ATP binding cassette, member 9 (ABCC9); subfamily D of the ATP binding cassette, member 1 (ABCD1); ATPase 1 transporting Ca2+ of the sarcoplasmic/endoplasmic reticulum (ATP2A1); ATM serine-threonine kinase (ATM); α-tocopherol transferase protein (TTPA); kinesin family member 21A (KIF21A); paired-like homeobox 2a protein (PHOX2A); heparan sulfate proteoglycan 2 (HSPG2); stromal interaction molecule 1 (STIM1); notch 1 protein (NOTCH1); notch 3 protein (NOTCH3); distrobrevin α (DTHA); protein kinase AMP-activated, non-catalytic γ2 (PRKAG2); cysteine and glycine rich protein 3 (CSRP3); viniculin (VCL); myosenin 2 (MyoZ2); myopalladin (MYPN); junctophilin, (junctophilin) 2 (JPH2); phospholamban (PLN); calreticulin 3 (CALR3); nexilin F-actin-binding protein (NEXN); protein 3, LIM domain binding 3 (LDB3); eye absence protein 4 (eyes absent 4 (EYA4)); huntingtin (HTT); androgen receptor (AR); protein tyrosine phosphate non-receptor type 11 (PTPN11); junctional plakoglobin (JUP); desmoplakin (DSP); plakophilin 2 (PKP2); desmoglein 2 (DSG2); desmocollin 2 (DSC2); catenin α3 (CTNNA3); NK2 homeobox protein 5 (NKX2-5); A-kinase anchor protein 9 (AKAP9); A-kinase anchor protein 10 (AKAP10); guanine nucleotide-binding protein activity inhibition polypeptide 2α-inhibiting activity polypeptide 2 (GNAI2)); ankyrin 2 (ANK2); syntrophin α-1 (SNTA1); calmodulin 1 (CALM1); calmodulin 2 (CALM2); HTRA serine peptidase 1 (HTRA1); fibrillin 1 (FBN1); fibrillin 2 (FBN2); xylosyltransferase 1 (XYLT1); xylosyltransferase 2 (XYLT2); tafazzin (TAZ); 1,2-dioxygenase homogentisate (HGD); glucose-6-phosphatase catalytic subunit (G6PC); 1,4-alpha-glucan enzyme 1 (GBE1); phosphofructokinase, muscle (PFKM); phosphorylase kinase alpha 1 regulatory subunit (PHKA1); phos regulatory subunit phorylase kinase alpha 2 (PHKA2); regulatory subunit of beta phosphorylase kinase (PHKB); catalytic subunit of phosphorylase kinase gamma 2 (PHKG2); phosphoglycerate mutase 2 (PGAM2); cystathion beta synthase (CBS); methylenetetrahydrofolate reductase (MTHFR); 5-methyltetrahydrofolate homocysteine methyltransferase (MTR); 5-methyltetrahydrofolate homocysteine methyltransferase reductase (MTRR); methylmalonic aciduria and homocystinuria cblD (MMADHC); mitochondrial DNA, including but not limited to: mitochondrially encoded NADH: ubiquinone oxidoreductase central subunit 1 (MT-ND1); mitochondrially encoded NADH: ubiquinone oxidoreductase major subunit 5 (MT-ND5); mitochondrially encoded glutamic acid tRNA (MT-TE); mitochondrially encoded histadin tRNA (MT-TH); mitochondrially encoded tRNA leucine 1 (MT-TL1); mitochondrially encoded tRNA lysine (MT-TK); mitochondrially encoded tRNA serine 1 (MT-TS1); mitochondrially encoded valine tRNA (MT-TV); mitogen-activated protein kinase 1 (MAP2K1); proto-oncogene B-Raf associated serine/threonine kinase (BRAF); proto-oncogene raf-1 associated serine/threonine kinase (RAF1); growth factors, including but not limited to: insulin growth factor 1 (IGF-1); transforming growth factor β3 (TGFβ3); transforming growth factor receptor β type I (TGFβR1); transforming growth factor receptor β type II (TGFβR2), fibroblast growth factor 2 (FGF2), fibroblast growth factor 4 (FGF4), vascular endothelial growth factor A (VEGF-A), vascular endothelial growth factor B (VEGF-B); vascular endothelial growth factor C (VEGF-C), vascular endothelial growth factor D (VEGF-D), vascular endothelial growth factor receptor 1 (VEGFR1) and vascular endothelial growth factor receptor 2 (VEGFR2); interleukins; immunoadhesins; cytokines; and antibodies.

### Example 8: Protein:Protein Interactions

Creating a synthetic fusion protein of Cas9 fused to zinc finger 5 as detailed in Example 1 is not the only way to achieve the goals of binding the cssDNA HDRT to the ribonucleoprotein genome editing complex. Cas9 which binds to and cuts the genome and zinc finger 5 which binds to the cssDNA HDRT can also be expressed separately and linked through protein:protein interactions after they have been expressed. This can be done by transcriptionally fusing GFP beta strand peptides 1-10 to the zinc finger and by transcriptionally fusing GFP beta strand peptide 11 to Cas9. After each protein fusion is expressed separately and mixed together in an in vivo or in vitro reaction GFP peptide 11 (residues 215-230), the proteins will auto-assemble to GFP peptides 1-10 (residues 1-214) to form a complete GFP protein thus linking Cas9 to the zinc finger (see FIG. 5). Cabantous et al., Protein tagging and detection with engineered self-assembling fragments of green fluorescent protein. Nat Biotechnol. 2005; 23: 102-107.

Likewise, this same effect can be accomplished with other proteins that are known to interact with each other. For example, the spy catcher/spy tag system could be used. Just as in the split GFP example above, the 13 amino acid spy tag peptide could be transcriptionally fused to Cas9 and the 12.3 kilodalton spy catcher protein could be transcriptionally fused to zinc finger 5. These two fusion proteins could be expressed separately and then mixed together either in vivo or in vitro at which point the spy catcher and spy tag polypeptides will interact with each other form an intermolecular isopeptide bond and link Cas9 to zinc finger 5. Zakeri et al., Howarth M. Peptide tag forming a rapid covalent bond to a protein, through engineering a bacterial adhesin. Proc Natl Acad Sci USA. 2012 Mar. 20; 109(12):E690-7.

### Example 9: In Vitro DNA Cleavage Assay

The present Example examines and characterizes the functionality of the nuclease:zinc finger fusion proteins synthesized in the earlier Examples. Specifically, an in vitro cleavage assay was performed to demonstrate that the nuclease domain retained its functionality to interact with a sgRNA to create double-strand breaks in DNA in a sequence-specific manner.

### Materials and Methods

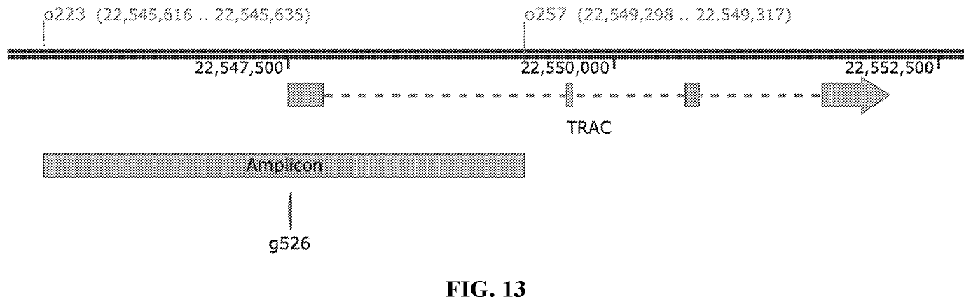

First, a 3702 bp portion of the TRAC locus (trac long NC_000014.9. SEQ ID NO: 12) was amplified via PCR from a genomic prep of HEK293T cells using oligos o223 and o257 as primers with sequences TTGTGTTCTGTGGCATIGCG (SEQ ID NO: 44) and GGCAGCGAGGCATACATAGT (SEQ ID NO: 45) respectively. The g526 protospacer sequence TCAGGGTTCTGGATATCTGT (SEQ ID NO: 42) falls roughly in the middle of this amplicon such that if the amplicon were cleaved by a Cas9 nuclease guided by the g526 spacer sequence one would expect the reaction to yield one, 1889 bp fragment and one 1813 fragment. The amplicon was verified by gel electrophoresis and then purified using the QIAquick PCR Purification Kit (catalog #28104, Qiagen, Venlo, Netherlands). It was then normalized to 100 ng/ul. FIG. 13 shows a schematic of the TRAC amplicon for in vitro cleavage assay.

o223 and o225 are the forward and reverse primer respectively used to amplify the 3.7 kb fragment of the TRAC locus. G526 is the protospacer sequence targeted by the g526 sgRNA. TRAC is the human T cell receptor alpha constant gene. Gray blocks in FIG. 13 represent exons and dotted lines represent introns for one of the isoforms of the TRAC mRNA.

Versions of HiFi Sp Cas9 nuclease with and without the zinc finger fusion were obtained (SEQ ID NOs: 51 and 16, respectively). Cas9 with the zinc finger fusion was synthesized by Genscript (Jiangsu Province, P.R. China) as described above. Cas9 without the zinc finger fusion was purchased from IDT (catalog number 1081061 Integrated DNA Technologies, Coralville, IA. Each version was diluted to 10 μM in buffer consisting of, 20 mM HEPES-KOH, 500 mM KCL, 1 mM DTT, and 250 nM ZnSO4.

g526 sgRNA (SEQ ID NO: 54) was diluted to 10 μM in ssDNA electroporation enhancer (catalog number 1075916 Integrated DNA Technologies. Coralville. IA). Cas9 and sgRNA were each diluted to 1.5 μM in cleavage buffer consisting of 20 mM HEPES pH 7.6, 100 mM KCl, 5% w/v glycerol, 1 mM DTT, 0.5 mM EDTA, 2 mM MgCl2 and 50 nM ZnSO4 and incubated for 15 minutes at room temperature to allow RNPs to form. 10 ng of the TRAC amplicon DNA was then added to each tube and tubes were incubated at 37 C to allow Cas9 to cleave the amplicon. 10 μL of each sample was removed from the reaction at the designated time points of 1 minute, 5 minutes, 15 minutes, 60 minutes, and 120 minutes. Upon removal, reactions were quenched by adding 1 μL of 500 mM EDTA and stored at −80 C. When all reactions were completed, quenched and frozen, they were then thawed and 1 μL of Proteinase K was added. They were incubated with Proteinase K for 20 minutes at room temperature to degrade Cas9 protein and liberate the DNA from Cas9 for analysis by gel electrophoresis. Samples were then analyzed on a 1% agarose TAE gel run at 125 volts for 120 minutes.

### Results

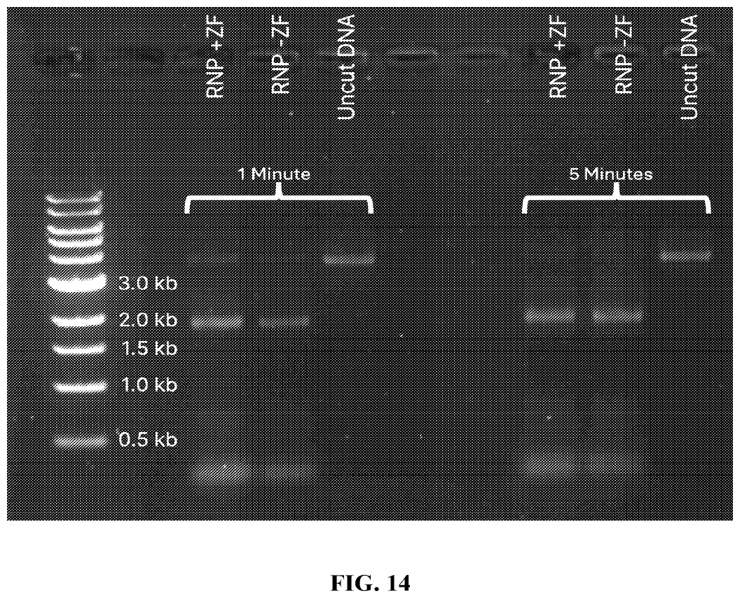

The resulting gel from the in vitro cleavage assay showed that virtually all of the 3.7 kb fragment had been digested to two, 1.8 KB fragments within the first minute of incubation at 37 C (FIG. 14). After 5 minutes of incubation, residual 3.7 kb DNA was almost undetectable. This was true for both the new Cas9:zinc finger fusion protein and for the traditional HiFi Sp Cas9 sold by IDT. Samples from other timepoints including 15 minutes, 60 minutes, and 120 minutes were also run on gel and they continued to show complete digestion of the 3.7 kb fragment (data not shown). This in vitro cleavage assay established that the engineered Cas9:zinc finger fusion protein was capable of interacting with the g526 sgRNA to cleave double-stranded DNA in a sequence specific fashion just as well as the same protein sequence sold by a common commercial vendor lacking the zinc finger fusion. Put another way, it showed that the zinc finger fusion did not affect the function of the Cas9 nuclease.

### Example 10: In Vitro Nuclease:cssDNA Binding Assay

Example 9 established that the Cas9 portion of the fusion proteins described in the previous example was functional. The present Example demonstrates that the zinc finger portion of the fusion proteins are capable of binding to cssDNA in a sequence specific manner (i.e., through the ZF DNA binding domain).

### Materials and Methods

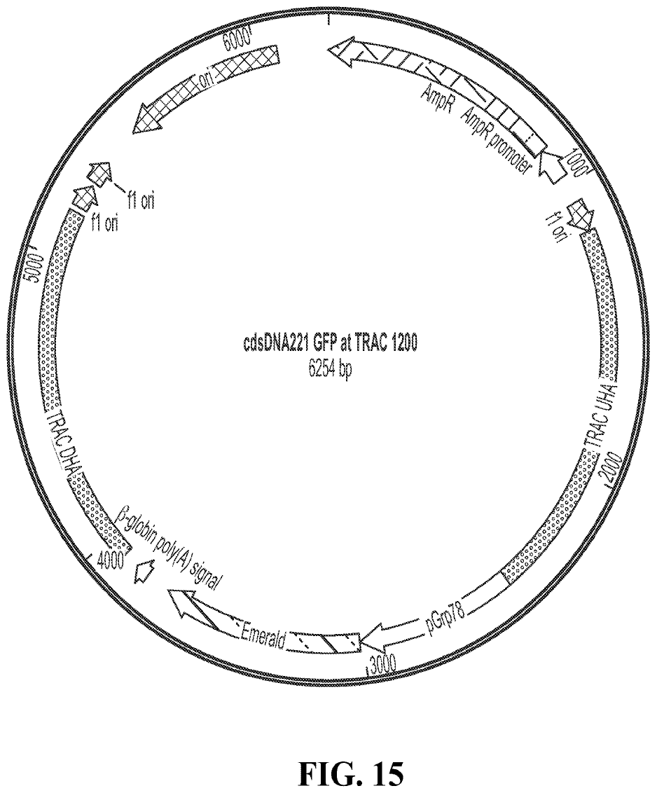

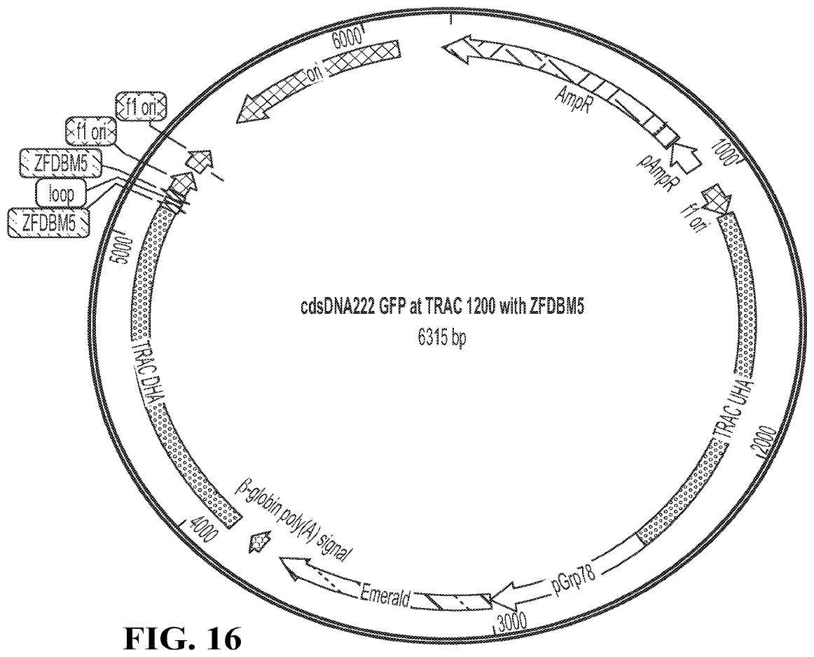

Two new phagemids cdsDNA221 and cdsDNA222 (schematics of which are shown in FIG. 15 and FIG. 16, respectively) were cloned and produced as cssDNA. They consisted of an ampicillin resistance marker, a pUC origin for replication for bacterial replication, an F1 origin for replication and packaging of cssDNA in M13 phage capsids, two, 1200 bp TRAC homology arms and an emGFP expression cassette. The two phagemids were identical to each other except that cdsDNA222 contained a zinc finger DNA binding motif number 5 (ZFDBM5) hairpin sequence and cdsDNA221 lacked this sequence. The DNA sequences of cdsDNA222 is represented in SEQ ID NO: 3 and the DNA sequence of cdsDNA221 is represented in SEQ ID NO: 4.

The ZFDBM5 hairpin sequence (SEQ ID NO: 23) was designed such that it consisted of 2, 18 nt ZFDBM5 sequences, 1 flanking nucleotide on both ends of the binding motif to increase the length of double-stranded DNA available to the zinc finger protein and a 16 nt loop sequence. The 2. ZFDBM5 sequences flanked the loop sequence and were reverse complements of each other such that when produced as cssDNA, they folded over at the loop sequence and base paired with each other to form a short, 20 bp dsDNA hairpin with the longer cssDNA construct. This dsDNA hairpin structure was necessary because zinc finger proteins are known to interact with dsDNA; but not ssDNA. The loop sequence (SEQ ID NO: 24) was designed to have little complementarity so that it remained single-stranded and flexible and allowed the binding motifs to fold over and base pair with each other.

The two versions of HiFi Sp Cas9 with and without the zinc finger fusion described above were mixed with the sgRNA g526 in a 2:1 molar ratio and incubated at 37 C for 20 minutes to form ribonucleoprotein complexes (RNPs). cssDNA221 and cssDNA222 were normalized to 100 nM in a zinc finger binding buffer consisting of 10 nM (NH4)₂SO₄, 0.2% w/v Tween 20, 30 mM KCl, and 250 nM ZnSO₄. The RNPs and cssDNA were then mixed together 1:1 based on volume and incubated at 25 C for 1 hour to allow the zinc finger protein to bind to the zinc finger binding motif number 5 hairpin on cssDNA222. The samples were then run on a 1% agarose gel with TAE at 120V for 75 minutes.

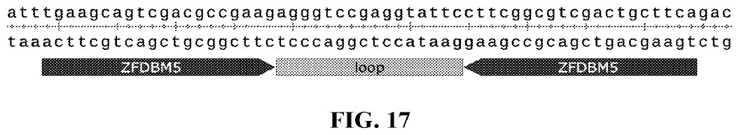

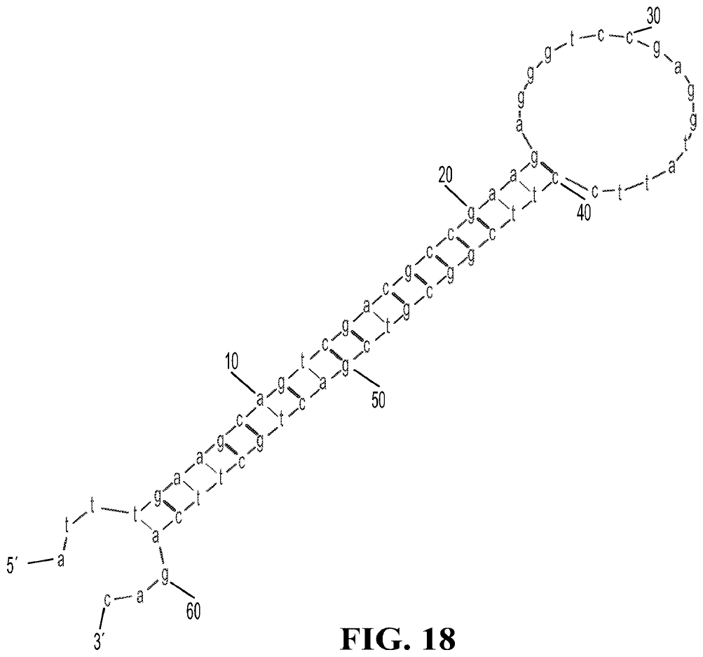

FIG. 15 shows a schematic of cdsDNA221 without zinc finger DNA binding motif number 5 (SEQ ID NO: 4). FIG. 16 shows a schematic of cdsDNA222 with zinc finger DNA binding motif number 5 (SEQ ID NO: 3). FIG. 17 shows a schematic of the zinc finger DNA binding motif number 5 of cdsDNA222 (SEQ ID NO: 23). FIG. 18 shows a schematic of the secondary structure of zinc finger DNA binding motif number 5 of cdsDNA222 (SEQ ID NO: 23).

Secondary structure was predicted by Unafold. N. R. Markham & M. Zuker. UNAFold: Software for Nucleic Acid Folding and Hybridization. In Data, Sequence Analysis. and Evolution, J. Keith, ed., Bioinformatics: Volume 2, Chapter 1, pp 3-31, Humana Press Inc., 2008. The 18 nt ZFDBM5 sequences and their two flanking bases form a 20 bp stem while the 16 nt intervening sequence forms the loop structure.

### Results

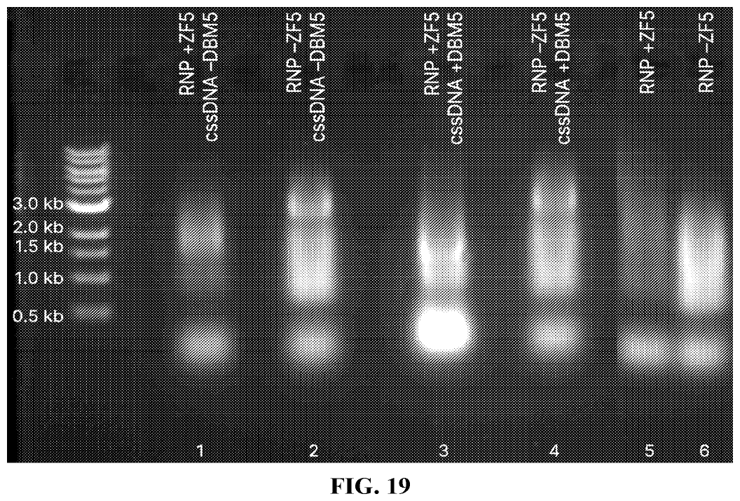

FIG. 19 shows a gel image from the in vitro zinc finger binding assay. Lanes 5 and 6 are the RNPs with and without the zinc finger fusion run without any cssDNA. In these lanes, there is a band below 500 bp representing the RNP as well as a smear without any distinct bands between 500 bp and 3 kb.

Lanes 1-4 were run with both RNPs and cssDNA and these lanes also have a band below 500 bp representing the RNP but they additionally have a somewhat defined band between 1 kb and 3 kb representing the cssDNA. In lanes 2 and 4, where the Cas9 protein lacks the zinc finger fusion, the cssDNA band runs slightly below 3 kb. In lane 1, where the Cas9 protein is fused to the zinc finger, the cssDNA runs slightly faster at around 2 kb indicating that there may be some non-specific interaction between the zinc finger and the cssDNA even though this cssDNA molecule lacks the ZFDBM5 hairpin sequence.

In lane 3, however, where Cas9 is fused to the zinc finger and the cssDNA possesses the ZFDBM5 hairpin sequence, the cssDNA runs even faster and forms a band around 1.5 kb. Moreover, the lower band representing the RNP is elevated in this lane and extremely bright relative to the other lanes even though the same amount of material was loaded into each lane. The elevation may be caused by binding of the RNP to the cssDNA at the ZFDBM5 sequence thus retarding the progress of the RNP across the gel. The brightness may be caused by the presence of cssDNA in the lower band which is intercalated by the fluorescent dye, Sybr Safe. The lanes of the gel were cropped from their original locations and pasted near each other for ease of viewing. The position of the bands relative to the ladder was not modified. The differences between lane 3 and the other 5 negative control lanes indicate that the zinc finger protein is serving its function and specifically binding cssDNA at the ZFDBM5 hairpin sequence.

### Example 11: Genomic Editing of Human HEK293T Cells

Example 10 showed that the HiFi Sp Cas9:zinc finger fusion protein was both functional in its RNA-guided nuclease domain and in its zinc finger guided DNA binding domain. The present Example demonstrates the utility of the described fusion proteins and cssDNA/DNA-binding domains in genome editing.

The present Example also employs two strategies for expression of the cssDNA portion and the Cas9/ZF fusion portion. The first approach includes pre-formulating the Cas9:zinc finger fusion protein with sgRNA and cssDNA in vitro prior to transfection of mammalian cells. The second approach includes expressing Cas9:zinc finger fusion protein and sgRNA in cells after co-transfection with cssDNA. Both methods were predicted to have advantages and disadvantages over each other. Pre-formulating the tripartite protein:sgRNA:DNA complex in vitro would help ensure that each component would be linked to each other component, but on the other hand it would create a large complex that may have difficulty crossing the cell membrane. Transfecting expression vectors encoding the Cas9:zinc finger fusion and the sgRNA separately alongside the cssDNA repair template would increase the chances of each individual molecule entering the cells, but would reduce the chances of the three molecules complexing with each other. Both methods were tested side-by-side in this Example.

### Materials and Methods

### Expression Vectors

A Cas9 mammalian expression vector was obtained from Vector Builder (catalog #VB900129-1111qhq, Vector Builder, Chicago, IL) (cdsDNA166 as shown in SEQ ID NO: 8). It included an EF1α promoter driving expression of Sp Cas9 with two NLS sequences in a standard pUC19/ampicillin resistant plasmid backbone. It also had a puromycin resistance cassette for selection in mammalian cells which was not used in this experiment. A 780 bp gBlock consisting of the zinc finger DNA sequence, two flexible protein linker DNA sequences, the DNA sequence for the SV40 NLS and homology to the Cas9 expression vector at the 3′ end of the Cas9 DNA sequence and at the SV40 poly(A) DNA sequence was synthesized as a gBlock at IDT (Integrated DNA Technologies, Coralville, IA) (as shown in SEQ ID NO: 13). The Cas9 expression vector was amplified by PCR in three, roughly 3-kb parts and assembled in a NEBuilder HiFi assembly reaction along with the 780 bp gBlock to create a fusion of the zinc finger 5 protein sequence to the Cas9 protein sequence. This new Cas9:zinc finger fusion expression vector was labeled cdsDNA177 (sequence shown in SEQ ID NO: 7). It expressed a similar protein sequence to the one described above that was cloned for E. coli expression at Genscript. The DNA sequence however was codon optimized for mammalian expression rather than bacterial expression. The E. coli expressed protein utilized an N-terminal His-tag while the mammalian expressed protein had an N-terminal flag tag. The E. coli expressed protein was the high fidelity version of Sp Cas9 with the R691A mutation (SEQ ID NO: 16) while the mammalian expressed version was wild type at R691 (SEQ ID NO: 18). These differences were purely due to the convenience of cloning into the Vector Builder standard mammalian Cas9 vector which was slightly different from the Genscript expressed version.

A Clustal Omega alignment of the Genscript E. coli expressed Cas9:zinc finger fusion protein (SEQ ID NO: 51) to the cloned and mammalian expressed Cas9 zinc finger fusion protein (SEQ ID NO: 53) described herein is provided below:

CLUSTAL O(1.2.4) multiple sequence alignment

| | | |
|---|---|---|
| SIN51_Bacterial | ---------------MHHHHHHASPPKKKRKV-----GSMDKKYSIGLDIGTNSVGWAVI | 40 |
| SIN53_mammalian | MDYKDHDGDYKDHDIDYKDDDDKMAPKKKRKVGIHGVPAADKKYSIGLDIGTNSVGWAVI | 60 |
| | ::.... ******* : ******************** | |
| SIN51_Bacterial | TDDYKVPSKKFKVLGNTDRHSIKKNLIGALLFGSGETAEATRLKRTARRRYTRRKNRICY | 100 |
| SIN53_mammalian | TDEYKVPSKKFKVLGNTDRHSIKKNLIGALLFDSGETAEATRLKRTARRRYTRRKNRICY | 120 |
| | **:*****************************.*************************** | |
| SIN51_Bacterial | LQEIFSNEMAKVDDSFFHRLEESFLVEEDKKHERHPIFGNIVDEVAYHEKYPTIYHLRKK | 160 |
| SIN53_mammalian | LQEIFSNEMAKVDDSFFHRLEESFLVEEDKKHERHPIFGNIVDEVAYHEKYPTIYHLRKK | 180 |
| | ************************************************************ | |
| SIN51_Bacterial | LADSTDKADLRLIYLALAHMIKFRGHFLIEGDLNPDNSDVDKLFIQLVQIYNQLFEENPI | 220 |
| SIN53_mammalian | LVDSTDKADLRLIYLALAHMIKFRGHFLIEGDLNPDNSDVDKLFIQLVQTYNQLFEENPI | 240 |
| | *.*********************************************** ********** | |
| SIN51_Bacterial | NASRVDAKAILSARLSKSRRLENLIAQLPGEKRNGLFGNLIALSLGLTPNFKSNFDLAED | 280 |
| SIN53_mammalian | NASGVDAKAILSARLSKSRRLENLIAQLPGEKKNGLFGNLIALSLGLTPNFKSNFDLAED | 300 |
| | *** ****************************:*************************** | |
| SIN51_Bacterial | AKLQLSKDTYDDDLDNLLAQIGDQYADLFLAAKNLSDAILLSDILRVNSEITKAPLSASM | 340 |
| SIN53_mammalian | AKLQLSKDTYDDDLDNLLAQIGDQYADLFLAAKNLSDAILLSDILRVNTEITKAPLSASM | 360 |
| | ************************************************:*********** | |
| SIN51_Bacterial | IKRYDEHHQDLTLLKALVRQQLPEKYKEIFFDQSKNGYAGYIDGGASQEEFYKFIKPILE | 400 |
| SIN53_mammalian | IKRYDEHHQDLTLLKALVRQQLPEKYKEIFFDQSKNGYAGYIDGGASQEEFYKFIKPILE | 420 |
| | ************************************************************ | |
| SIN51_Bacterial | KMDGTEELLVKLNREDLLRKQRTFDNGSIPHQIHLGELHAILRRQEDFYPFLKDNREKIE | 460 |
| SIN53_mammalian | KMDGTEELLVKLNREDLLRKQRTFDNGSIPHQIHLGELHAILRRQEDFYPFLKDNREKIE | 480 |
| | ************************************************************ | |
| SIN51_Bacterial | KILTFRIPYYVGPLARGNSRFAWMTRKSEETITPWNFEEVVDKGASAQSFIERMINFDKN | 520 |
| SIN53_mammalian | KILTFRIPYYVGPLARGNSRFAWMTRKSEETITPWNFEEVVDKGASAQSFIERMTNFDKN | 540 |
| | ************************************************************ | |
| SIN51_Bacterial | LPNEKVLPKHSLLYEYFTVYNELTKVKYVTEGMRKPAFLSGEQKKAIVDLLFKTNRKVTV | 580 |
| SIN53_mammalian | LPNEKVLPKHSLLYEYFTVYNELTKVKYVTEGMRKPAFLSGEQKKAIVDLLFKTNRKVTV | 600 |
| | ************************************************************ | |
| SIN51_Bacterial | KQLKEDYFKKIECFDSVEISGVEDRFNASLGAYHDLLKIIKDKDFLDNEENEDILEDIVL | 640 |
| SIN53_mammalian | KQLKEDYFKKIECFDSVEISGVEDRFNASLGTYHDLLKIIKDKDFLDNEENEDILEDIVL | 660 |
| | *******************************:**************************** | |
| SIN51_Bacterial | TLTLFEDRGMIEERLKTYAHLFDDKVMKQLKRRRYTGWGRLSRKLINGIRDKQSGKTILD | 700 |
| SIN53_mammalian | TLTLFEDREMIEERLKTYAHLFDDKVMKQLKRRRYTGWGRLSRKLINGIRDKQSGKTILD | 720 |
| | ******** *************************************************** | |
| SIN51_Bacterial | FLKSDGEANANFMQLIHDDSLTFKEDIQKAQVSGQGHSLHEQIANLAGSPAIKKGILQTV | 760 |
| SIN53_mammalian | FLKSDGFANRNFMQLIHDDSLTFKEDIQKAQVSGQGDSLHEHIANLAGSPAIKKGILQTV | 780 |
| | ********* **************************.****:****************** | |
| SIN51_Bacterial | KIVDELVKVMG-HKPENIVIEMARENQTTQKGQKNSRERMKRIEEGIKELGSQILKEHPV | 819 |
| SIN53_mammalian | KVVDELVKVMGRHKPENIVIEMARENQTTQKGQKNSRERMKRIEEGIKELGSQILKEHPV | 840 |
| | *:********* ************************************************ | |
| SIN51_Bacterial | ENTQLQNEKLYLYYLQNGRDMYVDQELDINRLSDYDVDHIVPQSFIKDDSIDNKVLTRSD | 879 |
| SIN53_mammalian | ENTQLQNEKLYLYYLQNGRDMYVDQELDINRLSDYDVDHIVPQSFLKDDSIDNKVLTRSD | 900 |
| | *********************************************:************** | |
| SIN51_Bacterial | KNRGKSDNVPSEEVVKKMKNYWRQLLNAKLITQRKFDNLTKAERGGLSELDKAGFIKRQL | 939 |
| SIN53_mammalian | KNRGKSDNVPSEEVVKKMKNYWRQLLNAKLITQRKFDNLTKAERGGLSELDKAGFIKRQL | 960 |
| | ************************************************************ | |
| SIN51_Bacterial | VETRQITKHVAQILDSRMNTKYDENDKLIREVKVITLKSKLVSDFRKDFQFYKVREINNY | 999 |
| SIN53_mammalian | VETRQITKHVAQILDSRMNTKYDENDKLIREVKVITLKSKLVSDFRKDFQFYKVREINNY | 1020 |
| | ************************************************************ | |
| SIN51_Bacterial | HHAHDAYLNAVVGTALIKKYPKLESEFVYGDYKVYDVRKMIAKSEQEIGKATAKYFFYSN | 1059 |
| SIN53_mammalian | HHAHDAYLNAVVGTALIKKYPKLESEFVYGDYKVYDVRKMIAKSEQEIGKATAKYFFYSN | 1080 |
| | ************************************************************ | |
| SIN51_Bacterial | IMNFFKTEITLANGEIRKRPLIETNGETGEIVWDKGRDFATVRKVLSMPQVNIVKKTEVQ | 1119 |
| SIN53_mammalian | IMNFFKTEITLANGEIRKRPLIETNGETGEIVWDKGRDFATVRKVLSMPQVNIVKKTEVQ | 1140 |
| | ************************************************************ | |
| SIN51_Bacterial | TGGFSKESILPKRNSDKLIARKKDWDPKKYGGFDSPTVAYSVLVVAKVEKGKSKKLKSVK | 1179 |
| SIN53_mammalian | TGGFSKESILPKRNSDKLIARKKDWDPKKYGGFDSPTVAYSVLVVAKVEKGKSKKLKSVK | 1200 |
| | ************************************************************ | |
| SIN51_Bacterial | ELLGITIMERSSFEKNPIDFLEAKGYKEVKKDLIIKLPKYSLFELENGRKRMLASAGELQ | 1239 |
| SIN53_mammalian | ELLGITIMERSSFEKNPIDFLEAKGYKEVKKDLIIKLPKYSLFELENGRKRMLASAGELQ | 1260 |
| | ************************************************************ | |
| SIN51_Bacterial | KGNELALPSKYVNFLYLASHYEKLKGSPEDNEQKQLFVEQHKHYLDEIIEQISEFSKRVI | 1299 |
| SIN53_mammalian | KGNELALPSKYVNFLYLASHYEKLKGSPEDNEQKQLFVEQHKHYLDEIIEQISEFSKRVI | 1320 |
| | ************************************************************ | |
| SIN51_Bacterial | LADANLDKVLSAYNKHRDKPIREQAENIIHLFTLTNLGAPAAFKYFDTTIDRKRYTSTKE | 1359 |
| SIN53_mammalian | LADANLDKVLSAYNKHRDKPIREQAENIIHLFTLTNLGAPAAFKYFDTTIDRKRYTSTKE | 1380 |
| | ************************************************************ | |
| SIN51_Bacterial | VLDATLIHQSITGLYETRIDLSQLGGDSPVRSSGGSSGGSSGSETPGTSESATPESSGGS | 1419 |
| SIN53_mammalian | VLDATLIHQSITGLYETRIDLSQLGGDSPVRSSGGSSGGSSGSETPGTSESATPESSGGS | 1440 |
| | ************************************************************ | |
| SIN51_Bacterial | SGGSSRPGERPFQCRICMRNFSNMSNLTRHTRTHTGEKPFQCRICMRNFSDRSVLRRHLR | 1479 |
| SIN53_mammalian | SGGSSRPGERPFQCRICMRNFSNMSNLTRHTRTHTGEKPFQCRICMRNFSDRSVLRRHLR | 1500 |
| | ************************************************************ | |
| SIN51_Bacterial | THTGSQKPFQCRICMRNFSDPSNLARHIRTHTGEKPFQCRICMRNFSDRSSLRRHLRTHT | 1539 |
| SIN53_mammalian | THTGSQKPFQCRICMRNFSDPSNLARHIRTHTGEKPFQCRICMRNFSDRSSLRRHLRTHT | 1560 |
| | ************************************************************ | |
| SIN51_Bacterial | GSQKPFQCRICMRNFSQSGTLHRHTRTHTGEKPFQCRICMRNFSQRPNLTRHLRTHLRGS | 1599 |
| SIN53_mammalian | GSQKPFQCRICMRNFSQSGTLHRHTRTHTGEKPFQCRICMRNFSQRPNLTRHLRTHLRGS | 1620 |
| | ************************************************************ | |
| SIN51_Bacterial | GSAGSAAGSGEFPKKKRKV* | 1618 |
| SIN53_mammalian | GSAGSAAGSGEFPKKKRKV* | 1639 |
| | ******************** | |

A sgRNA expression vector labeled cdsDNA164 was purchased from Vector Builder (Chicago, IL). It features the g526 spacer sequence cloned into Vector Builder's standard mammalian gRNA expression vector consisting of the U6 promoter driving the transcription of the spacer and gRNA scaffold sequence and a standard pUC19/ampicillin resistant plasmid backbone. The sequence of cdsDNA164 is shown in SEQ ID NO: 9.

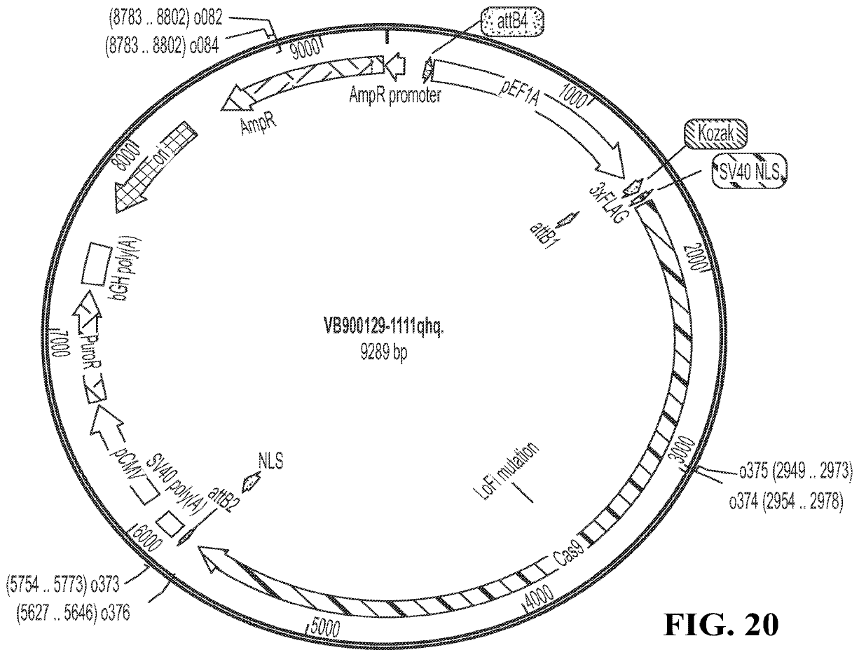

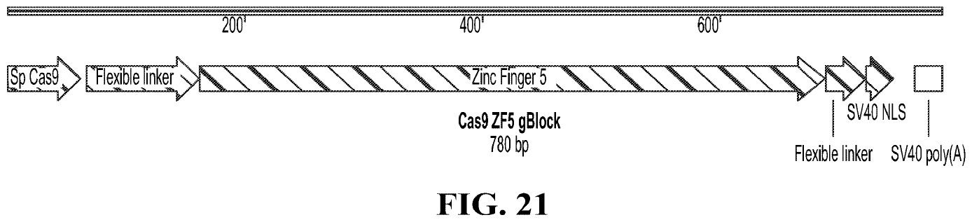

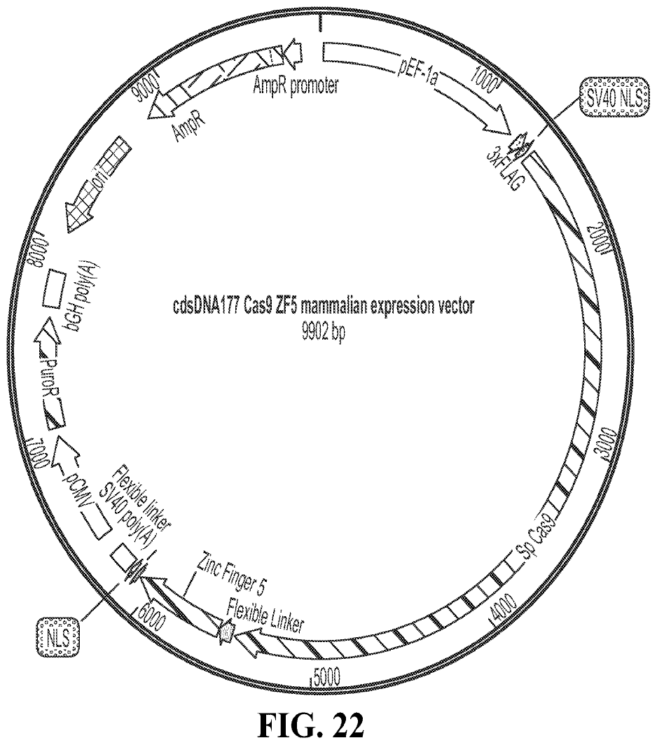

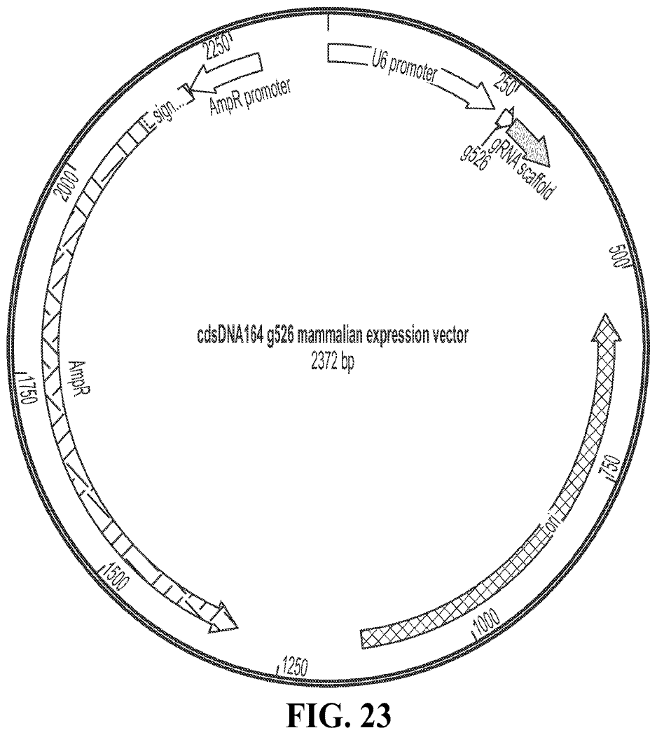

FIG. 20 shows a schematic of cdsDNA166 Standard Cas9 mammalian expression vector from Vector Builder (SEQ ID NO: 8). FIG. 21 shows a schematic of a Zinc finger 5 gBlock (SEQ ID NO: 13). FIG. 22 shows a schematic of cdsDNA177 Cas9 ZF5 mammalian expression vector (SEQ ID NO: 7). FIG. 23 shows a schematic of cdsDNA164 g526 sgRNA mammalian expression vector (SEQ ID NO: 9).

### Transfection Sample Setup

Samples were produced in triplicate.

First approach: HiFi Sp Cas9:zinc finger fusion protein was incubated with sgRNA g526 at a 1:2 molar ratio at 37 C for 20 minutes to form an RNP complex. cssDNA222 was diluted to 700 ng/μL in zinc binding buffer (see above example) and 2000 ng of cssDNA was mixed with 26 μmoles of RNPs in 200 μL PCR tube and incubated at 25 C for 1 hour to allow the zinc finger to bind to the ZFDBM5 hairpin sequence on the cssDNA.

Second approach: 2000 ng of cssDNA222 emGFP at TRAC integrating cssDNA with ZFDBM5 was mixed with 1000 ng of cdsDNA177 Cas9:zinc finger fusion expression vector and 1000 ng of cdsDNA164 g526 sgRNA expression vector.

The negative controls in this genome editing experiment were buffer only, cssDNA221 only, cssDNA222 only, RNPs only, and expression vectors only. The experimental samples were RNPs with cssDNA222 and expression vectors with cssDNA222.

### Transfection Protocol

HEK293T cells (Catalog number CRL-3216 ATCC, Manassas. VA) were cultured for at least one week in Dulbecco's Modified Eagle Medium (DMEM) (catalog #11960044, Thermofisher, Waltham, MA) supplemented with Glutamax (catalog #25050061. Thermofisher, Waltham, MA), Penicillin-Streptomycin-Glutamine (catalog #10378016. Thermofisher, Waltham, MA) and 10% Fetal Bovine Serum (catalog #A5256701, Thermofisher, Waltham, MA). Components were mixed and sterile filtered through a 0.22 micron PES vacuum filter. To prepare for transfection, cells were seeded at 2.6e5 cells per cm² in a T-75 tissue culture flask and incubated at 37 C in 5% C02 for 48 hours.

Cells were harvested when they were 85-90% confluent. Cells were then dissociated from the flask by aspirating spent media and washing with equilibrated Dulbecco's phosphate buffered saline (dPBS) (catalog #14190144, Thermofisher, Waltham, MA). Cells were then treated with 2 ml equilibrated 0.12% Trypsin-EDTA in dPBS (catalog #25200114, Thermofisher, Waltham, MA). After two minutes, the trypsin reaction was quenched with DMEM and cells were counted on a hemocytometer with 0.4% Trypan blue. Cells were then centrifuged at 90 RCF for 10 minutes then resuspended to 1e7 cells per mL in SF buffer with supplement (SF Cell Line 4D-Nucleofector X Kit S) (catalog #V4XC-2032, Lonza Walkersville, MD). 20 μL containing 2e5 cells was then mixed with the RNPs, expression vectors and cssDNA samples described above and transferred into a 16-well cuvette strip (SF Cell Line 4D-Nucleofector X Kit S) (catalog #V4XC-2032, Lonza Walkersville, MD). The cells were then electroporated in the 4d Nucleofector X Unit (Lonza, Walkersville, MD) using program CM-120. 80 μL of DMEM was immediately added to the cuvettes and cells were transferred to a 96-well tissue culture plate containing 150 μL of DMEM. Cells were then incubated at 37 C in 5% C02 and split one to two every day for 5 days after the transfection. Cells were then stained with 1 μg/mL propidium iodide for viability and then analyzed via flow cytometry for GFP expression and viability using the Attune NxT flow cytometer (Thermofisher, Waltham, MA). FCS files were analyzed in FlowJo software (FlowJo LLC., Ashland, OR). Flow cytometry events were gated for live, single cells and the percent of GFP fluorescent cells out of this population was recorded for each sample.

### Results

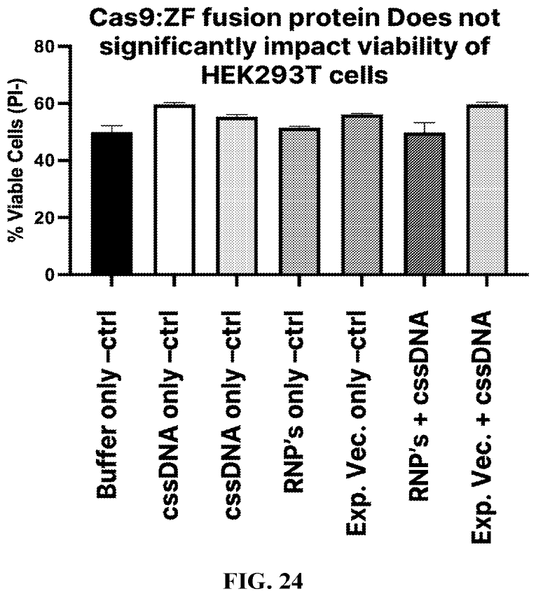

FIG. 24 shows the viability of HEK293T cells after transfection with only buffer, only cssDNA, only RNPs, only expression vector, RNPs and cssDNA, and expression vector and cssDNA. Electroporated cells from all conditions have between 50% and 60% viability. Cells transfected with RNPs have a slightly lower viability than cells transfected with DNA only, but even the RNP samples have higher viability than the buffer only negative control indicating the neither the RNPs nor the DNA needed for genomics integration are reducing the viability of the cells greater than the electroporation process itself.

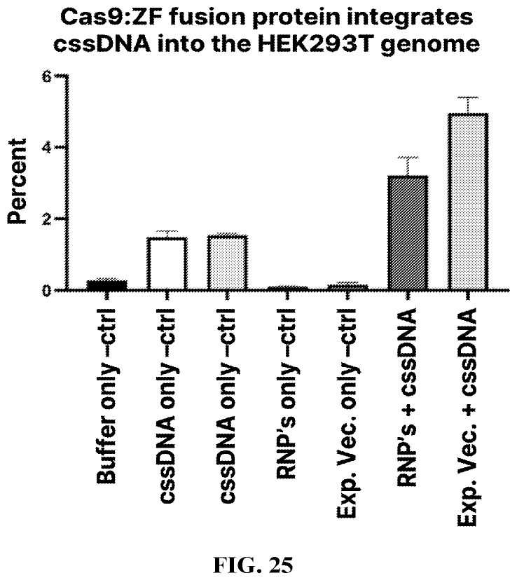

FIG. 25 shows that a Cas9:ZF fusion protein described herein integrates cssDNA into the HEK293T genome.

HEK293T cells transfected with cssDNA222 (which includes a ZFDBM5 hairpin sequence and a GFP at TRAC integration cassette) and either (i) a Cas9:zinc finger fusion protein RNP targeted to the TRAC locus by sgRNA g526 or (ii) a Cas9:zinc finger fusion protein expression vector targeted to the TRAC locus by a sgRNA g526 expression vector integrate the GFP at TRAC sequence encoded on the cssDNA at rates significantly higher than the controls using any of these components alone. This shows that the engineered Cas9:zinc finger fusion protein used either of the approaches (in its protein form as part of an RNP or in its DNA form in a mammalian expression vector) is capable of driving the integration of cssDNA sequences in mammalian cells. Not wishing to be bound by any particular theory, we anticipate that this system would have even greater benefits in non-dividing or quiescent cells due the relative difficulty of bringing DNA into the nucleus the HEK293T cells used in this Example. HEK293T cells used in this experiment are one of the fastest growing human cell lines and therefore one of the easiest in which to bring DNA into the nucleus due to the fact that during mitosis, the nuclear membrane must open and therefore allow DNA to enter. In non-dividing cells, the nuclear membrane does not open so some other mechanism must be used for DNA to enter the nucleus such as tethering the DNA to a protein that possesses a nuclear localization signal as was done here.

LISTING OF SEQUENCES

| SEQ ID | | |
|---|---|---|
| NO: | Description | Sequence |
| 1 | Cas9- | ATGGCTTCCCCACCCAAGAAAAAACGTAAGGTAGGGTCTATGGACAAAAAATATAGCATCGGGCTGGAT |
| | ZF_fusion_ | ATCGGAACCAATAGCGTTGGGTGGGCCGTCATTACCGATGACTATAAGGTCCCTTCTAAAAAATTCAAG |
| | g 4839 bp | GTTTTAGGAAATACCGACCGCCATTCAATTAAGAAAAATTTGATCGGCGCGTTGCTGTTTGGTAGTGGG |
| | DNA linear | GAAACTGCGGAGGCAACTCGCCTTAAGCGTACAGCACGTCGCCGCTACACCCGCCGCAAGAATCGTATC |
| | Natural DNA | TGCTACTTGCAGGAGATCTTTAGTAATGAGATGGCCAAAGTAGACGATTCATTTTTTCACCGCCTGGAA |
| | sequence | GAATCTTTCTTGGTAGAGGAGGACAAAAAGCACGAGCGTCATCCAATTTTTGGTAACATTGTAGATGAA |
| | | GTGGCTTATCATGAAAAGTACCCCACGATTTACCATTTACGCAAGAAGTTGGCAGATTCAACTGATAAA |
| | | GCGGATCTTCGCCTGATTTACCTTGCGCTGGCACATATGATCAAGTTCCGCGGTCACTTCTTGATCGAG |
| | | GGCGATCTGAATCCAGACAATTCCGACGTCGACAAATTGTTTATCCAATTGGTTCAGATCTACAATCAA |
| | | CTGTTCGAGGAGAATCCCATCAATGCCAGCCGCGTAGACGCTAAGGCCATTTTATCAGCGCGTCTTAGT |
| | | AAAAGTCGTCGTCTGGAAAACTTAATCGCCCAGCTTCCCGGGGAGAAGCGTAATGGGTTATTCGGCAAC |
| | | TTGATCGCTCTTTCCCTGGGATTGACCCCGAACTTCAAATCGAACTTCGACCTTGCTGAGGATGCAAAG |
| | | TTGCAATTGTCTAAAGATACCTACGACGATGATCTGGATAACCTGTTGGCGCAGATCGGCGACCAATAT |
| | | GCCGACCTTTTTCTTGCCGCGAAGAACCTTTCTGATGCCATTTTGCTGTCAGATATCTTGCGTGTGAAT |
| | | TCTGAAATCACGAAGGCCCCCTTAAGCGCGTCAATGATTAAGCGCTATGATGAGCATCATCAGGACCTG |
| | | ACATTATTGAAAGCCCTGGTTCGCCAACAGTTGCCGGAGAAATATAAAGAAATTTTTTTCGATCAAAGC |
| | | AAAAATGGGTACGCGGGCTATATTGACGGCGGTGCTTCCCAGGAAGAGTTTTACAAATTCATCAAACCA |
| | | ATCTTGGAAAAAATGGACGGCACCGAGGAGTTACTGGTCAAACTGAACCGTGAAGATCTGCTTCGCAAA |
| | | CAGCGCACCTTCGATAATGGCAGCATTCCCCACCAGATTCACCTGGGCGAATTACATGCCATTTTGCGT |
| | | CGTCAGGAAGACTTTTATCCCTTTTTGAAAGATAACCGTGAAAAAATTGAAAAGATCCTGACCTTCCGC |
| | | ATTCCGTACTATGTCGGACCCCTGGCCCGCGGTAACTCACGTTTCGCGTGGATGACTCGCAAGTCCGAG |
| | | GAAACAATTACGCCATGGAACTTCGAGGAAGTGGTGGACAAAGGGGCGTCTGCTCAGTCCTTCATTGAG |
| | | CGTATGACTAATTTTGACAAGAACCTGCCGAACGAGAAGGTCTTACCGAAGCATAGTTTGCTGTATGAG |
| | | TACTTCACAGTGTACAACGAACTTACTAAGGTGAAATATGTGACTGAAGGGATGCGTAAGCCCGCCTTT |
| | | TTAAGTGGTGAGCAAAAGAAAGCAATCGTCGATTTGCTTTTTAAGACAAATCGTAAAGTCACAGTTAAA |
| | | CAGCTGAAGGAGGACTACTTTAAAAAAATTGAATGTTTCGATTCAGTCGAAATTTCGGGAGTAGAGGAT |
| | | CGTTTTAACGCGAGCCTGGGAGCTTACCATGATTTACTTAAGATCATTAAAGATAAAGATTTTTTGGAC |
| | | AACGAGGAAAACGAAGATATCTTGGAAGATATCGTGTTGACATTAACCCTGTTTGAGGACCGCGGCATG |
| | | ATCGAGGAACGTCTGAAAACTTACGCGCATTTGTTCGATGACAAAGTCATGAAACAGCTGAAACGCCGT |
| | | CGCTACACTGGCTGGGGACGTCTTAGCCGTAAGCTTATTAATGGTATCCGCGACAAACAATCCGGCAAG |
| | | ACCATCCTGGACTTTTTAAAAAGCGACGGATTCGCTAACGCCAACTTTATGCAATTAATCCATGACGAC |
| | | AGTCTTACCTTCAAAGAAGACATCCAAAAAGCTCAAGTCTCGGGCCAAGGTCACTCTTTGCATGAGCAA |
| | | ATTGCGAACTTAGCCGGCTCACCAGCTATTAAGAAAGGGATCCTTCAGACTGTTAAGATCGTAGATGAG |
| | | TTAGTGAAAGTAATGGGACATAAGCCAGAAAATATTGTCATCGAAATGGCACGCGAGAACCAGACAACA |
| | | CAGAAGGGACAAAAAAACTCGCGTGAACGTATGAAGCGCATTGAAGAAGGGATCAAAGAATTAGGGTCT |
| | | CAGATTTTGAAAGAACATCCGGTTGAGAATACGCAACTGCAAAATGAAAAGTTATATTTGTATTATTTG |
| | | CAAAACGGACGCGACATGTACGTAGATCAGGAACTGGATATCAACCGTCTGTCCGATTATGATGTAGAT |
| | | CACATCGTGCCACAATCTTTCATTAAAGACGACTCTATCGACAACAAGGTACTTACACGTTCTGATAAA |
| | | AACCGTGGTAAGTCGGACAATGTACCGTCCGAGGAGGTGGTTAAGAAAATGAAGAATTACTGGCGTCAA |
| | | TTACTTAATGCCAAATTGATCACCCAACGTAAGTTCGATAACCTGACTAAGGCCGAACGTGGGGGGTTA |
| | | TCGGAACTTGACAAGGCCGGATTCATTAAGCGTCAACTTGTTGAAACGCGCCAAATCACGAAACACGTT |
| | | GCTCAAATTCTTGATAGCCGCATGAACACTAAGTACGATGAGAATGATAAGCTTATCCGTGAAGTTAAG |
| | | GTGATTACCCTTAAGTCCAAGTTGGTGTCTGACTTCCGCAAGGATTTTCAGTTCTATAAGGTTCGCGAG |
| | | ATTAATAATTACCATCATGCGCACGATGCATATCTTAATGCCGTGGTTGGCACCGCTTTAATCAAAAAG |
| | | TATCCTAAATTGGAGAGTGAGTTCGTTTACGGCGACTATAAAGTTTACGATGTGCGTAAGATGATTGCT |
| | | AAGTCCGAACAGGAAATCGGTAAGGCGACAGCCAAATATTTCTTCTACTCAAACATTATGAATTTTTTT |
| | | AAAACGGAGATTACATTAGCTAATGGTGAAATCCGTAAGCGCCCGTTGATTGAGACAAACGGCGAGACC |
| | | GGCGAGATTGTCTGGGATAAAGGACGTGATTTCGCCACCGTCCGCAAGGTCCTGAGCATGCCCCAAGTT |
| | | AACATTGTGAAGAAGACAGAAGTTCAGACCGGCGGATTCAGTAAGGAGTCGATCTTGCCAAAACGCAAT |
| | | AGCGATAAGTTAATTGCTCGCAAGAAAGACTGGGATCCGAAAAAGTATGGAGGGTTCGACAGCCCCACA |
| | | GTCGCGTACTCTGTACTTGTTGTAGCGAAAGTTGAAAAGGGCAAATCTAAGAAATTAAAATCCGTAAAA |
| | | GAGCTGTTGGGAATCACAATTATGGAGCGCAGCTCGTTTGAAAAAAACCCCATCGACTTCCTGGAGGCG |
| | | AAGGGTTATAAAGAAGTTAAGAAGGACTTGATCATTAAATTACCTAAATACAGTTTATTTGAGTTGGAG |
| | | AACGGTCGTAAGCGCATGTTAGCCTCGGCAGGGGAGTTACAAAAGGGCAACGAACTGGCGTTACCAAGC |
| | | AAGTACGTCAATTTCCTTTACTTAGCCTCGCATTATGAGAAGTTGAAAGGAAGTCCTGAAGACAACGAG |
| | | CAGAAGCAATTATTTGTGGAGCAACATAAACATTACCTTGACGAGATCATCGAGCAGATCTCGGAGTTT |
| | | TCGAAGCGCGTAATTTTAGCGGATGCCAACTTAGACAAGGTATTGTCGGCCTATAATAAACACCGTGAC |
| | | AAGCCTATTCGCGAGCAAGCAGAGAACATTATTCACTTGTTTACCTTAACCAACCTTGGGGCTCCTGCC |
| | | GCTTTCAAGTATTTCGATACTACAATCGACCGTAAGCGTTATACGTCAACTAAGGAAGTGTTAGACGCG |
| | | ACACTGATTCATCAATCCATCACTGGGTTATACGAAACCCGTATTGACTTATCTCAGCTTGGCGGGGAT |
| | | TCACCCGTCCGTTCATCCGGAGGTAGCTCAGGGGGCTCGTCTGGCTCCGAGACTCCCGGCACCTCCGAA |
| | | TCGGCAACGCCGGAGTCAAGCGGAGGCAGCTCTGGAGGCAGCTCGCGCCCCGGTGAGCGTCCCTTTCAA |
| | | TGCCGTATTTGTATGCGCAACTTCTCAAATATGTCGAACCTGACACGTCATACCCGTACACATACTGGC |
| | | GAGAAGCCATTTCAGTGTCGCATTTGCATGCGCAACTTTAGTGATCGTTCAGTATTACGCCGCCACCTT |
| | | CGCACGCACACTGGGTCTCAGAAGCCTTTCCAATGCCGCATCTGCATGCGTAACTTTTCGGATCCCTCC |
| | | AATCTGGCACGCCATACACGTACCCATACGGGGGAAAAGCCTTTTCAGTGTCGCATTTGTATGCGCAAC |
| | | TTTTCCGATCGCTCTTCGCTTCGCCGCCATTTACGCACGCATACGGGCTCTCAAAAACCCTTCCAGTGC |
| | | CGCATCTGCATGCGTAACTTTTCGCAGTCAGGGACGTTGCACCGTCACACACGCACCCATACCGGTGAA |
| | | AAGCCCTTTCAGTGTCGTATTTGCATGCGCAATTTTAGTCAGCGCCCCAACTTAACCCGTCACTTGCGC |
| | | ACACATCTGCGTGGAAGTGGATCTGCGGGGTCGGCAGCGGGATCCGGAGAATTTCCCAAGAAAAAGCGT |
| | | AAAGTTTAA |
| 2 | phagemid | TGAAGCAGTCGACGCCGAAGAGGGTCCGAGGTATTCCTTCGGCGTCGACTGCTTCAGCGCCCTGTAGCG |
| | 9966 bp | GCGCATTAAGCGCGGCGGGTGTGGTGGTTACGCGCAGCGTGACCGCTACACTTGCCAGCGCCCTAGCGC |
| | DNA | CCGCTCCTTTCGCTTTCTTCCCTTCCTTTCTCGCCACGTTCGCCGGCTTTCCCCGTCAAGCTCTAAATC |
| | circular | GGGGGCTCCCTTTAGGGTTCCGATTTAGTGCTTTACGGCACCTCGACCCCAAAAAACTTGATTTGGGTG |
| | synthetic | ATGGTTCACGTAGTGGGCCATCGCCCTGATAGACGGTTTTTCGCCCTTTGACGTTGGAGTCCACGTTCT |
| | circular | TTAATAGTGGACTCTTGTTCCAAACTGGAACAACACTCAACCCTATCTCGGGTTTCCATAGGCTCCGCC |
| | DNA | CCCCTGACGAGCATCACAAAAATCGACGCTCAAGTCAGAGGTGGCGAAACCCGACAGGACTATAAAGAT |
| | | ACCAGGCGTTTCCCCCTGGAAGCTCCCTCGTGCGCTCTCCTGTTCCGACCCTGCCGCTTACCGGATACC |
| | | TGTCCGCCTTTCTCCCTTCGGGAAGCGTGGCGCTTTCTCATAGCTCACGCTGTAGGTATCTCAGTTCGG |
| | | TGTAGGTCGTTCGCTCCAAGCTGGGCTGTGTGCACGAACCCCCCGTTCAGCCCGACCGCTGCGCCTTAT |
| | | CCGGTAACTATCGTCTTGAGTCCAACCCGGTAAGACACGACTTATCGCCACTGGCAGCAGCCACTGGTA |
| | | ACAGGATTAGCAGAGCGAGGTATGTAGGCGGTGCTACAGAGTTCTTGAAGTGGTGGCCTAACTACGGCT |
| | | ACACTAGAAGGACAGTATTTGGTATCTGCGCTCTGCTGAAGCCAGTTACCTTCGGAAAAAGAGTTGGTA |
| | | GCTCTTGATCCGGCAAACAAACCACCGCTGGTAGCGGTGGTTTTTTTGTTTGCAAGCAGCAGATTACGC |
| | | GCAGAAAAAAAGGATCTCAAGATCTATGGATTTATCAATGAACTGATAAAATTCAGTCCAGAAATTTGT |
| | | TTCTAAGGACATTGTTTCTTTTTGAAAGTCCACCCTATCTCGTAATTCATTCCCAGATCAACTCTCCAA |
| | | ACGAATATAATCCTTTACTCCTTTTTCATTTCCTGTAAAGTATCGCCAGGAGGTAAATCTATGACTTTA |
| | | ACCGCTAGTTCATCATCCCGCGCAGTGACGAATTCGCCGGTTGTAGTTGCACTTGACTATCATAATCGC |
| | | GATGACGCATTAGCTTTTGTCGACAAAATTGATCCACGCGATTGTCGTCTTAAAGTAGGTAAGGAGATG |
| | | TTTACGCTTTTTGGACCGCAATTCGTGCGCGAACTGCAACAACGTGGTTTTGACATTTTCCTTGACCTT |
| | | AAGTTTCACGATATCCCGAACACTGCAGCTCATGCGGTAGCTGCGGCTGCTGACCTTGGTGTTTGGATG |
| | | GTAAATGTTCACGCCTCTGGTGGGGCACGTATGATGACCGCAGCTCGTGAAGCACTTGTCCCTTTCGGA |
| | | AAGGATGCCCCATTGTTAATTGCCGTCACTGTCCTTACTTCGATGGAGGCTTCAGACTTAGTCGATTTA |
| | | GGCATGACATTGTCACCAGCCGATTATGCGGAACGTTTGGCGGCGTTGACTCAAAAATGTGGGTTAGAC |
| | | GGCGTCGTTTGTAGCGCCCAGGAAGCTGTTCGCTTTAAACAAGTCTTCGGACAGGAGTTCAAGTTAGTT |
| | | ACTCCCGGAATCCGCCCCCAGGGTAGTGAGGCAGGAGACCAACGCCGCATCATGACACCTGAGCAGGCA |
| | | CTTTCCGCTGGGGTCGACTATATGGTAATCGGCCGTCCCGTTACTCAATCAGTCGACCCTGCACAAACA |
| | | TTGAAGGCAATCAATGCTTCCCTGCAACGCAGCGCATGACTCGGTACCAAATTCCAGAAAAGAGGCCTC |
| | | CCGAAAGGGGGGCCTTTTTTCGTTTTGGTCCGGTCTGGGAAGGGAAAAGCATTACTATCTATCTTGAAT |
| | | ATTCATGTTTCTCTAGGTCAAACACATTAAAATTTGACTTTAATCATTCAATGGGTATTGTAAAATGCC |
| | | TTCTATGTGACTATCACTCTATAAAATGTTAGACTGAGTATGAAGTGTGAGATAGATTCCTGTCCTGTC |
| | | CTCAAGTGGTATAAAAACTAGACAAAGGTACTGAACTATTGTAAATTAAGCAGCCAGAAAACTATTTTA |
| | | GTATTGACCAGTTGATGATGTCAATGGACACAATAGAGATTCAGAGGGGATAGGGTGCTGGCTGAGAAG |
| | | TTGGCCTAGACTGAGAAGTTTCCTGTGGTACAAAGGATTCATTGAGCCCTGAAGGATGGATAAGATCTG |
| | | TATGGGCAGAGAAAGGAGAGAAGGGAAGTTCTGGGCGTAGGGAACGACAAGAAAGAAGGCATGATCTTG |
| | | GGAATAATCAAGGCACATGCAAAGTAGCCTAAGTATACATCTGATAATAAAATTGGTTGAAAAGTAGTC |
| | | AGAGAAGATGTCTTTTTAGGCATGGAAAAAGGAAATACTAGAGCATTCAACAGAATACAGAAATTAGGG |
| | | CCAGGGCCAGCCATTGGGAAACTGAGAATCCGATTTAGAGATGCAGACTAGAAGTGAAGGTGAGAGCAG |
| | | CCAGCTATGGTGCCGCAGACCTCCCCTCTCCTTCCTCAGTGGGCTCTGAGAGGGGTCATCCCACACCTT |
| | | AGAGGAGGAGAAACCTAAGGGATTCTGTAATAGAGACACGGGGCATGGTATGAAAGTATTACCTCCCAG |
| | | TTGCAATTTGGCAAAGGAACCAGAGTTTCCACTTCTCCCCGTACGTCTGCCCATGCCCACAGTTTCCTG |
| | | ATGCTCACTAAAGCCTCGGTGGGACCCAGAGTGACTGTCACTAATTCTGATTTCTGGGTCCCTAGTGCC |
| | | CAAACACGGGGGACAGATTTAATGGTAAGGAAGCTTTCAATCACTGCTGTGTCCCTAGGGATCTAAAGC |
| | | ACTAGAGCACATGTGCTTCTGCAGTTCATTTTGAATTTAAAGGACAGCTTAGGATCTAGAATAGCTGAA |
| | | TTTCCACCTCAAAACATTGGTTCCGTCTTGCCAAGCCTACCTTCTGATATCATCAGTGATGGGATGTGT |
| | | TTTTCTTACTAGGGTAGAATAGGATGTCTCTCCCCAAAGGACTCTGGCAGACAGACCCCTAAACACCTC |
| | | CAAATTAAAAGCGGCAAAGAGATAAGGTTGAACTAGACGTACATGGGGATAAAAAGTATAAAAGGTACA |
| | | TGGGAATGAAAGGATAAAAAGGCTAAAAAAATTAAGTACCTCTAACTCAGCCCCTGTTGCCATTTCTCA |
| | | GAGTCTTGTGTTCTGTGGCATTGCGCTTTCTAGACCAACAGTGTCCAATAGAACTTTCTGTGGCAATGG |
| | | AAATGTCCTGTCAATCTGCACTGTCCCATACAATAGCCACCAGCTACATGTGGCTATTGAGCTCTTGAA |
| | | ATGAAGTTTCCATTTTTAATTGAAAACATTTTATTTCACATTGACTAATTTTTATTTCAACAGCCACAT |
| | | GTAGCTAGAGACTATTATACCAGACAGAGCAGCCTAGATCTTCTCCAGTCTGACACCCACCAGCCCCAG |
| | | GACTTGAGTGAGTGTTTAACCAGGACTCAAAGTTGGGTTTCTGCCCCACAAGGCCACCCCCTTTCCTCT |
| | | TTAAAGCCAACCTGCATCTGGTGGCCCCTGATCCCCTGCCTTGAGGATCGGCACTTCCAGACTCCTCTC |
| | | CCCCTCTGCAGTGCTGTCCAGTACCCCCACTGATGACTAACAATCAGGGGGATGTGTTGGTAGAGCTAA |
| | | TGGCTTTCTGTCTGTCCCTTCCCAGCAAAGGAACTATGCCTTAGGGCCTTCACCCAGAGTGATGTCAGG |
| | | CTGCCCAAGCATGAGGAGGGAAGTAGGCAGAATCCTCTGGAGCCAAAGCTCTGGATGTCTCTCCCCTCT |
| | | GACCATGGAGCCCACCCCTGCTCCACTGCTCCAGGGACAGCCCTATGCTGCAGGCAGCTCTGCCCCCAC |
| | | TCAGCATCCCAGGGGCTGATTTCTTTGGTTTTGGATCCAGCTGGATGTCTGCATTGCCGAGGCCACCAG |
| | | GGCTGGCTCAGCAACTGTCGGGGAATCACCAGGGTCTGAGAAATCTTGTGCGCATGTGAGGGGCTGTGG |
| | | GAGCAGAGAACCACTGGGTGGGAAATTCTAATCCCCACCCTGCTGGAAACTCTCTGGGTGGCCCCAACA |
| | | TGCTAATCCTCCGGCAAACCTCTGTTTCCTCCTCAAAAGGCAGGAGGTCGGAAAGAATAAACAATGAGA |
| | | GTCACATTAAAAACACAAAATCCTACGGAAATACTGAAGAATGAGTCTCAGCACTAAGGAAAAGCCTCC |
| | | AGCAGCTCCTGCTTTCTGAGGGTGAAGGATAGACGCTGTGGCTCTGCATGACTCACTAGCACTCTATCA |
| | | CGGCCATATTCTGGCAGGGTCAGTGGCTCCAACTAACATTTGTTTGGTACTTTACAGTTTATTAAATAG |
| | | ATGTTTATATGGAGAAGCTCTCATTTCTTTCTCAGAAGAGCCTGGCTAGGAAGGTGGATGAGGCACCAT |
| | | ATTCATTTTGCAGGTGAAATTCCTGAGATGTAAGGAGCTGCTGTGACTTGCTCAAGGCCTTATATCGAG |
| | | TAAACGGTAGTGCTGGGGCTTAGACGCAGGTGTTCTGATTTATAGTTCAAAACCTCTATCAATGAGAGA |
| | | GCAATCTCCTGGTAATGTGATAGATTTCCCAACTTAATGCCAACATACCATAAACCTCCCATTCTGCTA |
| | | ATGCCCAGCCTAAGTTGGGGAGACCACTCCAGATTCCAAGATGTACAGTTTGCTTTGCTGGGCCTTTTT |
| | | CCCATGCCTGCCTTTACTCTGCCAGAGTTATATTGCTGGGGTTTTGAAGAAGATCCTATTAAATAAAAG |
| | | AATAAGCAGTATTATTAAGTAGCCCTGCATTTCAGGTTTCCTTGAGTGGCAGGCCAGGCCTGGCCGTGA |
| | | ACGTTCACTGAAATCATGGCCTCTTGGCCAAGATTGATAGCTTGTGCCTGTCCCTGAGTCCCAGTCCAT |
| | | CACGAGCAGCTGGTTTCTAAGATGCTATTTCCCGTATAAAGCATGAGACCGTGACTTGCCAGCCCCACA |
| | | GAGCCCCGCCCTTGTCCATCACTGGCATCTGGACTCCAGCCTGGGTTGGGGCAAAGAGGGAAATGAGAT |
| | | CATGTCCTAACCCTGATCCTCTTGTCCCACAGCGTCTCGAAGGGGATCCTACTGACGAGGGGCTTGTCC |
| | | AAACAAGCCCGGGCATGCCTAAACATGCCCTGATGCAATCCTGACGTCGTGAAGCAATGATTATGCAAT |
| | | TTGAGCATGTCCAGACTAGCCCTGACGGATGACGCTTGACGCAATTCCTGAGGCAAGTCTGAGCTTGTT |
| | | CAAACTTGTCTTGAAGAAATTATGACGGACTGACGTATGGTGCAATATTGGGGCAATGCTTGACGTTCG |
| | | CGGTAGGCGTGTACGGTGGGAGGTCTATATAAGCAGAGCTCGTTTAGTGAACCGTCAGATCGCCTGGTG |
| | | ACGCCATCCACGCTGTTTTGACCTCCATAGAAGACACCGGGACCGATCCAGCCTCTCGACATTCGTGCC |
| | | ACCATGGTCTCCAAAGGAGAAGAGGATAACATGGCAATTATCAAAGAATTTATGAGATTTAAGGTCCAC |
| | | ATGGAAGGGTCAGTGAACGGCCACGAATTTGAAATAGAAGGAGAAGGAGAGGGACGACCATACGAAGGC |
| | | ACACAGACTGCAAAATTGAAGGTGACAAAGGGGGGTCCTCTCCCATTTGCATGGGATATACTGAGTCCT |
| | | CAATTCATGTATGGTAGCAAGGCGTATGTAAAACACCCAGCTGATATTCCGGATTATCTCAAACTCTCA |
| | | TTTCCGGAAGGGTTCAAATGGGAAAGAGTCATGAATTTTGAAGATGGAGGTGTAGTCACGGTAACTCAG |
| | | GATTCCAGTCTCCAAGACGGAGAATTTATTTACAAAGTTAAGTTGAGGGGTACCAACTTTCCGAGTGAT |
| | | GGCCCGGTAATGCAAAAGAAGACTATGGGTTGGGAAGCCAGCTCTGAAAGAATGTATCCTGAGGACGGC |
| | | GCCCTCAAGGGAGAGATTAAGCAGAGACTGAAACTTAAGGACGGAGGGCACTACGATGCAGAAGTAAAG |
| | | ACAACCTACAAAGCCAAAAAACCAGTACAGCTTCCGGGTGCATACAATGTGAACATTAAATTGGATATT |
| | | ACCTCTCATAACGAAGACTACACAATCGTCGAACAATACGAGAGAGCCGAAGGCCGCCATTCAACAGGA |
| | | GGAATGGACGAGCTTTACAAGTAAGCGGGACTCTGGGGTTCGAAATGACCGACCAAGCGACGCCCAACC |
| | | TGCCATCACGAGATTTCGATTCCACCGCCGCCTTCTATGAAAGGTTGGGCTTCGGAATCGTTTTCCGGG |
| | | ACGCCGGCTGGATGATCCTCCAGCGCGGGGATCTCATGCTGGAGTTCTTCGCCCACCCTAGGGGGAGGC |
| | | TAACTGAAACACGGAAGGAGACAATACCGGAAGGAACCCGCGCTATGACGGCAATAAAAAGACAGAATA |
| | | AAACGCACGGTGTTGGGTCGTTTGTTCACTCGGAGACGCGATATCCAGAACCCTGACCCTGCCGTGTAC |
| | | CAGCTGAGAGACTCTAAATCCAGTGACAAGTCTGTCTGCCTATTCACCGATTTTGATTCTCAAACAAAT |
| | | GTGTCACAAAGTAAGGATTCTGATGTGTATATCACAGACAAAACTGTGCTAGACATGAGGTCTATGGAC |
| | | TTCAAGAGCAACAGTGCTGTGGCCTGGAGCAACAAATCTGACTTTGCATGTGCAAACGCCTTCAACAAC |
| | | AGCATTATTCCAGAAGACACCTTCTTCCCCAGCCCAGGTAAGGGCAGCTTTGGTGCCTTCGCAGGCTGT |
| | | TTCCTTGCTTCAGGAATGGCCAGGTTCTGCCCAGAGCTCTGGTCAATGATGTCTAAAACTCCTCTGATT |
| | | GGTGGTCTCGGCCTTATCCATTGCCACCAAAACCCTCTTTTTACTAAGAAACAGTGAGCCTTGTTCTGG |
| | | CAGTCCAGAGAATGACACGGGAAAAAAGCAGATGAAGAGAAGGTGGCAGGAGAGGGCACGTGGCCCAGC |
| | | CTCAGTCTCTCCAACTGAGTTCCTGCCTGCCTGCCTTTGCTCAGACTGTTTGCCCCTTACTGCTCTTCT |
| | | AGGCCTCATTCTAAGCCCCTTCTCCAAGTTGCCTCTCCTTATTTCTCCCTGTCTGCCAAAAAATCTTTC |
| | | CCAGCTCACTAAGTCAGTCTCACGCAGTCACTCATTAACCCACCAATCACTGATTGTGCCGGCACATGA |
| | | ATGCACCAGGTGTTGAAGTGGAGGAATTAAAAAGTCAGATGAGGGGTGTGCCCAGAGGAAGCACCATTC |
| | | TAGTTGGGGGAGCCCATCTGTCAGCTGGGAAAAGTCCAAATAACTTCAGATTGGAATGTGTTTTAACTC |
| | | AGGGTTGAGAAAACAGCTACCTTCAGGACAAAAGTCAGGGAAGGGCTCTCTGAAGAAATGCTACTTGAA |
| | | GATACCAGCCCTACCAAGGGCAGGGAGAGGACCCTATAGAGGCCTGGGACAGGAGCTCAATGAGAAAGG |
| | | AGAAGAGCAGCAGGCATGAGTTGAATGAAGGAGGCAGGGCCGGGTCACAGGGCCTTCTAGGCCATGAGA |
| | | GGGTAGACAGTATTCTAAGGACGCCAGAAAGCTGTTGATCGGCTTCAAGCAGGGGAGGGACACCTAATT |
| | | TGCTTTTCTTTTTTTTTTTTTTTTTTTTTTTTTTTTTTGAGATGGAGTTTTGCTCTTGTTGCCCAGGCT |
| | | GGAGTGCAATGGTGCATCTTGGCTCACTGCAACCTCCGCCTCCCAGGTTCAAGTGATTCTCCTGCCTCA |
| | | GCCTCCCGAGTAGCTGAGATTACAGGCACCCGCCACCATGCCTGGCTAATTTTTTGTATTTTTAGTAGA |
| | | GACAGGGTTTCACTATGTTGGCCAGGCTGGTCTCGAACTCCTGACCTCAGGTGATCCACCCGCTTCAGC |
| | | CTCCCAAAGTGCTGGGATTACAGGCGTGAGCCACCACACCCGGCCTGCTTTTCTTAAAGATCAATCTGA |
| | | GTGCTGTACGGAGAGTGGGTTGTAAGCCAAGAGTAGAAGCAGAAAGGGAGCAGTTGCAGCAGAGAGATG |
| | | ATGGAGGCCTGGGCAGGGTGGTGGCAGGGAGGTAACCAACACCATTCAGGTTTCAAAGGTAGAACCATG |
| | | CAGGGATGAGAAAGCAAAGAGGGGATCAAGGAAGGCAGCTGGATTTTGGCCTGAGCAGCTGAGTCAATG |
| | | ATAGTGCCGTTTACTAAGAAGAAACCAAGGAAAAAATTTGGGGTGCAGGGATCAAAACTTTTTGGAACA |
| | | TATGAAAGTACGTGTTTATACTCTTTATGGCCCTTGTCACTATGTATGCCTCGCTGCCTCCATTGGACT |
| | | CTAGAATGAAGCCAGGCAAGAGCAGGGTCTATGTGTGATGGCACATGTGGCCAGGGTCATGCAACATGT |
| | | ACTTTGTACAAACAGTGTATATTGAGTAAATAGAAATGGTGTCCAGGAGCCGAGGTATCGGTCCTGCCA |
| | | GGGCCAGGGGCTCTCCCTAGCAGGTGCTCATATGCTGTAAGTTCCCTCCAGATCTCTCCACAAGGAGGC |
| | | ATGGAAAGGCTGTAGTTGTTCACCTGCCCAAGAACTAGGAGGTCTGGGGTGGGAGAGTCAGCCTGCTCT |
| | | GGATGCTGAAAGAATGTCTGTTTTTCCTTTTAGAAAGTTCCTGTGATGTCAAGCTGGTCGAGAAAAGCT |
| | | TTGAAACAGGTAAGACAGGGGTCTAGCCTGGGTTTGCACAGGATTGCGGAAGTGATGAACCCGCAATAA |
| | | CCCTGCCTGGATGAGGGAGTGGGAAGAAATTAGTAGATGTGGGAATGAATGATGAGGAATGGAAACAGC |
| | | GGTTCAAGACCTGCCCAGAGCTGGGTGGGGTCTCTCCTGAATCCCTCTCACCATCTCTGACTTTCCATT |
| | | CTAAGCACTTTGAGGATGAGTTTCTAGCTTCAATAGACCAAGGACTCTCTCCTAGGCCTCTGTATTCCT |
| | | TTCAACAGCTCCACTGTCAAGAGAGCCAGAGAGAGCTTCTGGGTGGCCCAGCTGTGAAATTTCTGAGTC |
| | | CCTTAGGGATAGCCCTAAACGAACCAGATCATCCTGAGGACAGCCAAGAGGTTTTGCCTTCTTTCAAGA |
| | | CAAGCAACAGTACTCACATAGGCTGTGGGCAATGGTCCTGTCTCTCAAGAATCCCCTGCCACTCCTCAC |
| | | ACCCACCCTGGGCCCATATTCATTTCCATTTGAGTTGTTCTTATTGAGTCATCCTTCCTGTGGTAGCGG |
| | | AACTCACTAAGGGGCCCATCTGGACCCGAGGTATTGTGATGATAAATTCTGAGCACCTACCCCATCCCC |
| | | AGAAGGGCTCAGAAATAAAATAAGAGCCAAGTCTAGTCGGTGTTTCCTGTCTTGAAACACAATACTGTT |
| | | GGCCCTGGAAGAATGCACAGAATCTGTTTGTAAGGGGATATGCACAGAAGCTGCAAGGGACAGGAGGTG |
| | | CAGGAGCTGCAGGCCTCCCCCACCCAGCCTGCTCTGCCTTGGGGAAAACCGTGGGTGTGTCCTGCAGGC |
| | | CATGCAGGCCTGGGACATGCAAGCCCATAACCGCTGTGGCCTCTTGGTTTTACAGATACGAACCTAAAC |
| | | TTTCAAAACCTGTCAGTGATTGGGTTCCGAATCCTCCTCCTGAAAGTGGCCGGGTTTAATCTGCTCATG |
| | | ACGCTGCGGCTGTGGTCCAGCTGAGGTGAGGGGCCTTGAAGCTGGGAGTGGGGTTTAGGGACGCGGGTC |
| | | TCTGGGTGCATCCTAAGCTCTGAGAGCAAACCTCCCTGCAGGGTCTTGCTTTTAAGTCCAAAGCCTGAG |
| | | CCCACCAAACTCTCCTACTTCTTCCTGTTACAAATTCCTCTTGTGCAATAATAATGGCCTGAAACGCTG |
| | | TAAAATATCCTCATTTCAGCCGCCTCAGTT |
| 3 | cdsDNA222 | TTACCAATGCTTAATCAGTGAGGCACCTATCTCAGCGATCTGTCTATTTCGTTCATCCATAGTTGCCTG |
| | GFP at | ACTCCCCGTCGTGTAGATAACTACGATACGGGAGGGCTTACCATCTGGCCCCAGTGCTGCAATGATACC |
| | TRAC 1200 | GCGAGACCCACGCTCACCGGCTCCAGATTTATCAGCAATAAACCAGCCAGCCGGAAGGGCCGAGCGCAG |
| | without | AAGTGGTCCTGCAACTTTATCCGCCTCCATCCAGTCTATTAATTGTTGCCGGGAAGCTAGAGTAAGTAG |
| | CTS with | TTCGCCAGTTAATAGTTTGCGCAACGTTGTTGCCATTGCTACAGGCATCGTGGTGTCACGCTCGTCGTT |
| | ZFDBM5 | TGGTATGGCTTCATTCAGCTCCGGTTCCCAACGATCAAGGCGAGTTACATGATCCCCCATGTTGTGCAA |
| | | AAAAGCGGTTAGCTCCTTCGGTCCTCCGATCGTTGTCAGAAGTAAGTTGGCCGCAGTGTTATCACTCAT |
| | | GGTTATGGCAGCACTGCATAATTCTCTTACTGTCATGCCATCCGTAAGATGCTTTTCTGTGACTGGTGA |
| | | GTACTCAACCAAGTCATTCTGAGAATAGTGTATGCGGCGACCGAGTTGCTCTTGCCCGGCGTCAATACG |
| | | GGATAATACCGCGCCACATAGCAGAACTTTAAAAGTGCTCATCATTGGAAAACGTTCTTCGGGGCGAAA |
| | | ACTCTCAAGGATCTTACCGCTGTTGAGATCCAGTTCGATGTAACCCACTCGTGCACCCAACTGATCTTC |
| | | AGCATCTTTTACTTTCACCAGCGTTTCTGGGTGAGCAAAAACAGGAAGGCAAAATGCCGCAAAAAAGGG |
| | | AATAAGGGCGACACGGAAATGTTGAATACTCATACTCTTCCTTTTTCAATATTATTGAAGCATTTATCA |
| | | GGGTTATTGTCTCATGAGCGGATACATATTTGAATGTATTTAGAAAAATAAACAAATAGGGGTTCCGCG |
| | | CACATTTCCCCGAAAAGTGCCACCTGACGTCTAAGAAACCATTATTATCATGACATTAACCTATAAAAA |
| | | TAGGCGTATCACGAGGCAGAATTTCAGCCATCGCCCTGATAGACGGTTTTTCGCCCTTTGACGTTGGAG |
| | | TCCACGTTCTTTAATAGTGGACTCTTGTTCCAAACTGGAACAACACTCAACCCTATCTCGGGCAAGCTT |
| | | GTCTCTCCCCTCTGACCATGGAGCCCACCCCTGCTCCACTGCTCCAGGGACAGCCCTATGCTGCAGGCA |
| | | GCTCTGCCCCCACTCAGCATCCCAGGGGCTGATTTCTTTGGTTTTGGATCCAGCTGGATGTCTGCATTG |
| | | CCGAGGCCACCAGGGCTGGCTCAGCAACTGTCGGGGAATCACCAGGGTCTGAGAAATCTTGTGCGCATG |
| | | TGAGGGGCTGTGGGAGCAGAGAACCACTGGGTGGGAAATTCTAATCCCCACCCTGCTGGAAACTCTCTG |
| | | GGTGGCCCCAACATGCTAATCCTCCGGTAAACCTCTGTTTCCTCCTCAAAAGGCAGGAGGTCGGAAAGA |
| | | ATAAACAATGAGAGTCACATTAAAAACACAAAATCCTACGGAAATACTGAAGAATGAGTCTCAGCACTA |
| | | AGGAAAAGCCTCCAGCAGCTCCTGCTTTCTGAGGGTGAAGGATAGACGCTGTGGCTCTGCATGACTCAC |
| | | TAGCACTCTATCACGGCCATATTCTGACAGGGTCAGTGGCTCCAACTAACATTTGTTTGGTACTTTACA |
| | | GTTTATTAAATAGATGTTTATATGGAGAAGCTCTCATTTCTTTCTCAGAAGAGCCTGGCTAGGAAGGTG |
| | | GATGAGGCACCATATTCATTTTGCAGGTGAAATTCCTGAGATGTAAGGAGCTGCTGTGACTTGCTCAAG |
| | | GCCTTATATCGAGTAAACGGTAGCGCTGGGGCTTAGACGCAGGTGTTCTGATTTATAGTTCAAAACCTC |
| | | TATCAATGAGAGAGCAATCTCCTGGTAATGTGATAGATTTCCCAACTTAATGCCAACATACCATAAACC |
| | | TCCCATTCTGCTAATGCCCAGCCTAAGTTGGGGAGACCACTCCAGATTCCAAGATGTACAGTTTGCTTT |
| | | GCTGGGCCTTTTTCCCATGCCTGCCTTTACTCTGCCAGAGTTATATTGCTGGGGTTTTGAAGAAGATCC |
| | | TATTAAATAAAAGAATAAGCAGTATTATTAAGTAGCCCTGCATTTCAGGTTTCCTTGAGTGGCAGGCCA |
| | | GGCCTGGCCGTGAACGTTCACTGAAATCATGGCCTCTTGGCCAAGATTGATAGCTTGTGCCTGTCCCTG |
| | | AGTCCCAGTCCATCACGAGCAGCTGGTTTCTAAGATGCTATTTCCCGTATAAAGCATGAGACCGTGACT |
| | | TGCCAGCCCCACAGAGCCCCGCCCTTGTCCATCACTGGCATCTGGACTCCAGCCTGGGTTGGGGCAAAG |
| | | AGGGAAATGAGATCATGTCCTAACCCTGCTTTGTGATGGGAATTCTGTGGACCTGCAGGGCCCACTAGT |
| | | GCGGTTACCAGCGGAAATGCCTCGGGGTCAGAAGTCGCAGGAGAGATAGACAGCTGCTGAACCAATGGG |
| | | ACCAGCGGATGGGGCGGATGTTATCTACCATTGGTGAACGTTAGAAACGAATAGCAGCCAATGAATCAG |
| | | CTGGGGGGGCGGAGCAGTGACGTTTATTGCGGAGGGGGCCGCTTCGAATCGGCGGCGGCCAGCTTGGTG |
| | | GCCTGGGCCAATGAACGGCCTCCAACGAGCAGGGCCTTCACCAATCGGCGGCCTCCACGACGGGGCTGG |
| | | GGGAGGGTATATAAGCCGAGTAGGCGACGGTGAGGTCGACGCCGGCCAAGACAGCACAGACAGATTGAC |
| | | CTATTGGGGTGTTTCGCGAGTGTGAGAGGGAAGCGCCGCGGCCTGTATTTCTAGACCTGCCCTTCGCCT |
| | | GGTTCGTGGCGCCTTGTGACCCCGGGCCCCTGCCGCCTGCAAGTCGGAAATTGCGCTGTGCTCCTGTGC |
| | | TACGGCCTGTGGCTGGACTGCCTGCTGCTGCCCAACTGGCTGCCACCATGGTCTCCAAAGGAGAAGAAC |
| | | TCTTCACCGGAGTCGTACCAATCCTCGTCGAACTTGATGGCGACGTCAATGGTCATAAATTTTTCGTGT |
| | | CTGGTGAGGGAGAGGGGGACGCGACTTATGGAAAGCTCACGCTCAAGTTTATCTGCACCACTGGAAAAC |
| | | TGCCGGTCCCGTGGCCAACTCTTGTGACCACGTTTACATACGGAGTCCAGTGCTTCGCCAGATATCCCG |
| | | ACCATATGAAGCAACATGACTTCTTTAAGAGTGCCATGCCGGAAGGGTACGTGCAAGAGAGAACTATCT |
| | | TTTTCAAGGATGACGGCAACTACAAAACTCGAGCTGAGGTCAAGTTTGAGGGTGATACCCTCGTCAATA |
| | | GAATTGAATTGAAGGGCATCGACTTTAAGGAGGACGGCAACATCCTGGGCCACAAGCTGGAATATAATT |
| | | ACAACAGCCACAAAGTCTACATTACGGCAGACAAGCAGAAGAACGGCATAAAAGTCAACTTTAAAACGA |
| | | GGCACAACATAGAAGATGGCTCTGTCCAGTTGGCTGACCACTATCAGCAGAATACGCCTATAGGGGATG |
| | | GGCCGGTTCTGCTCCCCGACAATCATTACCTCAGTACCCAATCTGCTTTGTCAAAGGACCCCAATGAGA |
| | | AAAGAGACCATATGGTCCTGCTCGAGTTTGTCACAGCCGCCGGTATAACACTTGGTATGGATGAGCTGT |
| | | ATAAGTAATGATAGAAGCTTGATCTTTTTCCCTCTGCCAAAAATTATGGGGACATCATGAAGCCCCTTG |
| | | AGCATCTGACTTCTGGCTAATAAAGGAAATTTATTTTCATTGCAATAGTGTGTTGGAATTTTTTGTGTC |
| | | TCTCACTCGACTCGAAAAGATACGCAGCGCTGGATGAGATGAGAAATATTCCGCGACGCAAGCAGGAAT |
| | | ATCCAGAACCCTGACCCTGCCGTGTACCAGCTGAGAGACTCTAAATCCAGTGACAAGTCTGTCTGCCTA |
| | | TTCACCGATTTTGATTCTCAAACAAATGTGTCACAAAGTAAGGATTCTGATGTGTATATCACAGACAAA |
| | | ACTGTGCTAGACATGAGGTCTATGGACTTCAAGAGCAACAGTGCTGTGGCCTGGAGCAACAAATCTGAC |
| | | TTTGCATGTGCAAACGCCTTCAACAACAGCATTATTCCAGAAGACACCTTCTTCCCCAGCCCAGGTAAG |
| | | GGCAGCTTTGGTGCCTTCGCAGGCTGTTTCCTTGCTTCAGGAATGGCCAGGTTCTGCCCAGAGCTCTGG |
| | | TCAATGATGTCTAAAACTCCTCTGATTGGTGGTCTCGGCCTTATCCATTGCCACCAAAACCCTCTTTTT |
| | | ACTAAGAAACAGTGAGCCTTGTTCTGGCAGTCCAGAGAATGACACGGGAAAAAAGCAGATGAAGAGAAG |
| | | GTGGCAGGAGAGGGCACGTGGCCCAGCCTCAGTCTCTCCAACTGAGTTCCTGCCTGCCTGCCTTTGCTC |
| | | AGACTGTTTGCCCCTTACTGCTCTTCTAGGCCTCATTCTAAGCCCCTTCTCCAAGTTGCCTCTCCTTAT |
| | | TTCTCCCTGTCTGCCAAAAAATCTTTCCCAGCTCACTAAGTCAGTCTCACGCAGTCACTCATTAACCCA |
| | | CCAATCACTGATTGTGCCGGCACATGAATGCACCAGGTGTTGAAGTGGAGGAATTAAAAAGTCAGATGA |
| | | GGGGTGTGCCCAGAGGAAGCACCATTCTAGTTGGGGGAGCCCATCTGTCAGCTGGGAAAAGTCCAAATA |
| | | ACTTCAGATTGGAATGTGTTTTAACTCAGGGTTGAGAAAACAGCCACCTTCAGGACAAAAGTCAGGGAA |
| | | GGGCTCTCTGAAGAAATGCTACTTGAAGATACCAGCCCTACCAAGGGCAGGGAGAGGACCCTATAGAGG |
| | | CCTGGGACAGGAGCTCAATGAGAAAGGAGAAGAGCAGCAGGCATGAGTTGAATGAAGGAGGCAGGGCCG |
| | | GGTCACAGGGCCTTCTAGGCCATGAGAGGGTAGACAGTATTCTAAGTACGCCAGAAAGCTGTTGATCGG |
| | | CTTCAAGCAGGGAAGGGACACCTAATTTGCTTTTCTTTTCTTTTTTTTTTTTTTTTTTTTTTTGAGATG |
| | | GAGTTTTGCTCTTGTTGCCCAGGCTGGAGTGCAATGGTGCATCTTGGCTCACTGCAACCATTTGAAGCA |
| | | GTCGACGCCGAAGAGGGTCCGAGGTATTCCTTCGGCGTCGACTGCTTCAGACCGGGAGCGCCCTGTAGC |
| | | GGCGAATTAAGCGCGGCGGGTGTGGTGGTTACGCGCAGCGTGACCGCTACACTTGCCAGCGCCCTAGCG |
| | | CCCGCTCCCGGGATCGGAATTCCGGCCATCGCCCTGATAGACGGTTTTTCGCCCTTTGACGTTGGAGTC |
| | | CACGTTCTTTAATAGTGGACTCTTGTTCCAAACTGGAAAACGTCTAGCGGTATCAGCTCACTCAAAGGC |
| | | GGTAATACGGTTATCCACATAATCAGGGGATAACGCAGGAAAGAACATGTGAGCAAAAGGCCAGCAAAA |
| | | GGCCAGGAACCGTAAAAAGGCCGCGTTGCTGGCGTTTTTCCATAGGCTCCGCCCCCCTGACGAGCATCA |
| | | CAAAAATCGACGCTCAAGTCAGAGGTGGCGAAACCCGACAGGACTATAAAGATACCAGGCGTTTCCCCC |
| | | TGGAAGCTCCCTCGTGCGCTCTCCTGTTCCGACCCTGCCGCTTACCGGATACCTGTCCGCCTTTCTCCC |
| | | TTCGGGAAGCGTGGCGCTTTCTCATAGCTCACGCTGTAGGTATCTCAGTTCGGTGTAGGTCGTTCGCTC |
| | | CAAGCTGGGCTGTGTGCACGAACCCCCCGTTCAGCCCGACCGCTGCGCCTTATCCGGTAACTATCGTCT |
| | | TGAGTCCAACCCGGTAAGACACGACTTATCGCCACTGGCAGCAGCCACTGGTAACAGGATTAGCAGAGC |
| | | GAGGTATGTAGGCGGTGCTACAGAGTTCTTGAAGTGGTGGCCTAACTACGGCTACACTAGAAGGACAGT |
| | | ATTTGGTATCTGCGCTCTGCTGAAGCCAGTTACCTTCGGAAAAAGAGTTGATAGCTCTTGATCCGGCAA |
| | | ACAAACCACCGTTGATAGCGGTGGTTTTTTTGTTTGCAAGCAGCAGATTACGCGCAGAAAAAAAGGATC |
| | | TCAAGAAGATCCTTTGATCTTTTCTACGGGGTCTGACGCTCAGTGGAACGAAAACTCACGTTAAGGGAT |
| | | TTTGGTCATGAGATTATCAAAAAGGATCTTCACCTAGATCCTTTTAAATTAAAAATGAAGTTTTAAATC |
| | | AATCTAAAGTATATATGAGTAAACTTGGTCTGACAG |
| 4 | cdsDNA221 | TTACCAATGCTTAATCAGTGAGGCACCTATCTCAGCGATCTGTCTATTTCGTTCATCCATAGTTGCCTG |
| | GFP at | ACTCCCCGTCGTGTAGATAACTACGATACGGGAGGGCTTACCATCTGGCCCCAGTGCTGCAATGATACC |
| | TRAC 1200 | GCGAGACCCACGCTCACCGGCTCCAGATTTATCAGCAATAAACCAGCCAGCCGGAAGGGCCGAGCGCAG |
| | without | AAGTGGTCCTGCAACTTTATCCGCCTCCATCCAGTCTATTAATTGTTGCCGGGAAGCTAGAGTAAGTAG |
| | CTS | TTCGCCAGTTAATAGTTTGCGCAACGTTGTTGCCATTGCTACAGGCATCGTGGTGTCACGCTCGTCGTT |
| | | TGGTATGGCTTCATTCAGCTCCGGTTCCCAACGATCAAGGCGAGTTACATGATCCCCCATGTTGTGCAA |
| | | AAAAGCGGTTAGCTCCTTCGGTCCTCCGATCGTTGTCAGAAGTAAGTTGGCCGCAGTGTTATCACTCAT |
| | | GGTTATGGCAGCACTGCATAATTCTCTTACTGTCATGCCATCCGTAAGATGCTTTTCTGTGACTGGTGA |
| | | GTACTCAACCAAGTCATTCTGAGAATAGTGTATGCGGCGACCGAGTTGCTCTTGCCCGGCGTCAATACG |
| | | GGATAATACCGCGCCACATAGCAGAACTTTAAAAGTGCTCATCATTGGAAAACGTTCTTCGGGGCGAAA |
| | | ACTCTCAAGGATCTTACCGCTGTTGAGATCCAGTTCGATGTAACCCACTCGTGCACCCAACTGATCTTC |
| | | AGCATCTTTTACTTTCACCAGCGTTTCTGGGTGAGCAAAAACAGGAAGGCAAAATGCCGCAAAAAAGGG |
| | | AATAAGGGCGACACGGAAATGTTGAATACTCATACTCTTCCTTTTTCAATATTATTGAAGCATTTATCA |
| | | GGGTTATTGTCTCATGAGCGGATACATATTTGAATGTATTTAGAAAAATAAACAAATAGGGGTTCCGCG |
| | | CACATTTCCCCGAAAAGTGCCACCTGACGTCTAAGAAACCATTATTATCATGACATTAACCTATAAAAA |
| | | TAGGCGTATCACGAGGCAGAATTTCAGCCATCGCCCTGATAGACGGTTTTTCGCCCTTTGACGTTGGAG |
| | | TCCACGTTCTTTAATAGTGGACTCTTGTTCCAAACTGGAACAACACTCAACCCTATCTCGGGCAAGCTT |
| | | GTCTCTCCCCTCTGACCATGGAGCCCACCCCTGCTCCACTGCTCCAGGGACAGCCCTATGCTGCAGGCA |
| | | GCTCTGCCCCCACTCAGCATCCCAGGGGCTGATTTCTTTGGTTTTGGATCCAGCTGGATGTCTGCATTG |
| | | CCGAGGCCACCAGGGCTGGCTCAGCAACTGTCGGGGAATCACCAGGGTCTGAGAAATCTTGTGCGCATG |
| | | TGAGGGGCTGTGGGAGCAGAGAACCACTGGGTGGGAAATTCTAATCCCCACCCTGCTGGAAACTCTCTG |
| | | GGTGGCCCCAACATGCTAATCCTCCGGTAAACCTCTGTTTCCTCCTCAAAAGGCAGGAGGTCGGAAAGA |
| | | ATAAACAATGAGAGTCACATTAAAAACACAAAATCCTACGGAAATACTGAAGAATGAGTCTCAGCACTA |
| | | AGGAAAAGCCTCCAGCAGCTCCTGCTTTCTGAGGGTGAAGGATAGACGCTGTGGCTCTGCATGACTCAC |
| | | TAGCACTCTATCACGGCCATATTCTGACAGGGTCAGTGGCTCCAACTAACATTTGTTTGGTACTTTACA |
| | | GTTTATTAAATAGATGTTTATATGGAGAAGCTCTCATTTCTTTCTCAGAAGAGCCTGGCTAGGAAGGTG |
| | | GATGAGGCACCATATTCATTTTGCAGGTGAAATTCCTGAGATGTAAGGAGCTGCTGTGACTTGCTCAAG |
| | | GCCTTATATCGAGTAAACGGTAGCGCTGGGGCTTAGACGCAGGTGTTCTGATTTATAGTTCAAAACCTC |
| | | TATCAATGAGAGAGCAATCTCCTGGTAATGTGATAGATTTCCCAACTTAATGCCAACATACCATAAACC |
| | | TCCCATTCTGCTAATGCCCAGCCTAAGTTGGGGAGACCACTCCAGATTCCAAGATGTACAGTTTGCTTT |
| | | GCTGGGCCTTTTTCCCATGCCTGCCTTTACTCTGCCAGAGTTATATTGCTGGGGTTTTGAAGAAGATCC |
| | | TATTAAATAAAAGAATAAGCAGTATTATTAAGTAGCCCTGCATTTCAGGTTTCCTTGAGTGGCAGGCCA |
| | | GGCCTGGCCGTGAACGTTCACTGAAATCATGGCCTCTTGGCCAAGATTGATAGCTTGTGCCTGTCCCTG |
| | | AGTCCCAGTCCATCACGAGCAGCTGGTTTCTAAGATGCTATTTCCCGTATAAAGCATGAGACCGTGACT |
| | | TGCCAGCCCCACAGAGCCCCGCCCTTGTCCATCACTGGCATCTGGACTCCAGCCTGGGTTGGGGCAAAG |
| | | AGGGAAATGAGATCATGTCCTAACCCTGCTTTGTGATGGGAATTCTGTGGACCTGCAGGGCCCACTAGT |
| | | GCGGTTACCAGCGGAAATGCCTCGGGGTCAGAAGTCGCAGGAGAGATAGACAGCTGCTGAACCAATGGG |
| | | ACCAGCGGATGGGGCGGATGTTATCTACCATTGGTGAACGTTAGAAACGAATAGCAGCCAATGAATCAG |
| | | CTGGGGGGGCGGAGCAGTGACGTTTATTGCGGAGGGGGCCGCTTCGAATCGGCGGCGGCCAGCTTGGTG |
| | | GCCTGGGCCAATGAACGGCCTCCAACGAGCAGGGCCTTCACCAATCGGCGGCCTCCACGACGGGGCTGG |
| | | GGGAGGGTATATAAGCCGAGTAGGCGACGGTGAGGTCGACGCCGGCCAAGACAGCACAGACAGATTGAC |
| | | CTATTGGGGTGTTTCGCGAGTGTGAGAGGGAAGCGCCGCGGCCTGTATTTCTAGACCTGCCCTTCGCCT |
| | | GGTTCGTGGCGCCTTGTGACCCCGGGCCCCTGCCGCCTGCAAGTCGGAAATTGCGCTGTGCTCCTGTGC |
| | | TACGGCCTGTGGCTGGACTGCCTGCTGCTGCCCAACTGGCTGCCACCATGGTCTCCAAAGGAGAAGAAC |
| | | TCTTCACCGGAGTCGTACCAATCCTCGTCGAACTTGATGGCGACGTCAATGGTCATAAATTTTTCGTGT |
| | | CTGGTGAGGGAGAGGGGGACGCGACTTATGGAAAGCTCACGCTCAAGTTTATCTGCACCACTGGAAAAC |
| | | TGCCGGTCCCGTGGCCAACTCTTGTGACCACGTTTACATACGGAGTCCAGTGCTTCGCCAGATATCCCG |
| | | ACCATATGAAGCAACATGACTTCTTTAAGAGTGCCATGCCGGAAGGGTACGTGCAAGAGAGAACTATCT |
| | | TTTTCAAGGATGACGGCAACTACAAAACTCGAGCTGAGGTCAAGTTTGAGGGTGATACCCTCGTCAATA |
| | | GAATTGAATTGAAGGGCATCGACTTTAAGGAGGACGGCAACATCCTGGGCCACAAGCTGGAATATAATT |
| | | ACAACAGCCACAAAGTCTACATTACGGCAGACAAGCAGAAGAACGGCATAAAAGTCAACTTTAAAACGA |
| | | GGCACAACATAGAAGATGGCTCTGTCCAGTTGGCTGACCACTATCAGCAGAATACGCCTATAGGGGATG |
| | | GGCCGGTTCTGCTCCCCGACAATCATTACCTCAGTACCCAATCTGCTTTGTCAAAGGACCCCAATGAGA |
| | | AAAGAGACCATATGGTCCTGCTCGAGTTTGTCACAGCCGCCGGTATAACACTTGGTATGGATGAGCTGT |
| | | ATAAGTAATGATAGAAGCTTGATCTTTTTCCCTCTGCCAAAAATTATGGGGACATCATGAAGCCCCTTG |
| | | AGCATCTGACTTCTGGCTAATAAAGGAAATTTATTTTCATTGCAATAGTGTGTTGGAATTTTTTGTGTC |
| | | TCTCACTCGACTCGAAAAGATACGCAGCGCTGGATGAGATGAGAAATATTCCGCGACGCAAGCAGGAAT |
| | | ATCCAGAACCCTGACCCTGCCGTGTACCAGCTGAGAGACTCTAAATCCAGTGACAAGTCTGTCTGCCTA |
| | | TTCACCGATTTTGATTCTCAAACAAATGTGTCACAAAGTAAGGATTCTGATGTGTATATCACAGACAAA |
| | | ACTGTGCTAGACATGAGGTCTATGGACTTCAAGAGCAACAGTGCTGTGGCCTGGAGCAACAAATCTGAC |
| | | TTTGCATGTGCAAACGCCTTCAACAACAGCATTATTCCAGAAGACACCTTCTTCCCCAGCCCAGGTAAG |
| | | GGCAGCTTTGGTGCCTTCGCAGGCTGTTTCCTTGCTTCAGGAATGGCCAGGTTCTGCCCAGAGCTCTGG |
| | | TCAATGATGTCTAAAACTCCTCTGATTGGTGGTCTCGGCCTTATCCATTGCCACCAAAACCCTCTTTTT |
| | | ACTAAGAAACAGTGAGCCTTGTTCTGGCAGTCCAGAGAATGACACGGGAAAAAAGCAGATGAAGAGAAG |
| | | GTGGCAGGAGAGGGCACGTGGCCCAGCCTCAGTCTCTCCAACTGAGTTCCTGCCTGCCTGCCTTTGCTC |
| | | AGACTGTTTGCCCCTTACTGCTCTTCTAGGCCTCATTCTAAGCCCCTTCTCCAAGTTGCCTCTCCTTAT |
| | | TTCTCCCTGTCTGCCAAAAAATCTTTCCCAGCTCACTAAGTCAGTCTCACGCAGTCACTCATTAACCCA |
| | | CCAATCACTGATTGTGCCGGCACATGAATGCACCAGGTGTTGAAGTGGAGGAATTAAAAAGTCAGATGA |
| | | GGGGTGTGCCCAGAGGAAGCACCATTCTAGTTGGGGGAGCCCATCTGTCAGCTGGGAAAAGTCCAAATA |
| | | ACTTCAGATTGGAATGTGTTTTAACTCAGGGTTGAGAAAACAGCCACCTTCAGGACAAAAGTCAGGGAA |
| | | GGGCTCTCTGAAGAAATGCTACTTGAAGATACCAGCCCTACCAAGGGCAGGGAGAGGACCCTATAGAGG |
| | | CCTGGGACAGGAGCTCAATGAGAAAGGAGAAGAGCAGCAGGCATGAGTTGAATGAAGGAGGCAGGGCCG |
| | | GGTCACAGGGCCTTCTAGGCCATGAGAGGGTAGACAGTATTCTAAGTACGCCAGAAAGCTGTTGATCGG |
| | | CTTCAAGCAGGGAAGGGACACCTAATTTGCTTTTCTTTTCTTTTTTTTTTTTTTTTTTTTTTGAGATGG |
| | | AGTTTTGCTCTTGTTGCCCAGGCTGGAGTGCAATGGTGCATCTTGGCTCACTGCAACGACCGGGAGCGC |
| | | CCTGTAGCGGCGAATTAAGCGCGGCGGGTGTGGTGGTTACGCGCAGCGTGACCGCTACACTTGCCAGCG |
| | | CCCTAGCGCCCGCTCCCGGGATCGGAATTCCGGCCATCGCCCTGATAGACGGTTTTTCGCCCTTTGACG |
| | | TTGGAGTCCACGTTCTTTAATAGTGGACTCTTGTTCCAAACTGGAAAACGTCTAGCGGTATCAGCTCAC |
| | | TCAAAGGCGGTAATACGGTTATCCACATAATCAGGGGATAACGCAGGAAAGAACATGTGAGCAAAAGGC |
| | | CAGCAAAAGGCCAGGAACCGTAAAAAGGCCGCGTTGCTGGCGTTTTTCCATAGGCTCCGCCCCCCTGAC |
| | | GAGCATCACAAAAATCGACGCTCAAGTCAGAGGTGGCGAAACCCGACAGGACTATAAAGATACCAGGCG |
| | | TTTCCCCCTGGAAGCTCCCTCGTGCGCTCTCCTGTTCCGACCCTGCCGCTTACCGGATACCTGTCCGCC |
| | | TTTCTCCCTTCGGGAAGCGTGGCGCTTTCTCATAGCTCACGCTGTAGGTATCTCAGTTCGGTGTAGGTC |
| | | GTTCGCTCCAAGCTGGGCTGTGTGCACGAACCCCCCGTTCAGCCCGACCGCTGCGCCTTATCCGGTAAC |
| | | TATCGTCTTGAGTCCAACCCGGTAAGACACGACTTATCGCCACTGGCAGCAGCCACTGGTAACAGGATT |
| | | AGCAGAGCGAGGTATGTAGGCGGTGCTACAGAGTTCTTGAAGTGGTGGCCTAACTACGGCTACACTAGA |
| | | AGGACAGTATTTGGTATCTGCGCTCTGCTGAAGCCAGTTACCTTCGGAAAAAGAGTTGATAGCTCTTGA |
| | | TCCGGCAAACAAACCACCGTTGATAGCGGTGGTTTTTTTGTTTGCAAGCAGCAGATTACGCGCAGAAAA |
| | | AAAGGATCTCAAGAAGATCCTTTGATCTTTTCTACGGGGTCTGACGCTCAGTGGAACGAAAACTCACGT |
| | | TAAGGGATTTTGGTCATGAGATTATCAAAAAGGATCTTCACCTAGATCCTTTTAAATTAAAAATGAAGT |
| | | TTTAAATCAATCTAAAGTATATATGAGTAAACTTGGTCTGACAG |
| 5 | cdsDNA193 | AAAAAAAAAAAAAAAGAAAAGAAAAGCAAATTAGGTGTCCCTTCCCTGCTTGAAGCCGATCAACAGCTT |
| | emGFP at | TCTGGCGTACTTAGAATACTGTCTACCCTCTCATGGCCTAGAAGGCCCTGTGACCCGGCCCTGCCTCCT |
| | TRAC 1200 | TCATTCAACTCATGCCTGCTGCTCTTCTCCTTTCTCATTGAGCTCCTGTCCCAGGCCTCTATAGGGTCC |
| | with | TCTCCCTGCCCTTGGTAGGGCTGGTATCTTCAAGTAGCATTTCTTCAGAGAGCCCTTCCCTGACTTTTG |
| | ZFDBM5 | TCCTGAAGGTGGCTGTTTTCTCAACCCTGAGTTAAAACACATTCCAATCTGAAGTTATTTGGACTTTTC |
| | | CCAGCTGACAGATGGGCTCCCCCAACTAGAATGGTGCTTCCTCTGGGCACACCCCTCATCTGACTTTTT |
| | | AATTCCTCCACTTCAACACCTGGTGCATTCATGTGCCGGCACAATCAGTGATTGGTGGGTTAATGAGTG |
| | | ACTGCGTGAGACTGACTTAGTGAGCTGGGAAAGATTTTTTGGCAGACAGGGAGAAATAAGGAGAGGCAA |
| | | CTTGGAGAAGGGGCTTAGAATGAGGCCTAGAAGAGCAGTAAGGGGCAAACAGTCTGAGCAAAGGCAGGC |
| | | AGGCAGGAACTCAGTTGGAGAGACTGAGGCTGGGCCACGTGCCCTCTCCTGCCACCTTCTCTTCATCTG |
| | | CTTTTTTCCCGTGTCATTCTCTGGACTGCCAGAACAAGGCTCACTGTTTCTTAGTAAAAAGAGGGTTTT |
| | | GGTGGCAATGGATAAGGCCGAGACCACCAATCAGAGGAGTTTTAGACATCATTGACCAGAGCTCTGGGC |
| | | AGAACCTGGCCATTCCTGAAGCAAGGAAACAGCCTGCGAAGGCACCAAAGCTGCCCTTACCTGGGCTGG |
| | | GGAAGAAGGTGTCTTCTGGAATAATGCTGTTGTTGAAGGCGTTTGCACATGCAAAGTCAGATTTGTTGC |
| | | TCCAGGCCACAGCACTGTTGCTCTTGAAGTCCATAGACCTCATGTCTAGCACAGTTTTGTCTGTGATAT |
| | | ACACATCAGAATCCTTACTTTGTGACACATTTGTTTGAGAATCAAAATCGGTGAATAGGCAGACAGACT |
| | | TGTCACTGGATTTAGAGTCTCTCAGCTGGTACACGGCAGGGTCAGGGTTCTGGATATTCCTGCTTGCGT |
| | | CGCGGAATATTTCTCATCTCATCCAGCGCTGCGTATCTTTTCGAGTCGAGTGAGAGACACAAAAAATTC |
| | | CAACACACTATTGCAATGAAAATAAATTTCCTTTATTAGCCAGAAGTCAGATGCTCAAGGGGCTTCATG |
| | | ATGTCCCCATAATTTTTGGCAGAGGGAAAAAGATCAAGCTTCTATCATTACTTATACAGCTCATCCATA |
| | | CCAAGTGTTATACCGGCGGCTGTGACAAACTCGAGCAGGACCATATGGTCTCTTTTCTCATTGGGGTCC |
| | | TTTGACAAAGCAGATTGGGTACTGAGGTAATGATTGTCGGGGAGCAGAACCGGCCCATCCCCTATAGGC |
| | | GTATTCTGCTGATAGTGGTCAGCCAACTGGACAGAGCCATCTTCTATGTTGTGCCTCGTTTTAAAGTTG |
| | | ACTTTTATGCCGTTCTTCTGCTTGTCTGCCGTAATGTAGACTTTGTGGCTGTTGTAATTATATTCCAGC |
| | | TTGTGGCCCAGGATGTTGCCGTCCTCCTTAAAGTCGATGCCCTTCAATTCAATTCTATTGACGAGGGTA |
| | | TCACCCTCAAACTTGACCTCAGCTCGAGTTTTGTAGTTGCCGTCATCCTTGAAAAAGATAGTTCTCTCT |
| | | TGCACGTACCCTTCCGGCATGGCACTCTTAAAGAAGTCATGTTGCTTCATATGGTCGGGATATCTGGCG |
| | | AAGCACTGGACTCCGTATGTAAACGTGGTCACAAGAGTTGGCCACGGGACCGGCAGTTTTCCAGTGGTG |
| | | CAGATAAACTTGAGCGTGAGCTTTCCATAAGTCGCGTCCCCCTCTCCCTCACCAGACACGAAAAATTTA |
| | | TGACCATTGACGTCGCCATCAAGTTCGACGAGGATTGGTACGACTCCGGTGAAGAGTTCTTCTCCTTTG |
| | | GAGACCATGGTGGCAGCCAGTTGGGCAGCAGCAGGCAGTCCAGCCACAGGCCGTAGCACAGGAGCAçAG |
| | | CGCAATTTCCGACTTGCAGGCGGCAGGGGCCCGGGGTCACAAGGCGCCACGAACCAGGCGAAGGGCAGG |
| | | TCTAGAAATACAGGCCGCGGCGCTTCCCTCTCACACTCGCGAAACACCCCAATAGGTCAATCTGTCTGT |
| | | GCTGTCTTGGCCGGCGTCGACCTCACCGTCGCCTACTCGGCTTATATACCCTCCCCCAGCCCCGTCGTG |
| | | GAGGCCGCCGATTGGTGAAGGCCCTGCTCGTTGGAGGCCGTTCATTGGCCCAGGCCACCAAGCTGGCCG |
| | | CCGCCGATTCGAAGCGGCCCCCTCCGCAATAAACGTCACTGCTCCGCCCCCCCAGCTGATTCATTGGCT |
| | | GCTATTCGTTTCTAACGTTCACCAATGGTAGATAACATCCGCCCCATCCGCTGGTCCCATTGGTTCAGC |
| | | AGCTGTCTATCTCTCCTGCGACTTCTGACCCCGAGGCATTTCCGCTGGTAACCGCACTAGTGGGCCCTG |
| | | CAGGTCCACAGAATTCCCATCACAAAGCAGGGTTAGGACATGATCTCATTTCCCTCTTTGCCCCAACCC |
| | | AGGCTGGAGTCCAGATGCCAGTGATGGACAAGGGCGGGGCTCTGTGGGGCTGGCAAGTCACGGTCTCAT |
| | | GCTTTATACGGGAAATAGCATCTTAGAAACCAGCTGCTCGTGATGGACTGGGACTCAGGGACAGGCACA |
| | | AGCTATCAATCTTGGCCAAGAGGCCATGATTTCAGTGAACGTTCACGGCCAGGCCTGGCCTGCCACTCA |
| | | AGGAAACCTGAAATGCAGGGCTACTTAATAATACTGCTTATTCTTTTATTTAATAGGATCTTCTTCAAA |
| | | ACCCCAGCAATATAACTCTGGCAGAGTAAAGGCAGGCATGGGAAAAAGGCCCAGCAAAGCAAACTGTAC |
| | | ATCTTGGAATCTGGAGTGGTCTCCCCAACTTAGGCTGGGCATTAGCAGAATGGGAGGTTTATGGTATGT |
| | | TGGCATTAAGTTGGGAAATCTATCACATTACCAGGAGATTGCTCTCTCATTGATAGAGGTTTTGAACTA |
| | | TAAATCAGAACACCTGCGTCTAAGCCCCAGCGCTACCGTTTACTCGATATAAGGCCTTGAGCAAGTCAC |
| | | AGCAGCTCCTTACATCTCAGGAATTTCACCTGCAAAATGAATATGGTGCCTCATCCACCTTCCTAGCCA |
| | | GGCTCTTCTGAGAAAGAAATGAGAGCTTCTCCATATAAACATCTATTTAATAAACTGTAAAGTACCAAA |
| | | CAAATGTTAGTTGGAGCCACTGACCCTGTCAGAATATGGCCGTGATAGAGTGCTAGTGAGTCATGCAGA |
| | | GCCACAGCGTCTATCCTTCACCCTCAGAAAGCAGGAGCTGCTGGAGGCTTTTCCTTAGTGCTGAGACTC |
| | | ATTCTTCAGTATTTCCGTAGGATTTTGTGTTTTTAATGTGACTCTCATTGTTTATTCTTTCCGACCTCC |
| | | TGCCTTTTGAGGAGGAAACAGAGGTTTACCGGAGGATTAGCATGTTGGGGCCACCCAGAGAGTTTCCAG |
| | | CAGGGTGGGGATTAGAATTTCCCACCCAGTGGTTCTCTGCTCCCACAGCCCCTCACATGCGCACAAGAT |
| | | TTCTCAGACCCTGGTGATTCCCCGACAGTTGCTGAGCCAGCCCTGGTGGCCTCGGCAATGCAGACATCC |
| | | AGCTGGATCCAAAACCAAAGAAATCAGCCCCTGGGATGCTGAGTGGGGGCAGAGCTGCCTGCAGCATAG |
| | | GGCTGTCCCTGGAGCAGTGGAGCAGGGGTGGGCTCCATGGTCAGAGGGGAGACCCAACAGATATCCAGA |
| | | ACCGACTGTGACAAGCTTGCCCGAGATAGGGTTGAGTGTTGTTCCAGTTTGGAACAAGAGTCCACTATT |
| | | AAAGAACGTGGACTCCAACGTCAAAGGGCGAAAAACCGTCTATCAGGGCGATGGCTGAAATTCTGCCTC |
| | | GTGATACGCCTATTTTTATAGGTTAATGTCATGATAATAATGGTTTCTTAGACGTCAGGTGGCACTTTT |
| | | CGGGGAAATGTGCGCGGAACCCCTATTTGTTTATTTTTCTAAATACATTCAAATATGTATCCGCTCATG |
| | | AGACAATAACCCTGATAAATGCTTCAATAATATTGAAAAAGGAAGAGTATGAGTATTCAACATTTCCGT |
| | | GTCGCCCTTATTCCCTTTTTTGCGGCATTTTGCCTTCCTGTTTTTGCTCACCCAGAAACGCTGGTGAAA |
| | | GTAAAAGATGCTGAAGATCAGTTGGGTGCACGAGTGGGTTACATCGAACTGGATCTCAACAGCGGTAAG |
| | | ATCCTTGAGAGTTTTCGCCCCGAAGAACGTTTTCCAATGATGAGCACTTTTAAAGTTCTGCTATGTGGC |
| | | GCGGTATTATCCCGTATTGACGCCGGGCAAGAGCAACTCGGTCGCCGCATACACTATTCTCAGAATGAC |
| | | TTGGTTGAGTACTCACCAGTCACAGAAAAGCATCTTACGGATGGCATGACAGTAAGAGAATTATGCAGT |
| | | GCTGCCATAACCATGAGTGATAACACTGCGGCCAACTTACTTCTGACAACGATCGGAGGACCGAAGGAG |
| | | CTAACCGCTTTTTTGCACAACATGGGGGATCATGTAACTCGCCTTGATCGTTGGGAACCGGAGCTGAAT |
| | | GAAGCCATACCAAACGACGAGCGTGACACCACGATGCCTGTAGCAATGGCAACAACGTTGCGCAAACTA |
| | | TTAACTGGCGAACTACTTACTCTAGCTTCCCGGCAACAATTAATAGACTGGATGGAGGCGGATAAAGTT |
| | | GCAGGACCACTTCTGCGCTCGGCCCTTCCGGCTGGCTGGTTTATTGCTGATAAATCTGGAGCCGGTGAG |
| | | CGTGGGTCTCGCGGTATCATTGCAGCACTGGGGCCAGATGGTAAGCCCTCCCGTATCGTAGTTATCTAC |
| | | ACGACGGGGAGTCAGGCAACTATGGATGAACGAAATAGACAGATCGCTGAGATAGGTGCCTCACTGATT |
| | | AAGCATTGGTAACTGTCAGACCAAGTTTACTCATATATACTTTAGATTGATTTAAAACTTCATTTTTAA |
| | | TTTAAAAGGATCTAGGTGAAGATCCTTTTTGATAATCTCATGACCAAAATCCCTTAACGTGAGTTTTCG |
| | | TTCCACTGAGCGTCAGACCCCGTAGAAAAGATCAAAGGATCTTCTTGAGATCCTTTTTTTCTGCGCGTA |
| | | ATCTGCTGCTTGCAAACAAAAAAACCACCGCTACCAGCGGTGGTTTGTTTGCCGGATCAAGAGCTACCA |
| | | ACTCTTTTTCCGAAGGTAACTGGCTTCAGCAGAGCGCAGATACCAAATACTGTCCTTCTAGTGTAGCCG |
| | | TAGTTAGGCCACCACTTCAAGAACTCTGTAGCACCGCCTACATACCTCGCTCTGCTAATCCTGTTACCA |
| | | GTGGCTGCTGCCAGTGGCGATAAGTCGTGTCTTACCGGGTTGGACTCAAGACGATAGTTACCGGATAAG |
| | | GCGCAGCGGTCGGGCTGAACGGGGGGTTCGTGCACACAGCCCAGCTTGGAGCGAACGACCTACACCGAA |
| | | CTGAGATACCTACAGCGTGAGCTATGAGAAAGCGCCACGCTTCCCGAAGGGAGAAAGGCGGACAGGTAT |
| | | CCGGTAAGCGGCAGGGTCGGAACAGGAGAGCGCACGAGGGAGCTTCCAGGGGGAAACGCCTGGTATCTT |
| | | TATAGTCCTGTCGGGTTTCGCCACCTCTGACTTGAGCGTCGATTTTTGTGATGCTCGTCAGGGGGGCGG |
| | | AGCCTATGGAAAAACGCCAGCAACGCGGCCTTTTTACGGTTCCTGGCCTTTTGCTGGCCTTTTGCTCAC |
| | | ATGTTCTTTCCTGCGTTATCCCCTGATTCTGTGGATAACCGTATTACCGCCTTTGAGTGAGCTGATACC |
| | | GCTAGACGTTTTCCAGTTTGGAACAAGAGTCCACTATTAAAGAACGTGGACTCCAACGTCAAAGGGCGA |
| | | AAAACCGTCTATCAGGGCGATGGCCGGAATTCCGATCCCGGGAGCGGGCGCTAGGGCGCTGGCAAGTGT |
| | | AGCGGTCACGCTGCGCGTAACCACCACACCCGCCGCGCTTAATGCGCCGCTACAGGGCGCTCCCGGTCT |
| | | GAAGCAGTCGACGCCGAAGGAATACCTCGGACCCTCTTCGGCGTCGACTGCTTCAAATGGGTTCTGGAT |
| | | ATCTGTTGGGGTTGCAGTGAGCCAAGATGCACCATTGCACTCCAGCCTGGGCAACAAGAGCAAAACTCC |
| | | ATCTCAAAAAAAA |
| 6 | cdsDNA188 | TGGCGAATGGGACGCGCCCTGTAGCGGCGCATTAAGCGCGGCGGGTGTGGTGGTTACGCGCAGCGTGAC |
| | Cas9 Zinc | CGCTACACTTGCCAGCGCCCTAGCGCCCGCTCCTTTCGCTTTCTTCCCTTCCTTTCTCGCCACGTTCGC |
| | FInger 5 | CGGCTTTCCCCGTCAAGCTCTAAATCGGGGGCTCCCTTTAGGGTTCCGATTTAGTGCTTTACGGCACCT |
| | fusion E | CGACCCCAAAAAACTTGATTAGGGTGATGGTTCACGTAGTGGGCCATCGCCCTGATAGACGGTTTTTCG |
| | coli | CCCTTTGACGTTGGAGTCCACGTTCTTTAATAGTGGACTCTTGTTCCAAACTGGAACAACACTCAACCC |
| | expression | TATCTCGGTCTATTCTTTTGATTTATAAGGGATTTTGCCGATTTCGGCCTATTGGTTAAAAAATGAGCT |
| | vector | GATTTAACAAAAATTTAACGCGAATTTTAACAAAATATTAACGTTTACAATTTCAGGTGGCACTTTTCG |
| | | GGGAAATGTGCGCGGAACCCCTATTTGTTTATTTTTCTAAATACATTCAAATATGTATCCGCTCATGAA |
| | | TTAATTCTTAGAAAAACTCATCGAGCATCAAATGAAACTGCAATTTATTCATATCAGGATTATCAATAC |
| | | CATATTTTTGAAAAAGCCGTTTCTGTAATGAAGGAGAAAACTCACCGAGGCAGTTCCATAGGATGGCAA |
| | | GATCCTGGTATCGGTCTGCGATTCCGACTCGTCCAACATCAATACAACCTATTAATTTCCCCTCGTCAA |
| | | AAATAAGGTTATCAAGTGAGAAATCACCATGAGTGACGACTGAATCCGGTGAGAATGGCAAAAGTTTAT |
| | | GCATTTCTTTCCAGACTTGTTCAACAGGCCAGCCATTACGCTCGTCATCAAAATCACTCGCATCAACCA |
| | | AACCGTTATTCATTCGTGATTGCGCCTGAGCGAGACGAAATACGCGATCGCTGTTAAAAGGACAATTAC |
| | | AAACAGGAATCGAATGCAACCGGCGCAGGAACACTGCCAGCGCATCAACAATATTTTCACCTGAATCAG |
| | | GATATTCTTCTAATACCTGGAATGCTGTTTTCCCGGGGATCGCAGTGGTGAGTAACCATGCATCATCAG |
| | | GAGTACGGATAAAATGCTTGATGGTCGGAAGAGGCATAAATTCCGTCAGCCAGTTTAGTCTGACCATCT |
| | | CATCTGTAACATCATTGGCAACGCTACCTTTGCCATGTTTCAGAAACAACTCTGGCGCATCGGGCTTCC |
| | | CATACAATCGATAGATTGTCGCACCTGATTGCCCGACATTATCGCGAGCCCATTTATACCCATATAAAT |
| | | CAGCATCCATGTTGGAATTTAATCGCGGCCTAGAGCAAGACGTTTCCCGTTGAATATGGCTCATAACAC |
| | | CCCTTGTATTACTGTTTATGTAAGCAGACAGTTTTATTGTTCATGACCAAAATCCCTTAACGTGAGTTT |
| | | TCGTTCCACTGAGCGTCAGACCCCGTAGAAAAGATCAAAGGATCTTCTTGAGATCCTTTTTTTCTGCGC |
| | | GTAATCTGCTGCTTGCAAACAAAAAAACCACCGCTACCAGCGGTGGTTTGTTTGCCGGATCAAGAGCTA |
| | | CCAACTCTTTTTCCGAAGGTAACTGGCTTCAGCAGAGCGCAGATACCAAATACTGTCCTTCTAGTGTAG |
| | | CCGTAGTTAGGCCACCACTTCAAGAACTCTGTAGCACCGCCTACATACCTCGCTCTGCTAATCCTGTTA |
| | | CCAGTGGCTGCTGCCAGTGGCGATAAGTCGTGTCTTACCGGGTTGGACTCAAGACGATAGTTACCGGAT |
| | | AAGGCGCAGCGGTCGGGCTGAACGGGGGGTTCGTGCACACAGCCCAGCTTGGAGCGAACGACCTACACC |
| | | GAACTGAGATACCTACAGCGTGAGCTATGAGAAAGCGCCACGCTTCCCGAAGGGAGAAAGGCGGACAGG |
| | | TATCCGGTAAGCGGCAGGGTCGGAACAGGAGAGCGCACGAGGGAGCTTCCAGGGGGAAACGCCTGGTAT |
| | | CTTTATAGTCCTGTCGGGTTTCGCCACCTCTGACTTGAGCGTCGATTTTTGTGATGCTCGTCAGGGGGG |
| | | CGGAGCCTATGGAAAAACGCCAGCAACGCGGCCTTTTTACGGTTCCTGGCCTTTTGCTGGCCTTTTGCT |
| | | CACATGTTCTTTCCTGCGTTATCCCCTGATTCTGTGGATAACCGTATTACCGCCTTTGAGTGAGCTGAT |
| | | ACCGCTCGCCGCAGCCGAACGACCGAGCGCAGCGAGTCAGTGAGCGAGGAAGCGGAAGAGCGCCTGATG |
| | | CGGTATTTTCTCCTTACGCATCTGTGCGGTATTTCACACCGCATATATGGTGCACTCTCAGTACAATCT |
| | | GCTCTGATGCCGCATAGTTAAGCCAGTATACACTCCGCTATCGCTACGTGACTGGGTCATGGCTGCGCC |
| | | CCGACACCCGCCAACACCCGCTGACGCGCCCTGACGGGCTTGTCTGCTCCCGGCATCCGCTTACAGACA |
| | | AGCTGTGACCGTCTCCGGGAGCTGCATGTGTCAGAGGTTTTCACCGTCATCACCGAAACGCGCGAGGCA |
| | | GCTGCGGTAAAGCTCATCAGCGTGGTCGTGAAGCGATTCACAGATGTCTGCCTGTTCATCCGCGTCCAG |
| | | CTCGTTGAGTTTCTCCAGAAGCGTTAATGTCTGGCTTCTGATAAAGCGGGCCATGTTAAGGGCGGTTTT |
| | | TTCCTGTTTGGTCACTGATGCCTCCGTGTAAGGGGGATTTCTGTTCATGGGGGTAATGATACCGATGAA |
| | | ACGAGAGAGGATGCTCACGATACGGGTTACTGATGATGAACATGCCCGGTTACTGGAACGTTGTGAGGG |
| | | TAAACAACTGGCGGTATGGATGCGGCGGGACCAGAGAAAAATCACTCAGGGTCAATGCCAGCGCTTCGT |
| | | TAATACAGATGTAGGTGTTCCACAGGGTAGCCAGCAGCATCCTGCGATGCAGATCCGGAACATAATGGT |
| | | GCAGGGCGCTGACTTCCGCGTTTCCAGACTTTACGAAACACGGAAACCGAAGACCATTCATGTTGTTGC |
| | | TCAGGTCGCAGACGTTTTGCAGCAGCAGTCGCTTCACGTTCGCTCGCGTATCGGTGATTCATTCTGCTA |
| | | ACCAGTAAGGCAACCCCGCCAGCCTAGCCGGGTCCTCAACGACAGGAGCACGATCATGCGCACCCGTGG |
| | | GGCCGCCATGCCGGCGATAATGGCCTGCTTCTCGCCGAAACGTTTGGTGGCGGGACCAGTGACGAAGGC |
| | | TTGAGCGAGGGCGTGCAAGATTCCGAATACCGCAAGCGACAGGCCGATCATCGTCGCGCTCCAGCGAAA |
| | | GCGGTCCTCGCCGAAAATGACCCAGAGCGCTGCCGGCACCTGTCCTACGAGTTGCATGATAAAGAAGAC |
| | | AGTCATAAGTGCGGCGACGATAGTCATGCCCCGCGCCCACCGGAAGGAGCTGACTGGGTTGAAGGCTCT |
| | | CAAGGGCATCGGTCGAGATCCCGGTGCCTAATGAGTGAGCTAACTTACATTAATTGCGTTGCGCTCACT |
| | | GCCCGCTTTCCAGTCGGGAAACCTGTCGTGCCAGCTGCATTAATGAATCGGCCAACGCGCGGGGAGAGG |
| | | CGGTTTGCGTATTGGGCGCCAGGGTGGTTTTTCTTTTCACCAGTGAGACGGGCAACAGCTGATTGCCCT |
| | | TCACCGCCTGGCCCTGAGAGAGTTGCAGCAAGCGGTCCACGCTGGTTTGCCCCAGCAGGCGAAAATCCT |
| | | GTTTGATGGTGGTTAACGGCGGGATATAACATGAGCTGTCTTCGGTATCGTCGTATCCCACTACCGAGA |
| | | TGTCCGCACCAACGCGCAGCCCGGACTCGGTAATGGCGCGCATTGCGCCCAGCGCCATCTGATCGTTGG |
| | | CAACCAGCATCGCAGTGGGAACGATGCCCTCATTCAGCATTTGCATGGTTTGTTGAAAACCGGACATGG |
| | | CACTCCAGTCGCCTTCCCGTTCCGCTATCGGCTGAATTTGATTGCGAGTGAGATATTTATGCCAGCCAG |
| | | CCAGACGCAGACGCGCCGAGACAGAACTTAATGGGCCCGCTAACAGCGCGATTTGCTGGTGACCCAATG |
| | | CGACCAGATGCTCCACGCCCAGTCGCGTACCGTCTTCATGGGAGAAAATAATACTGTTGATGGGTGTCT |
| | | GGTCAGAGACATCAAGAAATAACGCCGGAACATTAGTGCAGGCAGCTTCCACAGCAATGGCATCCTGGT |
| | | CATCCAGCGGATAGTTAATGATCAGCCCACTGACGCGTTGCGCGAGAAGATTGTGCACCGCCGCTTTAC |
| | | AGGCTTCGACGCCGCTTCGTTCTACCATCGACACCACCACGCTGGCACCCAGTTGATCGGCGCGAGATT |
| | | TAATCGCCGCGACAATTTGCGACGGCGCGTGCAGGGCCAGACTGGAGGTGGCAACGCCAATCAGCAACG |
| | | ACTGTTTGCCCGCCAGTTGTTGTGCCACGCGGTTGGGAATGTAATTCAGCTCCGCCATCGCCGCTTCCA |
| | | CTTTTTCCCGCGTTTTCGCAGAAACGTGGCTGGCCTGGTTCACCACGCGGGAAACGGTCTGATAAGAGA |
| | | CACCGGCATACTCTGCGACATCGTATAACGTTACTGGTTTCACATTCACCACCCTGAATTGACTCTCTT |
| | | CCGGGCGCTATCATGCCATACCGCGAAAGGTTTTGCGCCATTCGATGGTGTCCGGGATCTCGACGCTCT |
| | | CCCTTATGCGACTCCTGCATTAGGAAGCAGCCCAGTAGTAGGTTGAGGCCGTTGAGCACCGCCGCCGCA |
| | | AGGAATGGTGCATGCAAGGAGATGGCGCCCAACAGTCCCCCGGCCACGGGGCCTGCCACCATACCCACG |
| | | CCGAAACAAGCGCTCATGAGCCCGAAGTGGCGAGCCCGATCTTCCCCATCGGTGATGTCGGCGATATAG |
| | | GCGCCAGCAACCGCACCTGTGGCGCCGGTGATGCCGGCCACGATGCGTCCGGCGTAGAGGATCGAGATC |
| | | GATCTCGATCCCGCGAAATTAATACGACTCACTATAGGGGAATTGTGAGCGGATAACAATTCCCCTCTA |
| | | GAAATAATTTTGTTTAACTTTAAGAAGGAGATATACATATGCATCACCACCACCACCATGCTTCACCCC |
| | | CAAAGAAGAAAAGAAAGGTGGGTTCGATGGATAAGAAATACAGCATCGGCCTGGACATTGGTACGAATA |
| | | GCGTTGGTTGGGCTGTGATCACCGATGACTACAAGGTGCCGAGTAAGAAATTCAAAGTGCTGGGCAACA |
| | | CTGACCGTCATTCCATTAAAAAGAATCTGATCGGCGCGCTGCTGTTTGGTAGTGGCGAAACGGCTGAGG |
| | | CGACGCGTCTGAAGCGTACAGCGCGTCGGCGCTACACCCGTCGTAAGAACCGTATCTGTTATCTGCAAG |
| | | AGATCTTTTCCAACGAAATGGCGAAAGTTGACGACTCCTTCTTCCATCGTCTCGAGGAGAGCTTCTTGG |
| | | TTGAGGAAGATAAAAAGCACGAGCGTCACCCGATTTTTGGTAACATCGTTGATGAAGTGGCCTATCACG |
| | | AAAAATACCCGACGATCTATCATTTGCGCAAAAAGCTGGCCGACAGCACCGACAAAGCAGATCTGCGCT |
| | | TGATCTACTTGGCCCTGGCGCACATGATTAAATTTCGTGGCCATTTTCTGATTGAGGGTGATCTGAACC |
| | | CGGATAACAGCGATGTTGACAAGTTGTTCATTCAGCTGGTACAGATTTATAACCAGCTATTTGAGGAGA |
| | | ACCCGATCAATGCATCGCGGGTGGACGCTAAAGCTATCCTGTCCGCGCGTCTGTCCAAGTCTCGTCGCC |
| | | TGGAAAATCTGATCGCGCAACTGCCGGGTGAAAAGCGCAATGGCTTATTCGGTAACCTTATCGCGCTGT |
| | | CGCTAGGCCTGACCCCGAATTTCAAGAGCAACTTCGACTTAGCGGAAGACGCAAAGCTGCAGCTGAGCA |
| | | AGGACACCTATGACGATGACCTGGATAACCTGCTAGCGCAGATCGGCGACCAGTATGCGGATCTGTTCC |
| | | TGGCTGCGAAGAACTTGTCTGACGCCATATTATTATCCGACATCCTGCGAGTTAATAGCGAGATTACCA |
| | | AAGCGCCACTGTCGGCGTCTATGATCAAGCGCTATGACGAACATCACCAAGATTTGACCTTGCTGAAAG |
| | | CGCTGGTTCGACAGCAGCTGCCGGAAAAGTACAAGGAGATCTTCTTCGACCAGAGCAAAAACGGTTACG |
| | | CCGGTTACATTGACGGTGGTGCTAGCCAGGAGGAGTTCTATAAGTTCATCAAGCCGATCCTGGAGAAAA |
| | | TGGATGGTACCGAAGAACTGTTAGTCAAACTGAACCGTGAAGACCTGCTGAGAAAGCAGCGTACCTTTG |
| | | ACAATGGCTCCATCCCGCATCAAATCCATCTGGGTGAGTTACACGCAATCCTGCGTCGCCAAGAGGACT |
| | | TCTACCCGTTTCTGAAAGACAACCGTGAAAAGATCGAAAAAATCTTGACCTTTCGTATCCCGTATTATG |
| | | TTGGTCCGCTGGCGCGTGGAAACTCACGCTTCGCATGGATGACCCGTAAGAGCGAAGAAACCATCACGC |
| | | CGTGGAATTTTGAAGAAGTTGTGGACAAGGGCGCCTCGGCGCAGTCTTTTATCGAACGCATGACCAATT |
| | | TTGATAAGAACCTGCCGAACGAGAAAGTTCTACCGAAGCACAGCCTGCTGTACGAGTATTTCACCGTTT |
| | | ATAATGAACTGACTAAAGTCAAATACGTGACCGAAGGTATGCGTAAGCCGGCGTTTCTCAGCGGTGAGC |
| | | AGAAAAAGGCCATCGTGGACCTGCTGTTTAAGACGAATCGTAAGGTGACCGTTAAACAATTGAAGGAGG |
| | | ACTACTTCAAAAAGATTGAGTGCTTTGACAGCGTCGAGATTAGCGGCGTTGAAGATCGCTTTAATGCAA |
| | | GCCTGGGGGCTTACCATGATCTGTTGAAGATCATCAAGGACAAAGACTTTCTCGACAACGAAGAAAATG |
| | | AGGATATTTTGGAAGATATCGTTCTGACTCTGACTCTGTTCGAAGACAGGGGTATGATCGAAGAGAGAT |
| | | TGAAGACGTACGCGCACCTGTTCGACGACAAGGTCATGAAACAACTGAAGCGCCGTCGTTACACCGGTT |
| | | GGGGCCGCCTGTCTCGTAAACTGATTAATGGTATTCGTGATAAGCAGAGTGGTAAAACCATATTAGATT |
| | | TTCTGAAAAGCGATGGTTTCGCGAACGCGAACTTCATGCAGTTGATTCACGATGATTCCCTGACCTTCA |
| | | AAGAAGACATCCAGAAGGCCCAAGTTAGCGGCCAGGGTCACAGCCTGCATGAACAGATCGCCAACCTGG |
| | | CTGGTAGCCCGGCGATCAAAAAGGGCATTTTGCAAACCGTGAAAATTGTTGATGAATTGGTGAAAGTGA |
| | | TGGGTCACAAACCGGAGAACATAGTCATCGAGATGGCTCGTGAAAACCAGACGACCCAGAAAGGCCAAA |
| | | AAAACTCTCGTGAACGTATGAAACGTATTGAAGAAGGCATTAAGGAGCTCGGTTCGCAAATTCTGAAGG |
| | | AGCACCCTGTTGAGAATACACAGCTGCAGAATGAGAAACTTTATCTGTACTACCTGCAAAACGGTCGTG |
| | | ATATGTATGTGGATCAAGAACTGGACATTAACCGCTTGTCTGACTACGACGTTGACCATATTGTTCCGC |
| | | AGTCTTTCATTAAAGACGACAGCATTGATAATAAGGTGCTGACGCGCAGTGATAAAAACCGTGGTAAAT |
| | | CCGATAATGTTCCGAGCGAGGAGGTGGTGAAGAAGATGAAAAACTATTGGCGTCAATTGCTTAATGCGA |
| | | AACTGATTACTCAGCGCAAATTCGATAACTTGACGAAAGCAGAACGGGGCGGTCTGTCCGAATTGGACA |
| | | AGGCGGGCTTTATCAAGAGGCAACTGGTGGAGACTCGTCAGATAACGAAGCACGTGGCACAGATCTTAG |
| | | ATTCTCGTATGAATACCAAGTACGATGAGAACGATAAATTGATCCGCGAAGTTAAGGTTATTACCTTGA |
| | | AGTCGAAGCTCGTCAGCGACTTCCGTAAGGACTTTCAATTTTACAAAGTTCGTGAGATCAACAACTACC |
| | | ATCATGCACACGATGCGTATTTGAATGCAGTCGTGGGCACTGCCTTGATTAAAAAATACCCGAAACTGG |
| | | AATCTGAGTTCGTCTATGGGGATTACAAGGTATATGACGTGCGTAAGATGATTGCCAAATCTGAGCAGG |
| | | AGATCGGCAAAGCGACGGCCAAGTACTTTTTCTACTCCAACATTATGAACTTTTTCAAAACGGAAATTA |
| | | CCCTGGCGAATGGCGAGATCCGTAAACGCCCACTGATCGAGACCAATGGTGAGACCGGCGAAATCGTGT |
| | | GGGACAAAGGTCGTGATTTCGCAACCGTTCGCAAAGTGCTGAGTATGCCGCAGGTGAACATCGTTAAAA |
| | | AAACCGAGGTGCAGACCGGAGGTTTTTCGAAAGAGAGCATTCTTCCGAAACGCAACAGCGACAAGCTGA |
| | | TCGCGCGTAAAAAGGACTGGGATCCGAAGAAGTATGGTGGCTTCGATAGCCCGACCGTTGCCTATAGCG |
| | | TTCTGGTAGTTGCTAAGGTTGAGAAAGGCAAAAGTAAAAAGCTGAAATCCGTGAAAGAATTGTTGGGCA |
| | | TTACCATTATGGAACGTAGCTCTTTCGAGAAGAACCCGATTGATTTCCTGGAGGCAAAAGGCTACAAAG |
| | | AGGTCAAAAAGGATCTAATTATTAAGCTGCCAAAGTACAGCCTGTTCGAGCTCGAAAATGGACGTAAAC |
| | | GTATGCTGGCGTCAGCGGGTGAACTGCAGAAGGGTAATGAACTGGCGCTGCCGAGCAAATACGTGAATT |
| | | TTCTGTATCTCGCTAGCCATTATGAAAAACTAAAGGGTTCCCCGGAAGACAACGAGCAGAAGCAGCTTT |
| | | TTGTTGAGCAGCATAAACACTACCTGGACGAGATCATTGAGCAAATCAGCGAGTTCTCTAAGCGTGTCA |
| | | TTTTAGCGGATGCTAATCTTGACAAAGTACTCAGCGCGTATAACAAACACCGGGACAAGCCGATCCGTG |
| | | AACAAGCGGAGAACATTATTCACTTGTTTACCCTGACGAACCTGGGTGCTCCGGCGGCATTTAAATACT |
| | | TCGATACCACCATTGACCGCAAGAGATACACCAGCACCAAAGAGGTGCTGGATGCTACCCTGATTCATC |
| | | AAAGCATCACCGGCCTGTATGAAACCCGTATCGATCTTTCCCAATTAGGCGGCGACAGCCCGGTTCGCT |
| | | CTAGCGGTGGCTCGAGTGGCGGGTCGAGCGGTTCCGAAACCCCCGGCACCAGCGAGTCAGCCACCCCGG |
| | | AAAGCTCTGGCGGGTCCTCCGGCGGCTCCTCGCGTCCGGGTGAGCGCCCTTTCCAATGCCGTATCTGCA |
| | | TGCGTAACTTTTCTAATATGAGCAATCTGACCCGTCACACCCGCACGCATACAGGCGAGAAGCCGTTTC |
| | | AATGTAGAATTTGCATGCGTAACTTCAGCGATCGTAGCGTCCTCAGGCGCCACCTTCGCACCCACACCG |
| | | GCAGCCAAAAGCCATTTCAGTGCCGGATCTGCATGCGTAATTTCTCCGACCCGTCCAACCTGGCGCGTC |
| | | ACACCCGTACCCATACCGGTGAGAAACCATTCCAGTGTCGTATTTGCATGCGTAACTTCAGCGATCGTA |
| | | GCAGCTTGCGCCGCCACCTGCGTACCCATACTGGTTCCCAGAAGCCGTTTCAGTGCCGTATCTGCATGA |
| | | GGAACTTCTCCCAGAGCGGTACCCTGCACCGACATACTAGAACGCACACGGGCGAAAAGCCGTTCCAAT |
| | | GCCGCATCTGTATGCGTAACTTCAGCCAACGTCCGAATCTTACTCGTCACCTGCGCACCCATCTGCGCG |
| | | GTTCGGGCAGCGCGGGTAGCGCGGCAGGTTCCGGCGAATTCCCGAAAAAGAAACGTAAAGTGTAATGAA |
| | | AGCTTGCGGCCGCACTCGAGCACCACCACCACCACCACTGAGATCCGGCTGCTAACAAAGCCCGAAAGG |
| | | AAGCTGAGTTGGCTGCTGCCACCGCTGAGCAATAACTAGCATAACCCCTTGGGGCCTCTAAACGGGTCT |
| | | TGAGGGGTTTTTTGCTGAAAGGAGGAACTATATCCGGAT |
| 7 | cdsDNA177 | CAACTTTGTATAGAAAAGTTGGGCTCCGGTGCCCGTCAGTGGGCAGAGCGCACATCGCCCACAGTCCCC |
| | Cas9 ZF5 | GAGAAGTTGGGGGGAGGGGTCGGCAATTGAACCGGTGCCTAGAGAAGGTGGCGCGGGGTAAACTGGGAA |
| | mammalian | AGTGATGTCGTGTACTGGCTCCGCCTTTTTCCCGAGGGTGGGGGAGAACCGTATATAAGTGCAGTAGTC |
| | expression | GCCGTGAACGTTCTTTTTCGCAACGGGTTTGCCGCCAGAACACAGGTAAGTGCCGTGTGTGGTTCCCGC |
| | vector | GGGCCTGGCCTCTTTACGGGTTATGGCCCTTGCGTGCCTTGAATTACTTCCACCTGGCTGCAGTACGTG |
| | | ATTCTTGATCCCGAGCTTCGGGTTGGAAGTGGGTGGGAGAGTTCGAGGCCTTGCGCTTAAGGAGCCCCT |
| | | TCGCCTCGTGCTTGAGTTGAGGCCTGGCCTGGGCGCTGGGGCCGCCGCGTGCGAATCTGGTGGCACCTT |
| | | CGCGCCTGTCTCGCTGCTTTCGATAAGTCTCTAGCCATTTAAAATTTTTGATGACCTGCTGCGACGCTT |
| | | TTTTTCTGGCAAGATAGTCTTGTAAATGCGGGCCAAGATCTGCACACTGGTATTTCGGTTTTTGGGGCC |
| | | GCGGGCGGCGACGGGGCCCGTGCGTCCCAGCGCACATGTTCGGCGAGGCGGGGCCTGCGAGCGCGGCCA |
| | | CCGAGAATCGGACGGGGGTAGTCTCAAGCTGGCCGGCCTGCTCTGGTGCCTGGTCTCGCGCCGCCGTGT |
| | | ATCGCCCCGCCCTGGGCGGCAAGGCTGGCCCGGTCGGCACCAGTTGCGTGAGCGGAAAGATGGCCGCTT |
| | | CCCGGCCCTGCTGCAGGGAGCTCAAAATGGAGGACGCGGCGCTCGGGAGAGCGGGCGGGTGAGTCACCC |
| | | ACACAAAGGAAAAGGGCCTTTCCGTCCTCAGCCGTCGCTTCATGTGACTCCACGGAGTACCGGGCGCCG |
| | | TCCAGGCACCTCGATTAGTTCTCGAGCTTTTGGAGTACGTCGTCTTTAGGTTGGGGGGAGGGGTTTTAT |
| | | GCGATGGAGTTTCCCCACACTGAGTGGGTGGAGACTGAAGTTAGGCCAGCTTGGCACTTGATGTAATTC |
| | | TCCTTGGAATTTGCCCTTTTTGAGTTTGGATCTTGGTTCATTCTCAAGCCTCAGACAGTGGTTCAAAGT |
| | | TTTTTTCTTCCATTTCAGGTGTCGTGACAAGTTTGTACAAAAAAGCAGGCTGCCACCATGGACTATAAG |
| | | GACCACGACGGAGACTACAAGGATCATGATATTGATTACAAAGACGATGACGATAAGATGGCCCCAAAG |
| | | AAGAAGCGGAAGGTCGGTATCCACGGAGTCCCAGCAGCCGACAAGAAGTACAGCATCGGCCTGGACATC |
| | | GGCACCAACTCTGTGGGCTGGGCCGTGATCACCGACGAGTACAAGGTGCCCAGCAAGAAATTCAAGGTG |
| | | CTGGGCAACACCGACCGGCACAGCATCAAGAAGAACCTGATCGGAGCCCTGCTGTTCGACAGCGGCGAA |
| | | ACAGCCGAGGCCACCCGGCTGAAGAGAACCGCCAGAAGAAGATACACCAGACGGAAGAACCGGATCTGC |
| | | TATCTGCAAGAGATCTTCAGCAACGAGATGGCCAAGGTGGACGACAGCTTCTTCCACAGACTGGAAGAG |
| | | TCCTTCCTGGTGGAAGAGGATAAGAAGCACGAGCGGCACCCCATCTTCGGCAACATCGTGGACGAGGTG |
| | | GCCTACCACGAGAAGTACCCCACCATCTACCACCTGAGAAAGAAACTGGTGGACAGCACCGACAAGGCC |
| | | GACCTGCGGCTGATCTATCTGGCCCTGGCCCACATGATCAAGTTCCGGGGCCACTTCCTGATCGAGGGC |
| | | GACCTGAACCCCGACAACAGCGACGTGGACAAGCTGTTCATCCAGCTGGTGCAGACCTACAACCAGCTG |
| | | TTCGAGGAAAACCCCATCAACGCCAGCGGCGTGGACGCCAAGGCCATCCTGTCTGCCAGACTGAGCAAG |
| | | AGCAGACGGCTGGAAAATCTGATCGCCCAGCTGCCCGGCGAGAAGAAGAATGGCCTGTTCGGAAACCTG |
| | | ATTGCCCTGAGCCTGGGCCTGACCCCCAACTTCAAGAGCAACTTCGACCTGGCCGAGGATGCCAAACTG |
| | | CAGCTGAGCAAGGACACCTACGACGACGACCTGGACAACCTGCTGGCCCAGATCGGCGACCAGTACGCC |
| | | GACCTGTTTCTGGCCGCCAAGAACCTGTCCGACGCCATCCTGCTGAGCGACATCCTGAGAGTGAACACC |
| | | GAGATCACCAAGGCCCCCCTGAGCGCCTCTATGATCAAGAGATACGACGAGCACCACCAGGACCTGACC |
| | | CTGCTGAAAGCTCTCGTGCGGCAGCAGCTGCCTGAGAAGTACAAAGAGATTTTCTTCGACCAGAGCAAG |
| | | AACGGCTACGCCGGCTACATTGACGGCGGAGCCAGCCAGGAAGAGTTCTACAAGTTCATCAAGCCCATC |
| | | CTGGAAAAGATGGACGGCACCGAGGAACTGCTCGTGAAGCTGAACAGAGAGGACCTGCTGCGGAAGCAG |
| | | CGGACCTTCGACAACGGCAGCATCCCCCACCAGATCCACCTGGGAGAGCTGCACGCCATTCTGCGGCGG |
| | | CAGGAAGATTTTTACCCATTCCTGAAGGACAACCGGGAAAAGATCGAGAAGATCCTGACCTTCCGCATC |
| | | CCCTACTACGTGGGCCCTCTGGCCAGGGGAAACAGCAGATTCGCCTGGATGACCAGAAAGAGCGAGGAA |
| | | ACCATCACCCCCTGGAACTTCGAGGAAGTGGTGGACAAGGGCGCTTCCGCCCAGAGCTTCATCGAGCGG |
| | | ATGACCAACTTCGATAAGAACCTGCCCAACGAGAAGGTGCTGCCCAAGCACAGCCTGCTGTACGAGTAC |
| | | TTCACCGTGTATAACGAGCTGACCAAAGTGAAATACGTGACCGAGGGAATGAGAAAGCCCGCCTTCCTG |
| | | AGCGGCGAGCAGAAAAAGGCCATCGTGGACCTGCTGTTCAAGACCAACCGGAAAGTGACCGTGAAGCAG |
| | | CTGAAAGAGGACTACTTCAAGAAAATCGAGTGCTTCGACTCCGTGGAAATCTCCGGCGTGGAAGATCGG |
| | | TTCAACGCCTCCCTGGGCACATACCACGATCTGCTGAAAATTATCAAGGACAAGGACTTCCTGGACAAT |
| | | GAGGAAAACGAGGACATTCTGGAAGATATCGTGCTGACCCTGACACTGTTTGAGGACAGAGAGATGATC |
| | | GAGGAACGGCTGAAAACCTATGCCCACCTGTTCGACGACAAAGTGATGAAGCAGCTGAAGCGGCGGAGA |
| | | TACACCGGCTGGGGCAGGCTGAGCCGGAAGCTGATCAACGGCATCCGGGACAAGCAGTCCGGCAAGAçA |
| | | ATCCTGGATTTCCTGAAGTCCGACGGCTTCGCCAACAGAAACTTCATGCAGCTGATCCACGACGACAGC |
| | | CTGACCTTTAAAGAGGACATCCAGAAAGCCCAGGTGTCCGGCCAGGGCGATAGCCTGCACGAGCACATT |
| | | GCCAATCTGGCCGGCAGCCCCGCCATTAAGAAGGGCATCCTGCAGACAGTGAAGGTGGTGGACGAGCTC |
| | | GTGAAAGTGATGGGCCGGCACAAGCCCGAGAACATCGTGATCGAAATGGCCAGAGAGAACCAGACCACC |
| | | CAGAAGGGACAGAAGAACAGCCGCGAGAGAATGAAGCGGATCGAAGAGGGCATCAAAGAGCTGGGCAGC |
| | | CAGATCCTGAAAGAACACCCCGTGGAAAACACCCAGCTGCAGAACGAGAAGCTGTACCTGTACTACCTG |
| | | CAGAATGGGCGGGATATGTACGTGGACCAGGAACTGGACATCAACCGGCTGTCCGACTACGATGTGGAC |
| | | CATATCGTGCCTCAGAGCTTTCTGAAGGACGACTCCATCGACAACAAGGTGCTGACCAGAAGCGACAAG |
| | | AACCGGGGCAAGAGCGACAACGTGCCCTCCGAAGAGGTCGTGAAGAAGATGAAGAACTACTGGCGGCAG |
| | | CTGCTGAACGCCAAGCTGATTACCCAGAGAAAGTTCGACAATCTGACCAAGGCCGAGAGAGGCGGCCTG |
| | | AGCGAACTGGATAAGGCCGGCTTCATCAAGAGACAGCTGGTGGAAACCCGGCAGATCACAAAGCACGTG |
| | | GCACAGATCCTGGACTCCCGGATGAACACTAAGTACGACGAGAATGACAAGCTGATCCGGGAAGTGAAA |
| | | GTGATCACCCTGAAGTCCAAGCTGGTGTCCGATTTCCGGAAGGATTTCCAGTTTTACAAAGTGCGCGAG |
| | | ATCAACAACTACCACCACGCCCACGACGCCTACCTGAACGCCGTCGTGGGAACCGCCCTGATCAAAAAG |
| | | TACCCTAAGCTGGAAAGCGAGTTCGTGTACGGCGACTACAAGGTGTACGACGTGCGGAAGATGATCGCC |
| | | AAGAGCGAGCAGGAAATCGGCAAGGCTACCGCCAAGTACTTCTTCTACAGCAACATCATGAACTTTTTC |
| | | AAGACCGAGATTACCCTGGCCAACGGCGAGATCCGGAAGCGGCCTCTGATCGAGACAAACGGCGAAACC |
| | | GGGGAGATCGTGTGGGATAAGGGCCGGGATTTTGCCACCGTGCGGAAAGTGCTGAGCATGCCCCAAGTG |
| | | AATATCGTGAAAAAGACCGAGGTGCAGACAGGCGGCTTCAGCAAAGAGTCTATCCTGCCCAAGAGGAAC |
| | | AGCGATAAGCTGATCGCCAGAAAGAAGGACTGGGACCCTAAGAAGTACGGCGGCTTCGACAGCCCCACC |
| | | GTGGCCTATTCTGTGCTGGTGGTGGCCAAAGTGGAAAAGGGCAAGTCCAAGAAACTGAAGAGTGTGAAA |
| | | GAGCTGCTGGGGATCACCATCATGGAAAGAAGCAGCTTCGAGAAGAATCCCATCGACTTTCTGGAAGCC |
| | | AAGGGCTACAAAGAAGTGAAAAAGGACCTGATCATCAAGCTGCCTAAGTACTCCCTGTTCGAGCTGGAA |
| | | AACGGCCGGAAGAGAATGCTGGCCTCTGCCGGCGAACTGCAGAAGGGAAACGAACTGGCCCTGCCCTCC |
| | | AAATATGTGAACTTCCTGTACCTGGCCAGCCACTATGAGAAGCTGAAGGGCTCCCCCGAGGATAATGAG |
| | | CAGAAACAGCTGTTTGTGGAACAGCACAAGCACTACCTGGACGAGATCATCGAGCAGATCAGCGAGTTC |
| | | TCCAAGAGAGTGATCCTGGCCGACGCTAATCTGGACAAAGTGCTGTCCGCCTACAACAAGCACCGGGAT |
| | | AAGCCCATCAGAGAGCAGGCCGAGAATATCATCCACCTGTTTACCCTGACCAATCTGGGAGCCCCTGCC |
| | | GCCTTCAAGTACTTTGACACCACCATCGACCGGAAGAGGTACACCAGCACCAAAGAGGTGCTGGACGCC |
| | | ACCCTGATCCACCAGAGCATCACCGGCCTGTACGAGACACGGATCGACCTGTCTCAGCTGGGAGGCGAC |
| | | TCCCCCGTCCGGAGTAGCGGGGGATCTTCTGGCGGCAGTTCCGGTTCTGAAACCCCCGGGACTTCTGAG |
| | | TCCGCCACCCCCGAATCCAGCGGAGGGTCTTCCGGAGGGTCTTCAAGACCAGGTGAACGCCCATTCCAG |
| | | TGTCGCATCTGCATGCGCAACTTTAGCAATATGAGCAATTTGACCAGACATACAAGAACACATACAGGT |
| | | GAGAAGCCCTTCCAGTGTCGAATATGTATGAGGAATTTCTCAGATAGGTCCGTTCTGAGGCGGCATCTC |
| | | AGAACACATACGGGTTCCCAGAAACCATTCCAATGTCGGATATGTATGCGGAACTTCAGTGACCCATCC |
| | | AATCTCGCTAGGCATACGCGGACCCACACAGGTGAAAAACCGTTTCAATGCCGCATATGCATGCGGAAT |
| | | TTCTCTGACCGATCAAGCCTCAGAAGACACCTCCGAACTCATACCGGCAGCCAGAAACCTTTCCAATGT |
| | | AGGATATGTATGCGAAACTTCTCTCAATCCGGAACCCTTCATCGACATACCAGAACCCATACAGGAGAA |
| | | AAGCCTTTCCAGTGTAGAATTTGCATGAGGAACTTTTCCCAACGCCCAAATCTTACGCGGCATCTCAGG |
| | | ACACATCTGAGGGGGTCTGGCAGCGCAGGCAGTGCAGCGGGGTCAGGAGAGTTCCCCAAAAAAAAGCGC |
| | | AAAGTATAACGCTTCGAGCAGACATGATAAGATACATTGATGAGTTTGGACAAACCACAACTAGAATGC |
| | | AGTGAAAAAAATGCTTTATTTGTGAAATTTGTGATGCTATTGCTTTATTTGTAACCATTATAAGCTGCA |
| | | ATAAACAAGTTAACAACAACAATTGCATTCATTTTATGTTTCAGGTTCAGGGGGAGGTGTGGGAGGTTT |
| | | TTTAAAGCAAGTAAAACCTCTACAAATGTGGTACGCGTTGACATTGATTATTGACTAGTTATTAATAGT |
| | | AATCAATTACGGGGTCATTAGTTCATAGCCCATATATGGAGTTCCGCGTTACATAACTTACGGTAAATG |
| | | GCCCGCCTGGCTGACCGCCCAACGACCCCCGCCCATTGACGTCAATAATGACGTATGTTCCCATAGTAA |
| | | CGCCAATAGGGACTTTCCATTGACGTCAATGGGTGGAGTATTTACGGTAAACTGCCCACTTGGCAGTAC |
| | | ATCAAGTGTATCATATGCCAAGTACGCCCCCTATTGACGTCAATGACGGTAAATGGCCCGCCTGGCATT |
| | | ATGCCCAGTACATGACCTTATGGGACTTTCCTACTTGGCAGTACATCTACGTATTAGTCATCGCTATTA |
| | | CCATGGTGATGCGGTTTTGGCAGTACATCAATGGGCGTGGATAGCGGTTTGACTCACGGGGATTTCCAA |
| | | GTCTCCACCCCATTGACGTCAATGGGAGTTTGTTTTGGCACCAAAATCAACGGGACTTTCCAAAATGTC |
| | | GTAACAACTCCGCCCCATTGACGCAAATGGGCGGTAGGCGTGTACGGTGGGAGGTCTATATAAGCAGAG |
| | | CTCTCTGGCTAACTAGAGAACCCACTGCGCCACCATGACCGAGTACAAGCCCACGGTGCGCCTCGCCAC |
| | | CCGCGACGACGTCCCCAGGGCCGTACGCACCCTCGCCGCCGCGTTCGCCGACTACCCCGCCACGCGCCA |
| | | CACCGTCGATCCGGACCGCCACATCGAGCGGGTCACCGAGCTGCAAGAACTCTTCCTCACGCGCGTCGG |
| | | GCTCGACATCGGCAAGGTGTGGGTCGCGGACGACGGCGCCGCGGTGGCGGTCTGGACCACGCCGGAGAG |
| | | CGTCGAAGCGGGGGCGGTGTTCGCCGAGATCGGCCCGCGCATGGCCGAGTTGAGCGGTTCCCGGCTGGC |
| | | CGCGCAGCAACAGATGGAAGGCCTCCTGGCGCCGCACCGGCCCAAGGAGCCCGCGTGGTTCCTGGCCAC |
| | | CGTCGGCGTCTCGCCCGACCACCAGGGCAAGGGTCTGGGCAGCGCCGTCGTGCTCCCCGGAGTGGAGGC |
| | | GGCCGAGCGCGCCGGGGTGCCCGCCTTCCTGGAGACCTCCGCGCCCCGCAACCTCCCCTTCTACGAGCG |
| | | GCTCGGCTTCACCGTCACCGCCGACGTCGAGGTGCCCGAAGGACCGCGCACCTGGTGCATGACCCGCAA |
| | | GCCCGGTGCCTGACTCGAGTCTAGAGGGCCCGTTTAAACCCGCTGATCAGCCTCGACTGTGCCTTCTAG |
| | | TTGCCAGCCATCTGTTGTTTGCCCCTCCCCCGTGCCTTCCTTGACCCTGGAAGGTGCCACTCCCACTGT |
| | | CCTTTCCTAATAAAATGAGGAAATTGCATCGCATTGTCTGAGTAGGTGTCATTCTATTCTGGGGGGTGG |
| | | GGTGGGGCAGGACAGCAAGGGGGAGGATTGGGAAGACAATAGCAGGCATGCTGGGGATGCGGTGGGCTC |
| | | TATGGGCGGCCGCGGCGCTCTTCCGCTTCCTCGCTCACTGACTCGCTGCGCTCGGTCGTTCGGCTGCGG |
| | | CGAGCGGTATCAGCTCACTCAAAGGCGGTAATACGGTTATCCACAGAATCAGGGGATAACGCAGGAAAG |
| | | AACATGTGAGCAAAAGGCCAGCAAAAGGCCAGGAACCGTAAAAAGGCCGCGTTGCTGGCGTTTTTCCAT |
| | | AGGCTCCGCCCCCCTGACGAGCATCACAAAAATCGACGCTCAAGTCAGAGGTGGCGAAACCCGACAGGA |
| | | CTATAAAGATACCAGGCGTTTCCCCCTGGAAGCTCCCTCGTGCGCTCTCCTGTTCCGACCCTGCCGCTT |
| | | ACCGGATACCTGTCCGCCTTTCTCTCTTCGGGAAGCGTGGCGCTTTCTCATAGCTCACGCTGTAGGTAT |
| | | CTCAGTTCGGTGTAGGTCGTTCGCTCCAAGCTGGGCTGTGTGCACGAACCCCCCGTTCAGCCCGACCGC |
| | | TGCGCCTTATCCGGTAACTATCGTCTTGAGTCCAACCCGGTAAGACACGACTTATCGCCACTGGCAGCA |
| | | GCCACTGGTAACAGGATTAGCAGAGCGAGGTATGTAGGCGGTGCTACAGAGTTCTTGAAGTGGTGGCCT |
| | | AACTACGGCTACACTAGAAGAACAGTATTTGGTATCTGCGCTCTGCTGAAGCCAGTTACCTTCGGAAAA |
| | | AGAGTTGGTAGCTCTTGATCCGGCAAACAAACCACCGCTGGTAGCGGTGGTTTTTTTGTTTGCAAGCAG |
| | | CAGATTACGCGCAGAAAAAAAGGATCTCAAGAAGATCCTTTGATCTTTTCTACGGGGTCTGACGCTCAG |
| | | TGGAACGAAAACTCACGTTAAGGGATTTTGGTCATGAGATTATCAAAAAGGATCTTCACCTAGATCCTT |
| | | TTAAATTAAAAATGAAGTTTTAAATCAATCTAAAGTATATATGAGTAAACTTGGTCTGACAGTTACCAA |
| | | TGCTTAATCAGTGAGGCACCTATCTCAGCGATCTGTCTATTTCGTTCATCCATAGTTGCCTGACTCCCC |
| | | GTCGTGTAGATAACTACGATACGGGAGGGCTTACCATCTGGCCCCAGTGCTGCAATGATACCGCGAGAC |
| | | CCACGCTCACCGGCTCCAGATTTATCAGCAATAAACCAGCCAGCCGGAAGGGCCGAGCGCAGAAGTGGT |
| | | CCTGCAACTTTATCCGCCTCCATCCAGTCTATTAATTGTTGCCGGGAAGCTAGAGTAAGTAGTTCGCCA |
| | | GTTAATAGTTTGCGCAACGTTGTTGCCATTGCTACAGGCATCGTGGTGTCACGCTCGTCGTTTGGTATG |
| | | GCTTCATTCAGCTCCGGTTCCCAACGATCAAGGCGAGTTACATGATCCCCCATGTTGTGCAAAAAAGCG |
| | | GTTAGCTCCTTCGGTCCTCCGATCGTTGTCAGAAGTAAGTTGGCCGCAGTGTTATCACTCATGGTTATG |
| | | GCAGCACTGCATAATTCTCTTACTGTCATGCCATCCGTAAGATGCTTTTCTGTGACTGGTGAGTACTCA |
| | | ACCAAGTCATTCTGAGAATAGTGTATGCGGCGACCGAGTTGCTCTTGCCCGGCGTCAATACGGGATAAT |
| | | ACCGCGCCACATAGCAGAACTTTAAAAGTGCTCATCATTGGAAAACGTTCTTCGGGGCGAAAACTCTCA |
| | | AGGATCTTACCGCTGTTGAGATCCAGTTCGATGTAACCCACTCGTGCACCCAACTGATCTTCAGCATCT |
| | | TTTACTTTCACCAGCGTTTCTGGGTGAGCAAAAACAGGAAGGCAAAATGCCGCAAAAAAGGGAATAAGG |
| | | GCGACACGGAAATGTTGAATACTCATACTCTTCCTTTTTCAATATTATTGAAGCATTTATCAGGGTTAT |
| | | TGTCTCATGAGCGGATACATATTTGAATGTATTTAGAAAAATAAACAAATAGGGGTTCCGCGCACATTT |
| | | CCCCGAAAAGTGCCACCTGACGTCTAAGAAACCATTATTATCATGACATTAACCTATAAAAATAGGCGT |
| | | ATCACGAGGCCCTTTCGTCGGCGCGCCGCGGCCGC |
| 8 | cdsDNA166 | ACTCTTCCTTTTTCAATATTATTGAAGCATTTATCAGGGTTATTGTCTCATGAGCGGATACATATTTGA |
| | pEF-1α | ATGTATTTAGAAAAATAAACAAATAGGGGTTCCGCGCACATTTCCCCGAAAAGTGCCACCTGACGTCTA |
| | Cas9 | AGAAACCATTATTATCATGACATTAACCTATAAAAATAGGCGTATCACGAGGCCCTTTCGTCGGCGCGC |
| | Expression | CGCGGCCGCCAACTTTGTATAGAAAAGTTGGGCTCCGGTGCCCGTCAGTGGGCAGAGCGCACATCGCCC |
| | Vector | ACAGTCCCCGAGAAGTTGGGGGGAGGGGTCGGCAATTGAACCGGTGCCTAGAGAAGGTGGCGCGGGGTA |
| | | AACTGGGAAAGTGATGTCGTGTACTGGCTCCGCCTTTTTCCCGAGGGTGGGGGAGAACCGTATATAAGT |
| | | GCAGTAGTCGCCGTGAACGTTCTTTTTCGCAACGGGTTTGCCGCCAGAACACAGGTAAGTGCCGTGTGT |
| | | GGTTCCCGCGGGCCTGGCCTCTTTACGGGTTATGGCCCTTGCGTGCCTTGAATTACTTCCACCTGGCTG |
| | | CAGTACGTGATTCTTGATCCCGAGCTTCGGGTTGGAAGTGGGTGGGAGAGTTCGAGGCCTTGCGCTTAA |
| | | GGAGCCCCTTCGCCTCGTGCTTGAGTTGAGGCCTGGCCTGGGCGCTGGGGCCGCCGCGTGCGAATCTGG |
| | | TGGCACCTTCGCGCCTGTCTCGCTGCTTTCGATAAGTCTCTAGCCATTTAAAATTTTTGATGACCTGCT |
| | | GCGACGCTTTTTTTCTGGCAAGATAGTCTTGTAAATGCGGGCCAAGATCTGCACACTGGTATTTCGGTT |
| | | TTTGGGGCCGCGGGCGGCGACGGGGCCCGTGCGTCCCAGCGCACATGTTCGGCGAGGCGGGGCCTGCGA |
| | | GCGCGGCCACCGAGAATCGGACGGGGGTAGTCTCAAGCTGGCCGGCCTGCTCTGGTGCCTGGTCTCGCG |
| | | CCGCCGTGTATCGCCCCGCCCTGGGCGGCAAGGCTGGCCCGGTCGGCACCAGTTGCGTGAGCGGAAAGA |
| | | TGGCCGCTTCCCGGCCCTGCTGCAGGGAGCTCAAAATGGAGGACGCGGCGCTCGGGAGAGCGGGCGGGT |
| | | GAGTCACCCACACAAAGGAAAAGGGCCTTTCCGTCCTCAGCCGTCGCTTCATGTGACTCCACGGAGTAC |
| | | CGGGCGCCGTCCAGGCACCTCGATTAGTTCTCGAGCTTTTGGAGTACGTCGTCTTTAGGTTGGGGGGAG |
| | | GGGTTTTATGCGATGGAGTTTCCCCACACTGAGTGGGTGGAGACTGAAGTTAGGCCAGCTTGGCACTTG |
| | | ATGTAATTCTCCTTGGAATTTGCCCTTTTTGAGTTTGGATCTTGGTTCATTCTCAAGCCTCAGACAGTG |
| | | GTTCAAAGTTTTTTTCTTCCATTTCAGGTGTCGTGACAAGTTTGTACAAAAAAGCAGGCTGCCACCATG |
| | | GACTATAAGGACCACGACGGAGACTACAAGGATCATGATATTGATTACAAAGACGATGACGATAAGATG |
| | | GCCCCAAAGAAGAAGCGGAAGGTCGGTATCCACGGAGTCCCAGCAGCCGACAAGAAGTACAGCATCGGC |
| | | CTGGACATCGGCACCAACTCTGTGGGCTGGGCCGTGATCACCGACGAGTACAAGGTGCCCAGCAAGAAA |
| | | TTCAAGGTGCTGGGCAACACCGACCGGCACAGCATCAAGAAGAACCTGATCGGAGCCCTGCTGTTCGAC |
| | | AGCGGCGAAACAGCCGAGGCCACCCGGCTGAAGAGAACCGCCAGAAGAAGATACACCAGACGGAAGAAC |
| | | CGGATCTGCTATCTGCAAGAGATCTTCAGCAACGAGATGGCCAAGGTGGACGACAGCTTCTTCCACAGA |
| | | CTGGAAGAGTCCTTCCTGGTGGAAGAGGATAAGAAGCACGAGCGGCACCCCATCTTCGGCAACATCGTG |
| | | GACGAGGTGGCCTACCACGAGAAGTACCCCACCATCTACCACCTGAGAAAGAAACTGGTGGACAGCACC |
| | | GACAAGGCCGACCTGCGGCTGATCTATCTGGCCCTGGCCCACATGATCAAGTTCCGGGGCCACTTCCTG |
| | | ATCGAGGGCGACCTGAACCCCGACAACAGCGACGTGGACAAGCTGTTCATCCAGCTGGTGCAGACCTAC |
| | | AACCAGCTGTTCGAGGAAAACCCCATCAACGCCAGCGGCGTGGACGCCAAGGCCATCCTGTCTGCCAGA |
| | | CTGAGCAAGAGCAGACGGCTGGAAAATCTGATCGCCCAGCTGCCCGGCGAGAAGAAGAATGGCCTGTTC |
| | | GGAAACCTGATTGCCCTGAGCCTGGGCCTGACCCCCAACTTCAAGAGCAACTTCGACCTGGCCGAGGAT |
| | | GCCAAACTGCAGCTGAGCAAGGACACCTACGACGACGACCTGGACAACCTGCTGGCCCAGATCGGCGAC |
| | | CAGTACGCCGACCTGTTTCTGGCCGCCAAGAACCTGTCCGACGCCATCCTGCTGAGCGACATCCTGAGA |
| | | GTGAACACCGAGATCACCAAGGCCCCCCTGAGCGCCTCTATGATCAAGAGATACGACGAGCACCACCAG |
| | | GACCTGACCCTGCTGAAAGCTCTCGTGCGGCAGCAGCTGCCTGAGAAGTACAAAGAGATTTTCTTCGAC |
| | | CAGAGCAAGAACGGCTACGCCGGCTACATTGACGGCGGAGCCAGCCAGGAAGAGTTCTACAAGTTCATC |
| | | AAGCCCATCCTGGAAAAGATGGACGGCACCGAGGAACTGCTCGTGAAGCTGAACAGAGAGGACCTGCTG |
| | | CGGAAGCAGCGGACCTTCGACAACGGCAGCATCCCCCACCAGATCCACCTGGGAGAGCTGCACGCCATT |
| | | CTGCGGCGGCAGGAAGATTTTTACCCATTCCTGAAGGACAACCGGGAAAAGATCGAGAAGATCCTGACC |
| | | TTCCGCATCCCCTACTACGTGGGCCCTCTGGCCAGGGGAAACAGCAGATTCGCCTGGATGACCAGAAAG |
| | | AGCGAGGAAACCATCACCCCCTGGAACTTCGAGGAAGTGGTGGACAAGGGCGCTTCCGCCCAGAGCTTC |
| | | ATCGAGCGGATGACCAACTTCGATAAGAACCTGCCCAACGAGAAGGTGCTGCCCAAGCACAGCCTGCTG |
| | | TACGAGTACTTCACCGTGTATAACGAGCTGACCAAAGTGAAATACGTGACCGAGGGAATGAGAAAGCCC |
| | | GCCTTCCTGAGCGGCGAGCAGAAAAAGGCCATCGTGGACCTGCTGTTCAAGACCAACCGGAAAGTGACC |
| | | GTGAAGCAGCTGAAAGAGGACTACTTCAAGAAAATCGAGTGCTTCGACTCCGTGGAAATCTCCGGCGTG |
| | | GAAGATCGGTTCAACGCCTCCCTGGGCACATACCACGATCTGCTGAAAATTATCAAGGACAAGGACTTC |
| | | CTGGACAATGAGGAAAACGAGGACATTCTGGAAGATATCGTGCTGACCCTGACACTGTTTGAGGACAGA |
| | | GAGATGATCGAGGAACGGCTGAAAACCTATGCCCACCTGTTCGACGACAAAGTGATGAAGCAGCTGAAG |
| | | CGGCGGAGATACACCGGCTGGGGCAGGCTGAGCCGGAAGCTGATCAACGGCATCCGGGACAAGCAGTCC |
| | | GGCAAGACAATCCTGGATTTCCTGAAGTCCGACGGCTTCGCCAACAGAAACTTCATGCAGCTGATCCAC |
| | | GACGACAGCCTGACCTTTAAAGAGGACATCCAGAAAGCCCAGGTGTCCGGCCAGGGCGATAGCCTGCAC |
| | | GAGCACATTGCCAATCTGGCCGGCAGCCCCGCCATTAAGAAGGGCATCCTGCAGACAGTGAAGGTGGTG |
| | | GACGAGCTCGTGAAAGTGATGGGCCGGCACAAGCCCGAGAACATCGTGATCGAAATGGCCAGAGAGAAC |
| | | CAGACCACCCAGAAGGGACAGAAGAACAGCCGCGAGAGAATGAAGCGGATCGAAGAGGGCATCAAAGAG |
| | | CTGGGCAGCCAGATCCTGAAAGAACACCCCGTGGAAAACACCCAGCTGCAGAACGAGAAGCTGTACCTG |
| | | TACTACCTGCAGAATGGGCGGGATATGTACGTGGACCAGGAACTGGACATCAACCGGCTGTCCGACTAC |
| | | GATGTGGACCATATCGTGCCTCAGAGCTTTCTGAAGGACGACTCCATCGACAACAAGGTGCTGACCAGA |
| | | AGCGACAAGAACCGGGGCAAGAGCGACAACGTGCCCTCCGAAGAGGTCGTGAAGAAGATGAAGAACTAC |
| | | TGGCGGCAGCTGCTGAACGCCAAGCTGATTACCCAGAGAAAGTTCGACAATCTGACCAAGGCCGAGAGA |
| | | GGCGGCCTGAGCGAACTGGATAAGGCCGGCTTCATCAAGAGACAGCTGGTGGAAACCCGGCAGATCACA |
| | | AAGCACGTGGCACAGATCCTGGACTCCCGGATGAACACTAAGTACGACGAGAATGACAAGCTGATCCGG |
| | | GAAGTGAAAGTGATCACCCTGAAGTCCAAGCTGGTGTCCGATTTCCGGAAGGATTTCCAGTTTTACAAA |
| | | GTGCGCGAGATCAACAACTACCACCACGCCCACGACGCCTACCTGAACGCCGTCGTGGGAACCGCCCTG |
| | | ATCAAAAAGTACCCTAAGCTGGAAAGCGAGTTCGTGTACGGCGACTACAAGGTGTACGACGTGCGGAAG |
| | | ATGATCGCCAAGAGCGAGCAGGAAATCGGCAAGGCTACCGCCAAGTACTTCTTCTACAGCAACATCATG |
| | | AACTTTTTCAAGACCGAGATTACCCTGGCCAACGGCGAGATCCGGAAGCGGCCTCTGATCGAGACAAAC |
| | | GGCGAAACCGGGGAGATCGTGTGGGATAAGGGCCGGGATTTTGCCACCGTGCGGAAAGTGCTGAGCATG |
| | | CCCCAAGTGAATATCGTGAAAAAGACCGAGGTGCAGACAGGCGGCTTCAGCAAAGAGTCTATCCTGCCC |
| | | AAGAGGAACAGCGATAAGCTGATCGCCAGAAAGAAGGACTGGGACCCTAAGAAGTACGGCGGCTTCGAC |
| | | AGCCCCACCGTGGCCTATTCTGTGCTGGTGGTGGCCAAAGTGGAAAAGGGCAAGTCCAAGAAACTGAAG |
| | | AGTGTGAAAGAGCTGCTGGGGATCACCATCATGGAAAGAAGCAGCTTCGAGAAGAATCCCATCGACTTT |
| | | CTGGAAGCCAAGGGCTACAAAGAAGTGAAAAAGGACCTGATCATCAAGCTGCCTAAGTACTCCCTGTTC |
| | | GAGCTGGAAAACGGCCGGAAGAGAATGCTGGCCTCTGCCGGCGAACTGCAGAAGGGAAACGAACTGGCC |
| | | CTGCCCTCCAAATATGTGAACTTCCTGTACCTGGCCAGCCACTATGAGAAGCTGAAGGGCTCCCCCGAG |
| | | GATAATGAGCAGAAACAGCTGTTTGTGGAACAGCACAAGCACTACCTGGACGAGATCATCGAGCAGATC |
| | | AGCGAGTTCTCCAAGAGAGTGATCCTGGCCGACGCTAATCTGGACAAAGTGCTGTCCGCCTACAACAAG |
| | | CACCGGGATAAGCCCATCAGAGAGCAGGCCGAGAATATCATCCACCTGTTTACCCTGACCAATCTGGGA |
| | | GCCCCTGCCGCCTTCAAGTACTTTGACACCACCATCGACCGGAAGAGGTACACCAGCACCAAAGAGGTG |
| | | CTGGACGCCACCCTGATCCACCAGAGCATCACCGGCCTGTACGAGACACGGATCGACCTGTCTCAGCTG |
| | | GGAGGCGACAAAAGGCCGGCGGCCACGAAAAAGGCCGGCCAGGCAAAAAAGAAAAAGTAAACCCAGCTT |
| | | TCTTGTACAAAGTGGTGATGGCCGGCCGCTTCGAGCAGACATGATAAGATACATTGATGAGTTTGGACA |
| | | AACCACAACTAGAATGCAGTGAAAAAAATGCTTTATTTGTGAAATTTGTGATGCTATTGCTTTATTTGT |
| | | AACCATTATAAGCTGCAATAAACAAGTTAACAACAACAATTGCATTCATTTTATGTTTCAGGTTCAGGG |
| | | GGAGGTGTGGGAGGTTTTTTAAAGCAAGTAAAACCTCTACAAATGTGGTACGCGTTGACATTGATTATT |
| | | GACTAGTTATTAATAGTAATCAATTACGGGGTCATTAGTTCATAGCCCATATATGGAGTTCCGCGTTAC |
| | | ATAACTTACGGTAAATGGCCCGCCTGGCTGACCGCCCAACGACCCCCGCCCATTGACGTCAATAATGAC |
| | | GTATGTTCCCATAGTAACGCCAATAGGGACTTTCCATTGACGTCAATGGGTGGAGTATTTACGGTAAAC |
| | | TGCCCACTTGGCAGTACATCAAGTGTATCATATGCCAAGTACGCCCCCTATTGACGTCAATGACGGTAA |
| | | ATGGCCCGCCTGGCATTATGCCCAGTACATGACCTTATGGGACTTTCCTACTTGGCAGTACATCTACGT |
| | | ATTAGTCATCGCTATTACCATGGTGATGCGGTTTTGGCAGTACATCAATGGGCGTGGATAGCGGTTTGA |
| | | CTCACGGGGATTTCCAAGTCTCCACCCCATTGACGTCAATGGGAGTTTGTTTTGGCACCAAAATCAACG |
| | | GGACTTTCCAAAATGTCGTAACAACTCCGCCCCATTGACGCAAATGGGCGGTAGGCGTGTACGGTGGGA |
| | | GGTCTATATAAGCAGAGCTCTCTGGCTAACTAGAGAACCCACTGCGCCACCATGACCGAGTACAAGCCC |
| | | ACGGTGCGCCTCGCCACCCGCGACGACGTCCCCAGGGCCGTACGCACCCTCGCCGCCGCGTTCGCCGAC |
| | | TACCCCGCCACGCGCCACACCGTCGATCCGGACCGCCACATCGAGCGGGTCACCGAGCTGCAAGAACTC |
| | | TTCCTCACGCGCGTCGGGCTCGACATCGGCAAGGTGTGGGTCGCGGACGACGGCGCCGCGGTGGCGGTC |
| | | TGGACCACGCCGGAGAGCGTCGAAGCGGGGGCGGTGTTCGCCGAGATCGGCCCGCGCATGGCCGAGTTG |
| | | AGCGGTTCCCGGCTGGCCGCGCAGCAACAGATGGAAGGCCTCCTGGCGCCGCACCGGCCCAAGGAGCCC |
| | | GCGTGGTTCCTGGCCACCGTCGGCGTCTCGCCCGACCACCAGGGCAAGGGTCTGGGCAGCGCCGTCGTG |
| | | CTCCCCGGAGTGGAGGCGGCCGAGCGCGCCGGGGTGCCCGCCTTCCTGGAGACCTCCGCGCCCCGCAAC |
| | | CTCCCCTTCTACGAGCGGCTCGGCTTCACCGTCACCGCCGACGTCGAGGTGCCCGAAGGACCGCGCACC |
| | | TGGTGCATGACCCGCAAGCCCGGTGCCTGACTCGAGTCTAGAGGGCCCGTTTAAACCCGCTGATCAGCC |
| | | TCGACTGTGCCTTCTAGTTGCCAGCCATCTGTTGTTTGCCCCTCCCCCGTGCCTTCCTTGACCCTGGAA |
| | | GGTGCCACTCCCACTGTCCTTTCCTAATAAAATGAGGAAATTGCATCGCATTGTCTGAGTAGGTGTCAT |
| | | TCTATTCTGGGGGGTGGGGTGGGGCAGGACAGCAAGGGGGAGGATTGGGAAGACAATAGCAGGCATGCT |
| | | GGGGATGCGGTGGGCTCTATGGGCGGCCGCGGCGCTCTTCCGCTTCCTCGCTCACTGACTCGCTGCGCT |
| | | CGGTCGTTCGGCTGCGGCGAGCGGTATCAGCTCACTCAAAGGCGGTAATACGGTTATCCACAGAATCAG |
| | | GGGATAACGCAGGAAAGAACATGTGAGCAAAAGGCCAGCAAAAGGCCAGGAACCGTAAAAAGGCCGCGT |
| | | TGCTGGCGTTTTTCCATAGGCTCCGCCCCCCTGACGAGCATCACAAAAATCGACGCTCAAGTCAGAGGT |
| | | GGCGAAACCCGACAGGACTATAAAGATACCAGGCGTTTCCCCCTGGAAGCTCCCTCGTGCGCTCTCCTG |
| | | TTCCGACCCTGCCGCTTACCGGATACCTGTCCGCCTTTCTCTCTTCGGGAAGCGTGGCGCTTTCTCATA |
| | | GCTCACGCTGTAGGTATCTCAGTTCGGTGTAGGTCGTTCGCTCCAAGCTGGGCTGTGTGCACGAACCCC |
| | | CCGTTCAGCCCGACCGCTGCGCCTTATCCGGTAACTATCGTCTTGAGTCCAACCCGGTAAGACACGACT |
| | | TATCGCCACTGGCAGCAGCCACTGGTAACAGGATTAGCAGAGCGAGGTATGTAGGCGGTGCTACAGAGT |
| | | TCTTGAAGTGGTGGCCTAACTACGGCTACACTAGAAGAACAGTATTTGGTATCTGCGCTCTGCTGAAGC |
| | | CAGTTACCTTCGGAAAAAGAGTTGGTAGCTCTTGATCCGGCAAACAAACCACCGCTGGTAGCGGTGGTT |
| | | TTTTTGTTTGCAAGCAGCAGATTACGCGCAGAAAAAAAGGATCTCAAGAAGATCCTTTGATCTTTTCTA |
| | | CGGGGTCTGACGCTCAGTGGAACGAAAACTCACGTTAAGGGATTTTGGTCATGAGATTATCAAAAAGGA |
| | | TCTTCACCTAGATCCTTTTAAATTAAAAATGAAGTTTTAAATCAATCTAAAGTATATATGAGTAAACTT |
| | | GGTCTGACAGTTACCAATGCTTAATCAGTGAGGCACCTATCTCAGCGATCTGTCTATTTCGTTCATCCA |
| | | TAGTTGCCTGACTCCCCGTCGTGTAGATAACTACGATACGGGAGGGCTTACCATCTGGCCCCAGTGCTG |
| | | CAATGATACCGCGAGACCCACGCTCACCGGCTCCAGATTTATCAGCAATAAACCAGCCAGCCGGAAGGG |
| | | CCGAGCGCAGAAGTGGTCCTGCAACTTTATCCGCCTCCATCCAGTCTATTAATTGTTGCCGGGAAGCTA |
| | | GAGTAAGTAGTTCGCCAGTTAATAGTTTGCGCAACGTTGTTGCCATTGCTACAGGCATCGTGGTGTCAC |
| | | GCTCGTCGTTTGGTATGGCTTCATTCAGCTCCGGTTCCCAACGATCAAGGCGAGTTACATGATCCCCCA |
| | | TGTTGTGCAAAAAAGCGGTTAGCTCCTTCGGTCCTCCGATCGTTGTCAGAAGTAAGTTGGCCGCAGTGT |
| | | TATCACTCATGGTTATGGCAGCACTGCATAATTCTCTTACTGTCATGCCATCCGTAAGATGCTTTTCTG |
| | | TGACTGGTGAGTACTCAACCAAGTCATTCTGAGAATAGTGTATGCGGCGACCGAGTTGCTCTTGCCCGG |
| | | CGTCAATACGGGATAATACCGCGCCACATAGCAGAACTTTAAAAGTGCTCATCATTGGAAAACGTTCTT |
| | | CGGGGCGAAAACTCTCAAGGATCTTACCGCTGTTGAGATCCAGTTCGATGTAACCCACTCGTGCACCCA |
| | | ACTGATCTTCAGCATCTTTTACTTTCACCAGCGTTTCTGGGTGAGCAAAAACAGGAAGGCAAAATGCCG |
| | | CAAAAAAGGGAATAAGGGCGACACGGAAATGTTGAATACTCAT |
| 9 | cdsDNA164 | GAGGGCCTATTTCCCATGATTCCTTCATATTTGCATATACGATACAAGGCTGTTAGAGAGATAATTGGA |
| | g526 | ATTAATTTGACTGTAAACACAAAGATATTAGTACAAAATACGTGACGTAGAAAGTAATAATTTCTTGGG |
| | mammalian | TAGTTTGCAGTTTTAAAATTATGTTTTAAAATGGACTATCATATGCTTACCGTAACTTGAAAGTATTTC |
| | expression | GATTTCTTGGCTTTATATATCTTGTGGAAAGGACGAAACACCGTCAGGGTTCTGGATATCTGTGTTTTA |
| | vector | GAGCTAGAAATAGCAAGTTAAAATAAGGCTAGTCCGTTATCAACTTGAAAAAGTGGCACCGAGTCGGTG |
| | | CTTTTTTGTCTAGAGGTACCGGCGCTCTTCCGCTTCCTCGCTCACTGACTCGCTGCGCTCGGTCGTTCG |
| | | GCTGCGGCGAGCGGTATCAGCTCACTCAAAGGCGGTAATACGGTTATCCACAGAATCAGGGGATAACGC |
| | | AGGAAAGAACATGTGAGCAAAAGGCCAGCAAAAGGCCAGGAACCGTAAAAAGGCCGCGTTGCTGGCGTT |
| | | TTTCCATAGGCTCCGCCCCCCTGACGAGCATCACAAAAATCGACGCTCAAGTCAGAGGTGGCGAAACCC |
| | | GACAGGACTATAAAGATACCAGGCGTTTCCCCCTGGAAGCTCCCTCGTGCGCTCTCCTGTTCCGACCCT |
| | | GCCGCTTACCGGATACCTGTCCGCCTTTCTCCCTTCGGGAAGCGTGGCGCTTTCTCATAGCTCACGCTG |
| | | TAGGTATCTCAGTTCGGTGTAGGTCGTTCGCTCCAAGCTGGGCTGTGTGCACGAACCCCCCGTTCAGCC |
| | | CGACCGCTGCGCCTTATCCGGTAACTATCGTCTTGAGTCCAACCCGGTAAGACACGACTTATCGCCACT |
| | | GGCAGCAGCCACTGGTAACAGGATTAGCAGAGCGAGGTATGTAGGCGGTGCTACAGAGTTCTTGAAGTG |
| | | GTGGCCTAACTACGGCTACACTAGAAGAACAGTATTTGGTATCTGCGCTCTGCTGAAGCCAGTTACCTT |
| | | CGGAAAAAGAGTTGGTAGCTCTTGATCCGGCAAACAAACCACCGCTGGTAGCGGTGGTTTTTTTGTTTG |
| | | CAAGCAGCAGATTACGCGCAGAAAAAAAGGATCTCAAGAAGATCCTTTGATCTTTTCTACGGGGTCTGA |
| | | CGCTCAGTGGAACGAAAACTCACGTTAAGGGATTTTGGTCATGAGATTATCAAAAAGGATCTTCACCTA |
| | | GATCCTTTTAAATTAAAAATGAAGTTTTAAATCAATCTAAAGTATATATGAGTAAACTTGGTCTGACAG |
| | | TTACCAATGCTTAATCAGTGAGGCACCTATCTCAGCGATCTGTCTATTTCGTTCATCCATAGTTGCCTG |
| | | ACTCCCCGTCGTGTAGATAACTACGATACGGGAGGGCTTACCATCTGGCCCCAGTGCTGCAATGATACC |
| | | GCGAGATCCACGCTCACCGGCTCCAGATTTATCAGCAATAAACCAGCCAGCCGGAAGGGCCGAGCGCAG |
| | | AAGTGGTCCTGCAACTTTATCCGCCTCCATCCAGTCTATTAATTGTTGCCGGGAAGCTAGAGTAAGTAG |
| | | TTCGCCAGTTAATAGTTTGCGCAACGTTGTTGCCATTGCTACAGGCATCGTGGTGTCACGCTCGTCGTT |
| | | TGGTATGGCTTCATTCAGCTCCGGTTCCCAACGATCAAGGCGAGTTACATGATCCCCCATGTTGTGCAA |
| | | AAAAGCGGTTAGCTCCTTCGGTCCTCCGATCGTTGTCAGAAGTAAGTTGGCCGCAGTGTTATCACTCAT |
| | | GGTTATGGCAGCACTGCATAATTCTCTTACTGTCATGCCATCCGTAAGATGCTTTTCTGTGACTGGTGA |
| | | GTACTCAACCAAGTCATTCTGAGAATAGTGTATGCGGCGACCGAGTTGCTCTTGCCCGGCGTCAATACG |
| | | GGATAATACCGCGCCACATAGCAGAACTTTAAAAGTGCTCATCATTGGAAAACGTTCTTCGGGGCGAAA |
| | | ACTCTCAAGGATCTTACCGCTGTTGAGATCCAGTTCGATGTAACCCACTCGTGCACCCAACTGATCTTC |
| | | AGCATCTTTTACTTTCACCAGCGTTTCTGGGTGAGCAAAAACAGGAAGGCAAAATGCCGCAAAAAAGGG |
| | | AATAAGGGCGACACGGAAATGTTGAATACTCATACTCTTCCTTTTTCAATATTATTGAAGCATTTATCA |
| | | GGGTTATTGTCTCATGAGCGGATACATATTTGAATGTATTTAGAAAAATAAACAAATAGGGGTTCCGCG |
| | | CACATTTCCCCGAAAAGTGCCACCTGACGTCTAAGAAACCATTATTATCATGACATTAACCTATAAAAA |
| | | TAGGCGTATCACGAGGCCCTTTCGTC |
| 10 | cdsDNA136 | CATGACATTAACCTATAAAAATAGGCGTATCACGAGGCAGAATTTCAGCCATCGCCCTGATAGACGGTT |
| | emGFP | TTTCGCCCTTTGACGTTGGAGTCCACGTTCTTTAATAGTGGACTCTTGTTCCAAACTGGAACAACACTC |
| | 1200 at | AACCCTATCTCGGGCAAGCTTGTCACAGTCGGTTCTGGATATCTGTTGGGTCTCCCCTCTGACCATGGA |
| | TRAC | GCCCACCCCTGCTCCACTGCTCCAGGGACAGCCCTATGCTGCAGGCAGCTCTGCCCCCACTCAGCATCC |
| | human | CAGGGGCTGATTTCTTTGGTTTTGGATCCAGCTGGATGTCTGCATTGCCGAGGCCACCAGGGCTGGCTC |
| | grp78 | AGCAACTGTCGGGGAATCACCAGGGTCTGAGAAATCTTGTGCGCATGTGAGGGGCTGTGGGAGCAGAGA |
| | | ACCACTGGGTGGGAAATTCTAATCCCCACCCTGCTGGAAACTCTCTGGGTGGCCCCAACATGCTAATCC |
| | | TCCGGTAAACCTCTGTTTCCTCCTCAAAAGGCAGGAGGTCGGAAAGAATAAACAATGAGAGTCACATTA |
| | | AAAACACAAAATCCTACGGAAATACTGAAGAATGAGTCTCAGCACTAAGGAAAAGCCTCCAGCAGCTCC |
| | | TGCTTTCTGAGGGTGAAGGATAGACGCTGTGGCTCTGCATGACTCACTAGCACTCTATCACGGCCATAT |
| | | TCTGACAGGGTCAGTGGCTCCAACTAACATTTGTTTGGTACTTTACAGTTTATTAAATAGATGTTTATA |
| | | TGGAGAAGCTCTCATTTCTTTCTCAGAAGAGCCTGGCTAGGAAGGTGGATGAGGCACCATATTCATTTT |
| | | GCAGGTGAAATTCCTGAGATGTAAGGAGCTGCTGTGACTTGCTCAAGGCCTTATATCGAGTAAACGGTA |
| | | GCGCTGGGGCTTAGACGCAGGTGTTCTGATTTATAGTTCAAAACCTCTATCAATGAGAGAGCAATCTCC |
| | | TGGTAATGTGATAGATTTCCCAACTTAATGCCAACATACCATAAACCTCCCATTCTGCTAATGCCCAGC |
| | | CTAAGTTGGGGAGACCACTCCAGATTCCAAGATGTACAGTTTGCTTTGCTGGGCCTTTTTCCCATGCCT |
| | | GCCTTTACTCTGCCAGAGTTATATTGCTGGGGTTTTGAAGAAGATCCTATTAAATAAAAGAATAAGCAG |
| | | TATTATTAAGTAGCCCTGCATTTCAGGTTTCCTTGAGTGGCAGGCCAGGCCTGGCCGTGAACGTTCACT |
| | | GAAATCATGGCCTCTTGGCCAAGATTGATAGCTTGTGCCTGTCCCTGAGTCCCAGTCCATCACGAGCAG |
| | | CTGGTTTCTAAGATGCTATTTCCCGTATAAAGCATGAGACCGTGACTTGCCAGCCCCACAGAGCCCCGC |
| | | CCTTGTCCATCACTGGCATCTGGACTCCAGCCTGGGTTGGGGCAAAGAGGGAAATGAGATCATGTCCTA |
| | | ACCCTGCTTTGTGATGGGAATTCTGTGGACCTGCAGGGCCCACTAGTGCGGTTACCAGCGGAAATGCCT |
| | | CGGGGTCAGAAGTCGCAGGAGAGATAGACAGCTGCTGAACCAATGGGACCAGCGGATGGGGCGGATGTT |
| | | ATCTACCATTGGTGAACGTTAGAAACGAATAGCAGCCAATGAATCAGCTGGGGGGGCGGAGCAGTGACG |
| | | TTTATTGCGGAGGGGGCCGCTTCGAATCGGCGGCGGCCAGCTTGGTGGCCTGGGCCAATGAACGGCCTC |
| | | CAACGAGCAGGGCCTTCACCAATCGGCGGCCTCCACGACGGGGCTGGGGGAGGGTATATAAGCCGAGTA |
| | | GGCGACGGTGAGGTCGACGCCGGCCAAGACAGCACAGACAGATTGACCTATTGGGGTGTTTCGCGAGTG |
| | | TGAGAGGGAAGCGCCGCGGCCTGTATTTCTAGACCTGCCCTTCGCCTGGTTCGTGGCGCCTTGTGACCC |
| | | CGGGCCCCTGCCGCCTGCAAGTCGGAAATTGCGCTGTGCTCCTGTGCTACGGCCTGTGGCTGGACTGCC |
| | | TGCTGCTGCCCAACTGGCTGCCACCATGGTCTCCAAAGGAGAAGAACTCTTCACCGGAGTCGTACCAAT |
| | | CCTCGTCGAACTTGATGGCGACGTCAATGGTCATAAATTTTTCGTGTCTGGTGAGGGAGAGGGGGACGC |
| | | GACTTATGGAAAGCTCACGCTCAAGTTTATCTGCACCACTGGAAAACTGCCGGTCCCGTGGCCAACTCT |
| | | TGTGACCACGTTTACATACGGAGTCCAGTGCTTCGCCAGATATCCCGACCATATGAAGCAACATGACTT |
| | | CTTTAAGAGTGCCATGCCGGAAGGGTACGTGCAAGAGAGAACTATCTTTTTCAAGGATGACGGCAACTA |
| | | CAAAACTCGAGCTGAGGTCAAGTTTGAGGGTGATACCCTCGTCAATAGAATTGAATTGAAGGGCATCGA |
| | | CTTTAAGGAGGACGGCAACATCCTGGGCCACAAGCTGGAATATAATTACAACAGCCACAAAGTCTACAT |
| | | TACGGCAGACAAGCAGAAGAACGGCATAAAAGTCAACTTTAAAACGAGGCACAACATAGAAGATGGCTC |
| | | TGTCCAGTTGGCTGACCACTATCAGCAGAATACGCCTATAGGGGATGGGCCGGTTCTGCTCCCCGACAA |
| | | TCATTACCTCAGTACCCAATCTGCTTTGTCAAAGGACCCCAATGAGAAAAGAGACCATATGGTCCTGCT |
| | | CGAGTTTGTCACAGCCGCCGGTATAACACTTGGTATGGATGAGCTGTATAAGTAATGATAGAAGCTTGA |
| | | TCTTTTTCCCTCTGCCAAAAATTATGGGGACATCATGAAGCCCCTTGAGCATCTGACTTCTGGCTAATA |
| | | AAGGAAATTTATTTTCATTGCAATAGTGTGTTGGAATTTTTTGTGTCTCTCACTCGACTCGAAAAGATA |
| | | CGCAGCGCTGGATGAGATGAGAAATATTCCGCGACGCAAGCAGGAATATCCAGAACCCTGACCCTGCCG |
| | | TGTACCAGCTGAGAGACTCTAAATCCAGTGACAAGTCTGTCTGCCTATTCACCGATTTTGATTCTCAAA |
| | | CAAATGTGTCACAAAGTAAGGATTCTGATGTGTATATCACAGACAAAACTGTGCTAGACATGAGGTCTA |
| | | TGGACTTCAAGAGCAACAGTGCTGTGGCCTGGAGCAACAAATCTGACTTTGCATGTGCAAACGCCTTCA |
| | | ACAACAGCATTATTCCAGAAGACACCTTCTTCCCCAGCCCAGGTAAGGGCAGCTTTGGTGCCTTCGCAG |
| | | GCTGTTTCCTTGCTTCAGGAATGGCCAGGTTCTGCCCAGAGCTCTGGTCAATGATGTCTAAAACTCCTC |
| | | TGATTGGTGGTCTCGGCCTTATCCATTGCCACCAAAACCCTCTTTTTACTAAGAAACAGTGAGCCTTGT |
| | | TCTGGCAGTCCAGAGAATGACACGGGAAAAAAGCAGATGAAGAGAAGGTGGCAGGAGAGGGCACGTGGC |
| | | CCAGCCTCAGTCTCTCCAACTGAGTTCCTGCCTGCCTGCCTTTGCTCAGACTGTTTGCCCCTTACTGCT |
| | | CTTCTAGGCCTCATTCTAAGCCCCTTCTCCAAGTTGCCTCTCCTTATTTCTCCCTGTCTGCCAAAAAAT |
| | | CTTTCCCAGCTCACTAAGTCAGTCTCACGCAGTCACTCATTAACCCACCAATCACTGATTGTGCCGGCA |
| | | CATGAATGCACCAGGTGTTGAAGTGGAGGAATTAAAAAGTCAGATGAGGGGTGTGCCCAGAGGAAGCAC |
| | | CATTCTAGTTGGGGGAGCCCATCTGTCAGCTGGGAAAAGTCCAAATAACTTCAGATTGGAATGTGTTTT |
| | | AACTCAGGGTTGAGAAAACAGCCACCTTCAGGACAAAAGTCAGGGAAGGGCTCTCTGAAGAAATGCTAC |
| | | TTGAAGATACCAGCCCTACCAAGGGCAGGGAGAGGACCCTATAGAGGCCTGGGACAGGAGCTCAATGAG |
| | | AAAGGAGAAGAGCAGCAGGCATGAGTTGAATGAAGGAGGCAGGGCCGGGTCACAGGGCCTTCTAGGCCA |
| | | TGAGAGGGTAGACAGTATTCTAAGTACGCCAGAAAGCTGTTGATCGGCTTCAAGCAGGGAAGGGACACC |
| | | TAATTTGCTTTTCTTTTCTTTTTTTTTTTTTTTTTTTTTTTGAGATGGAGTTTTGCTCTTGTTGCCCAG |
| | | GCTGGAGTGCAATGGTGCATCTTGGCTCACTGCAACCCCAACAGATATCCAGAACCCATTGACCGGGAG |
| | | CGCCCTGTAGCGGCGCATTAAGCGCGGCGGGTGTGGTGGTTACGCGCAGCGTGACCGCTACACTTGCCA |
| | | GCGCCCTAGCGCCCGCTCCCGGGATCGGAATTCCGGCCATCGCCCTGATAGACGGTTTTTCGCCCTTTG |
| | | ACGTTGGAGTCCACGTTCTTTAATAGTGGACTCTTGTTCCAAACTGGAAAACGTCTAGCGGTATCAGCT |
| | | CACTCAAAGGCGGTAATACGGTTATCCACAGAATCAGGGGATAACGCAGGAAAGAACATGTGAGCAAAA |
| | | GGCCAGCAAAAGGCCAGGAACCGTAAAAAGGCCGCGTTGCTGGCGTTTTTCCATAGGCTCCGCCCCCCT |
| | | GACGAGCATCACAAAAATCGACGCTCAAGTCAGAGGTGGCGAAACCCGACAGGACTATAAAGATACCAG |
| | | GCGTTTCCCCCTGGAAGCTCCCTCGTGCGCTCTCCTGTTCCGACCCTGCCGCTTACCGGATACCTGTCC |
| | | GCCTTTCTCCCTTCGGGAAGCGTGGCGCTTTCTCATAGCTCACGCTGTAGGTATCTCAGTTCGGTGTAG |
| | | GTCGTTCGCTCCAAGCTGGGCTGTGTGCACGAACCCCCCGTTCAGCCCGACCGCTGCGCCTTATCCGGT |
| | | AACTATCGTCTTGAGTCCAACCCGGTAAGACACGACTTATCGCCACTGGCAGCAGCCACTGGTAACAGG |
| | | ATTAGCAGAGCGAGGTATGTAGGCGGTGCTACAGAGTTCTTGAAGTGGTGGCCTAACTACGGCTACACT |
| | | AGAAGGACAGTATTTGGTATCTGCGCTCTGCTGAAGCCAGTTACCTTCGGAAAAAGAGTTGGTAGCTCT |
| | | TGATCCGGCAAACAAACCACCGCTGGTAGCGGTGGTTTTTTTGTTTGCAAGCAGCAGATTACGCGCAGA |
| | | AAAAAAGGATCTCAAGAAGATCCTTTGATCTTTTCTACGGGGTCTGACGCTCAGTGGAACGAAAACTCA |
| | | CGTTAAGGGATTTTGGTCATGAGATTATCAAAAAGGATCTTCACCTAGATCCTTTTAAATTAAAAATGA |
| | | AGTTTTAAATCAATCTAAAGTATATATGAGTAAACTTGGTCTGACAGTTACCAATGCTTAATCAGTGAG |
| | | GCACCTATCTCAGCGATCTGTCTATTTCGTTCATCCATAGTTGCCTGACTCCCCGTCGTGTAGATAACT |
| | | ACGATACGGGAGGGCTTACCATCTGGCCCCAGTGCTGCAATGATACCGCGAGACCCACGCTCACCGGCT |
| | | CCAGATTTATCAGCAATAAACCAGCCAGCCGGAAGGGCCGAGCGCAGAAGTGGTCCTGCAACTTTATCC |
| | | GCCTCCATCCAGTCTATTAATTGTTGCCGGGAAGCTAGAGTAAGTAGTTCGCCAGTTAATAGTTTGCGC |
| | | AACGTTGTTGCCATTGCTACAGGCATCGTGGTGTCACGCTCGTCGTTTGGTATGGCTTCATTCAGCTCC |
| | | GGTTCCCAACGATCAAGGCGAGTTACATGATCCCCCATGTTGTGCAAAAAAGCGGTTAGCTCCTTCGGT |
| | | CCTCCGATCGTTGTCAGAAGTAAGTTGGCCGCAGTGTTATCACTCATGGTTATGGCAGCACTGCATAAT |
| | | TCTCTTACTGTCATGCCATCCGTAAGATGCTTTTCTGTGACTGGTGAGTACTCAACCAAGTCATTCTGA |
| | | GAATAGTGTATGCGGCGACCGAGTTGCTCTTGCCCGGCGTCAATACGGGATAATACCGCGCCACATAGC |
| | | AGAACTTTAAAAGTGCTCATCATTGGAAAACGTTCTTCGGGGCGAAAACTCTCAAGGATCTTACCGCTG |
| | | TTGAGATCCAGTTCGATGTAACCCACTCGTGCACCCAACTGATCTTCAGCATCTTTTACTTTCACCAGC |
| | | GTTTCTGGGTGAGCAAAAACAGGAAGGCAAAATGCCGCAAAAAAGGGAATAAGGGCGACACGGAAATGT |
| | | TGAATACTCATACTCTTCCTTTTTCAATATTATTGAAGCATTTATCAGGGTTATTGTCTCATGAGCGGA |
| | | TACATATTTGAATGTATTTAGAAAAATAAACAAATAGGGGTTCCGCGCACATTTCCCCGAAAAGTGCCA |
| | | CCTGACGTCTAAGAAACCATTATTAT |
| 11 | cdsDNA114 | CCAATTCTGATTAGAAAAACTCATCGAGCATCAAATGAAACTGCAATTTATTCATATCAGGATTATCAA |
| | KHP 0 | TACCATATTTTTGAAAAAGCCGTTTCTGTAATGAAGGAGAAAACTCACCGAGGCAGTTCCATAGGATGG |
| | without | CAAGATCCTGGTATCGGTCTGCGATTCCGACTCGTCCAACATCAATACAACCTATTAATTTCCCCTCGT |
| | gene V | CAAAAATAAGGTTATCAAGTGAGAAATCACCATGAGTGACGACTGAATCCGGTGAGAATGGCAAAAGCT |
| | attenuation | TATGCATTTCTTTCCAGACTTGTTCAACAGGCCAGCCATTACGCTCGTCATCAAAATCACTCGCATCAA |
| | | CCAAACCGTTATTCATTCGTGATTGCGCCTGAGCGAGACGAAATACGCGATCGCTGTTAAAAGGACAAT |
| | | TACAAACAGGAATCGAATGCAACCGGCGCAGGAACACTGCCAGCGCATCAACAATATTTTCACCTGAAT |
| | | CAGGATATTCTTCTAATACCTGGAATGCTGTTTTCCCGGGGATCGCAGTGGTGAGTAACCATGCATCAT |
| | | CAGGAGTACGGATAAAATGCTTGATGGTCGGAAGAGGCATAAATTCCGTCAGCCAGTTTAGTCTGACCA |
| | | TCTCATCTGTAACATCATTGGCAACGCTACCTTTGCCATGTTTCAGAAACAACTCTGGCGCATCGGGCT |
| | | TCCCATACAATCGATAGATTGTCGCACCTGATTGCCCGACATTATCGCGAGCCCATTTATACCCATATA |
| | | AATCAGCATCCATGTTGGAATTTAATCGCGGCCTCGAGCAAGACGTTTCCCGTTGAATATGGCTCATAA |
| | | CACCCCTTGTATTACTGTTTATGTAAGCAGACAGTTTTATTGTTCATGATGATATATTTTTATCTTGTG |
| | | CAATGTAACATCAGAGATTTTGAGACACAACGTGGCTTTCCCCCCCCCCCCCTGCAGGTCTCGGGCCTA |
| | | TTGGTTAAAAAATGAGCTGATTTAACAAAAATTTAACGCGAATTTTAACAAAATATTAACGTTTAçAAT |
| | | TTAAATATTTGCTTATACAATCTTCCTGTTTTTGGGGCTTTTCTGATTATCAACCGGGGTACATATGAT |
| | | TGACATGCTAGTTTTACGATTACCGTTCATCGATTCTCTTGTTTGCTCCAGACTCTCAGGCAATGACCT |
| | | GATAGCCTTTGTAGACCTCTCAAAAATAGCTACCCTCTCCGGCATGAATTTATCAGCTAGAACGGTTGA |
| | | ATATCATATTGATGGTGATTTGACTGTCTCCGGCCTTTCTCACCCTTTTGAATCTTTACCTACACATTA |
| | | CTCAGGCATTGCATTTAAAATATATGAGGGTTCTAAAAATTTTTATCCTTGCGTTGAAATAAAGGCTTC |
| | | TCCCGCAAAAGTATTACAGGGTCATAATGTTTTTGGTACAACCGATTTAGCTTTATGCTCTGAGGCTTT |
| | | ATTGCTTAATTTTGCTAATTCTTTGCCTTGCCTGTATGATTTATTGGATGTTAACGCTACTACTATTAG |
| | | TAGAATTGATGCCACCTTTTCAGCTCGCGCCCCAAATGAAAATATAGCTAAACAGGTTATTGACCATTT |
| | | GCGAAATGTATCTAATGGTCAAACTAAATCTACTCGTTCGCAGAATTGGGAATCAACTGTTACATGGAA |
| | | TGAAACTTCCAGACACCGTACTTTAGTTGCATATTTAAAACATGTTGAGCTACAGCACCAGATTCAGCA |
| | | ATTAAGCTCTAAGCCATCCGCAAAAATGACCTCTTATCAAAAGGAGCAATTAAAGGTACTCTCTAATCC |
| | | TGACCTGTTGGAGTTTGCTTCCGGTCTGGTTCGCTTTGAAGCTCGAATTAAAACGCGATATTTGAAGTC |
| | | TTTCGGGCTTCCTCTTAATCTTTTTGATGCAATCCGCTTTGCTTCTGACTATAATAGTCAGGGTAAAGA |
| | | CCTGATTTTTGATTTATGGTCATTCTCGTTTTCTGAACTGTTTAAAGCATTTGAGGGGGATTCAATGAA |
| | | TATTTATGACGATTCCGCAGTATTGGACGCTATCCAGTCTAAACATTTTACTATTACCCCCTCTGGCAA |
| | | AACTTCTTTTGCAAAAGCCTCTCGCTATTTTGGTTTTTATCGTCGTCTGGTAAACGAGGGTTATGATAG |
| | | TGTTGCTCTTACTATGCCTCGTAATTCCTTTTGGCGTTATGTATCTGCATTAGTTGAATGTGGTATTCC |
| | | TAAATCTCAACTGATGAATCTTTCTACCTGTAATAATGTTGTTCCGTTAGTTCGTTTTATTAACGTAGA |
| | | TTTTTCTTCCCAACGTCCTGACTGGTATAATGAGCCAGTTCTTAAAATCGCATAAGGTAATTCACAATG |
| | | ATTAAAGTTGAAATTAAACCATCTCAAGCCCAATTTACTACTCGTTCTGGTGTTTCTCGTCAGGGCAAG |
| | | CCTTATTCACTGAATGAGCAGCTTTGTTACGTTGATTTGGGTAATGAATATCCGGTTCTTGTCAAGATT |
| | | ACTCTTGATGAAGGTCAGCCAGCCTATGCGCCTGGTCTGTACACCGTTCATCTGTCCTCTTTCAAAGTT |
| | | GGTCAGTTCGGTTCCCTTATGATTGACCGTCTGCGCCTCGTTCCGGCTAAGTAACATGGAGCAGGTCGC |
| | | GGATTTCGACACAATTTATCAGGCGATGATACAAATCTCCGTTGTACTTTGTTTCGCGCTTGGTATAAT |
| | | CGCTGGGGGTCAAAGATGAGTGTTTTAGTGTATTCTTTCGCCTCTTTCGTTTTAGGTTGGTGCCTTCGT |
| | | AGTGGCATTACGTATTTTACCCGTTTAATGGAAACTTCCTCATGAAAAAGTCTTTAGCCCTCAAAGCCT |
| | | CTGTAGCCGTTGCTACCCTCGTTCCGATGCTGTCTTTCGCTGCTGAGGGTGACGATCCCGCAAAAGCGG |
| | | CCTTTAACTCCCTGCAAGCCTCAGCGACCGAATATATCGGTTATGCGTGGGCGATGGTTGTTGTCATTG |
| | | TCGGCGCAACTATCGGTATCAAGCTGTTTAAGAAATTCACCTCGAAAGCAAGCTGATAAACCGATACAA |
| | | TTAAAGGCTCCTTTTGGAGCCTTTTTTTTTGGAGATTTTCAACGTGAAAAAATTATTATTCGCAATTCC |
| | | TTTAGTTGTTCCTTTCTATTCTCACTCCGCTGAAACTGTTGAAAGTTGTTTAGCAAAACCCCATACAGA |
| | | AAATTCATTTACTAACGTCTGGAAAGACGACAAAACTTTAGATCGTTACGCTAACTATGAGGGCTGTCT |
| | | GTGGAATGCTACAGGCGTTGTAGTTTGTACTGGTGACGAAACTCAGTGTTACGGTACATGGGTTCCTAT |
| | | TGGGCTTGCTATCCCTGAAAATGAGGGTGGTGGCTCTGAGGGTGGCGGTTCTGAGGGTGGCGGTTCTGA |
| | | GGGTGGCGGTACTAAACCTCCTGAGTACGGTGATACACCTATTCCGGGCTATACTTATATCAACCCTCT |
| | | CGACGGCACTTATCCGCCTGGTACTGAGCAAAACCCCGCTAATCCTAATCCTTCTCTTGAGGAGTCTCA |
| | | GCCTCTTAATACTTTCATGTTTCAGAATAATAGGTTCCGAAATAGGCAGGGGGCATTAACTGTTTATAC |
| | | GGGCACTGTTACTCAAGGCACTGACCCCGTTAAAACTTATTACCAGTACACTCCTGTATCATCAAAAGC |
| | | CATGTATGACGCTTACTGGAACGGTAAATTCAGAGACTGCGCTTTCCATTCTGGCTTTAATGAGGATCC |
| | | ATTCGTTTGTGAATATCAAGGCCAATCGTCTGACCTGCCTCAACCTCCTGTCAATGCTGGCGGCGGCTC |
| | | TGGTGGTGGTTCTGGTGGCGGCTCTGAGGGTGGTGGCTCTGAGGGTGGCGGTTCTGAGGGTGGCGGCTC |
| | | TGAGGGAGGCGGTTCCGGTGGTGGCTCTGGTTCCGGTGATTTTGATTATGAAAAGATGGCAAACGCTAA |
| | | TAAGGGGGCTATGACCGAAAATGCCGATGAAAACGCGCTACAGTCTGACGCTAAAGGCAAACTTGATTC |
| | | TGTCGCTACTGATTACGGTGCTGCTATCGATGGTTTCATTGGTGACGTTTCCGGCCTTGCTAATGGTAA |
| | | TGGTGCTACTGGTGATTTTGCTGGCTCTAATTCCCAAATGGCTCAAGTCGGTGACGGTGATAATTCACC |
| | | TTTAATGAATAATTTCCGTCAATATTTACCTTCCCTCCCTCAATCGGTTGAATGTCGCCCTTTTGTCTT |
| | | TGGCGCTGGTAAACCATATGAATTTTCTATTGATTGTGACAAAATAAACTTATTCCGTGGTGTCTTTGC |
| | | GTTTCTTTTATATGTTGCCACCTTTATGTATGTATTTTCTACGTTTGCTAACATACTGCGTAATAAGGA |
| | | GTCTTAATCATGCCAGTTCTTTTGGGTATTCCGTTATTATTGCGTTTCCTCGGTTTCCTTCTGGTAACT |
| | | TTGTTCGGCTATCTGCTTACTTTTCTTAAAAAGGGCTTCGGTAAGATAGCTATTGCTATTTCATTGTTT |
| | | CTTGCTCTTATTATTGGGCTTAACTCAATTCTTGTGGGTTATCTCTCTGATATTAGCGCTCAATTACCC |
| | | TCTGACTTTGTTCAGGGTGTTCAGTTAATTCTCCCGTCTAATGCGCTTCCCTGTTTTTATGTTATTCTC |
| | | TCTGTAAAGGCTGCTATTTTCATTTTTGACGTTAAACAAAAAATCGTTTCTTATTTGGATTGGGATAAA |
| | | TAATATGGCTGTTTATTTTGTAACTGGCAAATTAGGCTCTGGAAAGACGCTCGTTAGCGTTGGTAAGAT |
| | | TCAGGATAAAATTGTAGCTGGGTGCAAAATAGCAACTAATCTTGATTTAAGGCTTCAAAACCTCCCGCA |
| | | AGTCGGGAGGTTCGCTAAAACGCCTCGCGTTCTTAGAATACCGGATAAGCCTTCTATATCTGATTTGCT |
| | | TGCTATTGGGCGCGGTAATGATTCCTACGATGAAAATAAAAACGGCTTGCTTGTTCTCGATGAGTGCGG |
| | | TACTTGGTTTAATACCCGTTCTTGGAATGATAAGGAAAGACAGCCGATTATTGATTGGTTTCTACATGC |
| | | TCGTAAATTAGGATGGGATATTATTTTTCTTGTTCAGGACTTATCTATTGTTGATAAACAGGCGCGTTC |
| | | TGCATTAGCTGAACATGTTGTTTATTGTCGTCGTCTGGACAGAATTACTTTACCTTTTGTCGGTACTTT |
| | | ATATTCTCTTATTACTGGCTCGAAAATGCCTCTGCCTAAATTACATGTTGGCGTTGTTAAATATGGCGA |
| | | TTCTCAATTAAGCCCTACTGTTGAGCGTTGGCTTTATACTGGTAAGAATTTGTATAACGCATATGATAC |
| | | TAAACAGGCTTTTTCTAGTAATTATGATTCCGGTGTTTATTCTTATTTAACGCCTTATTTATCACACGG |
| | | TCGGTATTTCAAACCATTAAATTTAGGTCAGAAGATGAAATTAACTAAAATATATTTGAAAAAGTTTTC |
| | | TCGCGTTCTTTGTCTTGCGATTGGATTTGCATCAGCATTTACATATAGTTATATAACCCAACCTAAGCC |
| | | GGAGGTTAAAAAGGTAGTCTCTCAGACCTATGATTTTGATAAATTCACTATTGACTCTTCTCAGCGTCT |
| | | TAATCTAAGCTATCGCTATGTTTTCAAGGATTCTAAGGGAAAATTAATTAATAGCGACGATTTACAGAA |
| | | GCAAGGTTATTCACTCACATATATTGATTTATGTACTGTTTCCATTAAAAAAGGTAATTCAAATGAAAT |
| | | TGTTAAATGTAATTAATTTTGTTTTCTTGATGTTTGTTTCATCATCTTCTTTTGCTCAGGTAATTGAAA |
| | | TGAATAATTCGCCTCTGCGCGATTTTGTAACTTGGTATTCAAAGCAATCAGGCGAATCCGTTATTGTTT |
| | | CTCCCGATGTAAAAGGTACTGTTACTGTATATTCATCTGACGTTAAACCTGAAAATCTACGCAATTTCT |
| | | TTATTTCTGTTTTACGTGCAAATAATTTTGATATGGTAGGTTCTAACCCTTCCATTATTCAGAAGTATA |
| | | ATCCAAACAATCAGGATTATATTGATGAATTGCCATCATCTGATAATCAGGAATATGATGATAATTCCG |
| | | CTCCTTCTGGTGGTTTCTTTGTTCCGCAAAATGATAATGTTACTCAAACTTTTAAAATTAATAACGTTC |
| | | GGGCAAAGGATTTAATACGAGTTGTCGAATTGTTTGTAAAGTCTAATACTTCTAAATCCTCAAATGTAT |
| | | TATCTATTGACGGCTCTAATCTATTAGTTGTTAGTGCTCCTAAAGATATTTTAGATAACCTTCCTCAAT |
| | | TCCTTTCAACTGTTGATTTGCCAACTGACCAGATATTGATTGAGGGTTTGATATTTGAGGTTCAGCAAG |
| | | GTGATGCTTTAGATTTTTCATTTGCTGCTGGCTCTCAGCGTGGCACTGTTGCAGGCGGTGTTAATACTG |
| | | ACCGCCTCACCTCTGTTTTATCTTCTGCTGGTGGTTCGTTCGGTATTTTTAATGGCGATGTTTTAGGGC |
| | | TATCAGTTCGCGCATTAAAGACTAATAGCCATTCAAAAATATTGTCTGTGCCACGTATTCTTACGCTTT |
| | | CAGGTCAGAAGGGTTCTATCTCTGTTGGCCAGAATGTCCCTTTTATTACTGGTCGTGTGACTGGTGAAT |
| | | CTGCCAATGTAAATAATCCATTTCAGACGATTGAGCGTCAAAATGTAGGTATTTCCATGAGCGTTTTTC |
| | | CTGTTGCAATGGCTGGCGGTAATATTGTTCTGGATATTACCAGCAAGGCCGATAGTTTGAGTTCTTCTA |
| | | CTCAGGCAAGTGATGTTATTACTAATCAAAGAAGTATTGCTACAACGGTTAATTTGCGTGATGGACAGA |
| | | CTCTTTTACTCGGTGGCCTCACTGATTATAAAAACACTTCTCAGGATTCTGGCGTACCGTTCCTGTCTA |
| | | AAATCCCTTTAATCGGCCTCCTGTTTAGCTCCCGCTCTGATTCTAACGAGGAAAGCACGTTATACGTGC |
| | | TCGTCAAAGCAACCATAGTACGCGCCCTGTAGCTCGGTACCAAATTCCAGAAAAGAGGCCGCGAAAGCG |
| | | GCCTTTTTTCGTTTTGGTCCACGGATCGCTTCATGTGGCAGGAGAAAAAAGGCTGCACCGGTGCGTCAG |
| | | CAGAATATGTGATACAGGATATATTCCGCTTCCTCGCTCACTGACTCGCTACGCTCGGTCGTTCGACTG |
| | | CGGCGAGCGGAAATGGCTTACGAACGGGGCGGAGATTTCCTGGAAGATGCCAGGAAGATACTTAACAGG |
| | | GAAGTGAGAGGGCCGCGGCAAAGCCGTTTTTCCATAGGCTCCGCCCCCCTGACAAGCATCACGAAATCT |
| | | GACGCTCAAATCAGTGGTGGCGAAACCCGACAGGACTATAAAGATACCAGGCGTTTCCCCCTGGCGGCT |
| | | CCCTCGTGCGCTCTCCTGTTCCTGCCTTTCGGTTTACCGGTGTCATTCCGCTGTTATGGCCGCGTTTGT |
| | | CTCATTCCACGCCTGACACTCAGTTCCGGGTAGGCAGTTCGCTCCAAGCTGGACTGTATGCACGAACCC |
| | | CCCGTTCAGTCCGACCGCTGCGCCTTATCCGGTAACTATCGTCTTGAGTCCAACCCGGAAAGACATGCA |
| | | AAAGCACCACTGGCAGCAGCCACTGGTAATTGATTTAGAGGAGTTAGTCTTGAAGTCATGCGCCGGTTA |
| | | AGGCTAAACTGAAAGGACAAGTTTTGGTGACTGCGCTCCTCCAAGCCAGTTACCTCGGTTCAAAGAGTT |
| | | GGTAGCTCAGAGAACCTTCGAAAAACCGCCCTGCAAGGCGGTTTTTTCGTTTTCAGAGCAAGAGATTAC |
| | | GCGCAGACCAAAACGATCTCAAGAAGATCATCTTATTAAGGGGTCTGACGCTCAGTGGAACGAAAACTC |
| | | ACGTTAAGGGATTTTGGTCATGAGATTATCAAAAAGGATCTTCACCTAGATCCTTTTAAATTAAAAATG |
| | | AAGTTTTAAATCAATCTAAAGTATATATGAGTAAACTTGGTCTGACAGTTACCAATGCTTAATCAGTGA |
| | | GGCACCTATCTCAGCGATCTGTCTATTTCGTTCATCCATAGTTGCCTGACTCCCCGTCGTGTAGATAAC |
| | | TACGATACGGGAGGGCTTACCATCTGGCCCCAGTGCTGCAATGATACCGCGAGACCCACGCTCACCGGC |
| | | TCCAGATTTATCAGCAATAAACCAGCCAGCCGATTCGAGCTCGCCCGGGGATCGACCAGTTGGTGATTT |
| | | TGAACTTTTGCTTTGCCACGGAACGGTCTGCGTTGTCGGGAAGATGCGTGATCTGATCCTTCAACTCAG |
| | | CAAAAGTTCGATTTATTCAACAAAGCCGCCGTCCCGTCAAGTCAGCGTAATGCTCTGCCAGTGTTACAA |
| | | CCAATTAA |
| 12 | trac long | AGTCTCAGCTACTCGGGAGACTGAGGCAGGAGAATTGTTGAACCCGGGAGGCAGAGATTGCAGTGAGCT |
| | NC_000014.9 | GAGATCGCATCATTGCACTCCAGCCTGGGCAACAGAGCAAGACTCCTTCTCAAAAAAACAAAAAAAAAA |
| | | AAAAAAAAGAAGGTCTAACCCTTAGGAGTGTGATTATCCTGTTCTCCTGCCTTGTGGGGGAGATCAGTG |
| | | TTTCTCTTATTTAAGTAATAGGTAGGTCCTGTGAGTTTGTGCAATGGTGTCACCTACGGTATGAATACT |
| | | GGAGGAACAATTGATAAACTCACATTTGGGAAAGGGACCCATGTATTCATTATATCTGGTGAGTCATCC |
| | | CAGGTGGCACCACGTGCAACCCCATGGGCCAGTGTCACTAATCCTTTCTCTGGAGATATCACTTATTAC |
| | | TATGGTGAGGCTTGCTGTAGATGTTGTAACTAATTTTCTTACAGAGGTCTGGGAAGGGAAAAGCATTAC |
| | | TATCTATCTTGAATATTCATGTTTCTCTAGGTCAAACACATTAAAATTTGACTTTAATCATTCAATGGG |
| | | TATTGTAAAATGCCTTCTATGTGACTATCACTCTATAAAATGTTAGACTGAGTATGAAGTGTGAGATAG |
| | | ATTCCTGTCCTGTCCTCAAGTGGTATAAAAACTAGACAAAGGTACTGAACTATTGTAAATTAAGCAGCC |
| | | AGAAAACTATTTTAGTATTGACCAGTTGATGATGTCAATGGACACAATAGAGATTCAGAGGGGATAGGG |
| | | TGCTGGCTGAGAAGTTGGCCTAGACTGAGAAGTTTCCTGTGGTACAAAGGATTCATTGAGCCCTGAAGG |
| | | ATGGATAAGATCTGTATGGGCAGAGAAAGGAGAGAAGGGAAGTTCTGGGCGTAGGGAACGACAAGAAAG |
| | | AAGGCATGATCTTGGGAATAATCAAGGCACATGCAAAGTAGCCTAAGTATACATCTGATAATAAAATTG |
| | | GTTGAAAAGTAGTCAGAGAAGATGTCTTTTTAGGCATGGAAAAAGGAAATACTAGAGCATTCAACAGAA |
| | | TACAGAAATTAGGGCCAGGGCCAGCCATTGGGAAACTGAGAATCCGATTTAGAGATGCAGACTAGAAGT |
| | | GAAGGTGAGAGCAGCCAGCTATGGTGCCGCAGACCTCCCCTCTCCTTCCTCAGTGGGCTCTGAGAGGGG |
| | | TCATCCCACACCTTAGAGGAGGAGAAACCTAAGGGATTCTGTAATAGAGACACGGGGCATGGTATGAAA |
| | | GTATTACCTCCCAGTTGCAATTTGGCAAAGGAACCAGAGTTTCCACTTCTCCCCGTACGTCTGCCCATG |
| | | CCCACAGTTTCCTGATGCTCACTAAAGCCTCGGTGGGACCCAGAGTGACTGTCACTAATTCTGATTTCT |
| | | GGGTCCCTAGTGCCCAAACACGGGGGACAGATTTAATGGTAAGGAAGCTTTCAATCACTGCTGTGTCCC |
| | | TAGGGATCTAAAGCACTAGAGCACATGTGCTTCTGCAGTTCATTTTGAATTTAAAGGACAGCTTAGGAT |
| | | CTAGAATAGCTGAATTTCCACCTCAAAACATTGGTTCCGTCTTGCCAAGCCTACCTTCTGATATCATCA |
| | | GTGATGGGATGTGTTTTTCTTACTAGGGTAGAATAGGATGTCTCTCCCCAAAGGACTCTGGCAGACAGA |
| | | CCCCTAAACACCTCCAAATTAAAAGCGGCAAAGAGATAAGGTTGAACTAGACGTACATGGGGATAAAAA |
| | | GTATAAAAGGTACATGGGAATGAAAGGATAAAAAGGCTAAAAAAATTAAGTACCTCTAACTCAGCCCCT |
| | | GTTGCCATTTCTCAGAGTCTTGTGTTCTGTGGCATTGCGCTTTCTAGACCAACAGTGTCCAATAGAACT |
| | | TTCTGTGGCAATGGAAATGTCCTGTCAATCTGCACTGTCCCATACAATAGCCACCAGCTACATGTGGCT |
| | | ATTGAGCTCTTGAAATGAAGTTTCCATTTTTAATTGAAAACATTTTATTTCACATTGACTAATTTTTAT |
| | | TTCAACAGCCACATGTAGCTAGAGACTATTATACCAGACAGAGCAGCCTAGATCTTCTCCAGTCTGACA |
| | | CCCACCAGCCCCAGGACTTGAGTGAGTGTTTAACCAGGACTCAAAGTTGGGTTTCTGCCCCACAAGGCC |
| | | ACCCCCTTTCCTCTTTAAAGCCAACCTGCATCTGGTGGCCCCTGATCCCCTGCCTTGAGGATCGGCACT |
| | | TCCAGACTCCTCTCCCCCTCTGCAGTGCTGTCCAGTACCCCCACTGATGACTAACAATCAGGGGGATGT |
| | | GTTGGTAGAGCTAATGGCTTTCTGTCTGTCCCTTCCCAGCAAAGGAACTATGCCTTAGGGCCTTCACCC |
| | | AGAGTGATGTCAGGCTGCCCAAGCATGAGGAGGGAAGTAGGCAGAATCCTCTGGAGCCAAAGCTCTGGA |
| | | TGTCTCTCCCCTCTGACCATGGAGCCCACCCCTGCTCCACTGCTCCAGGGACAGCCCTATGCTGCAGGC |
| | | AGCTCTGCCCCCACTCAGCATCCCAGGGGCTGATTTCTTTGGTTTTGGATCCAGCTGGATGTCTGCATT |
| | | GCCGAGGCCACCAGGGCTGGCTCAGCAACTGTCGGGGAATCACCAGGGTCTGAGAAATCTTGTGCGCAT |
| | | GTGAGGGGCTGTGGGAGCAGAGAACCACTGGGTGGGAAATTCTAATCCCCACCCTGCTGGAAACTCTCT |
| | | GGGTGGCCCCAACATGCTAATCCTCCGGCAAACCTCTGTTTCCTCCTCAAAAGGCAGGAGGTCGGAAAG |
| | | AATAAACAATGAGAGTCACATTAAAAACACAAAATCCTACGGAAATACTGAAGAATGAGTCTCAGCACT |
| | | AAGGAAAAGCCTCCAGCAGCTCCTGCTTTCTGAGGGTGAAGGATAGACGCTGTGGCTCTGCATGACTCA |
| | | CTAGCACTCTATCACGGCCATATTCTGGCAGGGTCAGTGGCTCCAACTAACATTTGTTTGGTACTTTAC |
| | | AGTTTATTAAATAGATGTTTATATGGAGAAGCTCTCATTTCTTTCTCAGAAGAGCCTGGCTAGGAAGGT |
| | | GGATGAGGCACCATATTCATTTTGCAGGTGAAATTCCTGAGATGTAAGGAGCTGCTGTGACTTGCTCAA |
| | | GGCCTTATATCGAGTAAACGGTAGTGCTGGGGCTTAGACGCAGGTGTTCTGATTTATAGTTCAAAACCT |
| | | CTATCAATGAGAGAGCAATCTCCTGGTAATGTGATAGATTTCCCAACTTAATGCCAACATACCATAAAC |
| | | CTCCCATTCTGCTAATGCCCAGCCTAAGTTGGGGAGACCACTCCAGATTCCAAGATGTACAGTTTGCTT |
| | | TGCTGGGCCTTTTTCCCATGCCTGCCTTTACTCTGCCAGAGTTATATTGCTGGGGTTTTGAAGAAGATC |
| | | CTATTAAATAAAAGAATAAGCAGTATTATTAAGTAGCCCTGCATTTCAGGTTTCCTTGAGTGGCAGGCC |
| | | AGGCCTGGCCGTGAACGTTCACTGAAATCATGGCCTCTTGGCCAAGATTGATAGCTTGTGCCTGTCCCT |
| | | GAGTCCCAGTCCATCACGAGCAGCTGGTTTCTAAGATGCTATTTCCCGTATAAAGCATGAGACCGTGAC |
| | | TTGCCAGCCCCACAGAGCCCCGCCCTTGTCCATCACTGGCATCTGGACTCCAGCCTGGGTTGGGGCAAA |
| | | GAGGGAAATGAGATCATGTCCTAACCCTGATCCTCTTGTCCCACAGATATCCAGAACCCTGACCCTGCC |
| | | GTGTACCAGCTGAGAGACTCTAAATCCAGTGACAAGTCTGTCTGCCTATTCACCGATTTTGATTCTCAA |
| | | ACAAATGTGTCACAAAGTAAGGATTCTGATGTGTATATCACAGACAAAACTGTGCTAGACATGAGGTCT |
| | | ATGGACTTCAAGAGCAACAGTGCTGTGGCCTGGAGCAACAAATCTGACTTTGCATGTGCAAACGCCTTC |
| | | AACAACAGCATTATTCCAGAAGACACCTTCTTCCCCAGCCCAGGTAAGGGCAGCTTTGGTGCCTTCGCA |
| | | GGCTGTTTCCTTGCTTCAGGAATGGCCAGGTTCTGCCCAGAGCTCTGGTCAATGATGTCTAAAACTCCT |
| | | CTGATTGGTGGTCTCGGCCTTATCCATTGCCACCAAAACCCTCTTTTTACTAAGAAACAGTGAGCCTTG |
| | | TTCTGGCAGTCCAGAGAATGACACGGGAAAAAAGCAGATGAAGAGAAGGTGGCAGGAGAGGGCACGTGG |
| | | CCCAGCCTCAGTCTCTCCAACTGAGTTCCTGCCTGCCTGCCTTTGCTCAGACTGTTTGCCCCTTACTGC |
| | | TCTTCTAGGCCTCATTCTAAGCCCCTTCTCCAAGTTGCCTCTCCTTATTTCTCCCTGTCTGCCAAAAAA |
| | | TCTTTCCCAGCTCACTAAGTCAGTCTCACGCAGTCACTCATTAACCCACCAATCACTGATTGTGCCGGC |
| | | ACATGAATGCACCAGGTGTTGAAGTGGAGGAATTAAAAAGTCAGATGAGGGGTGTGCCCAGAGGAAGCA |
| | | CCATTCTAGTTGGGGGAGCCCATCTGTCAGCTGGGAAAAGTCCAAATAACTTCAGATTGGAATGTGTTT |
| | | TAACTCAGGGTTGAGAAAACAGCTACCTTCAGGACAAAAGTCAGGGAAGGGCTCTCTGAAGAAATGCTA |
| | | CTTGAAGATACCAGCCCTACCAAGGGCAGGGAGAGGACCCTATAGAGGCCTGGGACAGGAGCTCAATGA |
| | | GAAAGGAGAAGAGCAGCAGGCATGAGTTGAATGAAGGAGGCAGGGCCGGGTCACAGGGCCTTCTAGGCC |
| | | ATGAGAGGGTAGACAGTATTCTAAGGACGCCAGAAAGCTGTTGATCGGCTTCAAGCAGGGGAGGGACAC |
| | | CTAATTTGCTTTTCTTTTTTTTTTTTTTTTTTTTTTTTTTTTTTGAGATGGAGTTTTGCTCTTGTTGCC |
| | | CAGGCTGGAGTGCAATGGTGCATCTTGGCTCACTGCAACCTCCGCCTCCCAGGTTCAAGTGATTCTCCT |
| | | GCCTCAGCCTCCCGAGTAGCTGAGATTACAGGCACCCGCCACCATGCCTGGCTAATTTTTTGTATTTTT |
| | | AGTAGAGACAGGGTTTCACTATGTTGGCCAGGCTGGTCTCGAACTCCTGACCTCAGGTGATCCACCCGC |
| | | TTCAGCCTCCCAAAGTGCTGGGATTACAGGCGTGAGCCACCACACCCGGCCTGCTTTTCTTAAAGATCA |
| | | ATCTGAGTGCTGTACGGAGAGTGGGTTGTAAGCCAAGAGTAGAAGCAGAAAGGGAGCAGTTGCAGCAGA |
| | | GAGATGATGGAGGCCTGGGCAGGGTGGTGGCAGGGAGGTAACCAACACCATTCAGGTTTCAAAGGTAGA |
| | | ACCATGCAGGGATGAGAAAGCAAAGAGGGGATCAAGGAAGGCAGCTGGATTTTGGCCTGAGCAGCTGAG |
| | | TCAATGATAGTGCCGTTTACTAAGAAGAAACCAAGGAAAAAATTTGGGGTGCAGGGATCAAAACTTTTT |
| | | GGAACATATGAAAGTACGTGTTTATACTCTTTATGGCCCTTGTCACTATGTATGCCTCGCTGCCTCCAT |
| | | TGGACTCTAGAATGAAGCCAGGCAAGAGCAGGGTCTATGTGTGATGGCACATGTGGCCAGGGTCATGCA |
| | | ACATGTACTTTGTACAAACAGTGTATATTGAGTAAATAGAAATGGTGTCCAGGAGCCGAGGTATCGGTC |
| | | CTGCCAGGGCCAGGGGCTCTCCCTAGCAGGTGCTCATATGCTGTAAGTTCCCTCCAGATCTCTCCACAA |
| | | GGAGGCATGGAAAGGCTGTAGTTGTTCACCTGCCCAAGAACTAGGAGGTCTGGGGTGGGAGAGTCAGCC |
| | | TGCTCTGGATGCTGAAAGAATGTCTGTTTTTCCTTTTAGAAAGTTCCTGTGATGTCAAGCTGGTCGAGA |
| | | AAAGCTTTGAAACAGGTAAGACAGGGGTCTAGCCTGGGTTTGCACAGGATTGCGGAAGTGATGAACCCG |
| | | CAATAACCCTGCCTGGATGAGGGAGTGGGAAGAAATTAGTAGATGTGGGAATGAATGATGAGGAATGGA |
| | | AACAGCGGTTCAAGACCTGCCCAGAGCTGGGTGGGGTCTCTCCTGAATCCCTCTCACCATCTCTGACTT |
| | | TCCATTCTAAGCACTTTGAGGATGAGTTTCTAGCTTCAATAGACCAAGGACTCTCTCCTAGGCCTCTGT |
| | | ATTCCTTTCAACAGCTCCACTGTCAAGAGAGCCAGAGAGAGCTTCTGGGTGGCCCAGCTGTGAAATTTC |
| | | TGAGTCCCTTAGGGATAGCCCTAAACGAACCAGATCATCCTGAGGACAGCCAAGAGGTTTTGCCTTCTT |
| | | TCAAGACAAGCAACAGTACTCACATAGGCTGTGGGCAATGGTCCTGTCTCTCAAGAATCCCCTGCCACT |
| | | CCTCACACCCACCCTGGGCCCATATTCATTTCCATTTGAGTTGTTCTTATTGAGTCATCCTTCCTGTGG |
| | | TAGCGGAACTCACTAAGGGGCCCATCTGGACCCGAGGTATTGTGATGATAAATTCTGAGCACCTACCCC |
| | | ATCCCCAGAAGGGCTCAGAAATAAAATAAGAGCCAAGTCTAGTCGGTGTTTCCTGTCTTGAAACACAAT |
| | | ACTGTTGGCCCTGGAAGAATGCACAGAATCTGTTTGTAAGGGGATATGCACAGAAGCTGCAAGGGACAG |
| | | GAGGTGCAGGAGCTGCAGGCCTCCCCCACCCAGCCTGCTCTGCCTTGGGGAAAACCGTGGGTGTGTCCT |
| | | GCAGGCCATGCAGGCCTGGGACATGCAAGCCCATAACCGCTGTGGCCTCTTGGTTTTACAGATACGAAC |
| | | CTAAACTTTCAAAACCTGTCAGTGATTGGGTTCCGAATCCTCCTCCTGAAAGTGGCCGGGTTTAATCTG |
| | | CTCATGACGCTGCGGCTGTGGTCCAGCTGAGGTGAGGGGCCTTGAAGCTGGGAGTGGGGTTTAGGGACG |
| | | CGGGTCTCTGGGTGCATCCTAAGCTCTGAGAGCAAACCTCCCTGCAGGGTCTTGCTTTTAAGTCCAAAG |
| | | CCTGAGCCCACCAAACTCTCCTACTTCTTCCTGTTACAAATTCCTCTTGTGCAATAATAATGGCCTGAA |
| | | ACGCTGTAAAATATCCTCATTTCAGCCGCCTCAGTTGCACTTCTCCCCTATGAGGTAGGAAGAACAGTT |
| | | GTTTAGAAACGAAGAAACTGAGGCCCCACAGCTAATGAGTGGAGGAAGAGAGACACTTGTGTACACCAC |
| | | ATGCCTTGTGTTGTACTTCTCTCACCGTGTAACCTCCTCATGTCCTCTCTCCCCAGTACGGCTCTCTTA |
| | | GCTCAGTAGAAAGAAGACATTACACTCATATTACACCCCAATCCTGGCTAGAGTCTCCGCACCCTCCTC |
| | | CCCCAGGGTCCCCAGTCGTCTTGCTGACAACTGCATCCTGTTCCATCACCATCAAAAAAAAACTCCAGG |
| | | CTGGGTGCGGGGGCTCACACCTGTAATCCCAGCACTTTGGGAGGCAGAGGCAGGAGGAGCACAGGAGCT |
| | | GGAGACCAGCCTGGGCAACACAGGGAGACCCCGCCTCTACAAAAAGTGAAAAAATTAACCAGGTGTGGT |
| | | GCTGCACACCTGTAGTCCCAGCTACTTAAGAGGCTGAGATGGGAGGATCGCTTGAGCCCTGGAATGTTG |
| | | AGGCTACAATGAGCTGTGATTGCGTCACTGCACTCCAGCCTGGAAGACAAAGCAAGATCCTGTCTCAAA |
| | | TAATAAAAAAAATAAGAACTCCAGGGTACATTTGCTCCTAGAACTCTACCACATAGCCCCAAACAGAGC |
| | | CATCACCATCACATCCCTAACAGTCCTGGGTCTTCCTCAGTGTCCAGCCTGACTTCTGTTCTTCCTCAT |
| | | TCCAGATCTGCAAGATTGTAAGACAGCCTGTGCTCCCTCGCTCCTTCCTCTGCATTGCCCCTCTTCTCC |
| | | CTCTCCAAACAGAGGGAACTCTCCTACCCCCAAGGAGGTGAAAGCTGCTACCACCTCTGTGCCCCCCCG |
| | | GCAATGCCACCAACTGGATCCTACCCGAATTTATGATTAAGATTGCTGAAGAGCTGCCAAACACTGCTG |
| | | CCACCCCCTCTGTTCCCTTATTGCTGCTTGTCACTGCCTGACATTCACGGCAGAGGCAAGGCTGCTGCA |
| | | GCCTCCCCTGGCTGTGCACATTCCCTCCTGCTCCCCAGAGACTGCCTCCGCCATCCCACAGATGATGGA |
| | | TCTTCAGTGGGTTCTCTTGGGCTCTAGGTCCTGCAGAATGTTGTGAGGGGTTTATTTTTTTTTAATAGT |
| | | GTTCATAAAGAAATACATAGTATTCTTCTTCTCAAGACGTGGGGGGAAATTATCTCATTATCGAGGCCC |
| | | TGCTATGCTGTGTATCTGGGCGTGTTGTATGTCCTGCTGCCGATGCCTTCATTAAAATGATTTGGAAGA |
| | | GCAGAGACTGTGCCTCTGTTTGACTGGGTTTGGTAGGAGTCATTTTCTGCTTGCTGGTGATCACTAGCT |
| | | GGGCAGAGAAAAACCAAGGCATTTGTCTATGATGCTGTCCAGGAAGCCTCATTCAACAAGCTGCCTAAG |
| | | TCAACCTCTTCTTGGAATAACCTCTAAAAGCTTCCGCTTAGCAGGCTATGCTGAGGGCCAGGAAAACCC |
| | | ACCTACCAGCTTGGACCCCTCCTCTCCCACTCTCATGCCACGCCACGGGACCACCCATAACAGGAGCCC |
| | | ACACACATGGGTGGCAGTGACCTGCGGCAGACAGGGACCACACAGCAAGTGTCCCCAAAATGCCACCCA |
| | | CTGTCTCCTGCCCTCCAGGAGCATTTCCTTTGCCTCTCCTCTCAGACTGGGTTTCCACTGAAACTGTGC |
| | | ATTGTCTCACAAATTCGTGGCTGGGGACCACCCACCACTCTGCTGCCTGATCCAGCCCCACGCCAGCCC |
| | | TTTGAGGTGCCCAAGCTGACACCAGGAGCAAGGTTGAGAGGAAGCTGTGACCCCAGCAGGACTTTATGT |
| | | TCCCACCATCCCGGATGTGAGAATGAGGAAAAAAGGAGATGAGCTGTCTCCCCACAAGCCCAGAGATTT |
| | | GACCGAGGAGAGTAGAGGCCTCGAGCTCTCACCTAAGAGAAAAGACATGGGGCTTCCTGGGGTCCACAG |
| | | CTCACTGCGCTCTCCCTCCTGAGACTCCTGCTGCCAGAGCACCTTTCCCCAGGGTCATGGATGCTGAGG |
| | | GAAACACAACTTAGAGACCACTCCACCATCCACCCAGCAAGCACAGCTACCAAGACCCAAAGCTGAGGC |
| | | TTACCAATGCCCAGGGTGGGAGGGGGTTCCATCCCTGAATAACTCCATGGTTCCCCTATGCGTCTGACC |
| | | ATCCCAGCCAGAAATACATAAATCATCTCAGCTACAATTCAGGCCTGCTTCTTTTCATAGGGATGAAGC |
| | | TACAGGTTGAGTATCCCTTATCTGAAATGCTTGGTACTAGAAGTGTTCAGGTTTTGGATTTTTTTTTTT |
| | | TTTTTTTGAGGCTGGGGGTGGAATATTAGCATTATACTTACCAATTCGGCATCCCTAATCTAAAAATCT |
| | | GAAATCCAAGATGCCCCAGTGAGTGAGCATTTCCTTTGAGCATCATGTTGGCATTTAAAAAGTTTCAGA |
| | | TTTTGGAGCATTTCCAATTTCAGATTTTTGAATTAGGGATGCTTAACCTGTACCAGCTTTAATAGGTGA |
| | | CCTAGAGCACATCCCTCCCCTCTACAGGCTTATGTGTTGCCACTTACGAAATGTCGGGTAGGACTGGAG |
| | | GCTACCTCCCACTCCCTGCACCTCTGATTCTGTGACCCCTGCAGAATTAAGAACCAGTGTCCCTTCTCC |
| | | TGGTCAGCCCTACTGACGGGAGTCACAGAATCCCCAGTTCTTTCTAAGCTGCCCCATCTCTCACCTAAA |
| | | TACAATCCCCTTAAATAACACCAAAGGGAAAGGGCTCAGACCTCCATCACAGCAGGGTCACTCTCGCAç |
| | | GTGGTAGGACCATACCACCTTCACAAAGGCGGGGTTTGCCACCTTTGGGGAGCTCTGGGGGGCCTCTAC |
| | | CTCCTCACCAGTCTGACACAATGCCAGAGATTCCACCACTGGGAATTTCTTATAATTAAAGCATTCTCT |
| | | TCCCTGTATTAAATGAAAATGCCCCTGGGAGAGTTAATAGAGCAAGCCTTTATCAACCATTAAAAACTG |
| | | AGGGCCAGTTAGTTTCTCTTTCTTTTCCCCCTGAAGTGGTACTTCATTTTGTTTTATAGAAAAAAGATT |
| | | CAGGCAAGGGAAGTGTGGGTGGCTGGGGAGGCAGGTCTGCTCCTTTGAGTTGGCTGCAGTGACATGGAA |
| | | GTCACAGGGCTGAGGGAAGGAGACAAGAGCCTGGACAGCAGTGAAGGGGTCAAAGACAGACCCCTCCAA |
| | | GAACCTCAGAGGAGACCCGGACTGCAGGAGACCTGCAGGAGGCCCGTGGGAGCCTGTGGAGGCCTGTGG |
| | | AGGCCCGCGGGAGCCTGTGGAGGCCTGTGGAGGCCCGCGGGAGCCTGTGGAGGCCTGTGGAGGCCTGTG |
| | | GAGGTCTGCGGGAGCCTGTGGAGGCCTGTGGAGGCCTGCGGGAGCCTGTGGAGGCCTGTGGAGGCCTGT |
| | | GGAGGTCTGCGGGAGCCTGTGGAGGTCTGCGGGAGCCTGTGGAGGCCTGTGGAGGCCTGTGGAGGCCTG |
| | | CGGGAGCCTGTGGAGGTCTGCGGGAGCCTGTGGAGGCCTGTGGAGGCCTGTGGAGGTCTGCGGGAGCCT |
| | | GTGGAGGCCTGTGGAGGCCTGTGGAGGTCTGCGGGAGCCTGTGGAGGCCTGTGGAGGCCTGCCAGCCCA |
| | | GTGCCCTCAGGCAGCAAAGCCCAGCAGTCACTGGTCCCCCAAGGCCGCCCACAGATCTATGCAAGGTGA |
| | | TGCAGCAGGGGCTATGGACCCACCCACACCCAAGCAGGGAGGCACGATGACAAGGCCCGGGCAGGTAGG |
| | | GGGACATCCGGAAAGCAGGCTCAGCTCCACCCCTGAGAAATTTCCGTCTAAATCCAGGAGATCCTTGAT |
| | | TCAGCAAGAACCTTCCCTCATGTCCGAGGCTGTTAAACCTAAAGCCAGTTAGGGCAGAGGTCAGAGGGG |
| | | TGGACCAGGAGCAAGTGGGTCGAGGGTGAGCAAGTGTTGGGAGTGGGGATAGAAACTCCATCCATCTCC |
| | | GACTGCTCTGCTCGCATATCTGGCTCCAGGGCCCACCTGGTATGTCAGAGGGAATTAGAACGGCCTTGT |
| | | GAGGAGGCTCAGGAGAAGGGCTCCTGTGCCCACCGGCCAAGTCAGCACTGGGCCTAACGCCACCACAGC |
| | | AAAGCCCCTCAGTGCAGAGGGCCCAGCTATACGGAACTGGGCATTGCTTAGCCTGGAGGGAATAGGCAG |
| | | CTGAGGAGACCTGGAAAACACAGTCATGAAGAGACTGGCCATGAAGACCCACAGACCCTGCGAGCCAAG |
| | | GCCTTGGGCTGCAGCTGCAGCTGCAGCTCTACCAGCCTGGACCCAACCTCAGGCCGACATCCCAGCAGG |
| | | GAACATGTTACTAAGGCCAGTTGTAGAAGGGACTTCTCTGGACACATGCCCTACTCTCCCAAAAGACTT |
| | | AAGCACAGGCCAGTCCCAAAGGCAGAAGAATGGACTAAATGAGCTCTTGTGGCCCCAGCCCTAAGATTC |
| | | TAGAGGACTCTCTGAAACTTCACTTGAAGACTTGACCTCTGAGTGCAACTGGAGTCACAGCCACTCAGG |
| | | AGAAAGGGCAGGCTATGCAAGCACGTGCTTCAGGAGCATCCTGGTGAGGTCTTCAGCTCTGACAGAGCA |
| | | ACCCCTCTGCTGCTACCCTCACTGGGGGCAGCCATCTGAGTCTTAAGGAGTTGCTTATTTTTTAGGTCC |
| | | AAACATAAAATTCAACTCCCCTGGGATCACGTATACAGACAGGCCTATAGCACCTTATTATCAGGTACC |
| | | ATCCTAGTGTGGAAGTCCCCTGGCCTGTGTGCTCTGTGGGATGGGAATGTCCCTTGTCATCAATGTGTC |
| | | CCCAGGAACTAGAACAGTGCCTGGCACACAACAGATGCCGTTAAATGAATAAGTGAATGACTCCTGCAG |
| | | TTAGGCTCTGGGTGGGGGGAGCTGAGTGTGAGGAGTGGGGAGTACAGGAGAAAGGAAAGTGGGGATTAG |
| | | TAGAGGAAGTGGTCAGGTGAGTCA |
| 13 | Cas9 ZF5 | ATCACCGGCCTGTACGAGACACGGATCGACCTGTCTCAGCTGGGAGGCGACTCCCCCGTCCGGAGTAGC |
| | gBlock | GGGGGATCTTCTGGCGGCAGTTCCGGTTCTGAAACCCCCGGGACTTCTGAGTCCGCCACCCCCGAATCC |
| | | AGCGGAGGGTCTTCCGGAGGGTCTTCAAGACCAGGTGAACGCCCATTCCAGTGTCGCATCTGCATGCGC |
| | | AACTTTAGCAATATGAGCAATTTGACCAGACATACAAGAACACATACAGGTGAGAAGCCCTTCCAGTGT |
| | | CGAATATGTATGAGGAATTTCTCAGATAGGTCCGTTCTGAGGCGGCATCTCAGAACACATACGGGTTCC |
| | | CAGAAACCATTCCAATGTCGGATATGTATGCGGAACTTCAGTGACCCATCCAATCTCGCTAGGCATACG |
| | | CGGACCCACACAGGTGAAAAACCGTTTCAATGCCGCATATGCATGCGGAATTTCTCTGACCGATCAAGC |
| | | CTCAGAAGACACCTCCGAACTCATACCGGCAGCCAGAAACCTTTCCAATGTAGGATATGTATGCGAAAC |
| | | TTCTCTCAATCCGGAACCCTTCATCGACATACCAGAACCCATACAGGAGAAAAGCCTTTCCAGTGTAGA |
| | | ATTTGCATGAGGAACTTTTCCCAACGCCCAAATCTTACGCGGCATCTCAGGACACATCTGAGGGGGTCT |
| | | GGCAGCGCAGGCAGTGCAGCGGGGTCAGGAGAGTTCCCCAAAAAAAAGCGCAAAGTATAACGCTTCGAG |
| | | CAGACATGATAAGATACATTG |
| 14 | sp Cas9 | ITGLYETRIDLSQLGGD |
| | region in | |
| | Cas9 ZF5 | |
| | gBlock | |
| 15 | HiFi Cas9 | ATGGATAAGAAATACAGCATCGGCCTGGACATTGGTACGAATAGCGTTGGTTGGGCTGTGATCACCGAT |
| | R691A | GACTACAAGGTGCCGAGTAAGAAATTCAAAGTGCTGGGCAACACTGACCGTCATTCCATTAAAAAGAAT |
| | (NT) | CTGATCGGCGCGCTGCTGTTTGGTAGTGGCGAAACGGCTGAGGCGACGCGTCTGAAGCGTACAGCGCGT |
| | | CGGCGCTACACCCGTCGTAAGAACCGTATCTGTTATCTGCAAGAGATCTTTTCCAACGAAATGGCGAAA |
| | | GTTGACGACTCCTTCTTCCATCGTCTCGAGGAGAGCTTCTTGGTTGAGGAAGATAAAAAGCACGAGCGT |
| | | CACCCGATTTTTGGTAACATCGTTGATGAAGTGGCCTATCACGAAAAATACCCGACGATCTATCATTTG |
| | | CGCAAAAAGCTGGCCGACAGCACCGACAAAGCAGATCTGCGCTTGATCTACTTGGCCCTGGCGCACATG |
| | | ATTAAATTTCGTGGCCATTTTCTGATTGAGGGTGATCTGAACCCGGATAACAGCGATGTTGACAAGTTG |
| | | TTCATTCAGCTGGTACAGATTTATAACCAGCTATTTGAGGAGAACCCGATCAATGCATCGCGGGTGGAC |
| | | GCTAAAGCTATCCTGTCCGCGCGTCTGTCCAAGTCTCGTCGCCTGGAAAATCTGATCGCGCAACTGCCG |
| | | GGTGAAAAGCGCAATGGCTTATTCGGTAACCTTATCGCGCTGTCGCTAGGCCTGACCCCGAATTTCAAG |
| | | AGCAACTTCGACTTAGCGGAAGACGCAAAGCTGCAGCTGAGCAAGGACACCTATGACGATGACCTGGAT |
| | | AACCTGCTAGCGCAGATCGGCGACCAGTATGCGGATCTGTTCCTGGCTGCGAAGAACTTGTCTGACGCC |
| | | ATATTATTATCCGACATCCTGCGAGTTAATAGCGAGATTACCAAAGCGCCACTGTCGGCGTCTATGATC |
| | | AAGCGCTATGACGAACATCACCAAGATTTGACCTTGCTGAAAGCGCTGGTTCGACAGCAGCTGCCGGAA |
| | | AAGTACAAGGAGATCTTCTTCGACCAGAGCAAAAACGGTTACGCCGGTTACATTGACGGTGGTGCTAGC |
| | | CAGGAGGAGTTCTATAAGTTCATCAAGCCGATCCTGGAGAAAATGGATGGTACCGAAGAACTGTTAGTC |
| | | AAACTGAACCGTGAAGACCTGCTGAGAAAGCAGCGTACCTTTGACAATGGCTCCATCCCGCATCAAATC |
| | | CATCTGGGTGAGTTACACGCAATCCTGCGTCGCCAAGAGGACTTCTACCCGTTTCTGAAAGACAACCGT |
| | | GAAAAGATCGAAAAAATCTTGACCTTTCGTATCCCGTATTATGTTGGTCCGCTGGCGCGTGGAAACTCA |
| | | CGCTTCGCATGGATGACCCGTAAGAGCGAAGAAACCATCACGCCGTGGAATTTTGAAGAAGTTGTGGAC |
| | | AAGGGCGCCTCGGCGCAGTCTTTTATCGAACGCATGACCAATTTTGATAAGAACCTGCCGAACGAGAAA |
| | | GTTCTACCGAAGCACAGCCTGCTGTACGAGTATTTCACCGTTTATAATGAACTGACTAAAGTCAAATAC |
| | | GTGACCGAAGGTATGCGTAAGCCGGCGTTTCTCAGCGGTGAGCAGAAAAAGGCCATCGTGGACCTGCTG |
| | | TTTAAGACGAATCGTAAGGTGACCGTTAAACAATTGAAGGAGGACTACTTCAAAAAGATTGAGTGCTTT |
| | | GACAGCGTCGAGATTAGCGGCGTTGAAGATCGCTTTAATGCAAGCCTGGGGGCTTACCATGATCTGTTG |
| | | AAGATCATCAAGGACAAAGACTTTCTCGACAACGAAGAAAATGAGGATATTTTGGAAGATATCGTTCTG |
| | | ACTCTGACTCTGTTCGAAGACAGGGGTATGATCGAAGAGAGATTGAAGACGTACGCGCACCTGTTCGAC |
| | | GACAAGGTCATGAAACAACTGAAGCGCCGTCGTTACACCGGTTGGGGCCGCCTGTCTCGTAAACTGATT |
| | | AATGGTATTCGTGATAAGCAGAGTGGTAAAACCATATTAGATTTTCTGAAAAGCGATGGTTTCGCGAAC |
| | | GCGAACTTCATGCAGTTGATTCACGATGATTCCCTGACCTTCAAAGAAGACATCCAGAAGGCCCAAGTT |
| | | AGCGGCCAGGGTCACAGCCTGCATGAACAGATCGCCAACCTGGCTGGTAGCCCGGCGATCAAAAAGGGC |
| | | ATTTTGCAAACCGTGAAAATTGTTGATGAATTGGTGAAAGTGATGGGTCACAAACCGGAGAACATAGTC |
| | | ATCGAGATGGCTCGTGAAAACCAGACGACCCAGAAAGGCCAAAAAAACTCTCGTGAACGTATGAAACGT |
| | | ATTGAAGAAGGCATTAAGGAGCTCGGTTCGCAAATTCTGAAGGAGCACCCTGTTGAGAATACACAGCTG |
| | | CAGAATGAGAAACTTTATCTGTACTACCTGCAAAACGGTCGTGATATGTATGTGGATCAAGAACTGGAC |
| | | ATTAACCGCTTGTCTGACTACGACGTTGACCATATTGTTCCGCAGTCTTTCATTAAAGACGACAGCATT |
| | | GATAATAAGGTGCTGACGCGCAGTGATAAAAACCGTGGTAAATCCGATAATGTTCCGAGCGAGGAGGTG |
| | | GTGAAGAAGATGAAAAACTATTGGCGTCAATTGCTTAATGCGAAACTGATTACTCAGCGCAAATTCGAT |
| | | AACTTGACGAAAGCAGAACGGGGCGGTCTGTCCGAATTGGACAAGGCGGGCTTTATCAAGAGGCAACTG |
| | | GTGGAGACTCGTCAGATAACGAAGCACGTGGCACAGATCTTAGATTCTCGTATGAATACCAAGTACGAT |
| | | GAGAACGATAAATTGATCCGCGAAGTTAAGGTTATTACCTTGAAGTCGAAGCTCGTCAGCGACTTCCGT |
| | | AAGGACTTTCAATTTTACAAAGTTCGTGAGATCAACAACTACCATCATGCACACGATGCGTATTTGAAT |
| | | GCAGTCGTGGGCACTGCCTTGATTAAAAAATACCCGAAACTGGAATCTGAGTTCGTCTATGGGGATTAC |
| | | AAGGTATATGACGTGCGTAAGATGATTGCCAAATCTGAGCAGGAGATCGGCAAAGCGACGGCCAAGTAC |
| | | TTTTTCTACTCCAACATTATGAACTTTTTCAAAACGGAAATTACCCTGGCGAATGGCGAGATCCGTAAA |
| | | CGCCCACTGATCGAGACCAATGGTGAGACCGGCGAAATCGTGTGGGACAAAGGTCGTGATTTCGCAACC |
| | | GTTCGCAAAGTGCTGAGTATGCCGCAGGTGAACATCGTTAAAAAAACCGAGGTGCAGACCGGAGGTTTT |
| | | TCGAAAGAGAGCATTCTTCCGAAACGCAACAGCGACAAGCTGATCGCGCGTAAAAAGGACTGGGATCCG |
| | | AAGAAGTATGGTGGCTTCGATAGCCCGACCGTTGCCTATAGCGTTCTGGTAGTTGCTAAGGTTGAGAAA |
| | | GGCAAAAGTAAAAAGCTGAAATCCGTGAAAGAATTGTTGGGCATTACCATTATGGAACGTAGCTCTTTC |
| | | GAGAAGAACCCGATTGATTTCCTGGAGGCAAAAGGCTACAAAGAGGTCAAAAAGGATCTAATTATTAAG |
| | | CTGCCAAAGTACAGCCTGTTCGAGCTCGAAAATGGACGTAAACGTATGCTGGCGTCAGCGGGTGAACTG |
| | | CAGAAGGGTAATGAACTGGCGCTGCCGAGCAAATACGTGAATTTTCTGTATCTCGCTAGCCATTATGAA |
| | | AAACTAAAGGGTTCCCCGGAAGACAACGAGCAGAAGCAGCTTTTTGTTGAGCAGCATAAACACTACCTG |
| | | GACGAGATCATTGAGCAAATCAGCGAGTTCTCTAAGCGTGTCATTTTAGCGGATGCTAATCTTGACAAA |
| | | GTACTCAGCGCGTATAACAAACACCGGGACAAGCCGATCCGTGAACAAGCGGAGAACATTATTCACTTG |
| | | TTTACCCTGACGAACCTGGGTGCTCCGGCGGCATTTAAATACTTCGATACCACCATTGACCGCAAGAGA |
| | | TACACCAGCACCAAAGAGGTGCTGGATGCTACCCTGATTCATCAAAGCATCACCGGCCTGTATGAAACC |
| | | CGTATCGATCTTTCCCAATTAGGCGGCGAC |
| 16 | HiFi Cas9 | MDKKYSIGLDIGTNSVGWAVITDDYKVPSKKFKVLGNTDRHSIKKNLIGALLFGSGETAEATRLKRTAR |
| | R691A | RRYTRRKNRICYLQEIFSNEMAKVDDSFFHRLEESFLVEEDKKHERHPIFGNIVDEVAYHEKYPTIYHL |
| | (AA) | RKKLADSTDKADLRLIYLALAHMIKFRGHFLIEGDLNPDNSDVDKLFIQLVQIYNQLFEENPINASRVD |
| | | AKAILSARLSKSRRLENLIAQLPGEKRNGLFGNLIALSLGLTPNFKSNFDLAEDAKLQLSKDTYDDDLD |
| | | NLLAQIGDQYADLFLAAKNLSDAILLSDILRVNSEITKAPLSASMIKRYDEHHQDLTLLKALVRQQLPE |
| | | KYKEIFFDQSKNGYAGYIDGGASQEEFYKFIKPILEKMDGTEELLVKLNREDLLRKQRTFDNGSIPHQI |
| | | HLGELHAILRRQEDFYPFLKDNREKIEKILTFRIPYYVGPLARGNSRFAWMTRKSEETITPWNFEEVVD |
| | | KGASAQSFIERMTNFDKNLPNEKVLPKHSLLYEYFTVYNELTKVKYVTEGMRKPAFLSGEQKKAIVDLL |
| | | FKTNRKVTVKQLKEDYFKKIECFDSVEISGVEDRFNASLGAYHDLLKIIKDKDFLDNEENEDILEDIVL |
| | | TLTLFEDRGMIEERLKTYAHLFDDKVMKQLKRRRYTGWGRLSRKLINGIRDKQSGKTILDFLKSDGFAN |
| | | ANFMQLIHDDSLTFKEDIQKAQVSGQGHSLHEQIANLAGSPAIKKGILQTVKIVDELVKVMGHKPENIV |
| | | IEMARENQTTQKGQKNSRERMKRIEEGIKELGSQILKEHPVENTQLQNEKLYLYYLQNGRDMYVDQELD |
| | | INRLSDYDVDHIVPQSFIKDDSIDNKVLTRSDKNRGKSDNVPSEEVVKKMKNYWRQLLNAKLITQRKFD |
| | | NLTKAERGGLSELDKAGFIKRQLVETRQITKHVAQILDSRMNTKYDENDKLIREVKVITLKSKLVSDFR |
| | | KDFQFYKVREINNYHHAHDAYLNAVVGTALIKKYPKLESEFVYGDYKVYDVRKMIAKSEQEIGKATAKY |
| | | FFYSNIMNFFKTEITLANGEIRKRPLIETNGETGEIVWDKGRDFATVRKVLSMPQVNIVKKTEVQTGGF |
| | | SKESILPKRNSDKLIARKKDWDPKKYGGFDSPTVAYSVLVVAKVEKGKSKKLKSVKELLGITIMERSSF |
| | | EKNPIDFLEAKGYKEVKKDLIIKLPKYSLFELENGRKRMLASAGELQKGNELALPSKYVNFLYLASHYE |
| | | KLKGSPEDNEQKQLFVEQHKHYLDEIIEQISEFSKRVILADANLDKVLSAYNKHRDKPIREQAENIIHL |
| | | FTLTNLGAPAAFKYFDTTIDRKRYTSTKEVLDATLIHQSITGLYETRIDLSQLGGD |
| 17 | HiFi Cas9 | GACAAGAAGTACAGCATCGGCCTGGACATCGGCACCAACTCTGTGGGCTGGGCCGTGATCACCGACGAG |
| | (wt)(NT) | TACAAGGTGCCCAGCAAGAAATTCAAGGTGCTGGGCAACACCGACCGGCACAGCATCAAGAAGAACCTG |
| | | ATCGGAGCCCTGCTGTTCGACAGCGGCGAAACAGCCGAGGCCACCCGGCTGAAGAGAACCGCCAGAAGA |
| | | AGATACACCAGACGGAAGAACCGGATCTGCTATCTGCAAGAGATCTTCAGCAACGAGATGGCCAAGGTG |
| | | GACGACAGCTTCTTCCACAGACTGGAAGAGTCCTTCCTGGTGGAAGAGGATAAGAAGCACGAGCGGCAC |
| | | CCCATCTTCGGCAACATCGTGGACGAGGTGGCCTACCACGAGAAGTACCCCACCATCTACCACCTGAGA |
| | | AAGAAACTGGTGGACAGCACCGACAAGGCCGACCTGCGGCTGATCTATCTGGCCCTGGCCCACATGATC |
| | | AAGTTCCGGGGCCACTTCCTGATCGAGGGCGACCTGAACCCCGACAACAGCGACGTGGACAAGCTGTTC |
| | | ATCCAGCTGGTGCAGACCTACAACCAGCTGTTCGAGGAAAACCCCATCAACGCCAGCGGCGTGGACGCC |
| | | AAGGCCATCCTGTCTGCCAGACTGAGCAAGAGCAGACGGCTGGAAAATCTGATCGCCCAGCTGCCCGGC |
| | | GAGAAGAAGAATGGCCTGTTCGGAAACCTGATTGCCCTGAGCCTGGGCCTGACCCCCAACTTCAAGAGC |
| | | AACTTCGACCTGGCCGAGGATGCCAAACTGCAGCTGAGCAAGGACACCTACGACGACGACCTGGACAAC |
| | | CTGCTGGCCCAGATCGGCGACCAGTACGCCGACCTGTTTCTGGCCGCCAAGAACCTGTCCGACGCCATC |
| | | CTGCTGAGCGACATCCTGAGAGTGAACACCGAGATCACCAAGGCCCCCCTGAGCGCCTCTATGATCAAG |
| | | AGATACGACGAGCACCACCAGGACCTGACCCTGCTGAAAGCTCTCGTGCGGCAGCAGCTGCCTGAGAAG |
| | | TACAAAGAGATTTTCTTCGACCAGAGCAAGAACGGCTACGCCGGCTACATTGACGGCGGAGCCAGCCAG |
| | | GAAGAGTTCTACAAGTTCATCAAGCCCATCCTGGAAAAGATGGACGGCACCGAGGAACTGCTCGTGAAG |
| | | CTGAACAGAGAGGACCTGCTGCGGAAGCAGCGGACCTTCGACAACGGCAGCATCCCCCACCAGATCCAC |
| | | CTGGGAGAGCTGCACGCCATTCTGCGGCGGCAGGAAGATTTTTACCCATTCCTGAAGGACAACCGGGAA |
| | | AAGATCGAGAAGATCCTGACCTTCCGCATCCCCTACTACGTGGGCCCTCTGGCCAGGGGAAACAGCAGA |
| | | TTCGCCTGGATGACCAGAAAGAGCGAGGAAACCATCACCCCCTGGAACTTCGAGGAAGTGGTGGACAAG |
| | | GGCGCTTCCGCCCAGAGCTTCATCGAGCGGATGACCAACTTCGATAAGAACCTGCCCAACGAGAAGGTG |
| | | CTGCCCAAGCACAGCCTGCTGTACGAGTACTTCACCGTGTATAACGAGCTGACCAAAGTGAAATACGTG |
| | | ACCGAGGGAATGAGAAAGCCCGCCTTCCTGAGCGGCGAGCAGAAAAAGGCCATCGTGGACCTGCTGTTC |
| | | AAGACCAACCGGAAAGTGACCGTGAAGCAGCTGAAAGAGGACTACTTCAAGAAAATCGAGTGCTTCGAC |
| | | TCCGTGGAAATCTCCGGCGTGGAAGATCGGTTCAACGCCTCCCTGGGCACATACCACGATCTGCTGAAA |
| | | ATTATCAAGGACAAGGACTTCCTGGACAATGAGGAAAACGAGGACATTCTGGAAGATATCGTGCTGACC |
| | | CTGACACTGTTTGAGGACAGAGAGATGATCGAGGAACGGCTGAAAACCTATGCCCACCTGTTCGACGAC |
| | | AAAGTGATGAAGCAGCTGAAGCGGCGGAGATACACCGGCTGGGGCAGGCTGAGCCGGAAGCTGATCAAC |
| | | GGCATCCGGGACAAGCAGTCCGGCAAGACAATCCTGGATTTCCTGAAGTCCGACGGCTTCGCCAACAGA |
| | | AACTTCATGCAGCTGATCCACGACGACAGCCTGACCTTTAAAGAGGACATCCAGAAAGCCCAGGTGTCC |
| | | GGCCAGGGCGATAGCCTGCACGAGCACATTGCCAATCTGGCCGGCAGCCCCGCCATTAAGAAGGGCATC |
| | | CTGCAGACAGTGAAGGTGGTGGACGAGCTCGTGAAAGTGATGGGCCGGCACAAGCCCGAGAACATCGTG |
| | | ATCGAAATGGCCAGAGAGAACCAGACCACCCAGAAGGGACAGAAGAACAGCCGCGAGAGAATGAAGCGG |
| | | ATCGAAGAGGGCATCAAAGAGCTGGGCAGCCAGATCCTGAAAGAACACCCCGTGGAAAACACCCAGCTG |
| | | CAGAACGAGAAGCTGTACCTGTACTACCTGCAGAATGGGCGGGATATGTACGTGGACCAGGAACTGGAC |
| | | ATCAACCGGCTGTCCGACTACGATGTGGACCATATCGTGCCTCAGAGCTTTCTGAAGGACGACTCCATC |
| | | GACAACAAGGTGCTGACCAGAAGCGACAAGAACCGGGGCAAGAGCGACAACGTGCCCTCCGAAGAGGTC |
| | | GTGAAGAAGATGAAGAACTACTGGCGGCAGCTGCTGAACGCCAAGCTGATTACCCAGAGAAAGTTCGAC |
| | | AATCTGACCAAGGCCGAGAGAGGCGGCCTGAGCGAACTGGATAAGGCCGGCTTCATCAAGAGACAGCTG |
| | | GTGGAAACCCGGCAGATCACAAAGCACGTGGCACAGATCCTGGACTCCCGGATGAACACTAAGTACGAC |
| | | GAGAATGACAAGCTGATCCGGGAAGTGAAAGTGATCACCCTGAAGTCCAAGCTGGTGTCCGATTTCCGG |
| | | AAGGATTTCCAGTTTTACAAAGTGCGCGAGATCAACAACTACCACCACGCCCACGACGCCTACCTGAAC |
| | | GCCGTCGTGGGAACCGCCCTGATCAAAAAGTACCCTAAGCTGGAAAGCGAGTTCGTGTACGGCGACTAC |
| | | AAGGTGTACGACGTGCGGAAGATGATCGCCAAGAGCGAGCAGGAAATCGGCAAGGCTACCGCCAAGTAC |
| | | TTCTTCTACAGCAACATCATGAACTTTTTCAAGACCGAGATTACCCTGGCCAACGGCGAGATCCGGAAG |
| | | CGGCCTCTGATCGAGACAAACGGCGAAACCGGGGAGATCGTGTGGGATAAGGGCCGGGATTTTGCCACC |
| | | GTGCGGAAAGTGCTGAGCATGCCCCAAGTGAATATCGTGAAAAAGACCGAGGTGCAGACAGGCGGCTTC |
| | | AGCAAAGAGTCTATCCTGCCCAAGAGGAACAGCGATAAGCTGATCGCCAGAAAGAAGGACTGGGACCCT |
| | | AAGAAGTACGGCGGCTTCGACAGCCCCACCGTGGCCTATTCTGTGCTGGTGGTGGCCAAAGTGGAAAAG |
| | | GGCAAGTCCAAGAAACTGAAGAGTGTGAAAGAGCTGCTGGGGATCACCATCATGGAAAGAAGCAGCTTC |
| | | GAGAAGAATCCCATCGACTTTCTGGAAGCCAAGGGCTACAAAGAAGTGAAAAAGGACCTGATCATCAAG |
| | | CTGCCTAAGTACTCCCTGTTCGAGCTGGAAAACGGCCGGAAGAGAATGCTGGCCTCTGCCGGCGAACTG |
| | | CAGAAGGGAAACGAACTGGCCCTGCCCTCCAAATATGTGAACTTCCTGTACCTGGCCAGCCACTATGAG |
| | | AAGCTGAAGGGCTCCCCCGAGGATAATGAGCAGAAACAGCTGTTTGTGGAACAGCACAAGCACTACCTG |
| | | GACGAGATCATCGAGCAGATCAGCGAGTTCTCCAAGAGAGTGATCCTGGCCGACGCTAATCTGGACAAA |
| | | GTGCTGTCCGCCTACAACAAGCACCGGGATAAGCCCATCAGAGAGCAGGCCGAGAATATCATCCACCTG |
| | | TTTACCCTGACCAATCTGGGAGCCCCTGCCGCCTTCAAGTACTTTGACACCACCATCGACCGGAAGAGG |
| | | TACACCAGCACCAAAGAGGTGCTGGACGCCACCCTGATCCACCAGAGCATCACCGGCCTGTACGAGACA |
| | | CGGATCGACCTGTCTCAGCTGGGAGGCGAC |
| 18 | HiFi Cas9 | DKKYSIGLDIGTNSVGWAVITDEYKVPSKKFKVLGNTDRHSIKKNLIGALLFDSGETAEATRLKRTARR |
| | (wt)(AA) | RYTRRKNRICYLQEIFSNEMAKVDDSFFHRLEESFLVEEDKKHERHPIFGNIVDEVAYHEKYPTIYHLR |
| | | KKLVDSTDKADLRLIYLALAHMIKFRGHFLIEGDLNPDNSDVDKLFIQLVQTYNQLFEENPINASGVDA |
| | | KAILSARLSKSRRLENLIAQLPGEKKNGLFGNLIALSLGLTPNFKSNFDLAEDAKLQLSKDTYDDDLDN |
| | | LLAQIGDQYADLFLAAKNLSDAILLSDILRVNTEITKAPLSASMIKRYDEHHQDLTLLKALVRQQLPEK |
| | | YKEIFFDQSKNGYAGYIDGGASQEEFYKFIKPILEKMDGTEELLVKLNREDLLRKQRTFDNGSIPHQIH |
| | | LGELHAILRRQEDFYPFLKDNREKIEKILTFRIPYYVGPLARGNSRFAWMTRKSEETITPWNFEEVVDK |
| | | GASAQSFIERMTNFDKNLPNEKVLPKHSLLYEYFTVYNELTKVKYVTEGMRKPAFLSGEQKKAIVDLLF |
| | | KTNRKVTVKQLKEDYFKKIECFDSVEISGVEDRFNASLGTYHDLLKIIKDKDFLDNEENEDILEDIVLT |
| | | LTLFEDREMIEERLKTYAHLFDDKVMKQLKRRRYTGWGRLSRKLINGIRDKQSGKTILDFLKSDGFANR |
| | | NFMQLIHDDSLTFKEDIQKAQVSGQGDSLHEHIANLAGSPAIKKGILQTVKVVDELVKVMGRHKPENIV |
| | | IEMARENQTTQKGQKNSRERMKRIEEGIKELGSQILKEHPVENTQLQNEKLYLYYLQNGRDMYVDQELD |
| | | INRLSDYDVDHIVPQSFLKDDSIDNKVLTRSDKNRGKSDNVPSEEVVKKMKNYWRQLLNAKLITQRKFD |
| | | NLTKAERGGLSELDKAGFIKRQLVETRQITKHVAQILDSRMNTKYDENDKLIREVKVITLKSKLVSDFR |
| | | KDFQFYKVREINNYHHAHDAYLNAVVGTALIKKYPKLESEFVYGDYKVYDVRKMIAKSEQEIGKATAKY |
| | | FFYSNIMNFFKTEITLANGEIRKRPLIETNGETGEIVWDKGRDFATVRKVLSMPQVNIVKKTEVQTGGF |
| | | SKESILPKRNSDKLIARKKDWDPKKYGGFDSPTVAYSVLVVAKVEKGKSKKLKSVKELLGITIMERSSF |
| | | EKNPIDFLEAKGYKEVKKDLIIKLPKYSLFELENGRKRMLASAGELQKGNELALPSKYVNFLYLASHYE |
| | | KLKGSPEDNEQKQLFVEQHKHYLDEIIEQISEFSKRVILADANLDKVLSAYNKHRDKPIREQAENIIHL |
| | | FTLTNLGAPAAFKYFDTTIDRKRYTSTKEVLDATLIHQSITGLYETRIDLSQLGGD |
| 20 | Flexible | SGGSSGGSSGSETPGTSESATPESSGGSSGGS |
| | Linker | |
| 21 | Flexible | GSAGSAAGSGEF |
| | Linker 2 | |
| 22 | Zinc finger | SRPGERPFQCRICMRNFSNMSNLTRHTRTHTGEKPFQCRICMRNFSDRSVLRRHLRTHTGSQKPFQCRI |
| | 5 | CMRNFSDPSNLARHTRTHTGEKPFQCRICMRNFSDRSSLRRHLRTHTGSQKPFQCRICMRNFSQSGTLH |
| | | RHTRTHTGEKPFQCRICMRNFSQRPNLTRHLRTHLRGS |
| 23 | ZFDBM5 | TGAAGCAGTCGACGCCGAAGAGGGTCCGAGGTATTCCTTCGGCGTCGACTGCTTCA |
| | (1)-loop- | |
| | ZFDBM5 | |
| | (2) or | |
| | ZFDBM5 | |
| | hairpin | |
| | sequence | |
| 24 | loop | AGGGTCCGAGGTATTC |
| 46 | DBM5 | GAAGCAGTCGACGCCGAA |
| | without | |
| | flanking bp | |
| 47 | DBM5 or | TGAAGCAGTCGACGCCGAAG |
| | ZFDBM5 | |
| | (1) | |
| 48 | DBM5 or | CTTCGGCGTCGACTGCTTCA |
| | ZFDBM5 | |
| | (2) | |
| 25 | ZFDBM5 | TGAAGCAGTCGACGCCGAAGGAATACCTCGGACCCTCTTCGGCGTCGACTGCTTCA |
| | with stem | |
| | loop | |
| 26 | loop | GAATACCTCGGACCCT |
| 27 | TRAC UHA | TCTCCCCTCTGACCATGGAGCCCACCCCTGCTCCACTGCTCCAGGGACAGCCCTATGCTGCAGGCAGCT |
| | | CTGCCCCCACTCAGCATCCCAGGGGCTGATTTCTTTGGTTTTGGATCCAGCTGGATGTCTGCATTGCCG |
| | | AGGCCACCAGGGCTGGCTCAGCAACTGTCGGGGAATCACCAGGGTCTGAGAAATCTTGTGCGCATGTGA |
| | | GGGGCTGTGGGAGCAGAGAACCACTGGGTGGGAAATTCTAATCCCCACCCTGCTGGAAACTCTCTGGGT |
| | | GGCCCCAACATGCTAATCCTCCGGTAAACCTCTGTTTCCTCCTCAAAAGGCAGGAGGTCGGAAAGAATA |
| | | AACAATGAGAGTCACATTAAAAACACAAAATCCTACGGAAATACTGAAGAATGAGTCTCAGCACTAAGG |
| | | AAAAGCCTCCAGCAGCTCCTGCTTTCTGAGGGTGAAGGATAGACGCTGTGGCTCTGCATGACTCACTAG |
| | | CACTCTATCACGGCCATATTCTGACAGGGTCAGTGGCTCCAACTAACATTTGTTTGGTACTTTACAGTT |
| | | TATTAAATAGATGTTTATATGGAGAAGCTCTCATTTCTTTCTCAGAAGAGCCTGGCTAGGAAGGTGGAT |
| | | GAGGCACCATATTCATTTTGCAGGTGAAATTCCTGAGATGTAAGGAGCTGCTGTGACTTGCTCAAGGCC |
| | | TTATATCGAGTAAACGGTAGCGCTGGGGCTTAGACGCAGGTGTTCTGATTTATAGTTCAAAACCTCTAT |
| | | CAATGAGAGAGCAATCTCCTGGTAATGTGATAGATTTCCCAACTTAATGCCAACATACCATAAACCTCC |
| | | CATTCTGCTAATGCCCAGCCTAAGTTGGGGAGACCACTCCAGATTCCAAGATGTACAGTTTGCTTTGCT |
| | | GGGCCTTTTTCCCATGCCTGCCTTTACTCTGCCAGAGTTATATTGCTGGGGTTTTGAAGAAGATCCTAT |
| | | TAAATAAAAGAATAAGCAGTATTATTAAGTAGCCCTGCATTTCAGGTTTCCTTGAGTGGCAGGCCAGGC |
| | | CTGGCCGTGAACGTTCACTGAAATCATGGCCTCTTGGCCAAGATTGATAGCTTGTGCCTGTCCCTGAGT |
| | | CCCAGTCCATCACGAGCAGCTGGTTTCTAAGATGCTATTTCCCGTATAAAGCATGAGACCGTGACTTGC |
| | | CAGCCCCACAGAGCCCCGCCCTTGTCCATCACTGGCATCTGGACTCCAGCCTGGGTTGGGGCAAAGAGG |
| | | GAAATGAGATCATGTCCTAACCCTG |
| 28 | TRAC DHA | AGGAATATCCAGAACCCTGACCCTGCCGTGTACCAGCTGAGAGACTCTAAATCCAGTGACAAGTCTGTC |
| | | TGCCTATTCACCGATTTTGATTCTCAAACAAATGTGTCACAAAGTAAGGATTCTGATGTGTATATCACA |
| | | GACAAAACTGTGCTAGACATGAGGTCTATGGACTTCAAGAGCAACAGTGCTGTGGCCTGGAGCAACAAA |
| | | TCTGACTTTGCATGTGCAAACGCCTTCAACAACAGCATTATTCCAGAAGACACCTTCTTCCCCAGCCCA |
| | | GGTAAGGGCAGCTTTGGTGCCTTCGCAGGCTGTTTCCTTGCTTCAGGAATGGCCAGGTTCTGCCCAGAG |
| | | CTCTGGTCAATGATGTCTAAAACTCCTCTGATTGGTGGTCTCGGCCTTATCCATTGCCACCAAAACCCT |
| | | CTTTTTACTAAGAAACAGTGAGCCTTGTTCTGGCAGTCCAGAGAATGACACGGGAAAAAAGCAGATGAA |
| | | GAGAAGGTGGCAGGAGAGGGCACGTGGCCCAGCCTCAGTCTCTCCAACTGAGTTCCTGCCTGCCTGCCT |
| | | TTGCTCAGACTGTTTGCCCCTTACTGCTCTTCTAGGCCTCATTCTAAGCCCCTTCTCCAAGTTGCCTCT |
| | | CCTTATTTCTCCCTGTCTGCCAAAAAATCTTTCCCAGCTCACTAAGTCAGTCTCACGCAGTCACTCATT |
| | | AACCCACCAATCACTGATTGTGCCGGCACATGAATGCACCAGGTGTTGAAGTGGAGGAATTAAAAAGTC |
| | | AGATGAGGGGTGTGCCCAGAGGAAGCACCATTCTAGTTGGGGGAGCCCATCTGTCAGCTGGGAAAAGTC |
| | | CAAATAACTTCAGATTGGAATGTGTTTTAACTCAGGGTTGAGAAAACAGCCACCTTCAGGACAAAAGTC |
| | | AGGGAAGGGCTCTCTGAAGAAATGCTACTTGAAGATACCAGCCCTACCAAGGGCAGGGAGAGGACCCTA |
| | | TAGAGGCCTGGGACAGGAGCTCAATGAGAAAGGAGAAGAGCAGCAGGCATGAGTTGAATGAAGGAGGCA |
| | | GGGCCGGGTCACAGGGCCTTCTAGGCCATGAGAGGGTAGACAGTATTCTAAGTACGCCAGAAAGCTGTT |
| | | GATCGGCTTCAAGCAGGGAAGGGACACCTAATTTGCTTTTCTTTTCTTTTTTTTTTTTTTTTTTTTTTT |
| | | GAGATGGAGTTTTGCTCTTGTTGCCCAGGCTGGAGTGCAATGGTGCATCTTGGCTCACTGCAAC |
| 29 | gRNA | GTTTTAGAGCTAGAAATAGCAAGTTAAAATAAGGCTAGTCCGTTATCAACTTGAAAAAGTGGCACCGAG |
| | scaffold | TCGGTGC |
| 30 | pEF-1α | GGCTCCGGTGCCCGTCAGTGGGCAGAGCGCACATCGCCCACAGTCCCCGAGAAGTTGGGGGGAGGGGTC |
| | | GGCAATTGAACCGGTGCCTAGAGAAGGTGGCGCGGGGTAAACTGGGAAAGTGATGTCGTGTACTGGCTC |
| | | CGCCTTTTTCCCGAGGGTGGGGGAGAACCGTATATAAGTGCAGTAGTCGCCGTGAACGTTCTTTTTCGC |
| | | AACGGGTTTGCCGCCAGAACACAGGTAAGTGCCGTGTGTGGTTCCCGCGGGCCTGGCCTCTTTACGGGT |
| | | TATGGCCCTTGCGTGCCTTGAATTACTTCCACCTGGCTGCAGTACGTGATTCTTGATCCCGAGCTTCGG |
| | | GTTGGAAGTGGGTGGGAGAGTTCGAGGCCTTGCGCTTAAGGAGCCCCTTCGCCTCGTGCTTGAGTTGAG |
| | | GCCTGGCCTGGGCGCTGGGGCCGCCGCGTGCGAATCTGGTGGCACCTTCGCGCCTGTCTCGCTGCTTTC |
| | | GATAAGTCTCTAGCCATTTAAAATTTTTGATGACCTGCTGCGACGCTTTTTTTCTGGCAAGATAGTCTT |
| | | GTAAATGCGGGCCAAGATCTGCACACTGGTATTTCGGTTTTTGGGGCCGCGGGCGGCGACGGGGCCCGT |
| | | GCGTCCCAGCGCACATGTTCGGCGAGGCGGGGCCTGCGAGCGCGGCCACCGAGAATCGGACGGGGGTAG |
| | | TCTCAAGCTGGCCGGCCTGCTCTGGTGCCTGGTCTCGCGCCGCCGTGTATCGCCCCGCCCTGGGCGGCA |
| | | AGGCTGGCCCGGTCGGCACCAGTTGCGTGAGCGGAAAGATGGCCGCTTCCCGGCCCTGCTGCAGGGAGC |
| | | TCAAAATGGAGGACGCGGCGCTCGGGAGAGCGGGCGGGTGAGTCACCCACACAAAGGAAAAGGGCCTTT |
| | | CCGTCCTCAGCCGTCGCTTCATGTGACTCCACGGAGTACCGGGCGCCGTCCAGGCACCTCGATTAGTTC |
| | | TCGAGCTTTTGGAGTACGTCGTCTTTAGGTTGGGGGGAGGGGTTTTATGCGATGGAGTTTCCCCACACT |
| | | GAGTGGGTGGAGACTGAAGTTAGGCCAGCTTGGCACTTGATGTAATTCTCCTTGGAATTTGCCCTTTTT |
| | | GAGTTTGGATCTTGGTTCATTCTCAAGCCTCAGACAGTGGTTCAAAGTTTTTTTCTTCCATTTCAGGTG |
| | | TCGTGA |
| 31 | SV40 NLS | PKKKRKV (nuclear localization signal of SV40 (simian virus |
| | | 40) large T antigen) |
| 32 | pyrF | MTLTASSSSRAVTNSPVVVALDYHNRDDALAFVDKIDPRDCRLKVGKEMFTLFGPQFVRELQQRGFDIF |
| | | LDLKFHDIPNTAAHAVAAAADLGVWMVNVHASGGARMMTAAREALVPFGKDAPLLIAVTVLTSMEASDL |
| | | VDLGMTLSPADYAERLAALTQKCGLDGVVCSAQEAVRFKQVFGQEFKLVTPGIRPQGSEAGDQRRIMTP |
| | | EQALSAGVDYMVIGRPVTQSVDPAQTLKAINASLQRSA |
| 33 | mRuby | MVSKGEEDNMAIIKEFMRFKVHMEGSVNGHEFEIEGEGEGRPYEGTQTAKLKVTKGGPLPFAWDILSPQ |
| | | FMYGSKAYVKHPADIPDYLKLSFPEGFKWERVMNFEDGGVVTVTQDSSLQDGEFIYKVKLRGTNFPSDG |
| | | PVMQKKTMGWEASSERMYPEDGALKGEIKQRLKLKDGGHYDAEVKTTYKAKKPVQLPGAYNVNIKLDIT |
| | | SHNEDYTIVEQYERAEGRHSTGGMDELYK |
| 34 | emGFP | MVSKGEELFTGVVPILVELDGDVNGHKFFVSGEGEGDATYGKLTLKFICTTGKLPVPWPTLVTTFTYGV |
| | | QCFARYPDHMKQHDFFKSAMPEGYVQERTIFFKDDGNYKTRAEVKFEGDTLVNRIELKGIDFKEDGNIL |
| | | GHKLEYNYNSHKVYITADKQKNGIKVNFKTRHNIEDGSVQLADHYQQNTPIGDGPVLLPDNHYLSTQSA |
| | | LSKDPNEKRDHMVLLEFVTAAGITLGMDELYK |
| 35 | AmpR | MSIQHFRVALIPFFAAFCLPVFAHPETLVKVKDAEDQLGARVGYIELDLNSGKILESFRPEERFPMMST |
| | | FKVLLCGAVLSRIDAGQEQLGRRIHYSQNDLVEYSPVTEKHLTDGMTVRELCSAAITMSDNTAANLLLT |
| | | TIGGPKELTAFLHNMGDHVTRLDRWEPELNEAIPNDERDTTMPVAMATTLRKLLTGELLTLASRQQLID |
| | | WMEADKVAGPLLRSALPAGWFIADKSGAGERGSRGIIAALGPDGKPSRIVVIYTTGSQATMDERNRQIA |
| | | EIGASLIKHW* |
| 36 | KanR | TTAGAAAAACTCATCGAGCATCAAATGAAACTGCAATTTATTCATATCAGGATTATCAATACCATATTT |
| | | TTGAAAAAGCCGTTTCTGTAATGAAGGAGAAAACTCACCGAGGCAGTTCCATAGGATGGCAAGATCCTG |
| | | GTATCGGTCTGCGATTCCGACTCGTCCAACATCAATACAACCTATTAATTTCCCCTCGTCAAAAATAAG |
| | | GTTATCAAGTGAGAAATCACCATGAGTGACGACTGAATCCGGTGAGAATGGCAAAAGCTTATGCATTTC |
| | | TTTCCAGACTTGTTCAACAGGCCAGCCATTACGCTCGTCATCAAAATCACTCGCATCAACCAAACCGTT |
| | | ATTCATTCGTGATTGCGCCTGAGCGAGACGAAATACGCGATCGCTGTTAAAAGGACAATTACAAACAGG |
| | | AATCGAATGCAACCGGCGCAGGAACACTGCCAGCGCATCAACAATATTTTCACCTGAATCAGGATATTC |
| | | TTCTAATACCTGGAATGCTGTTTTCCCGGGGATCGCAGTGGTGAGTAACCATGCATCATCAGGAGTACG |
| | | GATAAAATGCTTGATGGTCGGAAGAGGCATAAATTCCGTCAGCCAGTTTAGTCTGACCATCTCATCTGT |
| | | AACATCATTGGCAACGCTACCTTTGCCATGTTTCAGAAACAACTCTGGCGCATCGGGCTTCCCATACAA |
| | | TCGATAGATTGTCGCACCTGATTGCCCGACATTATCGCGAGCCCATTTATACCCATATAAATCAGCATC |
| | | CATGTTGGAATTTAATCGCGGCCTCGAGCAAGACGTTTCCCGTTGAATATGGCTCAT |
| 37 | PuroR | MTEYKPTVRLATRDDVPRAVRTLAAAFADYPATRHTVDPDRHIERVTELQELFLTRVGLDIGKVWVADD |
| | | GAAVAVWTTPESVEAGAVFAEIGPRMAELSGSRLAAQQQMEGLLAPHRPKEPAWFLATVGVSPDHQGKG |
| | | LGSAVVLPGVEAAERAGVPAFLETSAPRNLPFYERLGFTVTADVEVPEGPRTWCMTRKPGA* |
| 38 | pGRP78 | CTTTGTGATGGGAATTCTGTGGACCTGCAGGGCCCACTAGTGCGGTTACCAGCGGAAATGCCTCGGGGT |
| | | CAGAAGTCGCAGGAGAGATAGACAGCTGCTGAACCAATGGGACCAGCGGATGGGGCGGATGTTATCTAC |
| | | CATTGGTGAACGTTAGAAACGAATAGCAGCCAATGAATCAGCTGGGGGGGCGGAGCAGTGACGTTTATT |
| | | GCGGAGGGGGCCGCTTCGAATCGGCGGCGGCCAGCTTGGTGGCCTGGGCCAATGAACGGCCTCCAACGA |
| | | GCAGGGCCTTCACCAATCGGCGGCCTCCACGACGGGGCTGGGGGAGGGTATATAAGCCGAGTAGGCGAC |
| | | GGTGAGGTCGACGCCGGCCAAGACAGCACAGACAGATTGACCTATTGGGGTGTTTCGCGAGTGTGAGAG |
| | | GGAAGCGCCGCGGCCTGTATTTCTAGACCTGCCCTTCGCCTGGTTCGTGGCGCCTTGTGACCCCGGGCC |
| | | CCTGCCGCCTGCAAGTCGGAAATTGCGCTGTGCTCCTGTGCTACGGCCTGTGGCTGGACTGCCTGCTGC |
| | | TGCCCAACTGGCTGCCACC |
| 39 | β-globin | AATAAAGGAAATTTATTTTCATTGCAATAGTGTGTTGGAATTTTTTGTGTCTCTCA |
| | poly(A) | |
| 40 | 6X His | HHHHHH |
| | Affinity Tag | |
| 41 | CTS | GGTTCTGGATATCTGTTGGG |
| 42 | g526 | TCAGGGTTCTGGATATCTGT |
| | protospacer | |
| | sequence | |
| 43 | 3X FLAG | DYKDHDGDYKDHDIDYKDDDDK |
| | three | |
| | tandem | |
| | FLAG® | |
| | epitope tags, | |
| | followed by | |
| | an | |
| | enterokinase | |
| | cleavage | |
| | site | |
| 44 | o223 primer | TTGTGTTCTGTGGCATTGCG |
| 45 | o257 primer | GGCAGCGAGGCATACATAGT |
| 49 | sgRNA | GGGGCCACTAGGGACAGGAT |
| | (DMD) | |
| 50 | Cas9:zinc | ATGCATCACCACCACCACCATGCTTCACCCCCAAAGAAGAAAAGAAAGGTGGGTTCGATGGATAAGAAA |
| | finger | TACAGCATCGGCCTGGACATTGGTACGAATAGCGTTGGTTGGGCTGTGATCACCGATGACTACAAGGTG |
| | fusion | CCGAGTAAGAAATTCAAAGTGCTGGGCAACACTGACCGTCATTCCATTAAAAAGAATCTGATCGGCGCG |
| | protein for | CTGCTGTTTGGTAGTGGCGAAACGGCTGAGGCGACGCGTCTGAAGCGTACAGCGCGTCGGCGCTACACC |
| | E. coli | CGTCGTAAGAACCGTATCTGTTATCTGCAAGAGATCTTTTCCAACGAAATGGCGAAAGTTGACGACTCC |
| | expression | TTCTTCCATCGTCTCGAGGAGAGCTTCTTGGTTGAGGAAGATAAAAAGCACGAGCGTCACCCGATTTTT |
| | NT | GGTAACATCGTTGATGAAGTGGCCTATCACGAAAAATACCCGACGATCTATCATTTGCGCAAAAAGCTG |
| | | GCCGACAGCACCGACAAAGCAGATCTGCGCTTGATCTACTTGGCCCTGGCGCACATGATTAAATTTCGT |
| | | GGCCATTTTCTGATTGAGGGTGATCTGAACCCGGATAACAGCGATGTTGACAAGTTGTTCATTCAGCTG |
| | | GTACAGATTTATAACCAGCTATTTGAGGAGAACCCGATCAATGCATCGCGGGTGGACGCTAAAGCTATC |
| | | CTGTCCGCGCGTCTGTCCAAGTCTCGTCGCCTGGAAAATCTGATCGCGCAACTGCCGGGTGAAAAGCGC |
| | | AATGGCTTATTCGGTAACCTTATCGCGCTGTCGCTAGGCCTGACCCCGAATTTCAAGAGCAACTTCGAC |
| | | TTAGCGGAAGACGCAAAGCTGCAGCTGAGCAAGGACACCTATGACGATGACCTGGATAACCTGCTAGCG |
| | | CAGATCGGCGACCAGTATGCGGATCTGTTCCTGGCTGCGAAGAACTTGTCTGACGCCATATTATTATCC |
| | | GACATCCTGCGAGTTAATAGCGAGATTACCAAAGCGCCACTGTCGGCGTCTATGATCAAGCGCTATGAC |
| | | GAACATCACCAAGATTTGACCTTGCTGAAAGCGCTGGTTCGACAGCAGCTGCCGGAAAAGTACAAGGAG |
| | | ATCTTCTTCGACCAGAGCAAAAACGGTTACGCCGGTTACATTGACGGTGGTGCTAGCCAGGAGGAGTTC |
| | | TATAAGTTCATCAAGCCGATCCTGGAGAAAATGGATGGTACCGAAGAACTGTTAGTCAAACTGAACCGT |
| | | GAAGACCTGCTGAGAAAGCAGCGTACCTTTGACAATGGCTCCATCCCGCATCAAATCCATCTGGGTGAG |
| | | TTACACGCAATCCTGCGTCGCCAAGAGGACTTCTACCCGTTTCTGAAAGACAACCGTGAAAAGATCGAA |
| | | AAAATCTTGACCTTTCGTATCCCGTATTATGTTGGTCCGCTGGCGCGTGGAAACTCACGCTTCGCATGG |
| | | ATGACCCGTAAGAGCGAAGAAACCATCACGCCGTGGAATTTTGAAGAAGTTGTGGACAAGGGCGCCTCG |
| | | GCGCAGTCTTTTATCGAACGCATGACCAATTTTGATAAGAACCTGCCGAACGAGAAAGTTCTACCGAAG |
| | | CACAGCCTGCTGTACGAGTATTTCACCGTTTATAATGAACTGACTAAAGTCAAATACGTGACCGAAGGT |
| | | ATGCGTAAGCCGGCGTTTCTCAGCGGTGAGCAGAAAAAGGCCATCGTGGACCTGCTGTTTAAGACGAAT |
| | | CGTAAGGTGACCGTTAAACAATTGAAGGAGGACTACTTCAAAAAGATTGAGTGCTTTGACAGCGTCGAG |
| | | ATTAGCGGCGTTGAAGATCGCTTTAATGCAAGCCTGGGGGCTTACCATGATCTGTTGAAGATCATCAAG |
| | | GACAAAGACTTTCTCGACAACGAAGAAAATGAGGATATTTTGGAAGATATCGTTCTGACTCTGACTCTG |
| | | TTCGAAGACAGGGGTATGATCGAAGAGAGATTGAAGACGTACGCGCACCTGTTCGACGACAAGGTCATG |
| | | AAACAACTGAAGCGCCGTCGTTACACCGGTTGGGGCCGCCTGTCTCGTAAACTGATTAATGGTATTCGT |
| | | GATAAGCAGAGTGGTAAAACCATATTAGATTTTCTGAAAAGCGATGGTTTCGCGAACGCGAACTTCATG |
| | | CAGTTGATTCACGATGATTCCCTGACCTTCAAAGAAGACATCCAGAAGGCCCAAGTTAGCGGCCAGGGT |
| | | CACAGCCTGCATGAACAGATCGCCAACCTGGCTGGTAGCCCGGCGATCAAAAAGGGCATTTTGCAAACC |
| | | GTGAAAATTGTTGATGAATTGGTGAAAGTGATGGGTCACAAACCGGAGAACATAGTCATCGAGATGGCT |
| | | CGTGAAAACCAGACGACCCAGAAAGGCCAAAAAAACTCTCGTGAACGTATGAAACGTATTGAAGAAGGC |
| | | ATTAAGGAGCTCGGTTCGCAAATTCTGAAGGAGCACCCTGTTGAGAATACACAGCTGCAGAATGAGAAA |
| | | CTTTATCTGTACTACCTGCAAAACGGTCGTGATATGTATGTGGATCAAGAACTGGACATTAACCGCTTG |
| | | TCTGACTACGACGTTGACCATATTGTTCCGCAGTCTTTCATTAAAGACGACAGCATTGATAATAAGGTG |
| | | CTGACGCGCAGTGATAAAAACCGTGGTAAATCCGATAATGTTCCGAGCGAGGAGGTGGTGAAGAAGATG |
| | | AAAAACTATTGGCGTCAATTGCTTAATGCGAAACTGATTACTCAGCGCAAATTCGATAACTTGACGAAA |
| | | GCAGAACGGGGCGGTCTGTCCGAATTGGACAAGGCGGGCTTTATCAAGAGGCAACTGGTGGAGACTCGT |
| | | CAGATAACGAAGCACGTGGCACAGATCTTAGATTCTCGTATGAATACCAAGTACGATGAGAACGATAAA |
| | | TTGATCCGCGAAGTTAAGGTTATTACCTTGAAGTCGAAGCTCGTCAGCGACTTCCGTAAGGACTTTCAA |
| | | TTTTACAAAGTTCGTGAGATCAACAACTACCATCATGCACACGATGCGTATTTGAATGCAGTCGTGGGC |
| | | ACTGCCTTGATTAAAAAATACCCGAAACTGGAATCTGAGTTCGTCTATGGGGATTACAAGGTATATGAC |
| | | GTGCGTAAGATGATTGCCAAATCTGAGCAGGAGATCGGCAAAGCGACGGCCAAGTACTTTTTCTACTCC |
| | | AACATTATGAACTTTTTCAAAACGGAAATTACCCTGGCGAATGGCGAGATCCGTAAACGCCCACTGATC |
| | | GAGACCAATGGTGAGACCGGCGAAATCGTGTGGGACAAAGGTCGTGATTTCGCAACCGTTCGCAAAGTG |
| | | CTGAGTATGCCGCAGGTGAACATCGTTAAAAAAACCGAGGTGCAGACCGGAGGTTTTTCGAAAGAGAGC |
| | | ATTCTTCCGAAACGCAACAGCGACAAGCTGATCGCGCGTAAAAAGGACTGGGATCCGAAGAAGTATGGT |
| | | GGCTTCGATAGCCCGACCGTTGCCTATAGCGTTCTGGTAGTTGCTAAGGTTGAGAAAGGCAAAAGTAAA |
| | | AAGCTGAAATCCGTGAAAGAATTGTTGGGCATTACCATTATGGAACGTAGCTCTTTCGAGAAGAACCCG |
| | | ATTGATTTCCTGGAGGCAAAAGGCTACAAAGAGGTCAAAAAGGATCTAATTATTAAGCTGCCAAAGTAC |
| | | AGCCTGTTCGAGCTCGAAAATGGACGTAAACGTATGCTGGCGTCAGCGGGTGAACTGCAGAAGGGTAAT |
| | | GAACTGGCGCTGCCGAGCAAATACGTGAATTTTCTGTATCTCGCTAGCCATTATGAAAAACTAAAGGGT |
| | | TCCCCGGAAGACAACGAGCAGAAGCAGCTTTTTGTTGAGCAGCATAAACACTACCTGGACGAGATCATT |
| | | GAGCAAATCAGCGAGTTCTCTAAGCGTGTCATTTTAGCGGATGCTAATCTTGACAAAGTACTCAGCGCG |
| | | TATAACAAACACCGGGACAAGCCGATCCGTGAACAAGCGGAGAACATTATTCACTTGTTTACCCTGACG |
| | | AACCTGGGTGCTCCGGCGGCATTTAAATACTTCGATACCACCATTGACCGCAAGAGATACACCAGCACC |
| | | AAAGAGGTGCTGGATGCTACCCTGATTCATCAAAGCATCACCGGCCTGTATGAAACCCGTATCGATCTT |
| | | TCCCAATTAGGCGGCGACAGCCCGGTTCGCTCTAGCGGTGGCTCGAGTGGCGGGTCGAGCGGTTCCGAA |
| | | ACCCCCGGCACCAGCGAGTCAGCCACCCCGGAAAGCTCTGGCGGGTCCTCCGGCGGCTCCTCGCGTCCG |
| | | GGTGAGCGCCCTTTCCAATGCCGTATCTGCATGCGTAACTTTTCTAATATGAGCAATCTGACCCGTCAC |
| | | ACCCGCACGCATACAGGCGAGAAGCCGTTTCAATGTAGAATTTGCATGCGTAACTTCAGCGATCGTAGC |
| | | GTCCTCAGGCGCCACCTTCGCACCCACACCGGCAGCCAAAAGCCATTTCAGTGCCGGATCTGCATGCGT |
| | | AATTTCTCCGACCCGTCCAACCTGGCGCGTCACACCCGTACCCATACCGGTGAGAAACCATTCCAGTGT |
| | | CGTATTTGCATGCGTAACTTCAGCGATCGTAGCAGCTTGCGCCGCCACCTGCGTACCCATACTGGTTCC |
| | | CAGAAGCCGTTTCAGTGCCGTATCTGCATGAGGAACTTCTCCCAGAGCGGTACCCTGCACCGACATACT |
| | | AGAACGCACACGGGCGAAAAGCCGTTCCAATGCCGCATCTGTATGCGTAACTTCAGCCAACGTCCGAAT |
| | | CTTACTCGTCACCTGCGCACCCATCTGCGCGGTTCGGGCAGCGCGGGTAGCGCGGCAGGTTCCGGCGAA |
| | | TTCCCGAAAAAGAAACGTAAAGTGTAA |
| 51 | Cas9:zinc | MHHHHHHASPPKKKRKVGSMDKKYSIGLDIGTNSVGWAVITDDYKVPSKKFKVLGNTDRHSIKKNLIGA |
| | finger | LLFGSGETAEATRLKRTARRRYTRRKNRICYLQEIFSNEMAKVDDSFFHRLEESFLVEEDKKHERHPIF |
| | fusion | GNIVDEVAYHEKYPTIYHLRKKLADSTDKADLRLIYLALAHMIKFRGHFLIEGDLNPDNSDVDKLFIQL |
| | protein for | VQIYNQLFEENPINASRVDAKAILSARLSKSRRLENLIAQLPGEKRNGLFGNLIALSLGLTPNFKSNFD |
| | E. coli | LAEDAKLQLSKDTYDDDLDNLLAQIGDQYADLFLAAKNLSDAILLSDILRVNSEITKAPLSASMIKRYD |
| | expression | EHHQDLTLLKALVRQQLPEKYKEIFFDQSKNGYAGYIDGGASQEEFYKFIKPILEKMDGTEELLVKLNR |
| | AA | EDLLRKQRTFDNGSIPHQIHLGELHAILRRQEDFYPFLKDNREKIEKILTFRIPYYVGPLARGNSRFAW |
| | | MTRKSEETITPWNFEEVVDKGASAQSFIERMTNFDKNLPNEKVLPKHSLLYEYFTVYNELTKVKYVTEG |
| | | MRKPAFLSGEQKKAIVDLLFKTNRKVTVKQLKEDYFKKIECFDSVEISGVEDRFNASLGAYHDLLKIIK |
| | | DKDFLDNEENEDILEDIVLTLTLFEDRGMIEERLKTYAHLFDDKVMKQLKRRRYTGWGRLSRKLINGIR |
| | | DKQSGKTILDFLKSDGFANANFMQLIHDDSLTFKEDIQKAQVSGQGHSLHEQIANLAGSPAIKKGILQT |
| | | VKIVDELVKVMGHKPENIVIEMARENQTTQKGQKNSRERMKRIEEGIKELGSQILKEHPVENTQLQNEK |
| | | LYLYYLQNGRDMYVDQELDINRLSDYDVDHIVPQSFIKDDSIDNKVLIRSDKNRGKSDNVPSEEVVKKM |
| | | KNYWRQLLNAKLITQRKFDNLTKAERGGLSELDKAGFIKRQLVETRQITKHVAQILDSRMNTKYDENDK |
| | | LIREVKVITLKSKLVSDFRKDFQFYKVREINNYHHAHDAYLNAVVGTALIKKYPKLESEFVYGDYKVYD |
| | | VRKMIAKSEQEIGKATAKYFFYSNIMNFFKTEITLANGEIRKRPLIETNGETGEIVWDKGRDFATVRKV |
| | | LSMPQVNIVKKTEVQTGGFSKESILPKRNSDKLIARKKDWDPKKYGGFDSPTVAYSVLVVAKVEKGKSK |
| | | KLKSVKELLGITIMERSSFEKNPIDFLEAKGYKEVKKDLIIKLPKYSLFELENGRKRMLASAGELQKGN |
| | | ELALPSKYVNFLYLASHYEKLKGSPEDNEQKQLFVEQHKHYLDEIIEQISEFSKRVILADANLDKVLSA |
| | | YNKHRDKPIREQAENIIHLFTLTNLGAPAAFKYFDTTIDRKRYTSTKEVLDATLIHQSITGLYETRIDL |
| | | SQLGGDSPVRSSGGSSGGSSGSETPGTSESATPESSGGSSGGSSRPGERPFQCRICMRNFSNMSNLTRH |
| | | TRTHTGEKPFQCRICMRNFSDRSVLRRHLRTHTGSQKPFQCRICMRNFSDPSNLARHTRTHTGEKPFQC |
| | | RICMRNFSDRSSLRRHLRTHTGSQKPFQCRICMRNFSQSGTLHRHTRTHTGEKPFQCRICMRNFSQRPN |
| | | LTRHLRTHLRGSGSAGSAAGSGEFPKKKRKV* |
| 52 | Cas9:zinc | ATGGACTATAAGGACCACGACGGAGACTACAAGGATCATGATATTGATTACAAAGACGATGACGATAAG |
| | finger | ATGGCCCCAAAGAAGAAGCGGAAGGTCGGTATCCACGGAGTCCCAGCAGCCGACAAGAAGTACAGCATC |
| | fusion | GGCCTGGACATCGGCACCAACTCTGTGGGCTGGGCCGTGATCACCGACGAGTACAAGGTGCCCAGCAAG |
| | protein for | AAATTCAAGGTGCTGGGCAACACCGACCGGCACAGCATCAAGAAGAACCTGATCGGAGCCCTGCTGTTC |
| | mammalian | GACAGCGGCGAAACAGCCGAGGCCACCCGGCTGAAGAGAACCGCCAGAAGAAGATACACCAGACGGAAG |
| | expression | AACCGGATCTGCTATCTGCAAGAGATCTTCAGCAACGAGATGGCCAAGGTGGACGACAGCTTCTTCCAC |
| | NT | AGACTGGAAGAGTCCTTCCTGGTGGAAGAGGATAAGAAGCACGAGCGGCACCCCATCTTCGGCAACATC |
| | | GTGGACGAGGTGGCCTACCACGAGAAGTACCCCACCATCTACCACCTGAGAAAGAAACTGGTGGACAGC |
| | | ACCGACAAGGCCGACCTGCGGCTGATCTATCTGGCCCTGGCCCACATGATCAAGTTCCGGGGCCACTTC |
| | | CTGATCGAGGGCGACCTGAACCCCGACAACAGCGACGTGGACAAGCTGTTCATCCAGCTGGTGCAGACC |
| | | TACAACCAGCTGTTCGAGGAAAACCCCATCAACGCCAGCGGCGTGGACGCCAAGGCCATCCTGTCTGCC |
| | | AGACTGAGCAAGAGCAGACGGCTGGAAAATCTGATCGCCCAGCTGCCCGGCGAGAAGAAGAATGGCCTG |
| | | TTCGGAAACCTGATTGCCCTGAGCCTGGGCCTGACCCCCAACTTCAAGAGCAACTTCGACCTGGCCGAG |
| | | GATGCCAAACTGCAGCTGAGCAAGGACACCTACGACGACGACCTGGACAACCTGCTGGCCCAGATCGGC |
| | | GACCAGTACGCCGACCTGTTTCTGGCCGCCAAGAACCTGTCCGACGCCATCCTGCTGAGCGACATCCTG |
| | | AGAGTGAACACCGAGATCACCAAGGCCCCCCTGAGCGCCTCTATGATCAAGAGATACGACGAGCACCAC |
| | | CAGGACCTGACCCTGCTGAAAGCTCTCGTGCGGCAGCAGCTGCCTGAGAAGTACAAAGAGATTTTCTTC |
| | | GACCAGAGCAAGAACGGCTACGCCGGCTACATTGACGGCGGAGCCAGCCAGGAAGAGTTCTACAAGTTC |
| | | ATCAAGCCCATCCTGGAAAAGATGGACGGCACCGAGGAACTGCTCGTGAAGCTGAACAGAGAGGACCTG |
| | | CTGCGGAAGCAGCGGACCTTCGACAACGGCAGCATCCCCCACCAGATCCACCTGGGAGAGCTGCACGCC |
| | | ATTCTGCGGCGGCAGGAAGATTTTTACCCATTCCTGAAGGACAACCGGGAAAAGATCGAGAAGATCCTG |
| | | ACCTTCCGCATCCCCTACTACGTGGGCCCTCTGGCCAGGGGAAACAGCAGATTCGCCTGGATGACCAGA |
| | | AAGAGCGAGGAAACCATCACCCCCTGGAACTTCGAGGAAGTGGTGGACAAGGGCGCTTCCGCCCAGAGC |
| | | TTCATCGAGCGGATGACCAACTTCGATAAGAACCTGCCCAACGAGAAGGTGCTGCCCAAGCACAGCCTG |
| | | CTGTACGAGTACTTCACCGTGTATAACGAGCTGACCAAAGTGAAATACGTGACCGAGGGAATGAGAAAG |
| | | CCCGCCTTCCTGAGCGGCGAGCAGAAAAAGGCCATCGTGGACCTGCTGTTCAAGACCAACCGGAAAGTG |
| | | ACCGTGAAGCAGCTGAAAGAGGACTACTTCAAGAAAATCGAGTGCTTCGACTCCGTGGAAATCTCCGGC |
| | | GTGGAAGATCGGTTCAACGCCTCCCTGGGCACATACCACGATCTGCTGAAAATTATCAAGGACAAGGAC |
| | | TTCCTGGACAATGAGGAAAACGAGGACATTCTGGAAGATATCGTGCTGACCCTGACACTGTTTGAGGAC |
| | | AGAGAGATGATCGAGGAACGGCTGAAAACCTATGCCCACCTGTTCGACGACAAAGTGATGAAGCAGCTG |
| | | AAGCGGCGGAGATACACCGGCTGGGGCAGGCTGAGCCGGAAGCTGATCAACGGCATCCGGGACAAGCAG |
| | | TCCGGCAAGACAATCCTGGATTTCCTGAAGTCCGACGGCTTCGCCAACAGAAACTTCATGCAGCTGATC |
| | | CACGACGACAGCCTGACCTTTAAAGAGGACATCCAGAAAGCCCAGGTGTCCGGCCAGGGCGATAGCCTG |
| | | CACGAGCACATTGCCAATCTGGCCGGCAGCCCCGCCATTAAGAAGGGCATCCTGCAGACAGTGAAGGTG |
| | | GTGGACGAGCTCGTGAAAGTGATGGGCCGGCACAAGCCCGAGAACATCGTGATCGAAATGGCCAGAGAG |
| | | AACCAGACCACCCAGAAGGGACAGAAGAACAGCCGCGAGAGAATGAAGCGGATCGAAGAGGGCATCAAA |
| | | GAGCTGGGCAGCCAGATCCTGAAAGAACACCCCGTGGAAAACACCCAGCTGCAGAACGAGAAGCTGTAC |
| | | CTGTACTACCTGCAGAATGGGCGGGATATGTACGTGGACCAGGAACTGGACATCAACCGGCTGTCCGAC |
| | | TACGATGTGGACCATATCGTGCCTCAGAGCTTTCTGAAGGACGACTCCATCGACAACAAGGTGCTGACC |
| | | AGAAGCGACAAGAACCGGGGCAAGAGCGACAACGTGCCCTCCGAAGAGGTCGTGAAGAAGATGAAGAAC |
| | | TACTGGCGGCAGCTGCTGAACGCCAAGCTGATTACCCAGAGAAAGTTCGACAATCTGACCAAGGCCGAG |
| | | AGAGGCGGCCTGAGCGAACTGGATAAGGCCGGCTTCATCAAGAGACAGCTGGTGGAAACCCGGCAGATC |
| | | ACAAAGCACGTGGCACAGATCCTGGACTCCCGGATGAACACTAAGTACGACGAGAATGACAAGCTGATC |
| | | CGGGAAGTGAAAGTGATCACCCTGAAGTCCAAGCTGGTGTCCGATTTCCGGAAGGATTTCCAGTTTTAC |
| | | AAAGTGCGCGAGATCAACAACTACCACCACGCCCACGACGCCTACCTGAACGCCGTCGTGGGAACCGCC |
| | | CTGATCAAAAAGTACCCTAAGCTGGAAAGCGAGTTCGTGTACGGCGACTACAAGGTGTACGACGTGCGG |
| | | AAGATGATCGCCAAGAGCGAGCAGGAAATCGGCAAGGCTACCGCCAAGTACTTCTTCTACAGCAACATC |
| | | ATGAACTTTTTCAAGACCGAGATTACCCTGGCCAACGGCGAGATCCGGAAGCGGCCTCTGATCGAGACA |
| | | AACGGCGAAACCGGGGAGATCGTGTGGGATAAGGGCCGGGATTTTGCCACCGTGCGGAAAGTGCTGAGC |
| | | ATGCCCCAAGTGAATATCGTGAAAAAGACCGAGGTGCAGACAGGCGGCTTCAGCAAAGAGTCTATCCTG |
| | | CCCAAGAGGAACAGCGATAAGCTGATCGCCAGAAAGAAGGACTGGGACCCTAAGAAGTACGGCGGCTTC |
| | | GACAGCCCCACCGTGGCCTATTCTGTGCTGGTGGTGGCCAAAGTGGAAAAGGGCAAGTCCAAGAAACTG |
| | | AAGAGTGTGAAAGAGCTGCTGGGGATCACCATCATGGAAAGAAGCAGCTTCGAGAAGAATCCCATCGAC |
| | | TTTCTGGAAGCCAAGGGCTACAAAGAAGTGAAAAAGGACCTGATCATCAAGCTGCCTAAGTACTCCCTG |
| | | TTCGAGCTGGAAAACGGCCGGAAGAGAATGCTGGCCTCTGCCGGCGAACTGCAGAAGGGAAACGAACTG |
| | | GCCCTGCCCTCCAAATATGTGAACTTCCTGTACCTGGCCAGCCACTATGAGAAGCTGAAGGGCTCCCCC |
| | | GAGGATAATGAGCAGAAACAGCTGTTTGTGGAACAGCACAAGCACTACCTGGACGAGATCATCGAGCAG |
| | | ATCAGCGAGTTCTCCAAGAGAGTGATCCTGGCCGACGCTAATCTGGACAAAGTGCTGTCCGCCTACAAC |
| | | AAGCACCGGGATAAGCCCATCAGAGAGCAGGCCGAGAATATCATCCACCTGTTTACCCTGACCAATCTG |
| | | GGAGCCCCTGCCGCCTTCAAGTACTTTGACACCACCATCGACCGGAAGAGGTACACCAGCACCAAAGAG |
| | | GTGCTGGACGCCACCCTGATCCACCAGAGCATCACCGGCCTGTACGAGACACGGATCGACCTGTCTCAG |
| | | CTGGGAGGCGACTCCCCCGTCCGGAGTAGCGGGGGATCTTCTGGCGGCAGTTCCGGTTCTGAAACCCCC |
| | | GGGACTTCTGAGTCCGCCACCCCCGAATCCAGCGGAGGGTCTTCCGGAGGGTCTTCAAGACCAGGTGAA |
| | | CGCCCATTCCAGTGTCGCATCTGCATGCGCAACTTTAGCAATATGAGCAATTTGACCAGACATACAAGA |
| | | ACACATACAGGTGAGAAGCCCTTCCAGTGTCGAATATGTATGAGGAATTTCTCAGATAGGTCCGTTCTG |
| | | AGGCGGCATCTCAGAACACATACGGGTTCCCAGAAACCATTCCAATGTCGGATATGTATGCGGAACTTC |
| | | AGTGACCCATCCAATCTCGCTAGGCATACGCGGACCCACACAGGTGAAAAACCGTTTCAATGCCGCATA |
| | | TGCATGCGGAATTTCTCTGACCGATCAAGCCTCAGAAGACACCTCCGAACTCATACCGGCAGCCAGAAA |
| | | CCTTTCCAATGTAGGATATGTATGCGAAACTTCTCTCAATCCGGAACCCTTCATCGACATACCAGAACC |
| | | CATACAGGAGAAAAGCCTTTCCAGTGTAGAATTTGCATGAGGAACTTTTCCCAACGCCCAAATCTTACG |
| | | CGGCATCTCAGGACACATCTGAGGGGGTCTGGCAGCGCAGGCAGTGCAGCGGGGTCAGGAGAGTTCCCC |
| | | AAAAAAAAGCGCAAAGTATAA |
| 53 | Cas9:zinc | MDYKDHDGDYKDHDIDYKDDDDKMAPKKKRKVGIHGVPAADKKYSIGLDIGTNSVGWAVITDEYKVPSK |
| | finger | KFKVLGNTDRHSIKKNLIGALLFDSGETAEATRLKRTARRRYTRRKNRICYLQEIFSNEMAKVDDSFFH |
| | fusion | RLEESFLVEEDKKHERHPIFGNIVDEVAYHEKYPTIYHLRKKLVDSTDKADLRLIYLALAHMIKFRGHF |
| | protein for | LIEGDLNPDNSDVDKLFIQLVQTYNQLFEENPINASGVDAKAILSARLSKSRRLENLIAQLPGEKKNGL |
| | mammalian | FGNLIALSLGLTPNFKSNFDLAEDAKLQLSKDTYDDDLDNLLAQIGDQYADLFLAAKNLSDAILLSDIL |
| | expression | RVNTEITKAPLSASMIKRYDEHHQDLTLLKALVRQQLPEKYKEIFFDQSKNGYAGYIDGGASQEEFYKF |
| | AA | IKPILEKMDGTEELLVKLNREDLLRKQRTFDNGSIPHQIHLGELHAILRRQEDFYPFLKDNREKIEKIL |
| | | TFRIPYYVGPLARGNSRFAWMTRKSEETITPWNFEEVVDKGASAQSFIERMTNFDKNLPNEKVLPKHSL |
| | | LYEYFTVYNELTKVKYVTEGMRKPAFLSGEQKKAIVDLLFKTNRKVTVKQLKEDYFKKIECFDSVEISG |
| | | VEDRFNASLGTYHDLLKIIKDKDFLDNEENEDILEDIVLTLTLFEDREMIEERLKTYAHLFDDKVMKQL |
| | | KRRRYTGWGRLSRKLINGIRDKQSGKTILDFLKSDGFANRNFMQLIHDDSLTFKEDIQKAQVSGQGDSL |
| | | HEHIANLAGSPAIKKGILQTVKVVDELVKVMGRHKPENIVIEMARENQTTQKGQKNSRERMKRIEEGIK |
| | | ELGSQILKEHPVENTQLQNEKLYLYYLQNGRDMYVDQELDINRLSDYDVDHIVPQSFLKDDSIDNKVLT |
| | | RSDKNRGKSDNVPSEEVVKKMKNYWRQLLNAKLITQRKFDNLTKAERGGLSELDKAGFIKRQLVETRQI |
| | | TKHVAQILDSRMNTKYDENDKLIREVKVITLKSKLVSDFRKDFQFYKVREINNYHHAHDAYLNAVVGTA |
| | | LIKKYPKLESEFVYGDYKVYDVRKMIAKSEQEIGKATAKYFFYSNIMNFFKTEITLANGEIRKRPLIET |
| | | NGETGEIVWDKGRDFATVRKVLSMPQVNIVKKTEVQTGGFSKESILPKRNSDKLIARKKDWDPKKYGGF |
| | | DSPTVAYSVLVVAKVEKGKSKKLKSVKELLGITIMERSSFEKNPIDFLEAKGYKEVKKDLIIKLPKYSL |
| | | FELENGRKRMLASAGELQKGNELALPSKYVNFLYLASHYEKLKGSPEDNEQKQLFVEQHKHYLDEIIEQ |
| | | ISEFSKRVILADANLDKVLSAYNKHRDKPIREQAENIIHLFTLTNLGAPAAFKYFDTTIDRKRYTSTKE |
| | | VLDATLIHQSITGLYETRIDLSQLGGDSPVRSSGGSSGGSSGSETPGTSESATPESSGGSSGGSSRPGE |
| | | RPFQCRICMRNFSNMSNLTRHTRTHTGEKPFQCRICMRNFSDRSVLRRHLRTHTGSQKPFQCRICMRNF |
| | | SDPSNLARHTRTHTGEKPFQCRICMRNFSDRSSLRRHLRTHTGSQKPFQCRICMRNFSQSGTLHRHTRT |
| | | HTGEKPFQCRICMRNFSQRPNLTRHLRTHLRGSGSAGSAAGSGEFPKKKRKV* |
| 54 | g526 | TCAGGGTTCTGGATATCTGTGTTTTAGAGCTAGAAATAGCAAGTTAAAATAAGGCTAGTCCGTTATCAA |
| | sgRNA | CTTGAAAAAGTGGCACCGAGTCGGTGCTTTT |

What is claimed is:

1. A composition comprising a cssDNA molecule, wherein the cssDNA molecule comprises a co-location sequence, a selectable sequence, a packaging signal, and a designed sequence, wherein the co-location sequence is capable of associating with a synthetic nuclease.

2. The composition according to claim 1, wherein the synthetic nuclease is associated with a synthetic binding domain and the cssDNA associates with the synthetic nuclease through the synthetic binding domain binding to the co-location sequence.

3. A composition comprising:
a cell, wherein the cell comprises
a synthetic nuclease, wherein the synthetic nuclease is associated with a synthetic binding domain, wherein the synthetic binding domain is capable of associating with a co-location sequence, and
a cssDNA molecule, wherein the cssDNA molecule comprises the co-location sequence.

4. The composition according to claim 3, wherein the cssDNA molecule further comprises a designed sequence.

5. The composition according to claim 3 or 4, wherein the cell is a eukaryotic cell.

6. The composition according to claim 5, wherein the cell is a mammalian cell.

7. The composition according to claim 5, wherein the cell is selected from a yeast cell or a fungal cell.

8. The composition according to claim 3 or 4, wherein the cell is an algal cell.

9. The composition according to claim 3 or 4, wherein the cell is a bacterial cell.

10. The composition according to any one of claims 3-9, wherein the cssDNA molecule does not contain a packaging signal.

11. The composition according to any one of claims 3-9, wherein the cssDNA molecule further comprises a packaging signal.

12. The composition according to any one of claims 3-11, wherein the cssDNA molecule comprises a selectable sequence.

13. The composition according to any one of claims 2-12, wherein the synthetic nuclease is linked to a first protein and the synthetic binding domain is linked to a second protein, wherein the first protein and second protein form a dimer, thereby associating the synthetic nuclease with the synthetic binding domain.

14. The composition according to claim 13, wherein the first protein comprises a first portion of a GFP protein and the second protein comprises a second portion of a GFP protein.

15. The composition according to claim 14, wherein the first portion of the GFP protein comprises beta strand peptides 1-10 of GFP and the second portion of the GFP protein comprises beta strand peptide 11 of the GFP protein, and wherein GFP beta strand peptide 11 and GFP beta strand peptides 1-10 associate with each other to form a complete GFP protein.

16. The composition according to any one of claims 2-12, wherein the synthetic nuclease is linked to the synthetic binding domain through a linker sequence.

17. The composition according to any one of claims 2-12, wherein the synthetic nuclease is linked to the synthetic binding domain through a flexible linker.

18. The composition according to claim 17, wherein the linker comprises an amino acid sequence according to SEQ ID NO: 20 or 21.

19. The composition according to any one of claims 2-18, wherein the synthetic binding domain comprises a DNA binding domain.

20. The composition according to any one of claims 2-18, wherein the synthetic binding domain comprises an amino acid sequence capable of forming a protein:protein interaction with a second amino acid sequence.

21. The composition according to claim 20, wherein the second amino acid sequence additionally comprises a DNA binding domain.

22. The composition according to claim 21, wherein the DNA binding domain binds to the co-location sequence.

23. The composition according to any one of claims 1-22, wherein the co-location sequence comprises a Zinc Finger DNA binding motif.

24. The composition according to any one of claims 1-23, wherein the co-location sequence comprises a Zinc Finger DNA binding motif sequence that is predicted to form a stem and loop secondary structure.

25. The composition according to any one of claims 1-24, wherein the co-location sequence comprises a Zinc Finger 5 DNA binding motif (ZFDBM5).

26. The composition according to claim 25, wherein the ZFDBM5 comprises a sequence that includes a nucleic acid sequence according to SEQ ID NO: 46.

27. The composition according to claim 25 or 26, wherein the ZFDBM5 sequence is predicted to form a stem and loop secondary structure that comprises a nucleic acid sequence according to SEQ ID NO: 24 or 26.

28. The composition according to any one of claims 25-27, wherein the ZFDBM5 comprises a nucleic acid sequence according to SEQ ID NO: 23 or 25.

29. The composition according to any one of claims 2-28, wherein the synthetic binding domain is a dsDNA binding domain.

30. The composition according to any one of claims 2-29, wherein the synthetic binding domain is a ssDNA binding domain.

31. The composition according to claim 29 or 30, wherein the synthetic binding domain is derived from an amino acid sequence selected from the group consisting of: a Cas protein, a zinc finger nuclease (ZFN), a meganuclease, a homing endonuclease, a transcription factor like effector nuclease (TALE); and a restriction endonuclease.

32. The composition according to claim 31, wherein the synthetic binding domain comprises a ZF.

33. The composition according to claim 32, wherein the ZF comprises ZF5 (e.g., as shown in SEQ ID NO: 22).

34. The composition according to any one of claims 1-33, wherein the synthetic nuclease is selected from the group consisting of: a Cas protein, a zinc finger nuclease (ZFN), a meganuclease, a homing endonuclease, a transcription factor like effector nuclease (TALEN), or another nuclease capable of creating double- or single-strand DNA breaks.

35. The composition according to claim 34, wherein the synthetic nuclease comprises a Cas9 protein.

36. The composition according to claim 35, wherein the Cas9 protein comprises an amino acid sequence according to SEQ ID NO: 16 or 18.

37. The composition according to any one of claims 16-36, wherein the synthetic binding domain comprises ZF5 and the synthetic nuclease comprises Cas9, wherein the ZF5 is linked to the Cas9 through a flexible linker.

38. The composition according to claim 37, wherein the ZF5 linked to a Cas9 nuclease comprises an amino acid sequence comprises SEQ ID NO: 51 or 53.

39. A vector comprising a nucleic acid sequence that encodes a ZF5-Cas9 fusion protein comprising an amino acid sequence according to SEQ ID NO: 51 or 53.

40. The vector according to claim 39, wherein the vector comprises a nucleic acid sequence according to SEQ ID NO: 6 or 7.

41. The composition according to any one of claims 1-40, wherein the cssDNA molecule comprises at least one region of homology, wherein the region of homology targets a region in the genome of a target cell.

42. The composition according to claim 41, wherein the region in the genome of a target cell comprises a region within or including the TRAC locus (NC_000014.9).

43. The composition according to claim 42, wherein the region of homology comprises two homology arms comprising an upstream homology arm (UHA) and a downstream homology arm (DHA), wherein the UHA comprises a nucleic acid sequence according to SEQ ID NO: 27 and the DHA comprises a nucleic acid sequence according to SEQ ID NO: 28.

44. The composition according to any one of claims 1-43, wherein the cssDNA molecule comprises a nucleic acid sequence according to any one of SEQ ID NOs: 3-5, and 10.

45. The composition according to any one of claims 1-44, further comprising a gRNA.

46. The composition according to claim 45, wherein the gRNA sequence comprises a sequence that is targeted to a region within or including the TRAC locus.

47. The composition according to claim 46, wherein the region within or including the TRAC locus comprises the g526 protospacer sequence according to SEQ ID NO: 42.

48. The composition according to claim 46 or 47, wherein the gRNA sequence comprises SEQ ID NO: 29.

49. The composition according to any one of claims 1-2 and 4-48, wherein the designed sequence functions to alter the genome of a mammalian cell.

50. The composition according to claim 49, wherein the designed sequence comprises a control sequence.

51. The composition according to claim 50, wherein the control sequence is selected from the group consisting of: introns, promoters, DNA binding sites, RNA binding sites, repressor binding sites, enhancer binding sites, transcription modifiers, and translation modifiers.

52. The composition according to claim 51, wherein the designed sequence alters a coding sequence in a gene.

53. The composition according to any one of claims 1-2 and 4-52, wherein the designed sequence is greater than 3 kb.

54. The composition according to claim 53, wherein the designed sequence comprises a coding region within any one of genes listing in Example 7.

55. A method of engineering a target dsDNA in a cell comprising: contacting a target dsDNA with a synthetic nuclease and a cssDNA comprising a co-location sequence; and culturing the cell.

56. A method of making a kit for editing dsDNA, comprising: selecting a cssDNA comprising a co-location sequence; selecting an synthetic nuclease wherein the synthetic nuclease comprises a synthetic binding domain and wherein the synthetic binding domain associates with the co-location sequence; and assembling the cssDNA and synthetic nuclease into a kit.

57. A kit made by the method according to claim 56.

58. A fusion protein comprising a Cas9 nuclease linked to a zinc finger (ZF) via a flexible linker.

59. The fusion protein according to claim 58, wherein the ZF comprises ZF5.

60. A complex comprising:
a Cas nuclease linked to a ZF via a flexible linker or through a protein:protein interaction; and
a cssDNA molecule comprising a designed sequence and a co-location sequence;
wherein the co-location sequence comprises a ZF DNA binding motif (ZFDBM); and
wherein the the ZF binds to the ZFDBM, thereby forming the complex.


SEQUENCE LISTING (59 sequences)

SEQ ID NO: 1 - DNA, 4839 bp, synthetic construct
  source 1..4839: mol_type = other DNA
atggcttccccacccaagaaaaaacgtaaggtagggtctatggacaaaaaatatagcatcgggctggatatcggaaccaatagcgttgggtgggccgtcattaccgatgactataaggtcccttctaaaaaattcaaggttttaggaaataccgaccgccattcaattaagaaaaatttgatcggcgcgttgctgtttggtagtggggaaactgcggaggcaactcgccttaagcgtacagcacgtcgccgctacacccgccgcaagaatcgtatctgctacttgcaggagatctttagtaatgagatggccaaagtagacgattcattttttcaccgcctggaagaatctttcttggtagaggaggacaaaaagcacgagcgtcatccaatttttggtaacattgtagatgaagtggcttatcatgaaaagtaccccacgatttaccatttacgcaagaagttggcagattcaactgataaagcggatcttcgcctgatttaccttgcgctggcacatatgatcaagttccgcggtcacttcttgatcgagggcgatctgaatccagacaattccgacgtcgacaaattgtttatccaattggttcagatctacaatcaactgttcgaggagaatcccatcaatgccagccgcgtagacgctaaggccattttatcagcgcgtcttagtaaaagtcgtcgtctggaaaacttaatcgcccagcttcccggggagaagcgtaatgggttattcggcaacttgatcgctctttccctgggattgaccccgaacttcaaatcgaacttcgaccttgctgaggatgcaaagttgcaattgtctaaagatacctacgacgatgatctggataacctgttggcgcagatcggcgaccaatatgccgacctttttcttgccgcgaagaacctttctgatgccattttgctgtcagatatcttgcgtgtgaattctgaaatcacgaaggcccccttaagcgcgtcaatgattaagcgctatgatgagcatcatcaggacctgacattattgaaagccctggttcgccaacagttgccggagaaatataaagaaatttttttcgatcaaagcaaaaatgggtacgcgggctatattgacggcggtgcttcccaggaagagttttacaaattcatcaaaccaatcttggaaaaaatggacggcaccgaggagttactggtcaaactgaaccgtgaagatctgcttcgcaaacagcgcaccttcgataatggcagcattccccaccagattcacctgggcgaattacatgccattttgcgtcgtcaggaagacttttatccctttttgaaagataaccgtgaaaaaattgaaaagatcctgaccttccgcattccgtactatgtcggacccctggcccgcggtaactcacgtttcgcgtggatgactcgcaagtccgaggaaacaattacgccatggaacttcgaggaagtggtggacaaaggggcgtctgctcagtccttcattgagcgtatgactaattttgacaagaacctgccgaacgagaaggtcttaccgaagcatagtttgctgtatgagtacttcacagtgtacaacgaacttactaaggtgaaatatgtgactgaagggatgcgtaagcccgcctttttaagtggtgagcaaaagaaagcaatcgtcgatttgctttttaagacaaatcgtaaagtcacagttaaacagctgaaggaggactactttaaaaaaattgaatgtttcgattcagtcgaaatttcgggagtagaggatcgttttaacgcgagcctgggagcttaccatgatttacttaagatcattaaagataaagattttttggacaacgaggaaaacgaagatatcttggaagatatcgtgttgacattaaccctgtttgaggaccgcggcatgatcgaggaacgtctgaaaacttacgcgcatttgttcgatgacaaagtcatgaaacagctgaaacgccgtcgctacactggctggggacgtcttagccgtaagcttattaatggtatccgcgacaaacaatccggcaagaccatcctggactttttaaaaagcgacggattcgctaacgccaactttatgcaattaatccatgacgacagtcttaccttcaaagaagacatccaaaaagctcaagtctcgggccaaggtcactctttgcatgagcaaattgcgaacttagccggctcaccagctattaagaaagggatccttcagactgttaagatcgtagatgagttagtgaaagtaatgggacataagccagaaaatattgtcatcgaaatggcacgcgagaaccagacaacacagaagggacaaaaaaactcgcgtgaacgtatgaagcgcattgaagaagggatcaaagaattagggtctcagattttgaaagaacatccggttgagaatacgcaactgcaaaatgaaaagttatatttgtattatttgcaaaacggacgcgacatgtacgtagatcaggaactggatatcaaccgtctgtccgattatgatgtagatcacatcgtgccacaatctttcattaaagacgactctatcgacaacaaggtacttacacgttctgataaaaaccgtggtaagtcggacaatgtaccgtccgaggaggtggttaagaaaatgaagaattactggcgtcaattacttaatgccaaattgatcacccaacgtaagttcgataacctgactaaggccgaacgtggggggttatcggaacttgacaaggccggattcattaagcgtcaacttgttgaaacgcgccaaatcacgaaacacgttgctcaaattcttgatagccgcatgaacactaagtacgatgagaatgataagcttatccgtgaagttaaggtgattacccttaagtccaagttggtgtctgacttccgcaaggattttcagttctataaggttcgcgagattaataattaccatcatgcgcacgatgcatatcttaatgccgtggttggcaccgctttaatcaaaaagtatcctaaattggagagtgagttcgtttacggcgactataaagtttacgatgtgcgtaagatgattgctaagtccgaacaggaaatcggtaaggcgacagccaaatatttcttctactcaaacattatgaatttttttaaaacggagattacattagctaatggtgaaatccgtaagcgcccgttgattgagacaaacggcgagaccggcgagattgtctgggataaaggacgtgatttcgccaccgtccgcaaggtcctgagcatgccccaagttaacattgtgaagaagacagaagttcagaccggcggattcagtaaggagtcgatcttgccaaaacgcaatagcgataagttaattgctcgcaagaaagactgggatccgaaaaagtatggagggttcgacagccccacagtcgcgtactctgtacttgttgtagcgaaagttgaaaagggcaaatctaagaaattaaaatccgtaaaagagctgttgggaatcacaattatggagcgcagctcgtttgaaaaaaaccccatcgacttcctggaggcgaagggttataaagaagttaagaaggacttgatcattaaattacctaaatacagtttatttgagttggagaacggtcgtaagcgcatgttagcctcggcaggggagttacaaaagggcaacgaactggcgttaccaagcaagtacgtcaatttcctttacttagcctcgcattatgagaagttgaaaggaagtcctgaagacaacgagcagaagcaattatttgtggagcaacataaacattaccttgacgagatcatcgagcagatctcggagttttcgaagcgcgtaattttagcggatgccaacttagacaaggtattgtcggcctataataaacaccgtgacaagcctattcgcgagcaagcagagaacattattcacttgtttaccttaaccaaccttggggctcctgccgctttcaagtatttcgatactacaatcgaccgtaagcgttatacgtcaactaaggaagtgttagacgcgacactgattcatcaatccatcactgggttatacgaaacccgtattgacttatctcagcttggcggggattcacccgtccgttcatccggaggtagctcagggggctcgtctggctccgagactcccggcacctccgaatcggcaacgccggagtcaagcggaggcagctctggaggcagctcgcgccccggtgagcgtccctttcaatgccgtatttgtatgcgcaacttctcaaatatgtcgaacctgacacgtcatacccgtacacatactggcgagaagccatttcagtgtcgcatttgcatgcgcaactttagtgatcgttcagtattacgccgccaccttcgcacgcacactgggtctcagaagcctttccaatgccgcatctgcatgcgtaacttttcggatccctccaatctggcacgccatacacgtacccatacgggggaaaagccttttcagtgtcgcatttgtatgcgcaacttttccgatcgctcttcgcttcgccgccatttacgcacgcatacgggctctcaaaaacccttccagtgccgcatctgcatgcgtaacttttcgcagtcagggacgttgcaccgtcacacacgcacccataccggtgaaaagccctttcagtgtcgtatttgcatgcgcaattttagtcagcgccccaacttaacccgtcacttgcgcacacatctgcgtggaagtggatctgcggggtcggcagcgggatccggagaatttcccaagaaaaagcgtaaagtttaa

SEQ ID NO: 2 - DNA, 9966 bp, synthetic construct
  source 1..9966: mol_type = other DNA
tgaagcagtcgacgccgaagagggtccgaggtattccttcggcgtcgactgcttcagcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggtttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagatctatggatttatcaatgaactgataaaattcagtccagaaatttgtttctaaggacattgtttctttttgaaagtccaccctatctcgtaattcattcccagatcaactctccaaacgaatataatcctttactcctttttcatttcctgtaaagtatcgccaggaggtaaatctatgactttaaccgctagttcatcatcccgcgcagtgacgaattcgccggttgtagttgcacttgactatcataatcgcgatgacgcattagcttttgtcgacaaaattgatccacgcgattgtcgtcttaaagtaggtaaggagatgtttacgctttttggaccgcaattcgtgcgcgaactgcaacaacgtggttttgacattttccttgaccttaagtttcacgatatcccgaacactgcagctcatgcggtagctgcggctgctgaccttggtgtttggatggtaaatgttcacgcctctggtggggcacgtatgatgaccgcagctcgtgaagcacttgtccctttcggaaaggatgccccattgttaattgccgtcactgtccttacttcgatggaggcttcagacttagtcgatttaggcatgacattgtcaccagccgattatgcggaacgtttggcggcgttgactcaaaaatgtgggttagacggcgtcgtttgtagcgcccaggaagctgttcgctttaaacaagtcttcggacaggagttcaagttagttactcccggaatccgcccccagggtagtgaggcaggagaccaacgccgcatcatgacacctgagcaggcactttccgctggggtcgactatatggtaatcggccgtcccgttactcaatcagtcgaccctgcacaaacattgaaggcaatcaatgcttccctgcaacgcagcgcatgactcggtaccaaattccagaaaagaggcctcccgaaaggggggccttttttcgttttggtccggtctgggaagggaaaagcattactatctatcttgaatattcatgtttctctaggtcaaacacattaaaatttgactttaatcattcaatgggtattgtaaaatgccttctatgtgactatcactctataaaatgttagactgagtatgaagtgtgagatagattcctgtcctgtcctcaagtggtataaaaactagacaaaggtactgaactattgtaaattaagcagccagaaaactattttagtattgaccagttgatgatgtcaatggacacaatagagattcagaggggatagggtgctggctgagaagttggcctagactgagaagtttcctgtggtacaaaggattcattgagccctgaaggatggataagatctgtatgggcagagaaaggagagaagggaagttctgggcgtagggaacgacaagaaagaaggcatgatcttgggaataatcaaggcacatgcaaagtagcctaagtatacatctgataataaaattggttgaaaagtagtcagagaagatgtctttttaggcatggaaaaaggaaatactagagcattcaacagaatacagaaattagggccagggccagccattgggaaactgagaatccgatttagagatgcagactagaagtgaaggtgagagcagccagctatggtgccgcagacctcccctctccttcctcagtgggctctgagaggggtcatcccacaccttagaggaggagaaacctaagggattctgtaatagagacacggggcatggtatgaaagtattacctcccagttgcaatttggcaaaggaaccagagtttccacttctccccgtacgtctgcccatgcccacagtttcctgatgctcactaaagcctcggtgggacccagagtgactgtcactaattctgatttctgggtccctagtgcccaaacacgggggacagatttaatggtaaggaagctttcaatcactgctgtgtccctagggatctaaagcactagagcacatgtgcttctgcagttcattttgaatttaaaggacagcttaggatctagaatagctgaatttccacctcaaaacattggttccgtcttgccaagcctaccttctgatatcatcagtgatgggatgtgtttttcttactagggtagaataggatgtctctccccaaaggactctggcagacagacccctaaacacctccaaattaaaagcggcaaagagataaggttgaactagacgtacatggggataaaaagtataaaaggtacatgggaatgaaaggataaaaaggctaaaaaaattaagtacctctaactcagcccctgttgccatttctcagagtcttgtgttctgtggcattgcgctttctagaccaacagtgtccaatagaactttctgtggcaatggaaatgtcctgtcaatctgcactgtcccatacaatagccaccagctacatgtggctattgagctcttgaaatgaagtttccatttttaattgaaaacattttatttcacattgactaatttttatttcaacagccacatgtagctagagactattataccagacagagcagcctagatcttctccagtctgacacccaccagccccaggacttgagtgagtgtttaaccaggactcaaagttgggtttctgccccacaaggccaccccctttcctctttaaagccaacctgcatctggtggcccctgatcccctgccttgaggatcggcacttccagactcctctccccctctgcagtgctgtccagtacccccactgatgactaacaatcagggggatgtgttggtagagctaatggctttctgtctgtcccttcccagcaaaggaactatgccttagggccttcacccagagtgatgtcaggctgcccaagcatgaggagggaagtaggcagaatcctctggagccaaagctctggatgtctctcccctctgaccatggagcccacccctgctccactgctccagggacagccctatgctgcaggcagctctgcccccactcagcatcccaggggctgatttctttggttttggatccagctggatgtctgcattgccgaggccaccagggctggctcagcaactgtcggggaatcaccagggtctgagaaatcttgtgcgcatgtgaggggctgtgggagcagagaaccactgggtgggaaattctaatccccaccctgctggaaactctctgggtggccccaacatgctaatcctccggcaaacctctgtttcctcctcaaaaggcaggaggtcggaaagaataaacaatgagagtcacattaaaaacacaaaatcctacggaaatactgaagaatgagtctcagcactaaggaaaagcctccagcagctcctgctttctgagggtgaaggatagacgctgtggctctgcatgactcactagcactctatcacggccatattctggcagggtcagtggctccaactaacatttgtttggtactttacagtttattaaatagatgtttatatggagaagctctcatttctttctcagaagagcctggctaggaaggtggatgaggcaccatattcattttgcaggtgaaattcctgagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagcgtctcgaaggggatcctactgacgaggggcttgtccaaacaagcccgggcatgcctaaacatgccctgatgcaatcctgacgtcgtgaagcaatgattatgcaatttgagcatgtccagactagccctgacggatgacgcttgacgcaattcctgaggcaagtctgagcttgttcaaacttgtcttgaagaaattatgacggactgacgtatggtgcaatattggggcaatgcttgacgttcgcggtaggcgtgtacggtgggaggtctatataagcagagctcgtttagtgaaccgtcagatcgcctggtgacgccatccacgctgttttgacctccatagaagacaccgggaccgatccagcctctcgacattcgtgccaccatggtctccaaaggagaagaggataacatggcaattatcaaagaatttatgagatttaaggtccacatggaagggtcagtgaacggccacgaatttgaaatagaaggagaaggagagggacgaccatacgaaggcacacagactgcaaaattgaaggtgacaaaggggggtcctctcccatttgcatgggatatactgagtcctcaattcatgtatggtagcaaggcgtatgtaaaacacccagctgatattccggattatctcaaactctcatttccggaagggttcaaatgggaaagagtcatgaattttgaagatggaggtgtagtcacggtaactcaggattccagtctccaagacggagaatttatttacaaagttaagttgaggggtaccaactttccgagtgatggcccggtaatgcaaaagaagactatgggttgggaagccagctctgaaagaatgtatcctgaggacggcgccctcaagggagagattaagcagagactgaaacttaaggacggagggcactacgatgcagaagtaaagacaacctacaaagccaaaaaaccagtacagcttccgggtgcatacaatgtgaacattaaattggatattacctctcataacgaagactacacaatcgtcgaacaatacgagagagccgaaggccgccattcaacaggaggaatggacgagctttacaagtaagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcactcggagacgcgatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaaggagaagagcagcaggcatgagttgaatgaaggaggcagggccgggtcacagggccttctaggccatgagagggtagacagtattctaaggacgccagaaagctgttgatcggcttcaagcaggggagggacacctaatttgcttttcttttttttttttttttttttttttttttttgagatggagttttgctcttgttgcccaggctggagtgcaatggtgcatcttggctcactgcaacctccgcctcccaggttcaagtgattctcctgcctcagcctcccgagtagctgagattacaggcacccgccaccatgcctggctaattttttgtatttttagtagagacagggtttcactatgttggccaggctggtctcgaactcctgacctcaggtgatccacccgcttcagcctcccaaagtgctgggattacaggcgtgagccaccacacccggcctgcttttcttaaagatcaatctgagtgctgtacggagagtgggttgtaagccaagagtagaagcagaaagggagcagttgcagcagagagatgatggaggcctgggcagggtggtggcagggaggtaaccaacaccattcaggtttcaaaggtagaaccatgcagggatgagaaagcaaagaggggatcaaggaaggcagctggattttggcctgagcagctgagtcaatgatagtgccgtttactaagaagaaaccaaggaaaaaatttggggtgcagggatcaaaactttttggaacatatgaaagtacgtgtttatactctttatggcccttgtcactatgtatgcctcgctgcctccattggactctagaatgaagccaggcaagagcagggtctatgtgtgatggcacatgtggccagggtcatgcaacatgtactttgtacaaacagtgtatattgagtaaatagaaatggtgtccaggagccgaggtatcggtcctgccagggccaggggctctccctagcaggtgctcatatgctgtaagttccctccagatctctccacaaggaggcatggaaaggctgtagttgttcacctgcccaagaactaggaggtctggggtgggagagtcagcctgctctggatgctgaaagaatgtctgtttttccttttagaaagttcctgtgatgtcaagctggtcgagaaaagctttgaaacaggtaagacaggggtctagcctgggtttgcacaggattgcggaagtgatgaacccgcaataaccctgcctggatgagggagtgggaagaaattagtagatgtgggaatgaatgatgaggaatggaaacagcggttcaagacctgcccagagctgggtggggtctctcctgaatccctctcaccatctctgactttccattctaagcactttgaggatgagtttctagcttcaatagaccaaggactctctcctaggcctctgtattcctttcaacagctccactgtcaagagagccagagagagcttctgggtggcccagctgtgaaatttctgagtcccttagggatagccctaaacgaaccagatcatcctgaggacagccaagaggttttgccttctttcaagacaagcaacagtactcacataggctgtgggcaatggtcctgtctctcaagaatcccctgccactcctcacacccaccctgggcccatattcatttccatttgagttgttcttattgagtcatccttcctgtggtagcggaactcactaaggggcccatctggacccgaggtattgtgatgataaattctgagcacctaccccatccccagaagggctcagaaataaaataagagccaagtctagtcggtgtttcctgtcttgaaacacaatactgttggccctggaagaatgcacagaatctgtttgtaaggggatatgcacagaagctgcaagggacaggaggtgcaggagctgcaggcctcccccacccagcctgctctgccttggggaaaaccgtgggtgtgtcctgcaggccatgcaggcctgggacatgcaagcccataaccgctgtggcctcttggttttacagatacgaacctaaactttcaaaacctgtcagtgattgggttccgaatcctcctcctgaaagtggccgggtttaatctgctcatgacgctgcggctgtggtccagctgaggtgaggggccttgaagctgggagtggggtttagggacgcgggtctctgggtgcatcctaagctctgagagcaaacctccctgcagggtcttgcttttaagtccaaagcctgagcccaccaaactctcctacttcttcctgttacaaattcctcttgtgcaataataatggcctgaaacgctgtaaaatatcctcatttcagccgcctcagtt

SEQ ID NO: 3 - DNA, 6315 bp, synthetic construct
  source 1..6315: mol_type = other DNA
ttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcagaatttcagccatcgccctgatagacggtttttcgccctttgacgttggagtccacgttctttaatagtggactcttgttccaaactggaacaacactcaaccctatctcgggcaagcttgtctctcccctctgaccatggagcccacccctgctccactgctccagggacagccctatgctgcaggcagctctgcccccactcagcatcccaggggctgatttctttggttttggatccagctggatgtctgcattgccgaggccaccagggctggctcagcaactgtcggggaatcaccagggtctgagaaatcttgtgcgcatgtgaggggctgtgggagcagagaaccactgggtgggaaattctaatccccaccctgctggaaactctctgggtggccccaacatgctaatcctccggtaaacctctgtttcctcctcaaaaggcaggaggtcggaaagaataaacaatgagagtcacattaaaaacacaaaatcctacggaaatactgaagaatgagtctcagcactaaggaaaagcctccagcagctcctgctttctgagggtgaaggatagacgctgtggctctgcatgactcactagcactctatcacggccatattctgacagggtcagtggctccaactaacatttgtttggtactttacagtttattaaatagatgtttatatggagaagctctcatttctttctcagaagagcctggctaggaaggtggatgaggcaccatattcattttgcaggtgaaattcctgagatgtaaggagctgctgtgacttgctcaaggccttatatcgagtaaacggtagcgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgctttgtgatgggaattctgtggacctgcagggcccactagtgcggttaccagcggaaatgcctcggggtcagaagtcgcaggagagatagacagctgctgaaccaatgggaccagcggatggggcggatgttatctaccattggtgaacgttagaaacgaatagcagccaatgaatcagctgggggggcggagcagtgacgtttattgcggagggggccgcttcgaatcggcggcggccagcttggtggcctgggccaatgaacggcctccaacgagcagggccttcaccaatcggcggcctccacgacggggctgggggagggtatataagccgagtaggcgacggtgaggtcgacgccggccaagacagcacagacagattgacctattggggtgtttcgcgagtgtgagagggaagcgccgcggcctgtatttctagacctgcccttcgcctggttcgtggcgccttgtgaccccgggcccctgccgcctgcaagtcggaaattgcgctgtgctcctgtgctacggcctgtggctggactgcctgctgctgcccaactggctgccaccatggtctccaaaggagaagaactcttcaccggagtcgtaccaatcctcgtcgaacttgatggcgacgtcaatggtcataaatttttcgtgtctggtgagggagagggggacgcgacttatggaaagctcacgctcaagtttatctgcaccactggaaaactgccggtcccgtggccaactcttgtgaccacgtttacatacggagtccagtgcttcgccagatatcccgaccatatgaagcaacatgacttctttaagagtgccatgccggaagggtacgtgcaagagagaactatctttttcaaggatgacggcaactacaaaactcgagctgaggtcaagtttgagggtgataccctcgtcaatagaattgaattgaagggcatcgactttaaggaggacggcaacatcctgggccacaagctggaatataattacaacagccacaaagtctacattacggcagacaagcagaagaacggcataaaagtcaactttaaaacgaggcacaacatagaagatggctctgtccagttggctgaccactatcagcagaatacgcctataggggatgggccggttctgctccccgacaatcattacctcagtacccaatctgctttgtcaaaggaccccaatgagaaaagagaccatatggtcctgctcgagtttgtcacagccgccggtataacacttggtatggatgagctgtataagtaatgatagaagcttgatctttttccctctgccaaaaattatggggacatcatgaagccccttgagcatctgacttctggctaataaaggaaatttattttcattgcaatagtgtgttggaattttttgtgtctctcactcgactcgaaaagatacgcagcgctggatgagatgagaaatattccgcgacgcaagcaggaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagccaccttcaggacaaaagtcagggaagggctctctgaagaaatgctacttgaagataccagccctaccaagggcagggagaggaccctatagaggcctgggacaggagctcaatgagaaaggagaagagcagcaggcatgagttgaatgaaggaggcagggccgggtcacagggccttctaggccatgagagggtagacagtattctaagtacgccagaaagctgttgatcggcttcaagcagggaagggacacctaatttgcttttcttttctttttttttttttttttttttttgagatggagttttgctcttgttgcccaggctggagtgcaatggtgcatcttggctcactgcaaccatttgaagcagtcgacgccgaagagggtccgaggtattccttcggcgtcgactgcttcagaccgggagcgccctgtagcggcgaattaagcgcggcgggtgtggtggttacgcgcagcgtgaccgctacacttgccagcgccctagcgcccgctcccgggatcggaattccggccatcgccctgatagacggtttttcgccctttgacgttggagtccacgttctttaatagtggactcttgttccaaactggaaaacgtctagcggtatcagctcactcaaaggcggtaatacggttatccacataatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttgatagctcttgatccggcaaacaaaccaccgttgatagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacag

SEQ ID NO: 4 - DNA, 6254 bp, synthetic construct
  source 1..6254: mol_type = other DNA
ttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcagaatttcagccatcgccctgatagacggtttttcgccctttgacgttggagtccacgttctttaatagtggactcttgttccaaactggaacaacactcaaccctatctcgggcaagcttgtctctcccctctgaccatggagcccacccctgctccactgctccagggacagccctatgctgcaggcagctctgcccccactcagcatcccaggggctgatttctttggttttggatccagctggatgtctgcattgccgaggccaccagggctggctcagcaactgtcggggaatcaccagggtctgagaaatcttgtgcgcatgtgaggggctgtgggagcagagaaccactgggtgggaaattctaatccccaccctgctggaaactctctgggtggccccaacatgctaatcctccggtaaacctctgtttcctcctcaaaaggcaggaggtcggaaagaataaacaatgagagtcacattaaaaacacaaaatcctacggaaatactgaagaatgagtctcagcactaaggaaaagcctccagcagctcctgctttctgagggtgaaggatagacgctgtggctctgcatgactcactagcactctatcacggccatattctgacagggtcagtggctccaactaacatttgtttggtactttacagtttattaaatagatgtttatatggagaagctctcatttctttctcagaagagcctggctaggaaggtggatgaggcaccatattcattttgcaggtgaaattcctgagatgtaaggagctgctgtgacttgctcaaggccttatatcgagtaaacggtagcgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgctttgtgatgggaattctgtggacctgcagggcccactagtgcggttaccagcggaaatgcctcggggtcagaagtcgcaggagagatagacagctgctgaaccaatgggaccagcggatggggcggatgttatctaccattggtgaacgttagaaacgaatagcagccaatgaatcagctgggggggcggagcagtgacgtttattgcggagggggccgcttcgaatcggcggcggccagcttggtggcctgggccaatgaacggcctccaacgagcagggccttcaccaatcggcggcctccacgacggggctgggggagggtatataagccgagtaggcgacggtgaggtcgacgccggccaagacagcacagacagattgacctattggggtgtttcgcgagtgtgagagggaagcgccgcggcctgtatttctagacctgcccttcgcctggttcgtggcgccttgtgaccccgggcccctgccgcctgcaagtcggaaattgcgctgtgctcctgtgctacggcctgtggctggactgcctgctgctgcccaactggctgccaccatggtctccaaaggagaagaactcttcaccggagtcgtaccaatcctcgtcgaacttgatggcgacgtcaatggtcataaatttttcgtgtctggtgagggagagggggacgcgacttatggaaagctcacgctcaagtttatctgcaccactggaaaactgccggtcccgtggccaactcttgtgaccacgtttacatacggagtccagtgcttcgccagatatcccgaccatatgaagcaacatgacttctttaagagtgccatgccggaagggtacgtgcaagagagaactatctttttcaaggatgacggcaactacaaaactcgagctgaggtcaagtttgagggtgataccctcgtcaatagaattgaattgaagggcatcgactttaaggaggacggcaacatcctgggccacaagctggaatataattacaacagccacaaagtctacattacggcagacaagcagaagaacggcataaaagtcaactttaaaacgaggcacaacatagaagatggctctgtccagttggctgaccactatcagcagaatacgcctataggggatgggccggttctgctccccgacaatcattacctcagtacccaatctgctttgtcaaaggaccccaatgagaaaagagaccatatggtcctgctcgagtttgtcacagccgccggtataacacttggtatggatgagctgtataagtaatgatagaagcttgatctttttccctctgccaaaaattatggggacatcatgaagccccttgagcatctgacttctggctaataaaggaaatttattttcattgcaatagtgtgttggaattttttgtgtctctcactcgactcgaaaagatacgcagcgctggatgagatgagaaatattccgcgacgcaagcaggaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagccaccttcaggacaaaagtcagggaagggctctctgaagaaatgctacttgaagataccagccctaccaagggcagggagaggaccctatagaggcctgggacaggagctcaatgagaaaggagaagagcagcaggcatgagttgaatgaaggaggcagggccgggtcacagggccttctaggccatgagagggtagacagtattctaagtacgccagaaagctgttgatcggcttcaagcagggaagggacacctaatttgcttttcttttcttttttttttttttttttttttgagatggagttttgctcttgttgcccaggctggagtgcaatggtgcatcttggctcactgcaacgaccgggagcgccctgtagcggcgaattaagcgcggcgggtgtggtggttacgcgcagcgtgaccgctacacttgccagcgccctagcgcccgctcccgggatcggaattccggccatcgccctgatagacggtttttcgccctttgacgttggagtccacgttctttaatagtggactcttgttccaaactggaaaacgtctagcggtatcagctcactcaaaggcggtaatacggttatccacataatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttgatagctcttgatccggcaaacaaaccaccgttgatagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacag

SEQ ID NO: 5 - DNA, 6361 bp, synthetic construct
  source 1..6361: mol_type = other DNA
aaaaaaaaaaaaaaagaaaagaaaagcaaattaggtgtcccttccctgcttgaagccgatcaacagctttctggcgtacttagaatactgtctaccctctcatggcctagaaggccctgtgacccggccctgcctccttcattcaactcatgcctgctgctcttctcctttctcattgagctcctgtcccaggcctctatagggtcctctccctgcccttggtagggctggtatcttcaagtagcatttcttcagagagcccttccctgacttttgtcctgaaggtggctgttttctcaaccctgagttaaaacacattccaatctgaagttatttggacttttcccagctgacagatgggctcccccaactagaatggtgcttcctctgggcacacccctcatctgactttttaattcctccacttcaacacctggtgcattcatgtgccggcacaatcagtgattggtgggttaatgagtgactgcgtgagactgacttagtgagctgggaaagattttttggcagacagggagaaataaggagaggcaacttggagaaggggcttagaatgaggcctagaagagcagtaaggggcaaacagtctgagcaaaggcaggcaggcaggaactcagttggagagactgaggctgggccacgtgccctctcctgccaccttctcttcatctgcttttttcccgtgtcattctctggactgccagaacaaggctcactgtttcttagtaaaaagagggttttggtggcaatggataaggccgagaccaccaatcagaggagttttagacatcattgaccagagctctgggcagaacctggccattcctgaagcaaggaaacagcctgcgaaggcaccaaagctgcccttacctgggctggggaagaaggtgtcttctggaataatgctgttgttgaaggcgtttgcacatgcaaagtcagatttgttgctccaggccacagcactgttgctcttgaagtccatagacctcatgtctagcacagttttgtctgtgatatacacatcagaatccttactttgtgacacatttgtttgagaatcaaaatcggtgaataggcagacagacttgtcactggatttagagtctctcagctggtacacggcagggtcagggttctggatattcctgcttgcgtcgcggaatatttctcatctcatccagcgctgcgtatcttttcgagtcgagtgagagacacaaaaaattccaacacactattgcaatgaaaataaatttcctttattagccagaagtcagatgctcaaggggcttcatgatgtccccataatttttggcagagggaaaaagatcaagcttctatcattacttatacagctcatccataccaagtgttataccggcggctgtgacaaactcgagcaggaccatatggtctcttttctcattggggtcctttgacaaagcagattgggtactgaggtaatgattgtcggggagcagaaccggcccatcccctataggcgtattctgctgatagtggtcagccaactggacagagccatcttctatgttgtgcctcgttttaaagttgacttttatgccgttcttctgcttgtctgccgtaatgtagactttgtggctgttgtaattatattccagcttgtggcccaggatgttgccgtcctccttaaagtcgatgcccttcaattcaattctattgacgagggtatcaccctcaaacttgacctcagctcgagttttgtagttgccgtcatccttgaaaaagatagttctctcttgcacgtacccttccggcatggcactcttaaagaagtcatgttgcttcatatggtcgggatatctggcgaagcactggactccgtatgtaaacgtggtcacaagagttggccacgggaccggcagttttccagtggtgcagataaacttgagcgtgagctttccataagtcgcgtccccctctccctcaccagacacgaaaaatttatgaccattgacgtcgccatcaagttcgacgaggattggtacgactccggtgaagagttcttctcctttggagaccatggtggcagccagttgggcagcagcaggcagtccagccacaggccgtagcacaggagcacagcgcaatttccgacttgcaggcggcaggggcccggggtcacaaggcgccacgaaccaggcgaagggcaggtctagaaatacaggccgcggcgcttccctctcacactcgcgaaacaccccaataggtcaatctgtctgtgctgtcttggccggcgtcgacctcaccgtcgcctactcggcttatataccctcccccagccccgtcgtggaggccgccgattggtgaaggccctgctcgttggaggccgttcattggcccaggccaccaagctggccgccgccgattcgaagcggccccctccgcaataaacgtcactgctccgcccccccagctgattcattggctgctattcgtttctaacgttcaccaatggtagataacatccgccccatccgctggtcccattggttcagcagctgtctatctctcctgcgacttctgaccccgaggcatttccgctggtaaccgcactagtgggccctgcaggtccacagaattcccatcacaaagcagggttaggacatgatctcatttccctctttgccccaacccaggctggagtccagatgccagtgatggacaagggcggggctctgtggggctggcaagtcacggtctcatgctttatacgggaaatagcatcttagaaaccagctgctcgtgatggactgggactcagggacaggcacaagctatcaatcttggccaagaggccatgatttcagtgaacgttcacggccaggcctggcctgccactcaaggaaacctgaaatgcagggctacttaataatactgcttattcttttatttaataggatcttcttcaaaaccccagcaatataactctggcagagtaaaggcaggcatgggaaaaaggcccagcaaagcaaactgtacatcttggaatctggagtggtctccccaacttaggctgggcattagcagaatgggaggtttatggtatgttggcattaagttgggaaatctatcacattaccaggagattgctctctcattgatagaggttttgaactataaatcagaacacctgcgtctaagccccagcgctaccgtttactcgatataaggccttgagcaagtcacagcagctccttacatctcaggaatttcacctgcaaaatgaatatggtgcctcatccaccttcctagccaggctcttctgagaaagaaatgagagcttctccatataaacatctatttaataaactgtaaagtaccaaacaaatgttagttggagccactgaccctgtcagaatatggccgtgatagagtgctagtgagtcatgcagagccacagcgtctatccttcaccctcagaaagcaggagctgctggaggcttttccttagtgctgagactcattcttcagtatttccgtaggattttgtgtttttaatgtgactctcattgtttattctttccgacctcctgccttttgaggaggaaacagaggtttaccggaggattagcatgttggggccacccagagagtttccagcagggtggggattagaatttcccacccagtggttctctgctcccacagcccctcacatgcgcacaagatttctcagaccctggtgattccccgacagttgctgagccagccctggtggcctcggcaatgcagacatccagctggatccaaaaccaaagaaatcagcccctgggatgctgagtgggggcagagctgcctgcagcatagggctgtccctggagcagtggagcaggggtgggctccatggtcagaggggagacccaacagatatccagaaccgactgtgacaagcttgcccgagatagggttgagtgttgttccagtttggaacaagagtccactattaaagaacgtggactccaacgtcaaagggcgaaaaaccgtctatcagggcgatggctgaaattctgcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctagacgttttccagtttggaacaagagtccactattaaagaacgtggactccaacgtcaaagggcgaaaaaccgtctatcagggcgatggccggaattccgatcccgggagcgggcgctagggcgctggcaagtgtagcggtcacgctgcgcgtaaccaccacacccgccgcgcttaatgcgccgctacagggcgctcccggtctgaagcagtcgacgccgaaggaatacctcggaccctcttcggcgtcgactgcttcaaatgggttctggatatctgttggggttgcagtgagccaagatgcaccattgcactccagcctgggcaacaagagcaaaactccatctcaaaaaaaa

SEQ ID NO: 6 - DNA, 10113 bp, synthetic construct
  source 1..10113: mol_type = other DNA
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatatacatatgcatcaccaccaccaccatgcttcacccccaaagaagaaaagaaaggtgggttcgatggataagaaatacagcatcggcctggacattggtacgaatagcgttggttgggctgtgatcaccgatgactacaaggtgccgagtaagaaattcaaagtgctgggcaacactgaccgtcattccattaaaaagaatctgatcggcgcgctgctgtttggtagtggcgaaacggctgaggcgacgcgtctgaagcgtacagcgcgtcggcgctacacccgtcgtaagaaccgtatctgttatctgcaagagatcttttccaacgaaatggcgaaagttgacgactccttcttccatcgtctcgaggagagcttcttggttgaggaagataaaaagcacgagcgtcacccgatttttggtaacatcgttgatgaagtggcctatcacgaaaaatacccgacgatctatcatttgcgcaaaaagctggccgacagcaccgacaaagcagatctgcgcttgatctacttggccctggcgcacatgattaaatttcgtggccattttctgattgagggtgatctgaacccggataacagcgatgttgacaagttgttcattcagctggtacagatttataaccagctatttgaggagaacccgatcaatgcatcgcgggtggacgctaaagctatcctgtccgcgcgtctgtccaagtctcgtcgcctggaaaatctgatcgcgcaactgccgggtgaaaagcgcaatggcttattcggtaaccttatcgcgctgtcgctaggcctgaccccgaatttcaagagcaacttcgacttagcggaagacgcaaagctgcagctgagcaaggacacctatgacgatgacctggataacctgctagcgcagatcggcgaccagtatgcggatctgttcctggctgcgaagaacttgtctgacgccatattattatccgacatcctgcgagttaatagcgagattaccaaagcgccactgtcggcgtctatgatcaagcgctatgacgaacatcaccaagatttgaccttgctgaaagcgctggttcgacagcagctgccggaaaagtacaaggagatcttcttcgaccagagcaaaaacggttacgccggttacattgacggtggtgctagccaggaggagttctataagttcatcaagccgatcctggagaaaatggatggtaccgaagaactgttagtcaaactgaaccgtgaagacctgctgagaaagcagcgtacctttgacaatggctccatcccgcatcaaatccatctgggtgagttacacgcaatcctgcgtcgccaagaggacttctacccgtttctgaaagacaaccgtgaaaagatcgaaaaaatcttgacctttcgtatcccgtattatgttggtccgctggcgcgtggaaactcacgcttcgcatggatgacccgtaagagcgaagaaaccatcacgccgtggaattttgaagaagttgtggacaagggcgcctcggcgcagtcttttatcgaacgcatgaccaattttgataagaacctgccgaacgagaaagttctaccgaagcacagcctgctgtacgagtatttcaccgtttataatgaactgactaaagtcaaatacgtgaccgaaggtatgcgtaagccggcgtttctcagcggtgagcagaaaaaggccatcgtggacctgctgtttaagacgaatcgtaaggtgaccgttaaacaattgaaggaggactacttcaaaaagattgagtgctttgacagcgtcgagattagcggcgttgaagatcgctttaatgcaagcctgggggcttaccatgatctgttgaagatcatcaaggacaaagactttctcgacaacgaagaaaatgaggatattttggaagatatcgttctgactctgactctgttcgaagacaggggtatgatcgaagagagattgaagacgtacgcgcacctgttcgacgacaaggtcatgaaacaactgaagcgccgtcgttacaccggttggggccgcctgtctcgtaaactgattaatggtattcgtgataagcagagtggtaaaaccatattagattttctgaaaagcgatggtttcgcgaacgcgaacttcatgcagttgattcacgatgattccctgaccttcaaagaagacatccagaaggcccaagttagcggccagggtcacagcctgcatgaacagatcgccaacctggctggtagcccggcgatcaaaaagggcattttgcaaaccgtgaaaattgttgatgaattggtgaaagtgatgggtcacaaaccggagaacatagtcatcgagatggctcgtgaaaaccagacgacccagaaaggccaaaaaaactctcgtgaacgtatgaaacgtattgaagaaggcattaaggagctcggttcgcaaattctgaaggagcaccctgttgagaatacacagctgcagaatgagaaactttatctgtactacctgcaaaacggtcgtgatatgtatgtggatcaagaactggacattaaccgcttgtctgactacgacgttgaccatattgttccgcagtctttcattaaagacgacagcattgataataaggtgctgacgcgcagtgataaaaaccgtggtaaatccgataatgttccgagcgaggaggtggtgaagaagatgaaaaactattggcgtcaattgcttaatgcgaaactgattactcagcgcaaattcgataacttgacgaaagcagaacggggcggtctgtccgaattggacaaggcgggctttatcaagaggcaactggtggagactcgtcagataacgaagcacgtggcacagatcttagattctcgtatgaataccaagtacgatgagaacgataaattgatccgcgaagttaaggttattaccttgaagtcgaagctcgtcagcgacttccgtaaggactttcaattttacaaagttcgtgagatcaacaactaccatcatgcacacgatgcgtatttgaatgcagtcgtgggcactgccttgattaaaaaatacccgaaactggaatctgagttcgtctatggggattacaaggtatatgacgtgcgtaagatgattgccaaatctgagcaggagatcggcaaagcgacggccaagtactttttctactccaacattatgaactttttcaaaacggaaattaccctggcgaatggcgagatccgtaaacgcccactgatcgagaccaatggtgagaccggcgaaatcgtgtgggacaaaggtcgtgatttcgcaaccgttcgcaaagtgctgagtatgccgcaggtgaacatcgttaaaaaaaccgaggtgcagaccggaggtttttcgaaagagagcattcttccgaaacgcaacagcgacaagctgatcgcgcgtaaaaaggactgggatccgaagaagtatggtggcttcgatagcccgaccgttgcctatagcgttctggtagttgctaaggttgagaaaggcaaaagtaaaaagctgaaatccgtgaaagaattgttgggcattaccattatggaacgtagctctttcgagaagaacccgattgatttcctggaggcaaaaggctacaaagaggtcaaaaaggatctaattattaagctgccaaagtacagcctgttcgagctcgaaaatggacgtaaacgtatgctggcgtcagcgggtgaactgcagaagggtaatgaactggcgctgccgagcaaatacgtgaattttctgtatctcgctagccattatgaaaaactaaagggttccccggaagacaacgagcagaagcagctttttgttgagcagcataaacactacctggacgagatcattgagcaaatcagcgagttctctaagcgtgtcattttagcggatgctaatcttgacaaagtactcagcgcgtataacaaacaccgggacaagccgatccgtgaacaagcggagaacattattcacttgtttaccctgacgaacctgggtgctccggcggcatttaaatacttcgataccaccattgaccgcaagagatacaccagcaccaaagaggtgctggatgctaccctgattcatcaaagcatcaccggcctgtatgaaacccgtatcgatctttcccaattaggcggcgacagcccggttcgctctagcggtggctcgagtggcgggtcgagcggttccgaaacccccggcaccagcgagtcagccaccccggaaagctctggcgggtcctccggcggctcctcgcgtccgggtgagcgccctttccaatgccgtatctgcatgcgtaacttttctaatatgagcaatctgacccgtcacacccgcacgcatacaggcgagaagccgtttcaatgtagaatttgcatgcgtaacttcagcgatcgtagcgtcctcaggcgccaccttcgcacccacaccggcagccaaaagccatttcagtgccggatctgcatgcgtaatttctccgacccgtccaacctggcgcgtcacacccgtacccataccggtgagaaaccattccagtgtcgtatttgcatgcgtaacttcagcgatcgtagcagcttgcgccgccacctgcgtacccatactggttcccagaagccgtttcagtgccgtatctgcatgaggaacttctcccagagcggtaccctgcaccgacatactagaacgcacacgggcgaaaagccgttccaatgccgcatctgtatgcgtaacttcagccaacgtccgaatcttactcgtcacctgcgcacccatctgcgcggttcgggcagcgcgggtagcgcggcaggttccggcgaattcccgaaaaagaaacgtaaagtgtaatgaaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 7 - DNA, 9902 bp, synthetic construct
  source 1..9902: mol_type = other DNA
caactttgtatagaaaagttgggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaagtttgtacaaaaaagcaggctgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgactcccccgtccggagtagcgggggatcttctggcggcagttccggttctgaaacccccgggacttctgagtccgccacccccgaatccagcggagggtcttccggagggtcttcaagaccaggtgaacgcccattccagtgtcgcatctgcatgcgcaactttagcaatatgagcaatttgaccagacatacaagaacacatacaggtgagaagcccttccagtgtcgaatatgtatgaggaatttctcagataggtccgttctgaggcggcatctcagaacacatacgggttcccagaaaccattccaatgtcggatatgtatgcggaacttcagtgacccatccaatctcgctaggcatacgcggacccacacaggtgaaaaaccgtttcaatgccgcatatgcatgcggaatttctctgaccgatcaagcctcagaagacacctccgaactcataccggcagccagaaacctttccaatgtaggatatgtatgcgaaacttctctcaatccggaacccttcatcgacataccagaacccatacaggagaaaagcctttccagtgtagaatttgcatgaggaacttttcccaacgcccaaatcttacgcggcatctcaggacacatctgagggggtctggcagcgcaggcagtgcagcggggtcaggagagttccccaaaaaaaagcgcaaagtataacgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcgccaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggcggccgcggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctctcttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcggcgcgccgcggccgc

SEQ ID NO: 8 - DNA, 9289 bp, synthetic construct
  source 1..9289: mol_type = other DNA
actcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcggcgcgccgcggccgccaactttgtatagaaaagttgggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaagtttgtacaaaaaagcaggctgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagtaaacccagctttcttgtacaaagtggtgatggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcgccaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggcggccgcggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctctcttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcat

SEQ ID NO: 9 - DNA, 2372 bp, synthetic construct
  source 1..2372: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtcagggttctggatatctgtgttttagagctagaaatagcaagttaaaataaggctagtccgttatcaacttgaaaaagtggcaccgagtcggtgcttttttgtctagaggtaccggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagatccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 10 - DNA, 6305 bp, synthetic construct
  source 1..6305: mol_type = other DNA
catgacattaacctataaaaataggcgtatcacgaggcagaatttcagccatcgccctgatagacggtttttcgccctttgacgttggagtccacgttctttaatagtggactcttgttccaaactggaacaacactcaaccctatctcgggcaagcttgtcacagtcggttctggatatctgttgggtctcccctctgaccatggagcccacccctgctccactgctccagggacagccctatgctgcaggcagctctgcccccactcagcatcccaggggctgatttctttggttttggatccagctggatgtctgcattgccgaggccaccagggctggctcagcaactgtcggggaatcaccagggtctgagaaatcttgtgcgcatgtgaggggctgtgggagcagagaaccactgggtgggaaattctaatccccaccctgctggaaactctctgggtggccccaacatgctaatcctccggtaaacctctgtttcctcctcaaaaggcaggaggtcggaaagaataaacaatgagagtcacattaaaaacacaaaatcctacggaaatactgaagaatgagtctcagcactaaggaaaagcctccagcagctcctgctttctgagggtgaaggatagacgctgtggctctgcatgactcactagcactctatcacggccatattctgacagggtcagtggctccaactaacatttgtttggtactttacagtttattaaatagatgtttatatggagaagctctcatttctttctcagaagagcctggctaggaaggtggatgaggcaccatattcattttgcaggtgaaattcctgagatgtaaggagctgctgtgacttgctcaaggccttatatcgagtaaacggtagcgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgctttgtgatgggaattctgtggacctgcagggcccactagtgcggttaccagcggaaatgcctcggggtcagaagtcgcaggagagatagacagctgctgaaccaatgggaccagcggatggggcggatgttatctaccattggtgaacgttagaaacgaatagcagccaatgaatcagctgggggggcggagcagtgacgtttattgcggagggggccgcttcgaatcggcggcggccagcttggtggcctgggccaatgaacggcctccaacgagcagggccttcaccaatcggcggcctccacgacggggctgggggagggtatataagccgagtaggcgacggtgaggtcgacgccggccaagacagcacagacagattgacctattggggtgtttcgcgagtgtgagagggaagcgccgcggcctgtatttctagacctgcccttcgcctggttcgtggcgccttgtgaccccgggcccctgccgcctgcaagtcggaaattgcgctgtgctcctgtgctacggcctgtggctggactgcctgctgctgcccaactggctgccaccatggtctccaaaggagaagaactcttcaccggagtcgtaccaatcctcgtcgaacttgatggcgacgtcaatggtcataaatttttcgtgtctggtgagggagagggggacgcgacttatggaaagctcacgctcaagtttatctgcaccactggaaaactgccggtcccgtggccaactcttgtgaccacgtttacatacggagtccagtgcttcgccagatatcccgaccatatgaagcaacatgacttctttaagagtgccatgccggaagggtacgtgcaagagagaactatctttttcaaggatgacggcaactacaaaactcgagctgaggtcaagtttgagggtgataccctcgtcaatagaattgaattgaagggcatcgactttaaggaggacggcaacatcctgggccacaagctggaatataattacaacagccacaaagtctacattacggcagacaagcagaagaacggcataaaagtcaactttaaaacgaggcacaacatagaagatggctctgtccagttggctgaccactatcagcagaatacgcctataggggatgggccggttctgctccccgacaatcattacctcagtacccaatctgctttgtcaaaggaccccaatgagaaaagagaccatatggtcctgctcgagtttgtcacagccgccggtataacacttggtatggatgagctgtataagtaatgatagaagcttgatctttttccctctgccaaaaattatggggacatcatgaagccccttgagcatctgacttctggctaataaaggaaatttattttcattgcaatagtgtgttggaattttttgtgtctctcactcgactcgaaaagatacgcagcgctggatgagatgagaaatattccgcgacgcaagcaggaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagccaccttcaggacaaaagtcagggaagggctctctgaagaaatgctacttgaagataccagccctaccaagggcagggagaggaccctatagaggcctgggacaggagctcaatgagaaaggagaagagcagcaggcatgagttgaatgaaggaggcagggccgggtcacagggccttctaggccatgagagggtagacagtattctaagtacgccagaaagctgttgatcggcttcaagcagggaagggacacctaatttgcttttcttttctttttttttttttttttttttttgagatggagttttgctcttgttgcccaggctggagtgcaatggtgcatcttggctcactgcaaccccaacagatatccagaacccattgaccgggagcgccctgtagcggcgcattaagcgcggcgggtgtggtggttacgcgcagcgtgaccgctacacttgccagcgccctagcgcccgctcccgggatcggaattccggccatcgccctgatagacggtttttcgccctttgacgttggagtccacgttctttaatagtggactcttgttccaaactggaaaacgtctagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattat

SEQ ID NO: 11 - DNA, 8357 bp, synthetic construct
  source 1..8357: mol_type = other DNA
ccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccctgcaggtctcgggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagacctctcaaaaatagctaccctctccggcatgaatttatcagctagaacggttgaatatcatattgatggtgatttgactgtctccggcctttctcacccttttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttaacgctactactattagtagaattgatgccaccttttcagctcgcgccccaaatgaaaatatagctaaacaggttattgaccatttgcgaaatgtatctaatggtcaaactaaatctactcgttcgcagaattgggaatcaactgttacatggaatgaaacttccagacaccgtactttagttgcatatttaaaacatgttgagctacagcaccagattcagcaattaagctctaagccatccgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagctttgttacgttgatttgggtaatgaatatccggttcttgtcaagattactcttgatgaaggtcagccagcctatgcgcctggtctgtacaccgttcatctgtcctctttcaaagttggtcagttcggttcccttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattctttcgcctctttcgttttaggttggtgccttcgtagtggcattacgtattttacccgtttaatggaaacttcctcatgaaaaagtctttagccctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagcctttttttttggagattttcaacgtgaaaaaattattattcgcaattcctttagttgttcctttctattctcactccgctgaaactgttgaaagttgtttagcaaaaccccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatccattcgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatcatgccagttcttttgggtattccgttattattgcgtttcctcggtttccttctggtaactttgttcggctatctgcttacttttcttaaaaagggcttcggtaagatagctattgctatttcattgtttcttgctcttattattgggcttaactcaattcttgtgggttatctctctgatattagcgctcaattaccctctgactttgttcagggtgttcagttaattctcccgtctaatgcgcttccctgtttttatgttattctctctgtaaaggctgctattttcatttttgacgttaaacaaaaaatcgtttcttatttggattgggataaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggcttgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatatgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattcactcacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttgtaacttggtattcaaagcaatcaggcgaatccgttattgtttctcccgatgtaaaaggtactgttactgtatattcatctgacgttaaacctgaaaatctacgcaatttctttatttctgttttacgtgcaaataattttgatatggtaggttctaacccttccattattcagaagtataatccaaacaatcaggattatattgatgaattgccatcatctgataatcaggaatatgatgataattccgctccttctggtggtttctttgttccgcaaaatgataatgttactcaaacttttaaaattaataacgttcgggcaaaggatttaatacgagttgtcgaattgtttgtaaagtctaatacttctaaatcctcaaatgtattatctattgacggctctaatctattagttgttagtgctcctaaagatattttagataaccttcctcaattcctttcaactgttgatttgccaactgaccagatattgattgagggtttgatatttgaggttcagcaaggtgatgctttagatttttcatttgctgctggctctcagcgtggcactgttgcaggcggtgttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctgttggccagaatgtcccttttattactggtcgtgtgactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgctacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagctcggtaccaaattccagaaaagaggccgcgaaagcggccttttttcgttttggtccacggatcgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaaggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccgattcgagctcgcccggggatcgaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaa

SEQ ID NO: 12 - DNA, 12030 bp, Homo sapiens
  source 1..12030: mol_type = genomic DNA
agtctcagctactcgggagactgaggcaggagaattgttgaacccgggaggcagagattgcagtgagctgagatcgcatcattgcactccagcctgggcaacagagcaagactccttctcaaaaaaacaaaaaaaaaaaaaaaaaagaaggtctaacccttaggagtgtgattatcctgttctcctgccttgtgggggagatcagtgtttctcttatttaagtaataggtaggtcctgtgagtttgtgcaatggtgtcacctacggtatgaatactggaggaacaattgataaactcacatttgggaaagggacccatgtattcattatatctggtgagtcatcccaggtggcaccacgtgcaaccccatgggccagtgtcactaatcctttctctggagatatcacttattactatggtgaggcttgctgtagatgttgtaactaattttcttacagaggtctgggaagggaaaagcattactatctatcttgaatattcatgtttctctaggtcaaacacattaaaatttgactttaatcattcaatgggtattgtaaaatgccttctatgtgactatcactctataaaatgttagactgagtatgaagtgtgagatagattcctgtcctgtcctcaagtggtataaaaactagacaaaggtactgaactattgtaaattaagcagccagaaaactattttagtattgaccagttgatgatgtcaatggacacaatagagattcagaggggatagggtgctggctgagaagttggcctagactgagaagtttcctgtggtacaaaggattcattgagccctgaaggatggataagatctgtatgggcagagaaaggagagaagggaagttctgggcgtagggaacgacaagaaagaaggcatgatcttgggaataatcaaggcacatgcaaagtagcctaagtatacatctgataataaaattggttgaaaagtagtcagagaagatgtctttttaggcatggaaaaaggaaatactagagcattcaacagaatacagaaattagggccagggccagccattgggaaactgagaatccgatttagagatgcagactagaagtgaaggtgagagcagccagctatggtgccgcagacctcccctctccttcctcagtgggctctgagaggggtcatcccacaccttagaggaggagaaacctaagggattctgtaatagagacacggggcatggtatgaaagtattacctcccagttgcaatttggcaaaggaaccagagtttccacttctccccgtacgtctgcccatgcccacagtttcctgatgctcactaaagcctcggtgggacccagagtgactgtcactaattctgatttctgggtccctagtgcccaaacacgggggacagatttaatggtaaggaagctttcaatcactgctgtgtccctagggatctaaagcactagagcacatgtgcttctgcagttcattttgaatttaaaggacagcttaggatctagaatagctgaatttccacctcaaaacattggttccgtcttgccaagcctaccttctgatatcatcagtgatgggatgtgtttttcttactagggtagaataggatgtctctccccaaaggactctggcagacagacccctaaacacctccaaattaaaagcggcaaagagataaggttgaactagacgtacatggggataaaaagtataaaaggtacatgggaatgaaaggataaaaaggctaaaaaaattaagtacctctaactcagcccctgttgccatttctcagagtcttgtgttctgtggcattgcgctttctagaccaacagtgtccaatagaactttctgtggcaatggaaatgtcctgtcaatctgcactgtcccatacaatagccaccagctacatgtggctattgagctcttgaaatgaagtttccatttttaattgaaaacattttatttcacattgactaatttttatttcaacagccacatgtagctagagactattataccagacagagcagcctagatcttctccagtctgacacccaccagccccaggacttgagtgagtgtttaaccaggactcaaagttgggtttctgccccacaaggccaccccctttcctctttaaagccaacctgcatctggtggcccctgatcccctgccttgaggatcggcacttccagactcctctccccctctgcagtgctgtccagtacccccactgatgactaacaatcagggggatgtgttggtagagctaatggctttctgtctgtcccttcccagcaaaggaactatgccttagggccttcacccagagtgatgtcaggctgcccaagcatgaggagggaagtaggcagaatcctctggagccaaagctctggatgtctctcccctctgaccatggagcccacccctgctccactgctccagggacagccctatgctgcaggcagctctgcccccactcagcatcccaggggctgatttctttggttttggatccagctggatgtctgcattgccgaggccaccagggctggctcagcaactgtcggggaatcaccagggtctgagaaatcttgtgcgcatgtgaggggctgtgggagcagagaaccactgggtgggaaattctaatccccaccctgctggaaactctctgggtggccccaacatgctaatcctccggcaaacctctgtttcctcctcaaaaggcaggaggtcggaaagaataaacaatgagagtcacattaaaaacacaaaatcctacggaaatactgaagaatgagtctcagcactaaggaaaagcctccagcagctcctgctttctgagggtgaaggatagacgctgtggctctgcatgactcactagcactctatcacggccatattctggcagggtcagtggctccaactaacatttgtttggtactttacagtttattaaatagatgtttatatggagaagctctcatttctttctcagaagagcctggctaggaaggtggatgaggcaccatattcattttgcaggtgaaattcctgagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaaggagaagagcagcaggcatgagttgaatgaaggaggcagggccgggtcacagggccttctaggccatgagagggtagacagtattctaaggacgccagaaagctgttgatcggcttcaagcaggggagggacacctaatttgcttttcttttttttttttttttttttttttttttttgagatggagttttgctcttgttgcccaggctggagtgcaatggtgcatcttggctcactgcaacctccgcctcccaggttcaagtgattctcctgcctcagcctcccgagtagctgagattacaggcacccgccaccatgcctggctaattttttgtatttttagtagagacagggtttcactatgttggccaggctggtctcgaactcctgacctcaggtgatccacccgcttcagcctcccaaagtgctgggattacaggcgtgagccaccacacccggcctgcttttcttaaagatcaatctgagtgctgtacggagagtgggttgtaagccaagagtagaagcagaaagggagcagttgcagcagagagatgatggaggcctgggcagggtggtggcagggaggtaaccaacaccattcaggtttcaaaggtagaaccatgcagggatgagaaagcaaagaggggatcaaggaaggcagctggattttggcctgagcagctgagtcaatgatagtgccgtttactaagaagaaaccaaggaaaaaatttggggtgcagggatcaaaactttttggaacatatgaaagtacgtgtttatactctttatggcccttgtcactatgtatgcctcgctgcctccattggactctagaatgaagccaggcaagagcagggtctatgtgtgatggcacatgtggccagggtcatgcaacatgtactttgtacaaacagtgtatattgagtaaatagaaatggtgtccaggagccgaggtatcggtcctgccagggccaggggctctccctagcaggtgctcatatgctgtaagttccctccagatctctccacaaggaggcatggaaaggctgtagttgttcacctgcccaagaactaggaggtctggggtgggagagtcagcctgctctggatgctgaaagaatgtctgtttttccttttagaaagttcctgtgatgtcaagctggtcgagaaaagctttgaaacaggtaagacaggggtctagcctgggtttgcacaggattgcggaagtgatgaacccgcaataaccctgcctggatgagggagtgggaagaaattagtagatgtgggaatgaatgatgaggaatggaaacagcggttcaagacctgcccagagctgggtggggtctctcctgaatccctctcaccatctctgactttccattctaagcactttgaggatgagtttctagcttcaatagaccaaggactctctcctaggcctctgtattcctttcaacagctccactgtcaagagagccagagagagcttctgggtggcccagctgtgaaatttctgagtcccttagggatagccctaaacgaaccagatcatcctgaggacagccaagaggttttgccttctttcaagacaagcaacagtactcacataggctgtgggcaatggtcctgtctctcaagaatcccctgccactcctcacacccaccctgggcccatattcatttccatttgagttgttcttattgagtcatccttcctgtggtagcggaactcactaaggggcccatctggacccgaggtattgtgatgataaattctgagcacctaccccatccccagaagggctcagaaataaaataagagccaagtctagtcggtgtttcctgtcttgaaacacaatactgttggccctggaagaatgcacagaatctgtttgtaaggggatatgcacagaagctgcaagggacaggaggtgcaggagctgcaggcctcccccacccagcctgctctgccttggggaaaaccgtgggtgtgtcctgcaggccatgcaggcctgggacatgcaagcccataaccgctgtggcctcttggttttacagatacgaacctaaactttcaaaacctgtcagtgattgggttccgaatcctcctcctgaaagtggccgggtttaatctgctcatgacgctgcggctgtggtccagctgaggtgaggggccttgaagctgggagtggggtttagggacgcgggtctctgggtgcatcctaagctctgagagcaaacctccctgcagggtcttgcttttaagtccaaagcctgagcccaccaaactctcctacttcttcctgttacaaattcctcttgtgcaataataatggcctgaaacgctgtaaaatatcctcatttcagccgcctcagttgcacttctcccctatgaggtaggaagaacagttgtttagaaacgaagaaactgaggccccacagctaatgagtggaggaagagagacacttgtgtacaccacatgccttgtgttgtacttctctcaccgtgtaacctcctcatgtcctctctccccagtacggctctcttagctcagtagaaagaagacattacactcatattacaccccaatcctggctagagtctccgcaccctcctcccccagggtccccagtcgtcttgctgacaactgcatcctgttccatcaccatcaaaaaaaaactccaggctgggtgcgggggctcacacctgtaatcccagcactttgggaggcagaggcaggaggagcacaggagctggagaccagcctgggcaacacagggagaccccgcctctacaaaaagtgaaaaaattaaccaggtgtggtgctgcacacctgtagtcccagctacttaagaggctgagatgggaggatcgcttgagccctggaatgttgaggctacaatgagctgtgattgcgtcactgcactccagcctggaagacaaagcaagatcctgtctcaaataataaaaaaaataagaactccagggtacatttgctcctagaactctaccacatagccccaaacagagccatcaccatcacatccctaacagtcctgggtcttcctcagtgtccagcctgacttctgttcttcctcattccagatctgcaagattgtaagacagcctgtgctccctcgctccttcctctgcattgcccctcttctccctctccaaacagagggaactctcctacccccaaggaggtgaaagctgctaccacctctgtgcccccccggcaatgccaccaactggatcctacccgaatttatgattaagattgctgaagagctgccaaacactgctgccaccccctctgttcccttattgctgcttgtcactgcctgacattcacggcagaggcaaggctgctgcagcctcccctggctgtgcacattccctcctgctccccagagactgcctccgccatcccacagatgatggatcttcagtgggttctcttgggctctaggtcctgcagaatgttgtgaggggtttatttttttttaatagtgttcataaagaaatacatagtattcttcttctcaagacgtggggggaaattatctcattatcgaggccctgctatgctgtgtatctgggcgtgttgtatgtcctgctgccgatgccttcattaaaatgatttggaagagcagagactgtgcctctgtttgactgggtttggtaggagtcattttctgcttgctggtgatcactagctgggcagagaaaaaccaaggcatttgtctatgatgctgtccaggaagcctcattcaacaagctgcctaagtcaacctcttcttggaataacctctaaaagcttccgcttagcaggctatgctgagggccaggaaaacccacctaccagcttggacccctcctctcccactctcatgccacgccacgggaccacccataacaggagcccacacacatgggtggcagtgacctgcggcagacagggaccacacagcaagtgtccccaaaatgccacccactgtctcctgccctccaggagcatttcctttgcctctcctctcagactgggtttccactgaaactgtgcattgtctcacaaattcgtggctggggaccacccaccactctgctgcctgatccagccccacgccagccctttgaggtgcccaagctgacaccaggagcaaggttgagaggaagctgtgaccccagcaggactttatgttcccaccatcccggatgtgagaatgaggaaaaaaggagatgagctgtctccccacaagcccagagatttgaccgaggagagtagaggcctcgagctctcacctaagagaaaagacatggggcttcctggggtccacagctcactgcgctctccctcctgagactcctgctgccagagcacctttccccagggtcatggatgctgagggaaacacaacttagagaccactccaccatccacccagcaagcacagctaccaagacccaaagctgaggcttaccaatgcccagggtgggagggggttccatccctgaataactccatggttcccctatgcgtctgaccatcccagccagaaatacataaatcatctcagctacaattcaggcctgcttcttttcatagggatgaagctacaggttgagtatcccttatctgaaatgcttggtactagaagtgttcaggttttggattttttttttttttttttgaggctgggggtggaatattagcattatacttaccaattcggcatccctaatctaaaaatctgaaatccaagatgccccagtgagtgagcatttcctttgagcatcatgttggcatttaaaaagtttcagattttggagcatttccaatttcagatttttgaattagggatgcttaacctgtaccagctttaataggtgacctagagcacatccctcccctctacaggcttatgtgttgccacttacgaaatgtcgggtaggactggaggctacctcccactccctgcacctctgattctgtgacccctgcagaattaagaaccagtgtcccttctcctggtcagccctactgacgggagtcacagaatccccagttctttctaagctgccccatctctcacctaaatacaatccccttaaataacaccaaagggaaagggctcagacctccatcacagcagggtcactctcgcacgtggtaggaccataccaccttcacaaaggcggggtttgccacctttggggagctctggggggcctctacctcctcaccagtctgacacaatgccagagattccaccactgggaatttcttataattaaagcattctcttccctgtattaaatgaaaatgcccctgggagagttaatagagcaagcctttatcaaccattaaaaactgagggccagttagtttctctttcttttccccctgaagtggtacttcattttgttttatagaaaaaagattcaggcaagggaagtgtgggtggctggggaggcaggtctgctcctttgagttggctgcagtgacatggaagtcacagggctgagggaaggagacaagagcctggacagcagtgaaggggtcaaagacagacccctccaagaacctcagaggagacccggactgcaggagacctgcaggaggcccgtgggagcctgtggaggcctgtggaggcccgcgggagcctgtggaggcctgtggaggcccgcgggagcctgtggaggcctgtggaggcctgtggaggtctgcgggagcctgtggaggcctgtggaggcctgcgggagcctgtggaggcctgtggaggcctgtggaggtctgcgggagcctgtggaggtctgcgggagcctgtggaggcctgtggaggcctgtggaggcctgcgggagcctgtggaggtctgcgggagcctgtggaggcctgtggaggcctgtggaggtctgcgggagcctgtggaggcctgtggaggcctgtggaggtctgcgggagcctgtggaggcctgtggaggcctgccagcccagtgccctcaggcagcaaagcccagcagtcactggtcccccaaggccgcccacagatctatgcaaggtgatgcagcaggggctatggacccacccacacccaagcagggaggcacgatgacaaggcccgggcaggtagggggacatccggaaagcaggctcagctccacccctgagaaatttccgtctaaatccaggagatccttgattcagcaagaaccttccctcatgtccgaggctgttaaacctaaagccagttagggcagaggtcagaggggtggaccaggagcaagtgggtcgagggtgagcaagtgttgggagtggggatagaaactccatccatctccgactgctctgctcgcatatctggctccagggcccacctggtatgtcagagggaattagaacggccttgtgaggaggctcaggagaagggctcctgtgcccaccggccaagtcagcactgggcctaacgccaccacagcaaagcccctcagtgcagagggcccagctatacggaactgggcattgcttagcctggagggaataggcagctgaggagacctggaaaacacagtcatgaagagactggccatgaagacccacagaccctgcgagccaaggccttgggctgcagctgcagctgcagctctaccagcctggacccaacctcaggccgacatcccagcagggaacatgttactaaggccagttgtagaagggacttctctggacacatgccctactctcccaaaagacttaagcacaggccagtcccaaaggcagaagaatggactaaatgagctcttgtggccccagccctaagattctagaggactctctgaaacttcacttgaagacttgacctctgagtgcaactggagtcacagccactcaggagaaagggcaggctatgcaagcacgtgcttcaggagcatcctggtgaggtcttcagctctgacagagcaacccctctgctgctaccctcactgggggcagccatctgagtcttaaggagttgcttattttttaggtccaaacataaaattcaactcccctgggatcacgtatacagacaggcctatagcaccttattatcaggtaccatcctagtgtggaagtcccctggcctgtgtgctctgtgggatgggaatgtcccttgtcatcaatgtgtccccaggaactagaacagtgcctggcacacaacagatgccgttaaatgaataagtgaatgactcctgcagttaggctctgggtggggggagctgagtgtgaggagtggggagtacaggagaaaggaaagtggggattagtagaggaagtggtcaggtgagtca

SEQ ID NO: 13 - DNA, 780 bp, synthetic construct
  source 1..780: mol_type = other DNA
atcaccggcctgtacgagacacggatcgacctgtctcagctgggaggcgactcccccgtccggagtagcgggggatcttctggcggcagttccggttctgaaacccccgggacttctgagtccgccacccccgaatccagcggagggtcttccggagggtcttcaagaccaggtgaacgcccattccagtgtcgcatctgcatgcgcaactttagcaatatgagcaatttgaccagacatacaagaacacatacaggtgagaagcccttccagtgtcgaatatgtatgaggaatttctcagataggtccgttctgaggcggcatctcagaacacatacgggttcccagaaaccattccaatgtcggatatgtatgcggaacttcagtgacccatccaatctcgctaggcatacgcggacccacacaggtgaaaaaccgtttcaatgccgcatatgcatgcggaatttctctgaccgatcaagcctcagaagacacctccgaactcataccggcagccagaaacctttccaatgtaggatatgtatgcgaaacttctctcaatccggaacccttcatcgacataccagaacccatacaggagaaaagcctttccagtgtagaatttgcatgaggaacttttcccaacgcccaaatcttacgcggcatctcaggacacatctgagggggtctggcagcgcaggcagtgcagcggggtcaggagagttccccaaaaaaaagcgcaaagtataacgcttcgagcagacatgataagatacattg

SEQ ID NO: 14 - protein, 17 aa, synthetic construct
ITGLYETRIDLSQLGGD

SEQ ID NO: 15 - DNA, 4101 bp, synthetic construct
  source 1..4101: mol_type = other DNA
atggataagaaatacagcatcggcctggacattggtacgaatagcgttggttgggctgtgatcaccgatgactacaaggtgccgagtaagaaattcaaagtgctgggcaacactgaccgtcattccattaaaaagaatctgatcggcgcgctgctgtttggtagtggcgaaacggctgaggcgacgcgtctgaagcgtacagcgcgtcggcgctacacccgtcgtaagaaccgtatctgttatctgcaagagatcttttccaacgaaatggcgaaagttgacgactccttcttccatcgtctcgaggagagcttcttggttgaggaagataaaaagcacgagcgtcacccgatttttggtaacatcgttgatgaagtggcctatcacgaaaaatacccgacgatctatcatttgcgcaaaaagctggccgacagcaccgacaaagcagatctgcgcttgatctacttggccctggcgcacatgattaaatttcgtggccattttctgattgagggtgatctgaacccggataacagcgatgttgacaagttgttcattcagctggtacagatttataaccagctatttgaggagaacccgatcaatgcatcgcgggtggacgctaaagctatcctgtccgcgcgtctgtccaagtctcgtcgcctggaaaatctgatcgcgcaactgccgggtgaaaagcgcaatggcttattcggtaaccttatcgcgctgtcgctaggcctgaccccgaatttcaagagcaacttcgacttagcggaagacgcaaagctgcagctgagcaaggacacctatgacgatgacctggataacctgctagcgcagatcggcgaccagtatgcggatctgttcctggctgcgaagaacttgtctgacgccatattattatccgacatcctgcgagttaatagcgagattaccaaagcgccactgtcggcgtctatgatcaagcgctatgacgaacatcaccaagatttgaccttgctgaaagcgctggttcgacagcagctgccggaaaagtacaaggagatcttcttcgaccagagcaaaaacggttacgccggttacattgacggtggtgctagccaggaggagttctataagttcatcaagccgatcctggagaaaatggatggtaccgaagaactgttagtcaaactgaaccgtgaagacctgctgagaaagcagcgtacctttgacaatggctccatcccgcatcaaatccatctgggtgagttacacgcaatcctgcgtcgccaagaggacttctacccgtttctgaaagacaaccgtgaaaagatcgaaaaaatcttgacctttcgtatcccgtattatgttggtccgctggcgcgtggaaactcacgcttcgcatggatgacccgtaagagcgaagaaaccatcacgccgtggaattttgaagaagttgtggacaagggcgcctcggcgcagtcttttatcgaacgcatgaccaattttgataagaacctgccgaacgagaaagttctaccgaagcacagcctgctgtacgagtatttcaccgtttataatgaactgactaaagtcaaatacgtgaccgaaggtatgcgtaagccggcgtttctcagcggtgagcagaaaaaggccatcgtggacctgctgtttaagacgaatcgtaaggtgaccgttaaacaattgaaggaggactacttcaaaaagattgagtgctttgacagcgtcgagattagcggcgttgaagatcgctttaatgcaagcctgggggcttaccatgatctgttgaagatcatcaaggacaaagactttctcgacaacgaagaaaatgaggatattttggaagatatcgttctgactctgactctgttcgaagacaggggtatgatcgaagagagattgaagacgtacgcgcacctgttcgacgacaaggtcatgaaacaactgaagcgccgtcgttacaccggttggggccgcctgtctcgtaaactgattaatggtattcgtgataagcagagtggtaaaaccatattagattttctgaaaagcgatggtttcgcgaacgcgaacttcatgcagttgattcacgatgattccctgaccttcaaagaagacatccagaaggcccaagttagcggccagggtcacagcctgcatgaacagatcgccaacctggctggtagcccggcgatcaaaaagggcattttgcaaaccgtgaaaattgttgatgaattggtgaaagtgatgggtcacaaaccggagaacatagtcatcgagatggctcgtgaaaaccagacgacccagaaaggccaaaaaaactctcgtgaacgtatgaaacgtattgaagaaggcattaaggagctcggttcgcaaattctgaaggagcaccctgttgagaatacacagctgcagaatgagaaactttatctgtactacctgcaaaacggtcgtgatatgtatgtggatcaagaactggacattaaccgcttgtctgactacgacgttgaccatattgttccgcagtctttcattaaagacgacagcattgataataaggtgctgacgcgcagtgataaaaaccgtggtaaatccgataatgttccgagcgaggaggtggtgaagaagatgaaaaactattggcgtcaattgcttaatgcgaaactgattactcagcgcaaattcgataacttgacgaaagcagaacggggcggtctgtccgaattggacaaggcgggctttatcaagaggcaactggtggagactcgtcagataacgaagcacgtggcacagatcttagattctcgtatgaataccaagtacgatgagaacgataaattgatccgcgaagttaaggttattaccttgaagtcgaagctcgtcagcgacttccgtaaggactttcaattttacaaagttcgtgagatcaacaactaccatcatgcacacgatgcgtatttgaatgcagtcgtgggcactgccttgattaaaaaatacccgaaactggaatctgagttcgtctatggggattacaaggtatatgacgtgcgtaagatgattgccaaatctgagcaggagatcggcaaagcgacggccaagtactttttctactccaacattatgaactttttcaaaacggaaattaccctggcgaatggcgagatccgtaaacgcccactgatcgagaccaatggtgagaccggcgaaatcgtgtgggacaaaggtcgtgatttcgcaaccgttcgcaaagtgctgagtatgccgcaggtgaacatcgttaaaaaaaccgaggtgcagaccggaggtttttcgaaagagagcattcttccgaaacgcaacagcgacaagctgatcgcgcgtaaaaaggactgggatccgaagaagtatggtggcttcgatagcccgaccgttgcctatagcgttctggtagttgctaaggttgagaaaggcaaaagtaaaaagctgaaatccgtgaaagaattgttgggcattaccattatggaacgtagctctttcgagaagaacccgattgatttcctggaggcaaaaggctacaaagaggtcaaaaaggatctaattattaagctgccaaagtacagcctgttcgagctcgaaaatggacgtaaacgtatgctggcgtcagcgggtgaactgcagaagggtaatgaactggcgctgccgagcaaatacgtgaattttctgtatctcgctagccattatgaaaaactaaagggttccccggaagacaacgagcagaagcagctttttgttgagcagcataaacactacctggacgagatcattgagcaaatcagcgagttctctaagcgtgtcattttagcggatgctaatcttgacaaagtactcagcgcgtataacaaacaccgggacaagccgatccgtgaacaagcggagaacattattcacttgtttaccctgacgaacctgggtgctccggcggcatttaaatacttcgataccaccattgaccgcaagagatacaccagcaccaaagaggtgctggatgctaccctgattcatcaaagcatcaccggcctgtatgaaacccgtatcgatctttcccaattaggcggcgac

SEQ ID NO: 16 - protein, 1367 aa, synthetic construct
MDKKYSIGLDIGTNSVGWAVITDDYKVPSKKFKVLGNTDRHSIKKNLIGALLFGSGETAEATRLKRTARRRYTRRKNRICYLQEIFSNEMAKVDDSFFHRLEESFLVEEDKKHERHPIFGNIVDEVAYHEKYPTIYHLRKKLADSTDKADLRLIYLALAHMIKFRGHFLIEGDLNPDNSDVDKLFIQLVQIYNQLFEENPINASRVDAKAILSARLSKSRRLENLIAQLPGEKRNGLFGNLIALSLGLTPNFKSNFDLAEDAKLQLSKDTYDDDLDNLLAQIGDQYADLFLAAKNLSDAILLSDILRVNSEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGAYHDLLKIIKDKDFLDNEENEDILEDIVLTLTLFEDRGMIEERLKTYAHLFDDKVMKQLKRRRYTGWGRLSRKLINGIRDKQSGKTILDFLKSDGFANANFMQLIHDDSLTFKEDIQKAQVSGQGHSLHEQIANLAGSPAIKKGILQTVKIVDELVKVMGHKPENIVIEMARENQTTQKGQKNSRERMKRIEEGIKELGSQILKEHPVENTQLQNEKLYLYYLQNGRDMYVDQELDINRLSDYDVDHIVPQSFIKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 17 - DNA, 4101 bp, unidentified
  source 1..4101: mol_type = unassigned DNA
gacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgac

SEQ ID NO: 18 - protein, 1367 aa, unidentified
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 19 - skipped

SEQ ID NO: 20 - protein, 32 aa, synthetic construct
SGGSSGGSSGSETPGTSESATPESSGGSSGGS

SEQ ID NO: 21 - protein, 12 aa, synthetic construct
GSAGSAAGSGEF

SEQ ID NO: 22 - protein, 176 aa, synthetic construct
SRPGERPFQCRICMRNFSNMSNLTRHTRTHTGEKPFQCRICMRNFSDRSVLRRHLRTHTGSQKPFQCRICMRNFSDPSNLARHTRTHTGEKPFQCRICMRNFSDRSSLRRHLRTHTGSQKPFQCRICMRNFSQSGTLHRHTRTHTGEKPFQCRICMRNFSQRPNLTRHLRTHLRGS

SEQ ID NO: 23 - DNA, 56 bp, synthetic construct
  source 1..56: mol_type = other DNA
tgaagcagtcgacgccgaagagggtccgaggtattccttcggcgtcgactgcttca

SEQ ID NO: 24 - DNA, 16 bp, synthetic construct
  source 1..16: mol_type = other DNA
agggtccgaggtattc

SEQ ID NO: 25 - DNA, 56 bp, synthetic construct
  source 1..56: mol_type = other DNA
tgaagcagtcgacgccgaaggaatacctcggaccctcttcggcgtcgactgcttca

SEQ ID NO: 26 - DNA, 16 bp, synthetic construct
  source 1..16: mol_type = other DNA
gaatacctcggaccct

SEQ ID NO: 27 - DNA, 1267 bp, synthetic construct
  source 1..1267: mol_type = other DNA
tctcccctctgaccatggagcccacccctgctccactgctccagggacagccctatgctgcaggcagctctgcccccactcagcatcccaggggctgatttctttggttttggatccagctggatgtctgcattgccgaggccaccagggctggctcagcaactgtcggggaatcaccagggtctgagaaatcttgtgcgcatgtgaggggctgtgggagcagagaaccactgggtgggaaattctaatccccaccctgctggaaactctctgggtggccccaacatgctaatcctccggtaaacctctgtttcctcctcaaaaggcaggaggtcggaaagaataaacaatgagagtcacattaaaaacacaaaatcctacggaaatactgaagaatgagtctcagcactaaggaaaagcctccagcagctcctgctttctgagggtgaaggatagacgctgtggctctgcatgactcactagcactctatcacggccatattctgacagggtcagtggctccaactaacatttgtttggtactttacagtttattaaatagatgtttatatggagaagctctcatttctttctcagaagagcctggctaggaaggtggatgaggcaccatattcattttgcaggtgaaattcctgagatgtaaggagctgctgtgacttgctcaaggccttatatcgagtaaacggtagcgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctg

SEQ ID NO: 28 - DNA, 1237 bp, synthetic construct
  source 1..1237: mol_type = other DNA
aggaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagccaccttcaggacaaaagtcagggaagggctctctgaagaaatgctacttgaagataccagccctaccaagggcagggagaggaccctatagaggcctgggacaggagctcaatgagaaaggagaagagcagcaggcatgagttgaatgaaggaggcagggccgggtcacagggccttctaggccatgagagggtagacagtattctaagtacgccagaaagctgttgatcggcttcaagcagggaagggacacctaatttgcttttcttttctttttttttttttttttttttttgagatggagttttgctcttgttgcccaggctggagtgcaatggtgcatcttggctcactgcaac

SEQ ID NO: 29 - DNA, 76 bp, synthetic construct
  source 1..76: mol_type = other DNA
gttttagagctagaaatagcaagttaaaataaggctagtccgttatcaacttgaaaaagtggcaccgagtcggtgc

SEQ ID NO: 30 - DNA, 1179 bp, synthetic construct
  source 1..1179: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 31 - protein, 7 aa, Simian virus 40
PKKKRKV

SEQ ID NO: 32 - protein, 245 aa, synthetic construct
MTLTASSSSRAVTNSPVVVALDYHNRDDALAFVDKIDPRDCRLKVGKEMFTLFGPQFVRELQQRGFDIFLDLKFHDIPNTAAHAVAAAADLGVWMVNVHASGGARMMTAAREALVPFGKDAPLLIAVTVLTSMEASDLVDLGMTLSPADYAERLAALTQKCGLDGVVCSAQEAVRFKQVFGQEFKLVTPGIRPQGSEAGDQRRIMTPEQALSAGVDYMVIGRPVTQSVDPAQTLKAINASLQRSA

SEQ ID NO: 33 - protein, 236 aa, synthetic construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 34 - protein, 239 aa, synthetic construct
MVSKGEELFTGVVPILVELDGDVNGHKFFVSGEGEGDATYGKLTLKFICTTGKLPVPWPTLVTTFTYGVQCFARYPDHMKQHDFFKSAMPEGYVQERTIFFKDDGNYKTRAEVKFEGDTLVNRIELKGIDFKEDGNILGHKLEYNYNSHKVYITADKQKNGIKVNFKTRHNIEDGSVQLADHYQQNTPIGDGPVLLPDNHYLSTQSALSKDPNEKRDHMVLLEFVTAAGITLGMDELYK

SEQ ID NO: 35 - protein, 286 aa, synthetic construct
MSIQHFRVALIPFFAAFCLPVFAHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHW

SEQ ID NO: 36 - DNA, 816 bp, synthetic construct
  source 1..816: mol_type = other DNA
ttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcat

SEQ ID NO: 37 - protein, 199 aa, synthetic construct
MTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 38 - DNA, 571 bp, Homo sapiens
  source 1..571: mol_type = genomic DNA
ctttgtgatgggaattctgtggacctgcagggcccactagtgcggttaccagcggaaatgcctcggggtcagaagtcgcaggagagatagacagctgctgaaccaatgggaccagcggatggggcggatgttatctaccattggtgaacgttagaaacgaatagcagccaatgaatcagctgggggggcggagcagtgacgtttattgcggagggggccgcttcgaatcggcggcggccagcttggtggcctgggccaatgaacggcctccaacgagcagggccttcaccaatcggcggcctccacgacggggctgggggagggtatataagccgagtaggcgacggtgaggtcgacgccggccaagacagcacagacagattgacctattggggtgtttcgcgagtgtgagagggaagcgccgcggcctgtatttctagacctgcccttcgcctggttcgtggcgccttgtgaccccgggcccctgccgcctgcaagtcggaaattgcgctgtgctcctgtgctacggcctgtggctggactgcctgctgctgcccaactggctgccacc

SEQ ID NO: 39 - DNA, 56 bp, Mus sp.
  source 1..56: mol_type = genomic DNA
aataaaggaaatttattttcattgcaatagtgtgttggaattttttgtgtctctca

SEQ ID NO: 40 - protein, 6 aa, synthetic construct
HHHHHH

SEQ ID NO: 41 - DNA, 20 bp, synthetic construct
  source 1..20: mol_type = other DNA
ggttctggatatctgttggg

SEQ ID NO: 42 - DNA, 20 bp, synthetic construct
  source 1..20: mol_type = other DNA
tcagggttctggatatctgt

SEQ ID NO: 43 - protein, 22 aa, synthetic construct
DYKDHDGDYKDHDIDYKDDDDK

SEQ ID NO: 44 - DNA, 20 bp, synthetic construct
  source 1..20: mol_type = other DNA
ttgtgttctgtggcattgcg

SEQ ID NO: 45 - DNA, 20 bp, synthetic construct
  source 1..20: mol_type = other DNA
ggcagcgaggcatacatagt

SEQ ID NO: 46 - DNA, 18 bp, synthetic construct
  source 1..18: mol_type = other DNA
gaagcagtcgacgccgaa

SEQ ID NO: 47 - DNA, 20 bp, synthetic construct
  source 1..20: mol_type = other DNA
tgaagcagtcgacgccgaag

SEQ ID NO: 48 - DNA, 20 bp, synthetic construct
  source 1..20: mol_type = other DNA
cttcggcgtcgactgcttca

SEQ ID NO: 49 - DNA, 20 bp, synthetic construct
  source 1..20: mol_type = other DNA
ggggccactagggacaggat

SEQ ID NO: 50 - DNA, 4857 bp, synthetic construct
  source 1..4857: mol_type = other DNA
atgcatcaccaccaccaccatgcttcacccccaaagaagaaaagaaaggtgggttcgatggataagaaatacagcatcggcctggacattggtacgaatagcgttggttgggctgtgatcaccgatgactacaaggtgccgagtaagaaattcaaagtgctgggcaacactgaccgtcattccattaaaaagaatctgatcggcgcgctgctgtttggtagtggcgaaacggctgaggcgacgcgtctgaagcgtacagcgcgtcggcgctacacccgtcgtaagaaccgtatctgttatctgcaagagatcttttccaacgaaatggcgaaagttgacgactccttcttccatcgtctcgaggagagcttcttggttgaggaagataaaaagcacgagcgtcacccgatttttggtaacatcgttgatgaagtggcctatcacgaaaaatacccgacgatctatcatttgcgcaaaaagctggccgacagcaccgacaaagcagatctgcgcttgatctacttggccctggcgcacatgattaaatttcgtggccattttctgattgagggtgatctgaacccggataacagcgatgttgacaagttgttcattcagctggtacagatttataaccagctatttgaggagaacccgatcaatgcatcgcgggtggacgctaaagctatcctgtccgcgcgtctgtccaagtctcgtcgcctggaaaatctgatcgcgcaactgccgggtgaaaagcgcaatggcttattcggtaaccttatcgcgctgtcgctaggcctgaccccgaatttcaagagcaacttcgacttagcggaagacgcaaagctgcagctgagcaaggacacctatgacgatgacctggataacctgctagcgcagatcggcgaccagtatgcggatctgttcctggctgcgaagaacttgtctgacgccatattattatccgacatcctgcgagttaatagcgagattaccaaagcgccactgtcggcgtctatgatcaagcgctatgacgaacatcaccaagatttgaccttgctgaaagcgctggttcgacagcagctgccggaaaagtacaaggagatcttcttcgaccagagcaaaaacggttacgccggttacattgacggtggtgctagccaggaggagttctataagttcatcaagccgatcctggagaaaatggatggtaccgaagaactgttagtcaaactgaaccgtgaagacctgctgagaaagcagcgtacctttgacaatggctccatcccgcatcaaatccatctgggtgagttacacgcaatcctgcgtcgccaagaggacttctacccgtttctgaaagacaaccgtgaaaagatcgaaaaaatcttgacctttcgtatcccgtattatgttggtccgctggcgcgtggaaactcacgcttcgcatggatgacccgtaagagcgaagaaaccatcacgccgtggaattttgaagaagttgtggacaagggcgcctcggcgcagtcttttatcgaacgcatgaccaattttgataagaacctgccgaacgagaaagttctaccgaagcacagcctgctgtacgagtatttcaccgtttataatgaactgactaaagtcaaatacgtgaccgaaggtatgcgtaagccggcgtttctcagcggtgagcagaaaaaggccatcgtggacctgctgtttaagacgaatcgtaaggtgaccgttaaacaattgaaggaggactacttcaaaaagattgagtgctttgacagcgtcgagattagcggcgttgaagatcgctttaatgcaagcctgggggcttaccatgatctgttgaagatcatcaaggacaaagactttctcgacaacgaagaaaatgaggatattttggaagatatcgttctgactctgactctgttcgaagacaggggtatgatcgaagagagattgaagacgtacgcgcacctgttcgacgacaaggtcatgaaacaactgaagcgccgtcgttacaccggttggggccgcctgtctcgtaaactgattaatggtattcgtgataagcagagtggtaaaaccatattagattttctgaaaagcgatggtttcgcgaacgcgaacttcatgcagttgattcacgatgattccctgaccttcaaagaagacatccagaaggcccaagttagcggccagggtcacagcctgcatgaacagatcgccaacctggctggtagcccggcgatcaaaaagggcattttgcaaaccgtgaaaattgttgatgaattggtgaaagtgatgggtcacaaaccggagaacatagtcatcgagatggctcgtgaaaaccagacgacccagaaaggccaaaaaaactctcgtgaacgtatgaaacgtattgaagaaggcattaaggagctcggttcgcaaattctgaaggagcaccctgttgagaatacacagctgcagaatgagaaactttatctgtactacctgcaaaacggtcgtgatatgtatgtggatcaagaactggacattaaccgcttgtctgactacgacgttgaccatattgttccgcagtctttcattaaagacgacagcattgataataaggtgctgacgcgcagtgataaaaaccgtggtaaatccgataatgttccgagcgaggaggtggtgaagaagatgaaaaactattggcgtcaattgcttaatgcgaaactgattactcagcgcaaattcgataacttgacgaaagcagaacggggcggtctgtccgaattggacaaggcgggctttatcaagaggcaactggtggagactcgtcagataacgaagcacgtggcacagatcttagattctcgtatgaataccaagtacgatgagaacgataaattgatccgcgaagttaaggttattaccttgaagtcgaagctcgtcagcgacttccgtaaggactttcaattttacaaagttcgtgagatcaacaactaccatcatgcacacgatgcgtatttgaatgcagtcgtgggcactgccttgattaaaaaatacccgaaactggaatctgagttcgtctatggggattacaaggtatatgacgtgcgtaagatgattgccaaatctgagcaggagatcggcaaagcgacggccaagtactttttctactccaacattatgaactttttcaaaacggaaattaccctggcgaatggcgagatccgtaaacgcccactgatcgagaccaatggtgagaccggcgaaatcgtgtgggacaaaggtcgtgatttcgcaaccgttcgcaaagtgctgagtatgccgcaggtgaacatcgttaaaaaaaccgaggtgcagaccggaggtttttcgaaagagagcattcttccgaaacgcaacagcgacaagctgatcgcgcgtaaaaaggactgggatccgaagaagtatggtggcttcgatagcccgaccgttgcctatagcgttctggtagttgctaaggttgagaaaggcaaaagtaaaaagctgaaatccgtgaaagaattgttgggcattaccattatggaacgtagctctttcgagaagaacccgattgatttcctggaggcaaaaggctacaaagaggtcaaaaaggatctaattattaagctgccaaagtacagcctgttcgagctcgaaaatggacgtaaacgtatgctggcgtcagcgggtgaactgcagaagggtaatgaactggcgctgccgagcaaatacgtgaattttctgtatctcgctagccattatgaaaaactaaagggttccccggaagacaacgagcagaagcagctttttgttgagcagcataaacactacctggacgagatcattgagcaaatcagcgagttctctaagcgtgtcattttagcggatgctaatcttgacaaagtactcagcgcgtataacaaacaccgggacaagccgatccgtgaacaagcggagaacattattcacttgtttaccctgacgaacctgggtgctccggcggcatttaaatacttcgataccaccattgaccgcaagagatacaccagcaccaaagaggtgctggatgctaccctgattcatcaaagcatcaccggcctgtatgaaacccgtatcgatctttcccaattaggcggcgacagcccggttcgctctagcggtggctcgagtggcgggtcgagcggttccgaaacccccggcaccagcgagtcagccaccccggaaagctctggcgggtcctccggcggctcctcgcgtccgggtgagcgccctttccaatgccgtatctgcatgcgtaacttttctaatatgagcaatctgacccgtcacacccgcacgcatacaggcgagaagccgtttcaatgtagaatttgcatgcgtaacttcagcgatcgtagcgtcctcaggcgccaccttcgcacccacaccggcagccaaaagccatttcagtgccggatctgcatgcgtaatttctccgacccgtccaacctggcgcgtcacacccgtacccataccggtgagaaaccattccagtgtcgtatttgcatgcgtaacttcagcgatcgtagcagcttgcgccgccacctgcgtacccatactggttcccagaagccgtttcagtgccgtatctgcatgaggaacttctcccagagcggtaccctgcaccgacatactagaacgcacacgggcgaaaagccgttccaatgccgcatctgtatgcgtaacttcagccaacgtccgaatcttactcgtcacctgcgcacccatctgcgcggttcgggcagcgcgggtagcgcggcaggttccggcgaattcccgaaaaagaaacgtaaagtgtaa

SEQ ID NO: 51 - protein, 1618 aa, synthetic construct
MHHHHHHASPPKKKRKVGSMDKKYSIGLDIGTNSVGWAVITDDYKVPSKKFKVLGNTDRHSIKKNLIGALLFGSGETAEATRLKRTARRRYTRRKNRICYLQEIFSNEMAKVDDSFFHRLEESFLVEEDKKHERHPIFGNIVDEVAYHEKYPTIYHLRKKLADSTDKADLRLIYLALAHMIKFRGHFLIEGDLNPDNSDVDKLFIQLVQIYNQLFEENPINASRVDAKAILSARLSKSRRLENLIAQLPGEKRNGLFGNLIALSLGLTPNFKSNFDLAEDAKLQLSKDTYDDDLDNLLAQIGDQYADLFLAAKNLSDAILLSDILRVNSEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGAYHDLLKIIKDKDFLDNEENEDILEDIVLTLTLFEDRGMIEERLKTYAHLFDDKVMKQLKRRRYTGWGRLSRKLINGIRDKQSGKTILDFLKSDGFANANFMQLIHDDSLTFKEDIQKAQVSGQGHSLHEQIANLAGSPAIKKGILQTVKIVDELVKVMGHKPENIVIEMARENQTTQKGQKNSRERMKRIEEGIKELGSQILKEHPVENTQLQNEKLYLYYLQNGRDMYVDQELDINRLSDYDVDHIVPQSFIKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSPVRSSGGSSGGSSGSETPGTSESATPESSGGSSGGSSRPGERPFQCRICMRNFSNMSNLTRHTRTHTGEKPFQCRICMRNFSDRSVLRRHLRTHTGSQKPFQCRICMRNFSDPSNLARHTRTHTGEKPFQCRICMRNFSDRSSLRRHLRTHTGSQKPFQCRICMRNFSQSGTLHRHTRTHTGEKPFQCRICMRNFSQRPNLTRHLRTHLRGSGSAGSAAGSGEFPKKKRKV

SEQ ID NO: 52 - DNA, 4920 bp, synthetic construct
  source 1..4920: mol_type = other DNA
atggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgactcccccgtccggagtagcgggggatcttctggcggcagttccggttctgaaacccccgggacttctgagtccgccacccccgaatccagcggagggtcttccggagggtcttcaagaccaggtgaacgcccattccagtgtcgcatctgcatgcgcaactttagcaatatgagcaatttgaccagacatacaagaacacatacaggtgagaagcccttccagtgtcgaatatgtatgaggaatttctcagataggtccgttctgaggcggcatctcagaacacatacgggttcccagaaaccattccaatgtcggatatgtatgcggaacttcagtgacccatccaatctcgctaggcatacgcggacccacacaggtgaaaaaccgtttcaatgccgcatatgcatgcggaatttctctgaccgatcaagcctcagaagacacctccgaactcataccggcagccagaaacctttccaatgtaggatatgtatgcgaaacttctctcaatccggaacccttcatcgacataccagaacccatacaggagaaaagcctttccagtgtagaatttgcatgaggaacttttcccaacgcccaaatcttacgcggcatctcaggacacatctgagggggtctggcagcgcaggcagtgcagcggggtcaggagagttccccaaaaaaaagcgcaaagtataa

SEQ ID NO: 53 - protein, 1639 aa, synthetic construct
MDYKDHDGDYKDHDIDYKDDDDKMAPKKKRKVGIHGVPAADKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSPVRSSGGSSGGSSGSETPGTSESATPESSGGSSGGSSRPGERPFQCRICMRNFSNMSNLTRHTRTHTGEKPFQCRICMRNFSDRSVLRRHLRTHTGSQKPFQCRICMRNFSDPSNLARHTRTHTGEKPFQCRICMRNFSDRSSLRRHLRTHTGSQKPFQCRICMRNFSQSGTLHRHTRTHTGEKPFQCRICMRNFSQRPNLTRHLRTHLRGSGSAGSAAGSGEFPKKKRKV

SEQ ID NO: 54 - DNA, 100 bp, synthetic construct
  source 1..100: mol_type = other DNA
tcagggttctggatatctgtgttttagagctagaaatagcaagttaaaataaggctagtccgttatcaacttgaaaaagtggcaccgagtcggtgctttt

SEQ ID NO: 55 - DNA, 19 bp, synthetic construct
  source 1..19: mol_type = other DNA
cagggttctggatatctgt

SEQ ID NO: 56 - DNA, 20 bp, synthetic construct
  source 1..20: mol_type = other DNA
ggggccactagggacaggat

SEQ ID NO: 57 - protein, 10 aa, synthetic construct
HLRTHTGSQK

SEQ ID NO: 58 - DNA, 56 bp, synthetic construct
  source 1..56: mol_type = other DNA
gatttgcgacgttctaagcgagggtccgaggtattccgcttagaacgtcgcaaatc

SEQ ID NO: 59 - DNA, 62 bp, synthetic construct
  source 1..62: mol_type = other DNA
atttgaagcagtcgacgccgaagagggtccgaggtattccttcggcgtcgactgcttcagac